SEQUENCE LISTING (2005 sequences)

SEQ ID NO: 1 - protein, 326 aa, Homo sapiens
MRTEAQVPALQPPEPGLEGAMGHRTLVLPWVLLTLCVTAGTPEVWVQVRMEATELSSFTIRCGFLGSGSISLVTVSWGGPNGAGGTTLAVLHPERGIRQWAPARQARWETQSSISLILEGSGASSPCANTTFCCKFASFPEGSWEACGSLPPSSDPGLSAPPTPAPILRADLAGILGVSGVLLFGCVYLLHLLRRHKHRPAPRLQPSRTSPQAPRARAWAPSQASQAALHVPYATINTSCRPATLDTAHPHGGPSWWASLPTHAAHRPQGPAAWASTPIPARGSFVSVENGLYAQAGERPPHTGPGLTLFPDPRGPRAMEGPLGVR

SEQ ID NO: 2 - protein, 479 aa, Homo sapiens
MARAAALLPSRSPPTPLLWPLLLLLLLETGAQDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPRASPRDVGPLVWGAVGGTLLVLLLLAGGSLAFILLRVRRRRKSPGGAGGGASGDGGFYDPKAQVLGNGDPVFWTPVVPGPMEPDGKDEEEEEEEEKAEKGLMLPPPPALEDDMESQLDGSLISRRAVYV

SEQ ID NO: 3 - protein, 518 aa, Homo sapiens
MARAAALLPSRSPPTPLLWPLLLLLLLETGAQDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGGIIGGIIAAIIATAVAATGILICRQQRKEQTLQGAEEDEDLEGPPSYKPPTPKAKLEAQEMPSQLFTLGASEHSPLKTPYFDAGASCTEQEMPRYHELPTLEERSGPLHPGATSLGSPIPVPPGPPAVEDVSLDLEDEEGEEEEEYLDKINPIYDALSY

SEQ ID NO: 4 - protein, 120 aa, synthetic construct
QVQLVQSGAEVKKPGASVKVSCKASGYTFTDYNINWVRQAPGQGLEWMGYIYPYIGGSGYAQKFQGRVTMTRDTSTSTVYMELSSLRSEDTAVYYCAREDKTARNAMDYWGQGTLVTVSS

SEQ ID NO: 5 - protein, 8 aa, synthetic construct
GYTFTDYN

SEQ ID NO: 6 - protein, 8 aa, synthetic construct
IYPYIGGS

SEQ ID NO: 7 - protein, 13 aa, synthetic construct
AREDKTARNAMDY

SEQ ID NO: 8 - protein, 447 aa, synthetic construct
QVQLVQSGAEVKKPGASVKVSCKASGYTFTDYNINWVRQAPGQGLEWMGYIYPYIGGSGYAQKFQGRVTMTRDTSTSTVYMELSSLRSEDTAVYYCAREDKTARNAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 9 - protein, 107 aa, synthetic construct
DIQMTQSPSSLSASVGDRVTITCRVSENIYSNLAWYQQKPGKAPKLLIYEATNLAEGVPSRFSGSGSGTDFTLTISSLQPEDFATYYCQHFWGTPYTFGQGTKLEIK

SEQ ID NO: 10 - protein, 6 aa, synthetic construct
ENIYSN

SEQ ID NO: 11 - skipped

SEQ ID NO: 12 - protein, 9 aa, synthetic construct
QHFWGTPYT

SEQ ID NO: 13 - protein, 214 aa, synthetic construct
DIQMTQSPSSLSASVGDRVTITCRVSENIYSNLAWYQQKPGKAPKLLIYEATNLAEGVPSRFSGSGSGTDFTLTISSLQPEDFATYYCQHFWGTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 330 aa, synthetic construct [sequence omitted]

SEQ ID NO: 15 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 377 aa, synthetic construct
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 17 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 120 aa, synthetic construct
EVQLQQSGAELVRSGASVKMSCKVNDYTFTNYNMHWLRQTPGQGLEWIGYIYPGNGDTNYNQKFKGKATLTADTSSSTAYMQIISLTSEDPAVYFCARQGIHYYYIDVWGAGTTVTVSSG

SEQ ID NO: 19 - protein, 10 aa, synthetic construct
DYTFTNYNMH

SEQ ID NO: 20 - protein, 13 aa, synthetic construct
YIYPGNGDTNYNQ

SEQ ID NO: 21 - protein, 10 aa, synthetic construct
QGIHYYYIDV

SEQ ID NO: 22 - protein, 110 aa, synthetic construct
DIEMTQSPATLSVTPGDRVSLSCRASQSIRDYLHWYQQKSHESPRLLIKYVSQSISGIPSRFSGSGSGSEFTLSINSVEPEDVGVYYCQNGHSLPLTFGSGTKLEIKRTV

SEQ ID NO: 23 - protein, 11 aa, synthetic construct
RASQSIRDYLH

SEQ ID NO: 24 - protein, 7 aa, synthetic construct
YVSQSIS

SEQ ID NO: 25 - protein, 9 aa, synthetic construct
QNGHSLPLT

SEQ ID NO: 26 - protein, 118 aa, synthetic construct
QVQLQQSGAELAKPGASVKMSCKASGYTFTSDWMHWVKQRPGQGLEWIGYINPSTGYTEYNQKFKDKATLTADKSSSTAYMQLRSLTSEDSAVYYCARGSSGSWFAYWGQGTLVTVSA

SEQ ID NO: 27 - protein, 5 aa, synthetic construct
SDWMH

SEQ ID NO: 28 - protein, 17 aa, synthetic construct
YINPSTGYTEYNQKFKD

SEQ ID NO: 29 - protein, 9 aa, synthetic construct
GSSGSWFAY

SEQ ID NO: 30 - protein, 111 aa, synthetic construct
DIVLTQSPASLAVSLGQRATISCRASQSVSTSSYSYMHWYQQKPGQPPKLLIKYASNLESGVPARFSGSGSGTDFTLNIHPVEEEDTATYYCQHSWEIPYTFGGGTKLEIK

SEQ ID NO: 31 - protein, 15 aa, synthetic construct
RASQSVSTSSYSYMH

SEQ ID NO: 32 - protein, 7 aa, synthetic construct
YASNLES

SEQ ID NO: 33 - protein, 9 aa, synthetic construct
QHSWEIPYT

SEQ ID NO: 34 - protein, 118 aa, synthetic construct; identical to SEQ ID NO: 26

SEQ ID NO: 35 - protein, 5 aa, synthetic construct
SDWMH

SEQ ID NO: 36 - protein, 17 aa, synthetic construct
YINPSTGYTEYNQKFKD

SEQ ID NO: 37 - protein, 9 aa, synthetic construct
GSSGSWFAY

SEQ ID NO: 38 - protein, 112 aa, synthetic construct
DVVMTQTPLTLSVTIGQPASISCKSSQSLLNSDGKTYLNWLLQRPGQSPKRLIYLVSKLDSGVPDRFTGSGSGTDFTLKISRVEAEDLGVYYCWQGTHFPRTFGGGTKLEIK

SEQ ID NO: 39 - protein, 16 aa, synthetic construct
KSSQSLLNSDGKTYLN

SEQ ID NO: 40 - protein, 7 aa, synthetic construct
LVSKLDS

SEQ ID NO: 41 - protein, 9 aa, synthetic construct
WQGTHFPRT

SEQ ID NO: 42 - protein, 114 aa, synthetic construct
EVQLQQSVAELVRPGTSVTISCKASGYTFTNYWLGWVKQRPGHGLEWIGDIFPGGDYPNYNEKFKGKATLTADTSSSTAYMQLSSLTSEDSSVYFCTRGYFDVWGAGTTVTVSS

SEQ ID NO: 43 - protein, 6 aa, synthetic construct
TNYWLG

SEQ ID NO: 44 - protein, 17 aa, synthetic construct
DIFPGGDYPNYNEKFKG

SEQ ID NO: 45 - protein, 7 aa, synthetic construct
TRGYFDV

SEQ ID NO: 46 - protein, 114 aa, synthetic construct
DIQMTQSPSSLSVSAGEKVTMSCKSSQSLLNSENQKNYLAWFQQKPGQPPKMLIYGASTRESGVPDRFTGSGSGTDFTLTISSVQAEDLAVYYCQNDHSYPFTFGSGTKLEIKR

SEQ ID NO: 47 - protein, 17 aa, synthetic construct
KSSQSLLNSENQKNYLA

SEQ ID NO: 48 - protein, 7 aa, synthetic construct
GASTRES

SEQ ID NO: 49 - protein, 7 aa, synthetic construct
QNDHSYP

SEQ ID NO: 50 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 51-658 - skipped (608 entries)

SEQ ID NO: 659 - protein, 119 aa, synthetic construct
DVKFVESGGGLVKVGGSLKLSCAASGFTFSTYYMSWVRQTPEKRLELVAVINPTGGRTDFLDSVRGRFTISRDNAKNTLYLQMTSLKSEDTALYHCARRGELGRALDYWGQGTSVTVSS

SEQ ID NO: 660 - protein, 5 aa, synthetic construct
TYYMS

SEQ ID NO: 661 - protein, 17 aa, synthetic construct
VINPTGGRTDFLDSVRG

SEQ ID NO: 662 - protein, 10 aa, synthetic construct
RGELGRALDY

SEQ ID NO: 663 - protein, 111 aa, synthetic construct
NIVLTQFPASLAVSLGQRASISCRASESVDSYGYSFIHWYQQKPGQPPKLLIYLASSLESGVPARFSGSGSRTDFTLTIDPVEANDAATYYCQQNNGDPWTFGGGTKLEIK

SEQ ID NO: 664 - protein, 15 aa, synthetic construct
RASESVDSYGYSFIH

SEQ ID NO: 665 - protein, 7 aa, synthetic construct
LASSLES

SEQ ID NO: 666 - protein, 9 aa, synthetic construct
QQNNGDPWT

SEQ ID NO: 667-811 - skipped (145 entries)

SEQ ID NO: 812 - protein, 121 aa, synthetic construct
DVQLVESGGDLVQPGGSRKLSCTASGFTFSNFGMHWVRQAPEKGLEWVAYISSGSSTIYYADTVKGRFTISRDNPENTLFLQMTSLRSEDTAMYYCTRLDYYTNSYSMDHWGQGTSVTVSS

SEQ ID NO: 813 - protein, 8 aa, synthetic construct
GFTFSNFG

SEQ ID NO: 814 - protein, 8 aa, synthetic construct
ISSGSSTI

SEQ ID NO: 815 - protein, 14 aa, synthetic construct
TRLDYYTNSYSMDH

SEQ ID NO: 816 - protein, 106 aa, synthetic construct
QIVLTQSPALMSASPGEKVTLTCSASSSLPYIYWYQQKPGSSPKPWIYLTSNLASGVPARFSGSRSGTSYSLTISSVEAEDAATYYCQQWSSNPFTFGSGTKLEIK

SEQ ID NO: 817 - protein, 5 aa, synthetic construct
SSLPY

SEQ ID NO: 818 - skipped

SEQ ID NO: 819 - protein, 9 aa, synthetic construct
QQWSSNPFT

SEQ ID NO: 820 - protein, 117 aa, synthetic construct
QVQLQQSGAELAKPGASVKMSCKASGYTFTSNWMHWVKQRPGQGLEWIGYINPSNGYTECNQKFRDKATLSADKSSSTAYMQLNSLTSADSAVYYCALMISAWLPYWGQGTLVTVSA

SEQ ID NO: 821 - protein, 8 aa, synthetic construct
GYTFTSNW

SEQ ID NO: 822 - protein, 8 aa, synthetic construct
INPSNGYT

SEQ ID NO: 823 - protein, 10 aa, synthetic construct
ALMISAWLPY

SEQ ID NO: 824 - protein, 111 aa, synthetic construct
DIVLTQSPASLAISLGQRATISCRASQSVSASSYSYVHWYQQKPGQPPKLLIKYASSLESGVPARFSGSGSGTDFTLNIHPVEEEDTATYYCLHTWEIPYTFGGGTKLEIK

SEQ ID NO: 825 - protein, 10 aa, synthetic construct
QSVSASSYSY

SEQ ID NO: 826 - skipped

SEQ ID NO: 827 - protein, 9 aa, synthetic construct
LHTWEIPYT

SEQ ID NO: 828 - protein, 116 aa, synthetic construct
QVQLQQSGAELTRPGASVNLSCKASGYTFTSYWMQWVKQRPGQGLEWIGAIYPGDGDTRFNQKFKGKATLTADESSSTAYMQLSSLASEDSAVYYCATYYRYDDYWGQGTTLTVSS

SEQ ID NO: 829 - protein, 8 aa, synthetic construct
GYTFTSYW

SEQ ID NO: 830 - protein, 8 aa, synthetic construct
IYPGDGDT

SEQ ID NO: 831 - protein, 9 aa, synthetic construct
ATYYRYDDY

SEQ ID NO: 832 - protein, 106 aa, synthetic construct
QIVLTQSPAIMSASPGEKVTMTCSASSSVSYMHWYQQKSGTSPKRWIYDTSKLASGVPTRFSGSGSGTSYSLTISSMEAEDAATYYCQQWSSNPYTFGGGTKLEIK

SEQ ID NO: 833 - protein, 5 aa, synthetic construct
SSVSY

SEQ ID NO: 834 - skipped

SEQ ID NO: 835 - protein, 9 aa, synthetic construct
QQWSSNPYT

SEQ ID NO: 836 - protein, 122 aa, synthetic construct
EVQLQQSGPDLVKPGASMKISCKASGYTFIDYNIHWVKQSQGKSLDWIGYIYPYNGGTGYNQKFKNKATLTVDSSSSTAYMEVRSLTFEDSAVYFCAREADYYGNRGQFDYWGQGTLVTVSA

SEQ ID NO: 837 - protein, 8 aa, synthetic construct
GYTFIDYN

SEQ ID NO: 838 - protein, 8 aa, synthetic construct
IYPYNGGT

SEQ ID NO: 839 - protein, 15 aa, synthetic construct
AREADYYGNRGQFDY

SEQ ID NO: 840 - protein, 107 aa, synthetic construct
DIQMTQSPASLSVSVGETVTITCRASENIFSNLAWYQQKQGKSPQLLVYGEANLADGVPSRFSGSGSGTQYSLKINSLQSEDFGNYYCQHFWGTPYTFGGGTTLEIK

SEQ ID NO: 841 - protein, 6 aa, synthetic construct
ENIFSN

SEQ ID NO: 842 - skipped

SEQ ID NO: 843 - protein, 9 aa, synthetic construct
QHFWGTPYT

SEQ ID NO: 844 - protein, 120 aa, synthetic construct
QVTLKESGPGILQPSQTLSLTCSFSGFSLNTSGTGVGWIRQPSGKGLEWLTHIWWNDNKFYNTFLKSRLTISKETSNNQVFLKIASVDTADAATYYCARMAYGNLWFVNWGQGTLVAVST

SEQ ID NO: 845 - protein, 10 aa, synthetic construct
GFSLNTSGTG

SEQ ID NO: 846 - protein, 7 aa, synthetic construct
IWWNDNK

SEQ ID NO: 847 - protein, 12 aa, synthetic construct
ARMAYGNLWFVN

SEQ ID NO: 848 - protein, 111 aa, synthetic construct
DIVLTQSPASLAVSLGQRASISCRASRSVTISGYSYMYWYQQKPGQPPRLLFYLASNLASGVPARFSGSGSGTDFTLNIHPVEEEDAAIYYCQHSRELPYTFGGGTKLEIK

SEQ ID NO: 849 - protein, 10 aa, synthetic construct
RSVTISGYSY

SEQ ID NO: 850 - skipped

SEQ ID NO: 851 - protein, 9 aa, synthetic construct
QHSRELPYT

SEQ ID NO: 852 - protein, 123 aa, synthetic construct
QIQLVQSGPELKKPRETVKISCKASGYTFTDYSMHWVKQAPGKGLKWMGWINTETGEPTYADDFKGRFAFSLEASASSAYLQINILKDEDTATYFCARSRGGYYEDYYALDYWGQGTSVTVSS

SEQ ID NO: 853 - protein, 8 aa, synthetic construct
GYTFTDYS

SEQ ID NO: 854 - protein, 8 aa, synthetic construct
INTETGEP

SEQ ID NO: 855 - protein, 16 aa, synthetic construct
ARSRGGYYEDYYALDY

SEQ ID NO: 856 - protein, 107 aa, synthetic construct
DIQMTQSPASLSASVGESVTITCRASGNIHYYLAWYQQKQGKSPQLLVYNAKNLADGVPSRFSGSGSGTQFSLKINSLQPEDFGSYYCQHFWISPPTFGGGTKLEIK

SEQ ID NO: 857 - protein, 6 aa, synthetic construct
GNIHYY

SEQ ID NO: 858 - skipped

SEQ ID NO: 859 - protein, 9 aa, synthetic construct
QHFWISPPT

SEQ ID NO: 860 - protein, 118 aa, synthetic construct
EVQLRQSGADLVKPGASVKLSCTASGFNIKDTYIDWVKQRPEQGLDWIGRIDPANGNTKYDPKFQGKATIITDTSSNTAYLQLSNLTSEDTAVYYCARYGSYPYFDYWGRGTTLAVSS

SEQ ID NO: 861 - protein, 8 aa, synthetic construct
GFNIKDTY

SEQ ID NO: 862 - protein, 8 aa, synthetic construct
IDPANGNT

SEQ ID NO: 863 - protein, 11 aa, synthetic construct
ARYGSYPYFDY

SEQ ID NO: 864 - protein, 107 aa, synthetic construct
SIVMTQTPKFLLISAGDRVTITCKASQSVRNDVAWYQQKPGQSPKLLMYYASNRYTGVPDRFTGSGYGTDFTFTISTVQAEDLAVYFCQQDYSSPPTFGGGTKLEIK

SEQ ID NO: 865 - protein, 6 aa, synthetic construct
QSVRND

SEQ ID NO: 866 - skipped

SEQ ID NO: 867 - protein, 9 aa, synthetic construct
QQDYSSPPT

SEQ ID NO: 868 - protein, 118 aa, synthetic construct
QVQLQQSGPELVRPGVSVKISCKVSGYTFTDYVMHWVKQSHAKSLEWIGIISPYSGNTNYNQNFKGKATMTVDKSSSTAYMALARLTSEDSAIYYCAREGDLPMFAYWGQGTLVTVSA

SEQ ID NO: 869 - protein, 8 aa, synthetic construct
GYTFTDYV

SEQ ID NO: 870 - protein, 8 aa, synthetic construct
ISPYSGNT

SEQ ID NO: 871 - protein, 11 aa, synthetic construct
AREGDLPMFAY

SEQ ID NO: 872 - protein, 106 aa, synthetic construct
QIVLTQSPTIMSASPGEKVTMTCSASSSVSYIYWYQQNPGSSPRLLIYDTSILASGVPFRFSGSGSGTSYSLTISRMEAEDAATYYCQQWTSYPLTFGSGTKLELK

SEQ ID NO: 873 - protein, 5 aa, synthetic construct
SSVSY

SEQ ID NO: 874 - skipped

SEQ ID NO: 875 - protein, 9 aa, synthetic construct
QQWTSYPLT

SEQ ID NO: 876 - protein, 120 aa, synthetic construct
EVQLQQSGPELVKPGASVKISCKASGYTFTDYNINWVKQSHGKSLEWIGYIYPYIGGSGYNQKFKSKATLSADNPSSTAYMELRSLTSEDSAVYYCAREDKTARNAMDYWGQGTPVTVSS

SEQ ID NO: 877 - protein, 8 aa, synthetic construct
GYTFTDYN

SEQ ID NO: 878 - protein, 8 aa, synthetic construct
IYPYIGGS

SEQ ID NO: 879 - protein, 13 aa, synthetic construct
AREDKTARNAMDY

SEQ ID NO: 880 - protein, 107 aa, synthetic construct
DIQMTQSPASLSVSVGETVTIICRVSENIYSNLAWYQQKQGKSPQLLVYEATNLAEGVPSRFSGSGSGTQYSLKINSLQSEDFGSYYCQHFWGTPYTFGGGTKLEIK

SEQ ID NO: 881 - protein, 6 aa, synthetic construct
ENIYSN

SEQ ID NO: 882 - skipped

SEQ ID NO: 883 - protein, 9 aa, synthetic construct
QHFWGTPYT

SEQ ID NO: 884 - protein, 120 aa, synthetic construct
DVQLQESGPGLVKPSQSLSLTCTVTGYSITSDYAWNWIRQFPGNKLEWMGYISYSGSTSYNPSLKSRISITRDTSKNQFFLQLNSVTTEDTATYYCARHYRYPPYAMDYWGQGTSVTVSS

SEQ ID NO: 885 - protein, 9 aa, synthetic construct
GYSITSDYA

SEQ ID NO: 886 - protein, 7 aa, synthetic construct
ISYSGST

SEQ ID NO: 887 - protein, 13 aa, synthetic construct
ARHYRYPPYAMDY

SEQ ID NO: 888 - protein, 113 aa, synthetic construct
DIVMTQSPSSLAMSVGQKVTMSCKSSQSLLNSSNQKNYLAWYQQKPGQSPKLLVYFASTRESGVPDRFIGSGSGTDFTLTITSVQAEDLADYFCQQHYSTPFTFGSGTKLEIK

SEQ ID NO: 889 - protein, 12 aa, synthetic construct
QSLLNSSNQKNY

SEQ ID NO: 890 - skipped

SEQ ID NO: 891 - protein, 9 aa, synthetic construct
QQHYSTPFT

SEQ ID NO: 892 - protein, 118 aa, synthetic construct
QVQLKQSGPGLVQPSQSLSITCTVSGFSLTSFGVHWVRQSPGKGLEWLGVIWSGGTTVYDAAFISRLSISKDNSKSQVFFKMNSLQTNDTAIYYCARKRGNFYVMDYWGQGTSVTVSS

SEQ ID NO: 893 - protein, 8 aa, synthetic construct
GFSLTSFG

SEQ ID NO: 894 - protein, 7 aa, synthetic construct
IWSGGTT

SEQ ID NO: 895 - protein, 12 aa, synthetic construct
ARKRGNFYVMDY

SEQ ID NO: 896 - protein, 113 aa, synthetic construct; identical to SEQ ID NO: 888

SEQ ID NO: 897 - protein, 12 aa, synthetic construct
QSLLNSSNQKNY

SEQ ID NO: 898 - skipped

SEQ ID NO: 899 - protein, 9 aa, synthetic construct
QQHYSTPFT

SEQ ID NO: 900 - protein, 118 aa, synthetic construct
QVQLQQPGSELVRPGTSVKLSCKASGYTFTSYWVHWVRQRHGQGLEWIGNVYPGSGSTNYDEKFKSKGTLTVDTSSSTAYMHLSSLTSEDSAVYYCTRGVLRFPLDYWGQGTTLTVSS

SEQ ID NO: 901 - protein, 8 aa, synthetic construct
GYTFTSYW

SEQ ID NO: 902 - protein, 8 aa, synthetic construct
VYPGSGST

SEQ ID NO: 903 - protein, 11 aa, synthetic construct
TRGVLRFPLDY

SEQ ID NO: 904 - protein, 112 aa, synthetic construct
DIVMTQAAPSVPVTPGESVSISCRSSKSLLHSNGNTYLYWFLQRPGQSPHLLIYRMSNLASGVPDRFSGSGSGTAFTLRISRVEAEDVGVYYCMQHLEYPLTFGAGTKLELK

SEQ ID NO: 905 - protein, 11 aa, synthetic construct
KSLLHSNGNTY

SEQ ID NO: 906 - skipped

SEQ ID NO: 907 - protein, 9 aa, synthetic construct
MQHLEYPLT

SEQ ID NO: 908 - protein, 118 aa, synthetic construct
QVQLQQSGPEVVRPGVSVKISCKGSGYKFPDYVMHWVKQSHAKSLEWIGIISIYSGNTNYNQKFKGKATMTVDKSSSTAYMELARLTSEDSAIYYCAREGDLPMFAYWGQGTLVTVSA

SEQ ID NO: 909 - protein, 8 aa, synthetic construct
GYKFPDYV

SEQ ID NO: 910 - protein, 8 aa, synthetic construct
ISIYSGNT

SEQ ID NO: 911 - protein, 11 aa, synthetic construct
AREGDLPMFAY

SEQ ID NO: 912 - protein, 106 aa, synthetic construct
QIVLTQSPAIMSASPGEKVTMTCNASSSVSYMYWYQQKPISSPRLLIYDTSNLASGVPVRFSGSGSGTSYSLTIGRMEAEDAATYYCQQWSSYPLTFGAGTKVEVK

SEQ ID NO: 913 - protein, 5 aa, synthetic construct
SSVSY

SEQ ID NO: 914 - skipped

SEQ ID NO: 915 - protein, 9 aa, synthetic construct
QQWSSYPLT

SEQ ID NO: 916 - protein, 122 aa, synthetic construct
QVQLKESGPGLVAPSQSLSITCTVSGFSLTAYNINWVRQPPGKGLEWLGMIWGDGNTDYNSPLKSRLTISKDNSKSQVFLKMDSLQTDDTARYYCARDLKVRRDSPYTMDYWGQGTSVTVSS

SEQ ID NO: 917 - protein, 8 aa, synthetic construct
GFSLTAYN

SEQ ID NO: 918 - protein, 7 aa, synthetic construct
IWGDGNT

SEQ ID NO: 919 - protein, 16 aa, synthetic construct
ARDLKVRRDSPYTMDY

SEQ ID NO: 920 - protein, 112 aa, synthetic construct
NIMMTQSPSSLAVSAGEKVTMSCKSSQSVLYSSNQKNYLAWYQQKPGQSPKLLIYWASNRESGVPDRFTGSGSGTDFTLTISSVQAEDLAVYYCHQYLSSYTFGGGTKLEIK

SEQ ID NO: 921 - protein, 12 aa, synthetic construct
QSVLYSSNQKNY

SEQ ID NO: 922 - skipped

SEQ ID NO: 923 - protein, 8 aa, synthetic construct
HQYLSSYT

SEQ ID NO: 924 - protein, 124 aa, synthetic construct
DVQLQESGPGLVKPSQSLSLTCTVTGYSLTSDYAWNWIRQFPGNKLEWMGYITYSGGTTYNPSLKSRISITRDTSKNQFFLQLTSVTTEDTATYYCARRGSGTTVVGDWYFDVWGAGTTVTVSS

SEQ ID NO: 925 - protein, 9 aa, synthetic construct
GYSLTSDYA

SEQ ID NO: 926 - protein, 7 aa, synthetic construct
ITYSGGT

SEQ ID NO: 927 - protein, 17 aa, synthetic construct
ARRGSGTTVVGDWYFDV

SEQ ID NO: 928 - protein, 113 aa, synthetic construct
DIVMSQSPSSLAVSVGEKVTMSCKSSQSLLYSFNQKYYLAWYQQKPGQSPKLLIYWASTRESGVPDRFTGSGSGTDFTPTISSVTAEDLAVYYCQQFYTYPYTFGGGTKLEMK

SEQ ID NO: 929 - protein, 12 aa, synthetic construct
QSLLYSFNQKYY

SEQ ID NO: 930 - skipped

SEQ ID NO: 931 - protein, 9 aa, synthetic construct
QQFYTYPYT

SEQ ID NO: 932 - protein, 122 aa, synthetic construct
EVQVQQSGPELVKPGASVKISCKASGYTFTKSNMHWVKQSHGKSLEWIGYIYPYNGGTGYNQNFKSKATLTVDISSSTAYMELRSLTLEDSAVYLCAREADYYGNRGQFDYWGQGTLVTVSA

SEQ ID NO: 933 - protein, 8 aa, synthetic construct
GYTFTKSN

SEQ ID NO: 934 - protein, 8 aa, synthetic construct
IYPYNGGT

SEQ ID NO: 935 - protein, 15 aa, synthetic construct
AREADYYGNRGQFDY

SEQ ID NO: 936 - protein, 107 aa, synthetic construct
DIQMTQSPASLSVSVGETVTITCRASDNIFSNLAWYHQKQGKSPHLLVYGATNLADGVPSRFSGSGSGTQYSLKINSLQSEDFGDYYCQHFWGTPYTFGGGTKLEIK

SEQ ID NO: 937 - protein, 6 aa, synthetic construct
DNIFSN

SEQ ID NO: 938 - skipped

SEQ ID NO: 939 - protein, 9 aa, synthetic construct
QHFWGTPYT

SEQ ID NO: 940 - protein, 119 aa, synthetic construct
QVQLQQSGAELMRPGTSVKVSCKASGYAFTNHLIEWIKQRPGQGLEWIGVINPGSDSTDYNEKFKDKATLTADKSSSTAYMQLSSLTSDDSAVYFCARSLYYNSWFVYWGQGTLVTVSA

SEQ ID NO: 941 - protein, 8 aa, synthetic construct
GYAFTNHL

SEQ ID NO: 942 - protein, 8 aa, synthetic construct
INPGSDST

SEQ ID NO: 943 - protein, 12 aa, synthetic construct
ARSLYYNSWFVY

SEQ ID NO: 944 - protein, 107 aa, synthetic construct
DIQMTQSPASLSASVGETVTITCRASENIYSYLAWYQQKRGKSPQLLVYNAKTLVEGVPSRFSGSGSGTQFSLKINSLQPEDFGSYYCQHHYGTPYTFGGGTKLEIK

SEQ ID NO: 945 - protein, 6 aa, synthetic construct
ENIYSY

SEQ ID NO: 946 - skipped

SEQ ID NO: 947 - protein, 9 aa, synthetic construct
QHHYGTPYT

SEQ ID NO: 948 - protein, 118 aa, synthetic construct
EVQLQQSGAELVKPGASVRLSCTASGFSIRDTYIHWVKQRPEQGLDWIGKIDPANGKSEYDPKFQGRATMTTDTSSNTAYLQLSSLTSEDTAVYYCTRYGYYPYFDVWGAGTTVTVFS

SEQ ID NO: 949 - protein, 8 aa, synthetic construct
GFSIRDTY

SEQ ID NO: 950 - protein, 8 aa, synthetic construct
IDPANGKS

SEQ ID NO: 951 - protein, 11 aa, synthetic construct
TRYGYYPYFDV

SEQ ID NO: 952 - protein, 107 aa, synthetic construct
SIVMTQTPKFLLVSAGDRVAITCKASQSVRHDVVWYQQKPGQSPKLLIYYASSRYTGVPDRFTGSGYGTDFTFTISTVQAEDLALYFCLQDFSSPWTFGGGTKLEIK

SEQ ID NO: 953 - protein, 6 aa, synthetic construct
QSVRHD

SEQ ID NO: 954 - skipped

SEQ ID NO: 955 - protein, 9 aa, synthetic construct
LQDFSSPWT

SEQ ID NO: 956 - protein, 120 aa, synthetic construct
EVQLQQSGPELVKPGASVKISCKTSGYTFTKNTMHWVRQSHGKSLEWIGGINPNSGGASFNQKFMGKATLTVDKSSSTAYMELRSLTSEDSAVYYCARDGYDGDWFFDVWGAGTTVTVSS

SEQ ID NO: 957 - protein, 8 aa, synthetic construct
GYTFTKNT

SEQ ID NO: 958 - protein, 8 aa, synthetic construct
INPNSGGA

SEQ ID NO: 959 - protein, 13 aa, synthetic construct
ARDGYDGDWFFDV

SEQ ID NO: 960 - protein, 107 aa, synthetic construct
DIQMNQSPFSLSASLGDTVTITCHASQNIYVWLSWYQQKPGNIPKLLIYKASDLHTGVPSRFSGSGSGTDFTLNISSLQPEDIATYYCQQGQSYPRTFGGGTKLEIK

SEQ ID NO: 961 - protein, 6 aa, synthetic construct
QNIYVW

SEQ ID NO: 962 - skipped

SEQ ID NO: 963 - protein, 9 aa, synthetic construct
QQGQSYPRT

SEQ ID NO: 964 - protein, 114 aa, synthetic construct
QVQLKESGPGLVAPSQSLSITCTVSGFSLIGHGVNWIRQPPGKGLEWLGVIWGDGNTDYNSALKSRLSISKDNSKSQVFLKMNSLQTDDTARYFCAVNSAMDYWGQGTAVTVSS

SEQ ID NO: 965 - protein, 8 aa, synthetic construct
GFSLIGHG

SEQ ID NO: 966 - protein, 7 aa, synthetic construct
IWGDGNT

SEQ ID NO: 967 - protein, 8 aa, synthetic construct
AVNSAMDY

SEQ ID NO: 968 - protein, 107 aa, synthetic construct
NIVMTQSPKSMSMSVGERVTLNCTASENVASFVSWYQQKPEQSPKLLIYGTSNRYTGVPDRFTGSGSATDFTLTISSVQAEDLGDYHCGQSYNYPFTFGSGTKLEIE

SEQ ID NO: 969 - protein, 6 aa, synthetic construct
ENVASF

SEQ ID NO: 970 - skipped

SEQ ID NO: 971 - protein, 9 aa, synthetic construct
GQSYNYPFT

SEQ ID NO: 972 - protein, 119 aa, synthetic construct
QVQLQQSGAELVRPGASVKVSCKTSGYAFTNYLIEWVKQRPGQGLEWIGVINPGSGGIYYNDKFKVKTTLTADKSSSTAYMQLSSLTSDDSAVYFCARSETHDTWFAYWGQGTLVTVSA

SEQ ID NO: 973 - protein, 8 aa, synthetic construct
GYAFTNYL

SEQ ID NO: 974 - protein, 8 aa, synthetic construct
INPGSGGI

SEQ ID NO: 975 - protein, 12 aa, synthetic construct
ARSETHDTWFAY

SEQ ID NO: 976 - protein, 108 aa, synthetic construct
DIVMTQSQKFISTSVGDRVSITCKASQSVRIAVAWFQQKPGQSPKALIYLASTRHTGVPDRFTGSGSGTDFTLTISNVQSEDLADYFCLQHWNYPYTFGGGTKLEIKR

SEQ ID NO: 977 - protein, 6 aa, synthetic construct
QSVRIA

SEQ ID NO: 978 - skipped

SEQ ID NO: 979 - protein, 9 aa, synthetic construct
LQHWNYPYT

SEQ ID NO: 980 - protein, 120 aa, synthetic construct
QVQLQQSGAELVRPGTSVKMSCKAAGYTFTNYWIGWVKQRPGHGLEWIGDIYPGGGYTNYNEKFKGKATLTADTSSSTAYMQLSSLTSEDSAIYYCASPYYGSSYGFAFWGQGTLVTVSA

SEQ ID NO: 981 - protein, 8 aa, synthetic construct
GYTFTNYW

SEQ ID NO: 982 - protein, 8 aa, synthetic construct
IYPGGGYT

SEQ ID NO: 983 - protein, 13 aa, synthetic construct
ASPYYGSSYGFAF

SEQ ID NO: 984 - protein, 108 aa, synthetic construct; identical to SEQ ID NO: 976

SEQ ID NO: 985 - protein, 6 aa, synthetic construct
QSVRIA

SEQ ID NO: 986 - skipped

SEQ ID NO: 987 - protein, 9 aa, synthetic construct
LQHWNYPYT

SEQ ID NO: 988 - protein, 117 aa, synthetic construct
QVQLKESGPGLVAPSQSLSITCTVSGFSLSGYGIKWVRQPPGKGLEWLGTIWGDGSTDYNSALKSRLSISKDNSKSQVFLKMTSLQTDDTARYYCASDSLGITFGYWGQGTLVTVSA

SEQ ID NO: 989 - protein, 8 aa, synthetic construct
GFSLSGYG

SEQ ID NO: 990 - protein, 7 aa, synthetic construct
IWGDGST

SEQ ID NO: 991 - protein, 11 aa, synthetic construct
ASDSLGITFGY

SEQ ID NO: 992 - protein, 107 aa, synthetic construct
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPLTFGAGTKLELK

SEQ ID NO: 993 - protein, 6 aa, synthetic construct
QDISNY

SEQ ID NO: 994 - skipped

SEQ ID NO: 995 - protein, 9 aa, synthetic construct
QQGNTLPLT

SEQ ID NO: 996 - protein, 120 aa, synthetic construct
QVQLKESGPGLVAPSQSLSITCTVSGFSLTSYAITWVRQPPGKGLEWLGVIWPGGGTNYNSALKSRLSISKDNSKSHLFLKMNSLQTDDTARYYCVRSYDGYLDWYFDVWGTGTTVTVSS

SEQ ID NO: 997 - protein, 8 aa, synthetic construct
GFSLTSYA

SEQ ID NO: 998 - protein, 7 aa, synthetic construct
IWPGGGT

SEQ ID NO: 999 - protein, 14 aa, synthetic construct
VRSYDGYLDWYFDV

SEQ ID NO: 1000 - protein, 107 aa, synthetic construct
NIVMTQSPKSMSMSVGERVTLSCKASENVGTYVSWYQQKPDQSPKLLIYGASNRYTGVPDRFTGSGSATDFTLIISSVQAEDLSDYHCGQSYSYPYTFGGGTKLEII

SEQ ID NO: 1001 - protein, 6 aa, synthetic construct
ENVGTY

SEQ ID NO: 1002 - skipped

SEQ ID NO: 1003 - protein, 9 aa, synthetic construct
GQSYSYPYT

SEQ ID NO: 1004 - protein, 123 aa, synthetic construct
QIQLVQSGPDLKKPGETVKISCKASGYTFTTYGMSWVKQAPGRGLKWMGWINTYSGVSTFPDDFKGRFAFSLETSASTAYLQINNLKNEDSATYFCARLGMGSTTGAGYFDVWGTGTTVTVSS

SEQ ID NO: 1005 - protein, 8 aa, synthetic construct
GYTFTTYG

SEQ ID NO: 1006 - protein, 8 aa, synthetic construct
INTYSGVS

SEQ ID NO: 1007 - protein, 16 aa, synthetic construct
ARLGMGSTTGAGYFDV

SEQ ID NO: 1008 - protein, 108 aa, synthetic construct
DIVLTQSPAIMSASPGEKVTMTCSASSSVSSWYLHWYQQKSGASPKLWIYGTSNLASGVPARFSGSGSGTSYSLTISSVEAEDAATYYCQQYRSDPYTFGSGTKLEIK

SEQ ID NO: 1009 - protein, 7 aa, synthetic construct
SSVSSWY

SEQ ID NO: 1010 - skipped

SEQ ID NO: 1011 - protein, 9 aa, synthetic construct
QQYRSDPYT

SEQ ID NO: 1012 - protein, 123 aa, synthetic construct
QIQLVQSGPELKKPGETVKISCKASGYTFTTYGMSWVKQAPGKGLKWMGWINTYSGVSTYADDFEGRFAFSLETSVSTAYLQINNLKNEDTATYFCARLGRGSTTGAGYLDVWGTGTTVTVSS

SEQ ID NO: 1013 - protein, 8 aa, synthetic construct
GYTFTTYG

SEQ ID NO: 1014 - protein, 8 aa, synthetic construct
INTYSGVS

SEQ ID NO: 1015 - protein, 16 aa, synthetic construct
ARLGRGSTTGAGYLDV

SEQ ID NO: 1016 - protein, 108 aa, synthetic construct
DIVLTQSPAIMSASPGEKVSMTCSASSSVSSWYLHWYQQKSGASPKLWIYGTSNLASGVPARFSGSGSGTSYSLTISSVEAEDAATYYCQQYHSDPYTFGSGTKLEIK

SEQ ID NO: 1017 - protein, 7 aa, synthetic construct
SSVSSWY

SEQ ID NO: 1018 - skipped

SEQ ID NO: 1019 - protein, 9 aa, synthetic construct
QQYHSDPYT

SEQ ID NO: 1020 - protein, 123 aa, synthetic construct; identical to SEQ ID NO: 1004

SEQ ID NO: 1021 - protein, 8 aa, synthetic construct
GYTFTTYG

SEQ ID NO: 1022 - protein, 8 aa, synthetic construct
INTYSGVS

SEQ ID NO: 1023 - protein, 16 aa, synthetic construct
ARLGMGSTTGAGYFDV

SEQ ID NO: 1024 - protein, 106 aa, synthetic construct
ENVLTQSPAIMSASLGEKVTLSCRASSSVNYMYWYQQKSDASPKLWIYYTSNLAPGVPARFSGSGSGNSYSLTISSVEGEDAATYYCQQFTSSPWTFGGGTKLEIK

SEQ ID NO: 1025 - protein, 5 aa, synthetic construct
SSVNY

SEQ ID NO: 1026 - skipped

SEQ ID NO: 1027 - protein, 9 aa, synthetic construct
QQFTSSPWT

SEQ ID NO: 1028 - protein, 114 aa, synthetic construct
EVQLQQSGPELVKPGASVKISCKASGYTFTDYSMNWVKQSHGKSLEWIGDITPNNGSPNYNQKFKDKATLTVDKSSSTVYMELRSLTSEDSAVYYCASLFFDYWGHGTTLTVSS

SEQ ID NO: 1029 - protein, 8 aa, synthetic construct
GYTFTDYS

SEQ ID NO: 1030 - protein, 8 aa, synthetic construct
ITPNNGSP

SEQ ID NO: 1031 - protein, 7 aa, synthetic construct
ASLFFDY

SEQ ID NO: 1032 - protein, 113 aa, synthetic construct
DIVMTQSPSSLSVSAGEKVTMSCKSSQSLLNSGNQKNYLAWYQQKPGQPPKLLIYGASTRDSGVPDRFTGSGSGTDFTLTITSVQAEDLAVYYCQNDHTYPYTFGGGTKLEIK

SEQ ID NO: 1033 - protein, 12 aa, synthetic construct
QSLLNSGNQKNY

SEQ ID NO: 1034 - skipped

SEQ ID NO: 1035 - protein, 9 aa, synthetic construct
QNDHTYPYT

SEQ ID NO: 1036 - protein, 120 aa, synthetic construct
QVQLQQPGTELVKPGASVKLSCKASGFTFTTHWMHWVRQRPGQGLEWIGNIYPSNGGSNYNEKFKTKATLTVDRSSSTAYMHLSSLTSEDSAVYYCARRVNWDGYYFDYWGQGTTLTVSS

SEQ ID NO: 1037 - protein, 8 aa, synthetic construct
GFTFTTHW

SEQ ID NO: 1038 - protein, 8 aa, synthetic construct
IYPSNGGS

SEQ ID NO: 1039 - protein, 13 aa, synthetic construct
ARRVNWDGYYFDY

SEQ ID NO: 1040 - protein, 107 aa, synthetic construct
DIVMTQSQKFMSTSVGDRVSVTCKASQNVGTNVAWYQQKPGQSPKLLIYSASYRYSGVPDRFTGSGSGTDFTLTISNVQSEDLADYFCQQYNSYPLTFGGGTKLEIK

SEQ ID NO: 1041 - protein, 6 aa, synthetic construct
QNVGTN

SEQ ID NO: 1042 - skipped

SEQ ID NO: 1043 - protein, 9 aa, synthetic construct
QQYNSYPLT

SEQ ID NO: 1044 - protein, 118 aa, synthetic construct
QVQLQESGTELVKPGASVKLSCKASGYTFTSYWIHWVKQRPGQGLEWIGNINPSNGGTNYNEKFKSKAALTVDKSSSTAYMQLSSLTSEDSAVYYCARRGLPYFFDYWGQGTTLTVSS

SEQ ID NO: 1045 - protein, 8 aa, synthetic construct
GYTFTSYW

SEQ ID NO: 1046 - protein, 8 aa, synthetic construct
INPSNGGT

SEQ ID NO: 1047 - protein, 11 aa, synthetic construct
ARRGLPYFFDY

SEQ ID NO: 1048 - protein, 107 aa, synthetic construct
DIVMTQSQKFMSTSVGDRVSVTCKGSQNVGYNVAWYQQKPGQSPKALVYSASDRHSGVPDRFAGSGSGTDFTLTISNVQSEDLAEYFCQQYNSYPLTFGAGTKLELK

SEQ ID NO: 1049 - protein, 6 aa, synthetic construct
QNVGYN

SEQ ID NO: 1050 - skipped

SEQ ID NO: 1051 - protein, 9 aa, synthetic construct
QQYNSYPLT

SEQ ID NO: 1052 - protein, 120 aa, synthetic construct; identical to SEQ ID NO: 4

SEQ ID NO: 1053 - protein, 8 aa, synthetic construct
GYTFTDYN

SEQ ID NO: 1054 - protein, 8 aa, synthetic construct
IYPYIGGS

SEQ ID NO: 1055 - protein, 13 aa, synthetic construct
AREDKTARNAMDY

SEQ ID NO: 1056 - protein, 107 aa, synthetic construct
DIQMTQSPSSLSASVGDRVTITCRVSENIYDVLAWYQQKPGKAPKLLIYEATNLAEGVPSRFSGSGSGTDFTLTISSLQPEDFATYYCQHFWGTPYTFGQGTKLEIK

SEQ ID NO: 1057 - protein, 6 aa, synthetic construct
ENIYDV

SEQ ID NO: 1058 - skipped

SEQ ID NO: 1059 - protein, 9 aa, synthetic construct
QHFWGTPYT

SEQ ID NO: 1060-1548 - skipped (489 entries)

SEQ ID NO: 1549 - protein, 119 aa, synthetic construct
QVQLVQSGAEVKKPGASVKVSCKASGYAFTNYLIEWVRQAPGQGLEWIGVINPGSGGIYYAQKFQGRVTMTADTSTSTVYMELSSLRSEDTAVYYCARSETHDTWFAYWGQGTLVTVSS

SEQ ID NO: 1550 - protein, 8 aa, synthetic construct
GYAFTNYL

SEQ ID NO: 1551 - protein, 8 aa, synthetic construct
INPGSGGI

SEQ ID NO: 1552 - protein, 12 aa, synthetic construct
ARSETHDTWFAY

SEQ ID NO: 1553 - protein, 446 aa, synthetic construct
QVQLVQSGAEVKKPGASVKVSCKASGYAFTNYLIEWVRQAPGQGLEWIGVINPGSGGIYYAQKFQGRVTMTADTSTSTVYMELSSLRSEDTAVYYCARSETHDTWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 1554 - protein, 107 aa, synthetic construct
DIQMTQSPSSLSASVGDRVTITCKASQSVRIAVAWFQQKPGKAPKALIYLASTRHTGVPSRFSGSGSGTDFTLTISSVQPEDFATYYCLQHWNYPYTFGQGTKLEIK

SEQ ID NO: 1555 - protein, 6 aa, synthetic construct
QSVRIA

SEQ ID NO: 1556 - skipped

SEQ ID NO: 1557 - protein, 9 aa, synthetic construct
LQHWNYPYT

SEQ ID NO: 1558 - protein, 214 aa, synthetic construct
DIQMTQSPSSLSASVGDRVTITCKASQSVRIAVAWFQQKPGKAPKALIYLASTRHTGVPSRFSGSGSGTDFTLTISSVQPEDFATYYCLQHWNYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1559-1999 - skipped (441 entries)

SEQ ID NO: 2000 - protein, 7 aa, synthetic construct
SGIYYWS

SEQ ID NO: 2001 - protein, 16 aa, synthetic construct
YIYYSGSTNYNPSLKS

SEQ ID NO: 2002 - protein, 20 aa, synthetic construct
DYYVSGNYYNVDYYFFGVDV

SEQ ID NO: 2003 - protein, 11 aa, synthetic construct
RASQSVSSYLA

SEQ ID NO: 2004 - protein, 7 aa, synthetic construct
DASNRAT

SEQ ID NO: 2005 - protein, 11 aa, synthetic construct
QQRSNWPPLFT

(19) United States
(12) Patent Application Publication
(10) Pub. No.: US 2026/0035460 A1
(43) Pub. Date: Feb. 5, 2026

(54) COMBINATION THERAPY WITH ANTI-PVRIG ANTIBODIES FORMULATIONS AND ANTI-PD-1 ANTIBODIES

(71) Applicants: Adeboye Henry ADEWOYE, MD, San Francisco, CA (US); Pierre FERRE, Toulouse (FR); Rehovot INBAL, Barbiro (IL); Eran OPHIR, Even Yehuda (IL); Gad S. COJOCARU, Holon (IL); Paul A. BASCIANO, Holon (IL); Brian D. LAMON, Holon (IL); Compugen Ltd., Holon (IL); Bristol-Myers Squibb Company, Princeton, NJ (US)

(72) Inventors: Adeboye Henry Adewoye, MD, San Francisco, CA (US); Pierre Ferre, Toulouse (FR); Inbal Barbiro, Rehovot (IL); Eran Ophir, Even Yehuda (IL); Gad S. Cojocaru, Holon (IL); Paul A. Basciano, Princeton, NJ (US); Brian D. Lamon, Princeton, NJ (US)

(21) Appl. No.: 19/099,045

(22) PCT Filed: Jul. 28, 2023
(86) PCT No.: PCT/US2023/071281

§ 371 Date: Jan. 27, 2025

(51) Int. Cl. C07K 16/28; A61K 39/00; A61K 47/02; A61K 47/18; A61K 47/22; A61K 47/26; A61P 35/00; G01N 33/569; G01N 33/574; G01N 33/68

(52) U.S. Cl. CPC C07K 16/2803; A61K 47/02; A61K 47/183; A61K 47/22; A61K 47/26; A61P 35/00; C07K 16/2818; G01N 33/56972; G01N 33/57488; G01N 33/57492; G01N 33/57496; G01N 33/6863; G01N 33/6872; A61K 2039/507; A61K 2039/545; C07K 2317/515; C07K 2317/522; C07K 2317/524; C07K 2317/526; C07K 2317/53; C07K 2317/565; G01N 2333/57; G01N 2333/70503; G01N 2333/7051; G01N 2333/70514; G01N 2333/70517; G01N 2333/70539; G01N 2333/70589; G01N 2333/70596; G01N 2333/7158

(57) ABSTRACT

The present invention is directed to anti-PVRIG antibodies and stable liquid pharmaceutical formulations thereof. The present invention is directed to monotherapy and combination treatments with anti-PVRIG antibodies and anti-PD-1 antibodies, in particular nivolumab, using stable liquid pharmaceutical formulations thereof. The present invention also provides biomarkers for use in determining populations for treatment with anti-PVRIG antibodies and such biomarkers include, for example PVRIG and/or PVRL2 expression.

### CROSS-REFERENCE TO RELATED APPLICATIONS

This application claims priority to U.S. Provisional Patent Application No. 63/393,151 filed Jul. 28, 2022, U.S. Provisional Patent Application No. 63/410,174 filed Sep. 26, 2022, U.S. Provisional Patent Application No. 63/380,560 filed Oct. 21, 2022, U.S. Provisional Patent Application No. 63/385,955 filed Dec. 2, 2022, U.S. Provisional Patent Application No. 63/484,714 filed Feb. 13, 2023, U.S. Provisional Patent Application No. 63/487,152 filed Feb. 27, 2023, U.S. Provisional Patent Application No. 63/502,362 filed May 15, 2023, and U.S. Provisional Patent Application No. 63/510,344 filed Jun. 26, 2023, which are hereby incorporated by reference in their entireties.

### BACKGROUND OF THE INVENTION

Naïve T cells must receive two independent signals from antigen-presenting cells (APC) in order to become productively activated. The first, Signal 1, is antigen-specific and occurs when T cell antigen receptors encounter the appropriate antigen-MHC complex on the APC. The fate of the immune response is determined by a second, antigen-independent signal (Signal 2) which is delivered through a T cell costimulatory molecule that engages its APC-expressed ligand. This second signal could be either stimulatory (positive costimulation) or inhibitory (negative costimulation or coinhibition). In the absence of a costimulatory signal, or in the presence of a coinhibitory signal, T-cell activation is impaired or aborted, which may lead to a state of antigen-specific unresponsiveness (known as T-cell anergy), or may result in T-cell apoptotic death.

Costimulatory molecule pairs usually consist of ligands expressed on APCs and their cognate receptors expressed on T cells. The prototype ligand/receptor pairs of costimulatory molecules are B7/CD28 and CD40/CD40L. The B7 family consists of structurally related, cell-surface protein ligands, which may provide stimulatory or inhibitory input to an immune response. Members of the B7 family are structurally related, with the extracellular domain containing at least one variable or constant immunoglobulin domain.

Both positive and negative costimulatory signals play critical roles in the regulation of cell-mediated immune responses, and molecules that mediate these signals have proven to be effective targets for immunomodulation. Based on this knowledge, several therapeutic approaches that involve targeting of costimulatory molecules have been developed, and were shown to be useful for prevention and treatment of cancer by turning on, or preventing the turning off, of immune responses in cancer patients and for prevention and treatment of autoimmune diseases and inflammatory diseases, as well as rejection of allogenic transplantation, each by turning off uncontrolled immune responses, or by induction of “off signal” by negative costimulation (or coinhibition) in subjects with these pathological conditions.

Manipulation of the signals delivered by B7 ligands has shown potential in the treatment of autoimmunity, inflammatory diseases, and transplant rejection. Therapeutic strategies include blocking of costimulation using monoclonal antibodies to the ligand or to the receptor of a costimulatory pair, or using soluble fusion proteins composed of the costimulatory receptor that may bind and block its appropriate ligand. Another approach is induction of co-inhibition using soluble fusion protein of an inhibitory ligand. These approaches rely, at least partially, on the eventual deletion of auto- or allo-reactive T cells (which are responsible for the pathogenic processes in autoimmune diseases or transplantation, respectively), presumably because in the absence of costimulation (which induces cell survival genes) T cells become highly susceptible to induction of apoptosis. Thus, novel agents that are capable of modulating costimulatory signals, without compromising the immune system's ability to defend against pathogens, are highly advantageous for treatment and prevention of such pathological conditions.

Costimulatory pathways play an important role in tumor development. Interestingly, tumors have been shown to evade immune destruction by impeding T cell activation through inhibition of co-stimulatory factors in the B7-CD28 and TNF families, as well as by attracting regulatory T cells, which inhibit anti-tumor T cell responses (see Wang (2006), “Immune Suppression by Tumor Specific CD4” Regulatory T cells in Cancer “, Semin. Cancer. Biol. 16:73-79; Greenwald, et al., (2005), “The B7 Family Revisited”, Ann. Rev. Immunol. 23:515-48; Watts (2005), “TNF/TNFR Family Members in Co-stimulation of T Cell Responses”, Ann. Rev. Immunol. 23:23-68; Sadum, et al., (2007) “Immune Signatures of Murine and Human Cancers Reveal Unique Mechanisms of Tumor Escape and New Targets for Cancer Immunotherapy”, Clin. Canc. Res. 13 (13): 4016-4025). Such tumor expressed co-stimulatory molecules have become attractive cancer biomarkers and may serve as tumor-associated antigens (TAAs). Furthermore, costimulatory pathways have been identified as immunologic checkpoints that attenuate T cell dependent immune responses, both at the level of initiation and effector function within tumor metastases. As engineered cancer vaccines continue to improve, it is becoming clear that such immunologic checkpoints are a major barrier to the vaccines' ability to induce therapeutic anti-tumor responses. In that regard, costimulatory molecules can serve as adjuvants for active (vaccination) and passive (antibody-mediated) cancer immunotherapy, providing strategies to thwart immune tolerance and stimulate the immune system.

Over the past decade, agonists and/or antagonists to various costimulatory proteins have been developed for treating autoimmune diseases, graft rejection, allergy and cancer. For example, CTLA4-Ig (Abatacept, Orencia®) is approved for treatment of RA, mutated CTLA4-Ig (Belatacept, Nulojix®) for prevention of acute kidney transplant rejection and by the anti-CTLA4 antibody (Ipilimumab, Yervoy®), recently approved for the treatment of melanoma. Other costimulation regulators have been approved, such as the anti-PD-1 antibodies of Merck (Keytruda®) and BMS (Opdivo®), have been approved for cancer treatments and are in testing for viral infections as well.

With regard to treatment therapies, a particular pathway and target of interest is PVRIG. PVRIG is a transmembrane domain protein of 326 amino acids in length, with a signal peptide (spanning from amino acid 1 to 40), an extracellular domain (spanning from amino acid 41 to 171), a transmembrane domain (spanning from amino acid 172 to 190) and a cytoplasmic domain (spanning from amino acid 191 to 326). The full length human PVRIG protein is shown in FIG. 1. There are two methionines that can be start codons, but the mature proteins are identical.

The PVRIG proteins contain an immunoglobulin (Ig) domain within the extracellular domain, which is a PVR-like Ig fold domain. The PVR-like Ig fold domain may be responsible for functional counterpart binding, by analogy to the other B7 family members. The PVR-like Ig fold domain of the extracellular domain includes one disulfide bond formed between intra domain cysteine residues, as is typical for this fold and may be important for structure-function. These cysteines are located at residues 22 and 93 (or 94). In one embodiment, there is provided a soluble fragment of PVRIG that can be used in testing of PVRIG antibodies. Included within the definition of PVRIG proteins are PVRIG ECD fragments, including know ECD fragments such as those described in U.S. Pat. No. 9,714,289.

PVRIG has also been identified as an inhibitory receptor which recognizes CD112 but not CD155, and it may be involved in negative regulation of the anti-tumor functions mediated by DNAM-1. PVRL2 was identified as the ligand for PVRIG, placing PVRIG in the DNAM/TIGIT immunoreceptor axis (see, Liang et al., Journal of Clinical Oncology 2017 35: 15_suppl, 3074-3074).

Anti-PVRIG antibodies (including antigen-binding fragments) that both bind to PVRIG and prevent activation by PVRL2 (e.g., most commonly by blocking the interaction of PVRIG and PVLR2), are used to enhance T cell and/or NK cell activation and be used in treating diseases such as cancer and pathogen infection. As such, formulations for administering such antibodies are needed.

### BRIEF SUMMARY OF THE INVENTION

Accordingly, it is an object of the invention to provide stable liquid pharmaceutical formulations comprising anti-PVRIG antibodies or use in combination with nivolumab in disease treatment (e.g., anti-PVRIG antibodies including those with CDRs identical to those shown in FIG. 3).

Furthermore, there remains a need in the art to develop biomarkers for determining which patient populations for which anti-PVRIG antibodies might find the most beneficial use in combination with nivolumab in treatment. As such, biomarkers for use in identifying these populations are needed.

Accordingly, it is an object of the invention to provide biomarkers for use in determining populations for treatment with anti-PVRIG antibodies (e.g., anti-PVRIG antibodies including those with CDRs identical to those shown in FIG. 3, also referred to herein as anti-PVRIG treatment antibodies) in combination with nivolumab. Such biomarkers include, for example PVRIG and/or PVRL2 expression, as described herein.

In some embodiments, the present invention disclosed herein provides a method of treatment for cancer comprising administering nivolumab and an anti-PVRIG antibody, wherein said anti-PVRIG antibody is administered as a stable liquid pharmaceutical formulation and, wherein the stable liquid pharmaceutical formulation of the anti-PVRIG antibody comprises:
(a) an anti-PVRIG antibody, wherein said anti-PVRIG antibody comprises:
i) a heavy chain variable domain comprising the vhCDR1, vhCDR2, and vhCDR3 from the heavy chain of CHA.7.518.1.H4 (S241P) (SEQ ID NO:4), and
ii) a light chain variable domain comprising the vlCDR1, vlCDR2, and vlCDR3 from the light chain of CHA.7.518.1.H4 (S241P) (SEQ ID NO:9);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the formulation has a pH from 5.5 to 7.0.

In some embodiments, said anti-PVRIG antibody comprises a CH1-hinge-CH2-CH3 sequence of IgG4 (SEQ ID NO:17 or SEQ ID NO:50), wherein said hinge region optionally comprises mutations. In some embodiments, said anti-PVRIG antibody comprises the CH1-hinge-CH2-CH3 region from IgG1, IgG2, IgG3, or IgG4, wherein said hinge region optionally comprises mutations.

In some embodiments, said heavy chain variable domain is from the heavy chain of CHA.7.518.1.H4 (S241P) (SEQ ID NO:4) and said light chain variable domain is from the light chain of CHA.7.518.1.H4 (S241P) (SEQ ID NO:9).

In some embodiments, said anti-PVRIG antibody comprises a CL region of human kappa 2 light chain.

In some embodiments, said pharmaceutical formulation comprises from 10 mM to 80 mM histidine, from 15 mM to 70 mM histidine, from 20 mM to 60 mM histidine, from 20 mM to 50 mM histidine, or from 20 mM to 30 mM histidine. In some embodiments, said pharmaceutical formulation comprises about 25 mM histidine.

In some embodiments, said pharmaceutical formulation comprises from 30 mM to 100 mM NaCl, from 30 mM to 90 mM NaCl, from 40 mM to 80 mM NaCl, from 30 mM to 70 mM NaCl, or from 45 mM to 70 mM NaCl. In some embodiments, said pharmaceutical formulation comprises about 60 mM NaCl.

In some embodiments, said pharmaceutical formulation comprises from 20 mM to 140 mM L-arginine, from 30 mM to 140 mM L-arginine, from 40 mM to 130 mM L-arginine, from 50 mM to 120 mM L-arginine, from 60 mM to 110 mM L-arginine, from 70 mM to 110 mM L-arginine, from 80 mM to 110 mM L-arginine, or from 90 mM to 110 mM L-arginine. In some embodiments, said pharmaceutical formulation comprises about 100 mM L-arginine.

In some embodiments, said pharmaceutical formulation comprises from 0.006% to 0.1% w/v polysorbate 80, from 0.007% to 0.09% w/v polysorbate 80, from 0.008% to 0.08% w/v polysorbate 80, from 0.009% to 0.09% w/v polysorbate 80, from 0.01% to 0.08% w/v polysorbate 80, from 0.01% to 0.07% w/v polysorbate 80, from 0.01% to 0.07% w/v polysorbate 80, or from 0.01% to 0.06% w/v polysorbate 80, or from 0.009% to 0.05% w/v polysorbate 80. In some embodiments, said pharmaceutical formulation comprises about 0.01% polysorbate 80.

In some embodiments, said pH is from 6 to 7.0. In some embodiments, said pH is from 6.3 to 6.8. In some embodiments, said pH is 6.5+/−0.2.

In some embodiments, said anti-PVRIG antibody is at a concentration of from 10 mg/mL to 40 mg/mL, 15 mg/mL to 40 mg/mL, 15 mg/mL to 30 mg/mL, 10 mg/mL to 25 mg/mL, or 15 mg/mL to 25 mg/mL.

In some embodiments, said formulation is stable at 2° C. to 8° C. for at least 1 week, 2 weeks, 3 weeks, 4 weeks, 5 weeks, 6 weeks, 7 weeks, 8 weeks, 9 weeks, or 10 weeks.

In some embodiments, said formulation is stable at about 20° C. to 25° C. for at least 1 week, 2 weeks, 3 weeks, 4 weeks, 5 weeks, or 6 weeks.

In some embodiments, said formulation is stable at 35° C. to 40° C. for at least 1 week, 2 weeks, 3 weeks, 4 weeks, or 5 weeks.

In some embodiments, said anti-PVRIG antibody is at a concentration of about 20 mg/mL.

In some embodiments, said anti-PVRIG antibody formulation comprises:
a) a heavy chain comprising:
i) a VH-CH1-hinge-CH2-CH3, wherein the VH is from CHA.7.518.1.H4 (S241P) (SEQ ID NO:4) and wherein the CH1-hinge-CH2-CH3 region is from IgG4; and
b) a light chain comprising:
i) a VL-CL, wherein the VL from CHA.7.518.1.H4 (S241P) (SEQ ID NO:9) and wherein the CL region is from human kappa 2 light chain.

In some embodiments, said hinge region optionally comprises mutations.

In some embodiments, said hinge region optionally comprises substitutions.

In some embodiments, said anti-PVRIG antibody formulation comprises:
i) a heavy chain comprising the heavy chain from CHA.7.518.1.H4 (S241P) (SEQ ID NO:8); and
ii) a light chain comprising the light chain from CHA.7.518.1.H4 (S241P) (SEQ ID NO:13).

In some embodiments, anti-PVRIG antibody formulation comprising:
(a) an anti-PVRIG antibody, wherein said anti-PVRIG antibody comprises:
i) a heavy chain variable domain comprising the vhCDR1, vhCDR2, and vhCDR3 from the heavy chain of CHA.7.518.1.H4 (S241P) (SEQ ID NO:4), and
ii) a light chain variable domain comprising the vlCDR1, vlCDR2, and vlCDR3 from the light chain of CHA.7.518.1.H4 (S241P) (SEQ ID NO:9);
(b) about 25 mM histidine;
(c) about 60 mM NaCl;
(d) about 100 mM L-Arginine; and
(e) about 0.01% % w/v polysorbate 80,
wherein the formulation has a pH from 6.5+/−0.2.

In some embodiments, said anti-PVRIG antibody formulation comprises:
(a) an anti-PVRIG antibody, wherein said anti-PVRIG antibody comprises:
i) a heavy chain comprising the heavy chain from CHA.7.518.1.H4 (S241P) (SEQ ID NO: 8); and
ii) a light chain comprising the light chain from CHA.7.518.1.H4 (S241P) (SEQ ID NO: 13);
(b) about 25 mM histidine;
(c) about 60 mM NaCl;
(d) about 100 mM L-Arginine; and
(e) about 0.01% % w/v polysorbate 80,
wherein the formulation has a pH from 6.5+/−0.2.

In some embodiments, said anti-PVRIG antibody is administered at a dosage of about 0.01 mg/kg to about 20 mg/kg of the anti-PVRIG antibody or about 0.01 mg/kg to about 10 mg/kg of the anti-PVRIG antibody.

In some embodiments, said anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg of the anti-PVRIG antibody.

In some embodiments, said nivolumab is administered at a dosage of about 360 mg of nivolumab or 480 mg of nivolumab.

In some embodiments, said anti-PVRIG antibody is administered 20 mg/kg every 4 weeks.

In some embodiments, said cancer is selected from the group consisting of prostate cancer, liver cancer (HCC), rectal cancer, colorectal cancer (CRC), colorectal cancer MSS (MSS-CRC; microsatellite stable colorectal carcinoma/carcinoma), metastatic MSS-CRC, refractory metastatic MSS-CRC, refractory MSS colorectal, CRC (MSS unknown), ovarian cancer (including ovarian carcinoma), platinum resistant ovarian cancer, high grade serous adenocarcinoma, endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), uveal melanoma, glioma, renal cell cancer (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cell cancer, MSI-high cancer, KRAS mutant tumors, adult T-cell leukemia/lymphoma, pleural mesothelioma, anal SCC, neuroendocrine lung cancer (including neuroendocrine lung carcinoma), small cell lung cancer, NSCLC, NSCLC large cell, NSCLC squamous cell, NSCLC adenocarcinoma, atypical carcinoid lung cancer, NSCLC with PDL1>=50% TPS, cervical SCC, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, Myelodysplastic syndromes (MDS), HNSCC, PD1 refractory or relapsing cancer, gastroesophageal junction cancer, gastric cancer, chordoma, sarcoma, endometrial sarcoma, chondrosarcoma, uterine sarcoma, plasma cell disorders, multiple myeloma, amyloidosis, AL-amyloidosis, glioblastoma, astrocytoma and fallopian tube cancer.

In some embodiments, the present invention disclosed herein provides the nivolumab and an anti-PVRIG antibody combination treatment according to any one of the method of treatment disclosed herein for use in a method of treating cancer.

In some embodiments, the present invention disclosed herein provides use of nivolumab and an anti-PVRIG antibody in the manufacture of a medicament for the treatment of cancer, wherein the anti-PVRIG antibody is formulated as the stable liquid pharmaceutical formulation according to the any of the embodiments disclosed herein.

In some embodiments, said cancer is selected from the group consisting of prostate cancer, liver cancer (HCC), rectal cancer, colorectal cancer (CRC), colorectal cancer MSS (MSS-CRC; microsatellite stable colorectal carcinoma/carcinoma), metastatic MSS-CRC, refractory metastatic MSS-CRC, refractory MSS colorectal, CRC (MSS unknown), ovarian cancer (including ovarian carcinoma), platinum resistant ovarian cancer, high grade serous adenocarcinoma, endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), uveal melanoma, glioma, renal cell cancer (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cell cancer, MSI-high cancer, KRAS mutant tumors, adult T-cell leukemia/lymphoma, pleural mesothelioma, anal SCC, neuroendocrine lung cancer (including neuroendocrine lung carcinoma), small cell lung cancer, NSCLC, NSCLC large cell, NSCLC squamous cell, NSCLC adenocarcinoma, atypical carcinoid lung cancer, NSCLC with PDL1>=50% TPS, cervical SCC, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, Myelodysplastic syndromes (MDS), HNSCC, PD1 refractory or relapsing cancer, gastroesophageal junction cancer, gastric cancer, chordoma, sarcoma, endometrial sarcoma, chondrosarcoma, uterine sarcoma, plasma cell disorders, multiple myeloma, amyloidosis, AL-amyloidosis, glioblastoma, astrocytoma and fallopian tube cancer.

In some embodiments, the present invention disclosed herein provides a method for determining or predicting the efficacy of treatment with nivolumab and an anti-PVRIG antibody in a cancer patient, the method comprising:
(a) measuring the level of one or more cellular components comprising activated DC cells, effector memory CD8 positive T cells, CD8 positive T cells, and NK-T cells, in a biological sample from of a cancer patient;
(b) quantitating the measurement of the level of the one or more cellular components; and
(c) correlating the level of the one or more cellular components with the efficacy of treatment.

In some embodiments, the present invention disclosed herein provides a method for determining a cancer patient population for treatment with nivolumab and an anti-PVRIG antibody, the method comprising:
(a) detecting the presence in a biological sample from the cancer patient one or more cellular components comprising activated DC cells, effector memory CD8 positive T cells, CD8 positive T cells, and NK-T cells;
(b) quantitating the measurement of the level of the one or more cellular components;
(c) treating the cancer patient with nivolumab and an anti-PVRIG antibody when the level of the one or more cellular components are present at an increased level as compared to a control or a patient that does not have detectable levels of the cells.

In some embodiments, the present invention disclosed herein provides a method for determining or predicting the efficacy of treatment with nivolumab and an anti-PVRIG antibody in a cancer patient, the method comprising:
(a) measuring the level of one or more biomarkers in a biological sample from the cancer patient;
(b) quantitating the measurement of the level of the one or more biomarkers; and
(c) correlating the level of the one or more biomarkers with the efficacy of treatment.

In some embodiments, the present invention disclosed herein provides a method for determining a cancer patient population for treatment with nivolumab and an anti-PVRIG antibody, the method comprising:
(a) detecting the presence in a biological sample from the cancer patient one or more biomarkers;
(b) quantitating the measurement of the level of the one or more biomarkers;
(c) treating the cancer patient with nivolumab and an anti-PVRIG antibody when the level of the one or more biomarkers is present at an increased or decreased level as compared to a control, pre-treatment sample, or a patient that does not have detectable levels of the one or more biomarkers.

In some embodiments, the biomarkers are proteins or protein levels and/or mRNAs or mRNA levels.

In some embodiments, the biological sample is obtained from a tumor, tumor microenvironment, and/or peripheral blood from the cancer patient.

In some embodiments, the activated DC cells being present and/or being present at an increased level as compared to a control, pre-treatment sample, or a patient that does not have detectable levels of the activated DC cells, is indicative of treatment efficacy for treatment with an anti-PVRIG antibody in combination with nivolumab.

In some embodiments, the activated DC cells express one or more biomarkers selected from the group consisting of LAMP3, HLA-DR and CD83.

In some embodiments, measuring the level of the activated DC cells comprises measuring expression level of one or more biomarkers selected from the group consisting of LAMP3, HLA-DR and CD83.

In some embodiments, the level of the activated DC cells is indicative of treatment efficacy for treatment with an anti-PVRIG antibody in combination with nivolumab when the level of the activated DC cells is increased by at least 1-fold, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, or 30-fold as compared to a control, pre-treatment sample, or a patient that does not respond to treatment with an anti-PVRIG antibody in combination with nivolumab.

In some embodiments, the effector memory CD8 positive T cells, or CD8 positive T cells, being present and/or being present at an increased level in the biological sample of the patient as compared to a control, pre-treatment sample, or a patient that does not have detectable levels of the effector memory CD8 positive T cells, or CD8 positive T cells, is indicative of treatment efficacy for treatment with an anti-PVRIG antibody in combination with nivolumab.

In some embodiments, the effector memory CD8 positive T cells are CD8+CD45RA−CCR7−.

In some embodiments, wherein measuring the level of the effector memory CD8 positive T cells comprises measuring the expression level of one or more of CD8, CD45RA and CCR7.

In some embodiments, the expression level of CD8 is indicative of treatment efficacy for treatment with an anti-PVRIG antibody in combination with nivolumab, when the expression level of CD8 is increased by at least 1-fold, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, or 30-fold as compared to a control, pre-treatment sample, or a patient that does not respond to treatment with an anti-PVRIG antibody in combination with nivolumab.

In some embodiments, the expression level of CD45RA is indicative of treatment efficacy for treatment with an anti-PVRIG antibody in combination with nivolumab, when the expression level of CD45RA is decreased by at least 5%, 10%, 15%, 20%, 25%, 30%, 35%, 40%, 45%, 50%, 55%, 60%, 65%, 70%, 75%, 80%, 85%, 90%, 95%, or more as compared to a control, pre-treatment sample, or a patient that does not respond to treatment with an anti-PVRIG antibody in combination with nivolumab.

In some embodiments, the expression level of CCR7 is indicative of treatment efficacy for treatment with an anti-PVRIG antibody in combination with nivolumab, when the expression level of CCR7 is decreased by at least 5%, 10%, 15%, 20%, 25%, 30%, 35%, 40%, 45%, 50%, 55%, 60%, 65%, 70%, 75%, 80%, 85%, 90%, 95%, or more as compared to a control, pre-treatment sample, or a patient that does not respond to treatment with an anti-PVRIG antibody in combination with nivolumab.

In some embodiments, the level of the effector memory CD8 positive T cells, or CD8 positive T cells is indicative of treatment efficacy for treatment with an anti-PVRIG antibody in combination with nivolumab when the level of effector memory CD8 positive T cells, or CD8 positive T cells is increased by at least 1-fold, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, or 50-fold, as compared to a control, pre-treatment sample, or a patient that does not respond to treatment with an anti-PVRIG antibody in combination with nivolumab.

In some embodiments, the one or more biomarkers are selected from the group consisting of LAMP3, HLA-DR and CD83.

In some embodiments, the method comprises measuring the expression level of one or more of LAMP3, HLA-DR and CD83.

In some embodiments, the level of the one or more biomarkers selected from the group consisting of LAMP3, HLA-DR and CD83 is indicative of treatment efficacy for treatment with an anti-PVRIG antibody in combination with nivolumab, when the expression level of the one or more of these biomarkers is increased by at least 1-fold, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, or 30-fold as compared to a control, pre-treatment sample, or a patient that does not respond to treatment with an anti-PVRIG antibody in combination with nivolumab.

In some embodiments, the biomarker is KI67.

In some embodiments, the method comprises measuring the expression level of KI67.

In some embodiments, the level of the biomarker, is indicative of treatment efficacy for treatment with an anti-PVRIG antibody in combination with nivolumab when the expression level of KI67 is increased by at least 1-fold, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, or 50-fold, as compared to a control, pre-treatment sample, or a patient that does not respond to treatment with an anti-PVRIG antibody in combination with nivolumab.

In some embodiments, the biomarker is CD8/CD4 ratio. In some embodiments, the method comprises measuring the CD8/CD4 ratio.

In some embodiments, the level of the CD8/CD4 ratio is indicative of treatment efficacy for treatment with an anti-PVRIG antibody in combination with nivolumab when the CD8/CD4 ratio is increased by at least about 10%, 20%, 30%, 40%, 50%, 60%, 70%, 80%, 90%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, or 1000%, as compared to a control, pre-treatment sample, or a patient that does not respond to treatment with an anti-PVRIG antibody in combination with nivolumab.

In some embodiments, the level of the CD8/CD4 ratio is indicative of treatment efficacy for treatment with an anti-PVRIG antibody in combination with nivolumab when the CD8/CD4 ratio is increased by about 1.1-fold, 1.2-fold, 1.3-fold, 1.4-fold, 1.5-fold, 1.6-fold, 1.7-fold, 1.8-fold, 1.9-fold, 2-fold, 2.25-fold, 2.5-fold, 2.75-fold, 3-fold, 3.25-fold, 3.5-fold, 3.75-fold, 4-fold, 4.25-fold, 4.5-fold, 4.75-fold, 5-fold, 5.25-fold, 5.5-fold, 5.75-fold, 6-fold, 6.25-fold, 6.5-fold, 6.75-fold, 7-fold, 7.25-fold, 7.5-fold, 7.75-fold, 8-fold, 8.25-fold, 8.5-fold, 8.75-fold, 9-fold, 9.25-fold, 9.5-fold, 9.75-fold, 10-fold, 10.25-fold, 10.5-fold, 10.75-fold or 11-fold, as compared to a control, pre-treatment sample, or a patient that does not respond to treatment with an anti-PVRIG antibody in combination with nivolumab.

In some embodiments, the NK-T cells being present and/or being present at an increased level in the biological sample of the patient as compared to a control, pre-treatment sample, or a patient that does not have detectable levels of the NK-T cells, is indicative of treatment efficacy for treatment with an anti-PVRIG antibody in combination with nivolumab.

In some embodiments, the level of the NK-T cells is indicative of treatment efficacy for treatment with an anti-PVRIG antibody in combination with nivolumab when the level of the NK-T cells is increased by at least 1-fold, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, or 50-fold, as compared to a control, pre-treatment sample, or a patient that does not respond to treatment with an anti-PVRIG antibody in combination with nivolumab.

In some embodiments, the biological sample is from and/or includes the tumor microenvironment (TME).

In some embodiments, the biological sample comprises a Tertiary Lymphoid Structure (TLS) within the tumor microenvironment.

In some embodiments, the activated DC cells express LAMP3 and/or PVRL2.

In some embodiments, measuring the level of the activated DC cells comprises measuring the level of LAMP3 and/or PVRL2.

In some embodiments, the CD8 positive T cells express one or more of PVRIG, TIGIT, CD28 and CD226.

In some embodiments, measuring the level of the CD8 positive T cells comprises measuring the level of one or more of PVRIG, TIGIT, CD28 and CD226.

In some embodiments, the cellular components further comprise B cells, early differentiated T stem cell-like memory cells (Tscm) and Naïve T cells.

In some embodiments, the one or more biomarkers comprise PVRIG, TIGIT, CD28, CD226, DNAM-1 axis, FOXP3, CD4, LAMP3, or PVRL2.

In some embodiments, the present invention disclosed herein provides a method for determining or predicting the efficacy of treatment with nivolumab and an anti-PVRIG antibody, the method comprising:
(a) measuring the level of TCR diversity and repertoire value in a biological sample of a cancer patient;
(b) quantitating the measurement of the level of TCR diversity and repertoire value; and
(c) correlating the level of the TCR diversity and repertoire value with the efficacy of treatment.

In some embodiments, the present invention disclosed herein provides a method for determining a cancer patient population for treatment with nivolumab and an anti-PVRIG antibody, the method comprising:
(a) measuring the level of TCR diversity and repertoire value in a biological sample from the cancer patient;
(b) quantitating the measurement of the level of TCR diversity and repertoire value;
(c) treating the cancer patient with an anti-PVRIG antibody in combination with nivolumab when there is a decrease in the TCR diversity index and increase in the number of expanded TCR repertoire level as compared to a control, pre-treatment sample, or a patient that does not respond to treatment with an anti-PVRIG antibody in combination with nivolumab.

In some embodiments, the biological sample is obtained from a tumor, tumor microenvironment, and/or peripheral blood from the cancer patient.

In some embodiments, the TCR diversity and repertoire value is measured before and/or during the treatment.

In some embodiments, the TCR diversity and repertoire value is indicated by number of unique clones of TCRα and/or TCRβ.

In some embodiments, measuring the TCR diversity and repertoire value comprises calculating a TCR diversity index from TCR repertoire analysis of T cells in the biological sample.

In some embodiments, the method comprises calculating the TCR diversity index from TCR repertoire analysis of the T cells in a biopsy taken between cycle 2 and cycle 3 of the treatment.

In some embodiments, the TCR diversity index and/or repertoire is selected from the group consisting of a Shannon index, a Simpson index, an inverse Simpson index, a normalized Shannon index, a Unique50 index, a DE30 index, a DE80 index, Gini Coefficient, proportion of TCR clone reads out of total, and a DE50 index. In some embodiments, the TCR diversity index is a Gini Coefficient.

In some embodiments, a Gini Coefficient of about 0.6 or more is indicative of treatment efficacy for treatment with the an anti-PVRIG antibody in combination with nivolumab.

In some embodiments, an increase of a Gini Coefficient of about 0.3 or more during the treatment, is indicative of treatment efficacy for treatment with an anti-PVRIG antibody in combination with nivolumab.

In some embodiments, an increase of top unique TCR clones (by ranking of TCR reads) the top deciles comprise about 50% or more of total TCR reads during the treatment is indicative of treatment efficacy for treatment with an anti-PVRIG antibody in combination with nivolumab.

In some embodiments, the TCR is TCRα and/or TCRβ.

In some embodiments, the method comprises:
(a) measuring the expression level of IFNγ in a biological sample of the cancer patient;
(b) quantitating the measurement of the expression level of IFNγ; and
(c) correlating the expression level of IFNγ with the efficacy of treatment.

In some embodiments, the present invention disclosed herein provides a method for determining a cancer patient population for treatment with nivolumab and an anti-PVRIG antibody, the method comprising:
(a) measuring the expression level of IFNγ in a biological sample from the cancer patient;
(b) quantitating the measurement of the expression level of IFNγ;
(c) treating the cancer patient with nivolumab and an anti-PVRIG antibody in combination with nivolumab when there is an increase the expression level of IFNγ as compared to a control, pre-treatment sample, or a patient that does not respond to treatment with an anti-PVRIG antibody in combination with nivolumab.

In some embodiments, measuring the expression level of IFNγ comprises measuring the expression level of IFNγ signature and/or Lymphoid signature.

In some embodiments, the IFNγ signature comprises one or more of CXCL10, CXCL9, IDO1, STAT1, HLA_DRA, and IFNG.

In some embodiments, the Lymphoid signature comprises one of more of PRF1, GZMB, CD8A, CD8B, CD3G, CD4, CD3D, and CD3E.

In some embodiments, measuring the expression level of IFNγ comprises measuring the expression level of one or more of the CXCL10, CXCL9, CD137, HLA_DRA, IFNG, PRF1, GZMB, GZMA, GZMH proteins or protein levels and/or mRNAs or mRNA levels.

In some embodiments, the expression level of the IFNγ, IFNγ signature and/or Lymphoid signature, is indicative of treatment efficacy for treatment with an anti-PVRIG antibody in combination with nivolumab when the expression level of any of the IFNγ, IFNγ signature and/or Lymphoid signature is increased by at least 1-fold, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, or 10-fold, as compared to a control, pre-treatment sample, or a patient that does not respond to treatment with an anti-PVRIG antibody in combination with nivolumab.

In some embodiments, the expression level of any one of the CXCL10, CXCL9, CD137, HLA_DRA, IFNG, PRF1, GZMB, GZMA, GZMH proteins or protein levels and/or mRNAs or mRNA levels, is indicative of treatment efficacy for treatment with an anti-PVRIG antibody in combination with nivolumab when the expression level of any of the of any one of the CXCL10, CXCL9, CD137, HLA_DRA, IFNG, PRF1, GZMB, GZMA, GZMH proteins or protein levels and/or mRNAs or mRNA levels is increased by at least 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, or 10-fold, as compared to a control, pre-treatment sample, or a patient that does not respond to treatment with an anti-PVRIG antibody in combination with nivolumab.

In some embodiments, the anti-PVRIG antibody comprises:
i) a heavy chain variable domain comprising the vhCDR1, vhCDR2, and vhCDR3 from the heavy chain of CHA.7.518.1.H4 (S241P) (SEQ ID NO:4), and
ii) a light chain variable domain comprising the vlCDR1, vlCDR2, and vlCDR3 from the light chain of CHA.7.518.1.H4 (S241P) (SEQ ID NO:9).

In some embodiments, said anti-PVRIG antibody comprises a CH1-hinge-CH2-CH3 sequence of IgG4 (SEQ ID NO:17 or SEQ ID NO:50), wherein said hinge region optionally comprises mutations.

In some embodiments, said anti-PVRIG antibody comprises the CH1-hinge-CH2-CH3 region from IgG1, IgG2, IgG3, or IgG4, wherein said hinge region optionally comprises mutations.

In some embodiments, said heavy chain variable domain is from the heavy chain of CHA.7.518.1.H4 (S241P) (SEQ ID NO:4) and said light chain variable domain is from the light chain of CHA.7.518.1.H4 (S241P) (SEQ ID NO:9).

In some embodiments, said anti-PVRIG antibody comprises a CL region of human kappa 2 light chain.

In some embodiments, the anti-PVRIG antibody is administered as a stable liquid pharmaceutical formulation.

In some embodiments, the anti-PVRIG antibody is administered as a stable liquid pharmaceutical formulation disclosed herein.

In some embodiments, upon administration to a cancer patient, an anti-PVRIG antibody in combination with nivolumab induce proliferation of one or more types of cells selected from the group consisting of activated DC cells, effector memory CD8 positive T cells, CD8 positive T cells, and NK-T cells.

In some embodiments, upon administration to a cancer patient, an anti-PVRIG antibody in combination with nivolumab there is a decrease in the TCR diversity index and increase in the number of expanded TCR repertoire value, and/or an increase in the expression level of IFNγ.

In some embodiments, the present invention disclosed herein provides a method for increasing infiltration of T cells in a tumor in a patient with PD-L1^{low} cancer, the method comprising administering to the patient an anti-PVRIG antibody in combination with nivolumab.

In some embodiments, the tumor has a PD-L1 CPS of less than 1, 2, or 3.

In some embodiments, the tumor is PVRL2+.

In some embodiments, the tumor has a PVRL2 H-score of at least 250, 255, 260, 265, 270, 275, 280, 285, 290, 295, 300, or more.

In some embodiments, the T cells comprise effector memory CD8 positive T cells and/or CD8 positive T cells.

In some embodiments, the increased infiltration of T cells in a tumor is characterized by one or more selected from the group consisting of
(a) presence of and/or presence of an increased level of effector memory CD8 positive T cells, or CD8 positive T cells in the tumor;
(b) an increase in TCR diversity index in the tumor; and
(c) an increase in the expression level of IFNγ signature in the tumor; as compared to a control, pre-treatment sample, or a patient that does not respond to treatment with an anti-PVRIG antibody in combination with nivolumab.

In some embodiments, the anti-PVRIG antibody comprises:
i) a heavy chain variable domain comprising the vhCDR1, vhCDR2, and vhCDR3 from the heavy chain of CHA.7.518.1.H4 (S241P) (SEQ ID NO:4), and
ii) a light chain variable domain comprising the vlCDR1, vlCDR2, and vlCDR3 from the light chain of CHA.7.518.1.H4 (S241P) (SEQ ID NO:9).

In some embodiments, said anti-PVRIG antibody comprises a CH1-hinge-CH2-CH3 sequence of IgG4 (SEQ ID NO:17 or SEQ ID NO:50), wherein said hinge region optionally comprises mutations.

In some embodiments, said anti-PVRIG antibody comprises the CH1-hinge-CH2-CH3 region from IgG1, IgG2, IgG3, or IgG4, wherein said hinge region optionally comprises mutations.

In some embodiments, said heavy chain variable domain is from the heavy chain of CHA.7.518.1.H4 (S241P) (SEQ ID NO:4) and said light chain variable domain is from the light chain of CHA.7.518.1.H4 (S241P) (SEQ ID NO:9).

In some embodiments, said anti-PVRIG antibody comprises a CL region of human kappa 2 light chain.

In some embodiments, the anti-PVRIG antibody is administered as a stable liquid pharmaceutical formulation.

In some embodiments, the anti-PVRIG antibody is administered as a stable liquid pharmaceutical formulation disclosed herein.

In some embodiments, the cancer is selected from the group consisting of prostate cancer, liver cancer (HCC), rectal cancer, colorectal cancer (CRC), colorectal cancer MSS (MSS-CRC; microsatellite stable colorectal carcinoma/carcinoma), metastatic MSS-CRC, refractory metastatic MSS-CRC, refractory MSS colorectal, CRC (MSS unknown), ovarian cancer (including ovarian carcinoma), platinum resistant ovarian cancer, high grade serous adenocarcinoma, endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), uveal melanoma, glioma, renal cell cancer (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cell cancer, MSI-high cancer, KRAS mutant tumors, adult T-cell leukemia/lymphoma, pleural mesothelioma, anal SCC, neuroendocrine lung cancer (including neuroendocrine lung carcinoma), small cell lung cancer, NSCLC, NSCLC large cell, NSCLC squamous cell, NSCLC adenocarcinoma, atypical carcinoid lung cancer, NSCLC with PDL1>=50% TPS, cervical SCC, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, Myelodysplastic syndromes (MDS), HNSCC, PD1 refractory or relapsing cancer, gastroesophageal junction cancer, gastric cancer, chordoma, sarcoma, endometrial sarcoma, chondrosarcoma, uterine sarcoma, plasma cell disorders, multiple myeloma, amyloidosis, AL-amyloidosis, glioblastoma, astrocytoma and fallopian tube cancer.

In some embodiments, the method further comprises administering an anti-TIGIT antibody to the patient, wherein the anti-TIGIT antibody comprises:
i) a heavy chain variable domain comprising the vhCDR1, vhCDR2, and vhCDR3 from the heavy chain of BMS-986207, and
ii) a light chain variable domain comprising the vlCDR1, vlCDR2, and vlCDR3 from the light chain of BMS-986207.

In some embodiments, the anti-TIGIT antibody comprises:
i) a heavy chain variable domain comprising a vhCDR1 comprising the sequence of SEQ ID NO:2000, a vhCDR2 comprising the sequence of SEQ ID NO:2001, and a vhCDR3 comprising the sequence of SEQ ID NO:2002, and
ii) a light chain variable domain comprising a vlCDR1 comprising the sequence of SEQ ID NO:2003, a vlCDR2 comprising the sequence of SEQ ID NO:2004, and a vlCDR3 comprising the sequence of SEQ ID NO:2005.

In some embodiments, the patient has received prior treatment with lenvatinib, pembrolizumab, or a combination treatment comprising lenvatinib and pembrolizumab.

In some embodiments, the patient has received prior treatment with one or more of capecitabine, irinotecan, abemaciclib, fulvestrant, tamoxifen, taxotere, adriamycin, cyclophosphamide, carboplatin, gemcitabine, etoposide, cisplatin, atezolizumab, doxil, bevacizumab, everolimus, abraxane, anastrozole, trodelvy, 5FU (fluorouracil), and any combination thereof.

### BRIEF DESCRIPTION OF THE DRAWINGS

FIG. 1 depicts the full-length sequence of human PVRIG.

FIG. 2 depicts the sequence of the human Poliovirus receptor-related 2 protein (PVLR2, also known as nectin-2, CD112 or herpesvirus entry mediator B, (HVEB)), the binding partner of PVRIG. PVLR2 is a human plasma membrane glycoprotein.

FIG. 3A-AG depicts the variable heavy and light chains as well as the vhCDR1, vhCDR2, vhCDR3, vlCDR1, vlCDR2 and vlCDR3 sequences of the anti-PVRIG antibodies of the invention.

FIG. 4 depicts the sequences of human IgG1, IgG2, IgG3 and IgG4.

FIGS. 5A-5D depicts the sequences of other PVRIG antibodies that can be formulated according to stable liquid formulations of an anti-PVRIG antibody of the present invention.

FIG. 6 depicts formulation parameters, including buffers and excipients for the CHA.7.518.1.H4 (S241P) antibody formulation.

FIG. 7 provides data showing the receptor occupancy at various dosages of CHA.7.518.1.H4 (S241P) (heavy chain: SEQ ID NO:8; light chain: SEQ ID NO:13).

FIG. 8 provides data showing the receptor occupancy at various dosages of CHA.7.518.1.H4 (S241P) (heavy chain: SEQ ID NO:8; light chain: SEQ ID NO:13).

FIG. 9 provides data showing PVRIG is a novel checkpoint in the TIGIT/DNAM-1 AXIS.

FIG. 10 provides data showing PVRIG inhibition reduces tumor growth in mouse cancer models.

FIG. 11 provides schematics of the study design.

FIG. 12 provides information regarding patient baseline characteristics.

FIG. 13 provides information regarding patient treatment disposition.

FIG. 14 provides information regarding treatment emergent adverse events.

FIG. 15 provides information regarding treatment emergent serious adverse events.

FIG. 16 provides a Swimmer's plot of the patient data.

FIG. 17 provides a Waterfall plot of the patient data.

FIG. 18 provides information regarding patients with stable disease and the dose-response relationship.

FIG. 19 provides best on treatment timepoint response information for patients with treatment refractory disease.

FIG. 20 provides a graph of treatment dosage data.

FIG. 21 provides a data regarding PVRIG engagement by anti-PVRIG using a receptor occupancy assay.

FIG. 22 provides patient baseline characteristics data.

FIG. 23 provides patient disposition summary.

FIG. 24 shows the dose escalation schema.

FIG. 25 provides the summary of adverse events-safety analysis set.

FIG. 26 provides the summary of serious adverse events leading to study treatment discontinuation (Arm A).

FIG. 27 provides the incidence of treatment emergent adverse events (TEAE) in ≥3 patients-monotherapy.

FIG. 28 provides the incidence of TEAEs in >3 patients-combination therapy.

FIG. 29 provides the incidence of serious TEAEs in all patients-monotherapy (n=18).

FIG. 30 provides the incidence of serious TEAEs in all patients-combination (n=13).

FIG. 31 provides the CHA. 7.518.1.H4 (S241P) PK profile following IV infusion at cycle 1 day 1-Arms A and B.

FIG. 32 provides the summary of investigator-assessed response (per recist v1.1 dlt-evaluable population) for Arms A and B.

FIGS. 33A-33B provide Swimmer Plots of data from Arms A and B. A summary plot is provided in FIG. 33C.

FIG. 34 provides a Waterfall Plot of data from Arms A and B.

FIG. 35 provides data regarding CHA.7.518.1.H4 (S241P)+nivolumab-confirmed PR in a patient with MSS (microsatellite stable status) colorectal cancer (ongoing study treatment 44 weeks).

FIG. 36 provides data regarding CHA.7.518.1.H4 (S241P) monotherapy-confirmed PR in a patient with MSS (microsatellite stable status) platinum resistant primary peritoneal cancer ongoing study treatment 25 weeks. Patient with primary peritoneal cancer (platinum resistant, MSS). Confirmed PR ongoing study treatment 18 months. Had 3 prior lines of SOC treatment (1^{st} line carboplatin/paclitaxel SD (best response), carboplatin/paclitaxel PD (best response), Doxorubicin/Bevacizumab, PR (best response, d/c due to toxicity) and enrolled into Arm A dose escalation (CHA.7.518.1.H4 (S241P) 20 mg/kg IV Q4 wks).

FIG. 37 provides data showing changes in Serum IFNγ among treatment groups from P1 arm B combination therapy treated with Nivolumab 360 mg (Q3W; every 3 weeks) for CHA.7.518.1.H4 (S241P) doses <10 mg/kg, nivolumab 480 mg (Q4W; every 4 weeks) for higher CHA.7.518.1.H4 (S241P) doses (10 and 20 mg/kg). Shown are the fold change in the serum levels of IFNγ at day 2 (C1D2) post-treatment compare to baseline (C1D1). Samples were assessed for levels of the cytokine IFNγ using a pro-inflammatory human cytokine 10-plex assay kit and MSD reader (Meso Scale Discovery). Sample signals were compared to calibration curves to determine the concentration of each analyte in serum samples.

FIG. 38 provides a schematic of the PVRIG pathway in the DNAM axis.

FIG. 39 provides a summary of the CHA.7.518.1.H4 (S241P) clinical programs.

FIG. 40 provides a summary of the CHA.7.518.1.H4 (S241P) signals of anti-Tumor activity.

FIG. 41 provides a summary of the data from monotherapy and combination dose escalation and monotherapy expansion studies.

FIG. 42 provides data related to the CHA.7.518.1.H4 (S241P) Arm A and Arm B studies.

FIG. 43 provides data related to the CHA.7.518.1.H4 (S241P) Arm A and Arm B studies.

FIG. 44 provides preliminary clinical data related to the CHA.7.518.1.H4 (S241P) study.

FIG. 45 provides data related to cellular proliferation profiles and cytokine expression.

FIG. 46 provides data related to CHA.7.518.1.H4 (S241P) Monotherapy showing increases in T-cells proliferation and cytokines level in peripheral blood of patients which demonstrated signs of clinical anti-tumor activity.

FIG. 47 provides data related to CHA.7.518.1.H4 (S241P)+Nivolumab showing increases in T-cell proliferation and cytokines level in peripheral blood of patients which demonstrated signs of clinical anti-tumor activity.

FIG. 48 provides the study design for the various study arms.

FIG. 49 provides the demographics for the study parameters.

FIG. 50 provides the patient disposition summary-DLT (dose-limiting toxicity)-evaluable population

FIG. 51 provides the summary of adverse events-safety analysis set.

FIG. 52 provides the patient incidence of treatment-emergent adverse events by maximum severity, monotherapy (n=38)-safety population (>4 pts).

FIG. 53 provides patient incidence of treatment-emergent adverse events by maximum severity, combination therapy (n=16) safety population (≥3 pts).

FIG. 54 provides the summary of investigator assessed response (per RECIST v1.1).

FIG. 55 provides a Swimmer Plot of data from the study arms.

FIG. 56 provides a Spider Plot of data from the study arms.

FIG. 57 provides a Waterfall Plot of data from the study arms.

FIG. 58 provides exemplary data from 2 patients with responses and ongoing study treatment. A 57 year old female patient with anal SCC, HPV+. Patient had evaluable disease but not measurable per RECIST v1.1 at study entry (not mandatory in dose escalation). Increasing adenopathy and SUV uptake at study entry. Confirmed CR (complete response) ongoing study treatment 96 wks (22 months). Node stable and felt to be reactive/resolved. Had 3 prior lines of chemotherapy (5-FU/mitomycin, SD (stable disease; best response), 5-FU/platinum, PR (best response for 8 months), nivolumab monotherapy, CR (best response for 9 months). Enrolled (within 1 month after progression on nivolumab monotherapy) to combination dose escalation arm: CHA.7.518.1.H4 (S241P) 0.3 mg/kg+nivolumab 360 mg both IV Q3 wks.

FIG. 59 provides data related to a trend of increasing proliferation of CD8+TEM (effector memory T cells) and NK-T cells in peripheral blood of both CHA.7.518.1.H4 (S241P) mono- and combo-therapy subjects.

FIG. 60 provides data related to a confirmed partial response in a patient with primary peritoneal cancer (platinum resistant), MSS, PDL1-negative-CHA.7.518.1.H4 (S241P) 20 mg/kg IV Q 4 wks. PR in patient with non inflamed TME also demonstrating immune activation in the blood following CHA.7.518.1.H4 (S241P) monotherapy study treatment.

FIG. 61 provides data related to PVRIG expression by early differentiated T stem cell-like memory cells (Tscm) and early differentiated CD8+ T cells.

FIG. 62 provides data regarding gene expression profile of cancer patients.

FIG. 63 provides data regarding PVRL2 expression on dendritic cells.

FIG. 64 provides study design for PVRIG monotherapy, dual combination and triple combination therapies.

FIG. 65 provides data regarding immune activation in peripheral blood of patients following CHA.7.518.1.H4 (S241P) monotherapy and combination therapy with nivolumab.

FIG. 66 provides data regarding immune activation in peripheral blood of patients with primary peritoneal PD-L1^{neg} cancer following CHA.7.518.1.H4 (S241P) monotherapy.

FIG. 67 provides data regarding immune activation in cancer patients following CHA.7.518.1.H4 (S241P) monotherapy treatment.

FIG. 68A-B provides data regarding TCR diversity (A) and immune activation (B) in cancer patients in response to CHA.7.518.1.H4 (S241P) and nivolumab combination treatment.

FIG. 69 provides data regarding expression of gene markers of activated dendritic cells in cancer patients in response to CHA.7.518.1.H4 (S241P) and nivolumab combination treatment.

FIG. 70 provides data showing elevated induction of activated-DCs markers in patients that clinically responded (RECIST criteria) to CHA.7.518.1.H4 (S241P)+nivolumab, compared to non-responders. Elevated Induction of Activated-DCs Markers in Patients that Clinically Responded to CHA.7.518.1.H4 (S241P)+Nivolumab vs Non-Responders. Finally, preliminary analysis of sera from CHA.7.518.1.H4 (S241P) (the most advanced anti-PVRIG antibody) and nivolumab-revealed elevated induction of several markers in 2 responding patients. Serum of 7 patients from the nivolumab+CHA.7.518.1.H4 (S241P) dose escalation arm, were analyzed using Olink Explore 1536. For each patient, the maximal difference of log 2 expression between all on-treatment time points and the pre-treatment value was calculated for each protein. Maximal log 2 differences were compared by Student's t-test, with patients grouped based on response, RECIST criteria (responders (R): CR+PR vs. non responders (NR): SD+PD). Out of 10 proteins most significant for on treatment up-regulation in responders, 3 are markers of activated DCs (LAMP3, HLA-DR and CD83). (Interestingly, 3 of the 10 top genes, induced post treatment in responding patients compared to non-responders, are markers of activated-DCs: LAMP3, HLA-DR, and CD83.)

FIG. 71 provides a Swimmer Plot with CHA.7.518.1.H4 (S241P) monotherapy and Nivolumab combination therapy analysis.

FIG. 72 provides data showing that CHA.7.518.1.H4 (S241P) mono and combo therapy shows signs of immune activation in peripheral blood.

FIG. 73 provides AB-635 PVRIG (6D8-1 clone) antibody sequences.

FIG. 74 provides analysis results showing PVRIG clustering with early differentiated/Tscm genes.

FIG. 75 provides results of analysis of CRC samples by MERFISH.

FIG. 76 illustrates DNA-1 axis expression in TLS region.

FIG. 77 provides data showing CHA.7.518.1.H4 (S241P) monotherapy induces TME immune modulation in patients with ovarian cancer.

FIG. 78 provides data showing CHA.7.518.1.H4 (S241P)+nivolumab combination induces TME immune modulation in patients with MSS-CRC.

FIG. 79 demonstrates extensive TME modulation in MSS-CRC patients partially responding to CHA.7.518.1.H4 (S241P)+nivolumab.

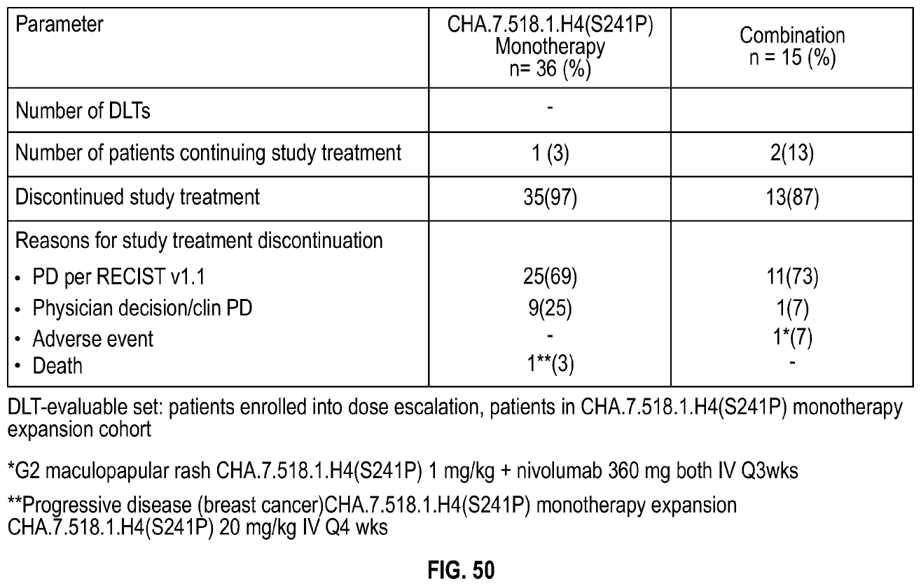

FIG. 50 provides a schematic of the PVRIG/TIGIT pathways in the DNAM axis.

FIG. 81 provides a schematic of the combination approach for immunotherapy.

FIG. 82 provides data showing T-stem like memory cells' response to checkpoint blockade.

FIG. 83 provides results of PCA analysis of scRNA of CD8+ T cells genes in NSCLC patient and unsupervised correlation analysis of scRNA of CRC patient.

FIG. 84 provides results of in-situ MERFISH analysis of tertiary lymphoid structures (TLS) in TME of CRC patients.

FIG. 85 provides results of in-situ MERFISH analysis of tertiary lymphoid structures (TLS) in TME of CRC patients.

FIG. 86 provides a schematic of PVRIG+Tscm interaction with PVRL2+DCs and related signaling pathways.

FIG. 87 demonstrates PD-L1^{neg} primary peritoneal cancer response to CHA.7.518.1.H4 (S241P) monotherapy.

FIG. 88 provides data showing TME immune modulation in ovarian cancer patient response to CHA.7.518.1.H4 (S241P) monotherapy.

FIG. 89 provide data showing immune activation in the TME of patient with ovarian cancer induced by CHA.7.518.1.H4 (S241P) monotherapy.

FIG. 90 shows TME modulation in patients with MSS-CRC following CHA.7.518.1.H4 (S241P)+nivolumab combination therapy.

FIG. 91 shows extensive TME modulation in MSS-CRC patients partially responding to CHA.7.518.1.H4 (S241P)+nivolumab combination therapy.

FIG. 92 shows induction of activated-DC markers in patients receiving CHA.7.518.1.H4 (S241P)+nivolumab combination therapy.

FIG. 93 illustrates study schema for combination dose escalation and expansion.

FIG. 94 provides the demographics of the patients.

FIG. 95 provides a patient disposition summary.

FIG. 96 illustrates incidence of treatment emergent adverse events in 4 or more patients.

FIG. 97 illustrates incidence of all serious adverse events.

FIG. 98 provides a summary of investigator assessed response (RECIST v1.1).

FIG. 99 provides a spider plot for MSS-CRC patients receiving CHA.7.518.1.H4 (S241P)+nivolumab combination treatment.

FIG. 100 provides a waterfall plot for MSS-CRC patients receiving CHA.7.518.1.H4 (S241P)+nivolumab combination treatment.

FIG. 101 provides a swimmer plot for all MSS-CRC patients.

FIG. 102 provides data regarding partial response of a patient with 32% reduction in target lesions.

FIG. 103 provides data showing TME immune modulation following CHA.7.518.1.H4 (S241P)+nivolumab combination therapy for MSS-CRC.

FIG. 104 provides data showing extensive TME modulation in MSS-CRC patients partially responding to CHA.7.518.1.H4 (S241P)+nivolumab.

FIG. 105 provides a summary of patient demographics.

FIG. 106 provides a patient disposition summary.

FIG. 107 provides a summary of investigator assessed response (recist v1.1).

FIG. 108 provides a swimmer plot for all NSCLC patients.

FIG. 109 provides a waterfall plot for all NSCLC patients.

FIG. 110 provides a spider plot for all NSCLC patients.

FIG. 111 provides a K-M plot of OS and PFS.

FIG. 112 provides a K-M plot of OS and PFS.

FIG. 113 provides a summary of clinical characteristics of all the NSCLC patients.

FIG. 114 provides a summary of patient demographics.

FIG. 115 provides a patient disposition summary.

FIG. 116 provides a summary of treatment related adverse events (TRAEs).

FIG. 117 provides a summary of the incidence of TRAEs (10% of patients).

FIG. 118 summarizes incidence of related serious adverse events-all patients.

FIG. 119 provides a summary of investigator assessed response (RECIST V1.1).

FIG. 120 provides a swimmer plot.

FIG. 121 provides a waterfall plot.

FIG. 122 provides a swimmer/spider plot.

FIG. 123 provides as summary of characteristics of the responders.

FIG. 124 provides exemplary results from a responding patient (patient 20).

FIG. 125 provides data showing pharmacodynamic activation of the immune system with study treatment.

FIG. 126A-B provides data showing TME modulation in a partial responding patient (patient 20).

FIG. 127 provides a summary of patient demographics.

FIG. 128 provides a patient disposition summary.

FIG. 129 provides a summary of treatment related adverse events (TRAEs).

FIG. 130 provides a summary of the incidence of TRAEs (15% of patients).

FIG. 131 summarizes incidence of related serious adverse events-all patients.

FIG. 132 provides a summary of investigator assessed response (RECIST V1.1).

FIG. 133 provides a swimmer plot.

FIG. 134 provides a waterfall plot.

FIG. 135 provides a spider plot.

FIG. 136 provides as summary of characteristics of the responders.

FIG. 137 provides exemplary results from a responding patient (patient 4).

FIG. 138 provides data showing pharmacodynamic activation of the immune system with study treatment.

FIG. 139 provides data showing ovarian tumors patients from both CHA.7.518.1.H4 (S241P)+Nivulomab and CHA.7.518.1.H4 (S241P)+Nivulomab+BMS-986207 treatments were pooled together. Pts with PR or SD>180 days (per RECIST) were defined as having clinical benefit (R pts) vs no clinical benefit NR (PD or SD<180). Non-parametric, Kruskal-Wallis test was performed on PVRL2 tumor H-score. High PVRL2 was more prevalent in responding patients.

FIG. 140 shows that in order to set the best cutoff for PVRL2 tumor H-score in ovarian treated patients a ROC curve analysis was performed and precision curve to select the best H-score cutoff. The 250 and 270 H-score seemed the best cutoff and both values were selected and validated by confusion matrix with calculations of specificity and sensitivity.

FIG. 141 show a schematic regarding blocking PVRIG may be the missing piece when current checkpoint inhibitors fail.

FIG. 142 shows the study design schematic.

FIG. 143 shows the patient demographics from the study.

FIG. 144 shows the patient disposition summary from the study.

FIG. 145 shows the summary of investigator assess response [RECIST V1.1] from the study.

FIG. 146 shows subject incidence of treatment-related adverse events (TRAEs)—all subjects.

FIG. 147 shows subject incidence of serious TRAE-all subjects.

FIG. 148 shows the swimmer plot of the data from the study.

FIG. 149 shows the waterfall plot of the data from the study.

FIG. 150 shows the spider plot of the data from the study.

FIG. 151 shows translational pharmacodynamic readouts.

FIG. 152 shows the clinical vignette—patient with confirmed partial response: 71 yr old female, ECOG 0 with stage III endometrial cancer initially diagnosed 2020, [John-histology] diagnosed stage IV 2022. MSS by IHC. Received 2 prior lines: adjuvant carboplatin/paclitaxel [2020]. 1st line metastatic [2021]: pembrolizumab+lenvatinib [best response PD].

FIG. 153 shows PVRL2 baseline levels correlate with clinical benefit (CB) in PROC patients treated with CHA.7.518.1.H4 (S241P)+BMS-986207 or CHA.7.518.1.H4 (S241P)+BMS-986207+nivolumab. PVRL2 baseline levels correlates with clinical benefit (CB). IHC staining of baseline biopsies of PROC patients treated with CHA.7.518.1.H4 (S241P)+nivolumab+/−BMS-986207 using A) antiPDL1 (clone 28-8, scoring by CPS), CD8 (clone C8/144B) and PVRIG (clone 6D8-1), for both precent positive cells are reported. baseline levels of these three markers do not correlate with clinical benefit. B) anti-PVRL2 (clone 181H3L2), tumor H-score (sum of IHC intensity multiplied by precent of positive cells in each intensity), shows a higher expression in CB patients. CB-Clinical Benefit=PRs+SD>=180 days on study, NCB—No Clinical Benefit=PDs+SD<180 days on study, Black square=CHA.7.518.1.H4 (S241P)+BMS-986207+nivolumab, black dots=CHA.7.518.1.H4 (S241P)+nivolumab, star=PDL 1 CPS from clinical record.

FIG. 154 shows PVRL2 genomic amplification observed in a patient with durable partial response following CHA.7.518.1.H4 (S241P)+BMS-986207+nivolumab treatment. PVRL2 genomic amplification observed in one sample from our clinical cohort and across Ovary and Gastric carcinoma in TCGA: A) One of the responding patients (PR) treated with CHA.7.518.1.H4 (S241P)+nivolumab+BMS-986207 had an amplification of the PVRL2 genomic locus which is also reflected by a PVRL2 IHC stain with a maximal tumor H-score of 300. Left micrograph X1 magnification of the core biopsy right X20 magnification of the red rectangle. B) Correlation of TCGA expression (Y axis in RESM units) by category of genomic status of PVRL2 in ovarian (OvCa) and Gastric (STAD) carcinoma. Deletion=one copy deletion, Gain=Two or three copies, Amplification=more than 4 copies. *=p-val<0.05, ****=p-val<0.0001)

FIG. 155 shows increased CD8 infiltration following doublet and triplet blockade. Increased CD8 T-cell infiltration following treatment CHA.7.518.1.H4 (S241P)+nivolumab+/−BMS-986207 (Triplet and Doublet respectively). CD8 IHC stain (clone C8/144B) measured as precent positivity of total cells. 8/13 patients increased CD8 post treatment. Most prominent increase in patients with clinical benefit. And the triplet shows a trend for higher CD8 infiltration post treatment (Medinan change of 2.66 for triplet vs. 1.16 for doublet, non-significant). On treatment biopsy taken between C2D1 to C3D1.

FIG. 156 shows TME increases in TCR clones following doublet and triplet blockade in CB patients. Increased TCR clones following doublet and triplet blockade: increased T-cells clone number due to infiltration of new T-cell clones and expansion of existing T-cell clones in the TME. A) Vann diagram representing the pre-treatment number of unique clones CDR3 TCRβ (green circle) and On treatment number of unique clones (blue cycle), the overlap region represents the number of clones shared between Pre and On treatment. B) counts of top 5 unique CDR3 TCRβ clones On treatment vs. the counts Pre-treatment. On treatment biopsy taken between C2D1 to C3D1.

FIG. 157 shows potent immune stimulation in a patient with PR following CHA.7.518.1.H4 (S241P)+BMS-986207+nivolumab treatment. Extensive immune modulation in patient with clinical response (PR) flowing CHA.7.518.1.H4 (S241P)+nivolumab+BMS-986207 treatment. A) Density heat map of CD8 infiltration in pre (left) and On (right) treatment biopsy, demonstrates massive infiltration of CD8 T cells flowing treatment, as can be seen also by CD8 density per μm². B) The CD8 infiltration is also demonstrated by RNAseq data, by CD8 deconvolution score increase as well as by IFNg signature (upper left graph). The number of clones and T-cell clonality is also increased, as reflected in clone number count and Gini coefficient (right graph), both hallmarks of CD8 anti-tumor activity. MI macrophages deconvolution score increase and M2 macrophage score decreases flowing treatment (lower left graph). Image analysis using HALO® AI modules. RNAseq deconvolution using mySOR. On treatment biopsy taken between C2D1 to C3D1.

FIG. 158 Exome DNAseq was performed on 20 ovarian biopsies of patients treated either with CHA.7.518.1.H4 (S241P)+Nivolumab or CHA.7.518.1.H4 (S241P)+BMS-986207+nivolumab (NCT03667716 and NCT04570839). Out of the 20 sample, one responding patient (partial response by RECIST1.1, treated with CHA.7.518.1.H4 (S241P)+BMS-986207+nivolumab had genomic amplification of the PVRL2 locus (19q13.31/2), with 10-12 predicted copies. Pathological tumor H-score of PVRL2 IHC of the patient is 300, which is the maximal score by this evaluation.

FIG. 159 illustrates swimmer plots of the data regarding platinum resistant ovarian cancer treated with CHA.7.518.1.H4 (S241P) combinations.

FIG. 160 illustrates TME modulation following doublet (CHA.7.518.1.H4 (S241P)+nivolumab) and triplet (CHA.7.518.1.H4 (S241P)+nivolumab+BMS-986207) blockade in patients with ovarian cancer.

FIG. 161 illustrates that PVRL2 baseline levels correlates with clinical benefit (CB).

FIG. 162 illustrates PVRL2 genomic amplification observed in a patient with durable partial response following triplet (CHA.7.518.1.H4 (S241P)+nivolumab+BMS-986207) treatment.

FIG. 163 illustrates data regarding breast cancer treatment with combination of CHA.7.518.1.H4 (S241P) and nivolumab.

FIG. 164 illustrates serum IFNγ levels in response to breast cancer treatment with the combination of CHA.7.518.1.H4 (S241P) and nivolumab.

### DETAILED DESCRIPTION OF THE INVENTION

### I. Introduction

Cancer can be considered as an inability of the patient to recognize and eliminate cancerous cells. In many instances, these transformed (e.g., cancerous) cells counteract immunosurveillance. There are natural control mechanisms that limit T-cell activation in the body to prevent unrestrained T-cell activity, which can be exploited by cancerous cells to evade or suppress the immune response. Restoring the capacity of immune effector cells especially T cells—to recognize and eliminate cancer is the goal of immunotherapy. The field of immuno-oncology, sometimes referred to as “immunotherapy” is rapidly evolving, with several recent approvals of T cell checkpoint inhibitory antibodies such as Yervoy, Keytruda and Opdivo. These antibodies are generally referred to as “checkpoint inhibitors” because they block normally negative regulators of T cell immunity. It is generally understood that a variety of immunomodulatory signals, both costimulatory and coinhibitory, can be used to orchestrate an optimal antigen-specific immune response. Generally, these antibodies bind to checkpoint inhibitor proteins such as CTLA-4 and PD-1, which under normal circumstances prevent or suppress activation of cytotoxic T cells (CTLs). By inhibiting the checkpoint protein, for example through the use of antibodies that bind these proteins, an increased T cell response against tumors can be achieved. That is, these cancer checkpoint proteins suppress the immune response; when the proteins are blocked, for example using antibodies to the checkpoint protein, the immune system is activated, leading to immune stimulation, resulting in treatment of conditions such as cancer and infectious disease.

The present invention is directed to formulations comprising antibodies to human Poliovirus Receptor Related Immunoglobulin Domain Containing Protein, or “PVRIG”, sometimes also referred to herein as “PV protein”. PVRIG is expressed on the cell surface of NK and T-cells and shares several similarities to other known immune checkpoints.

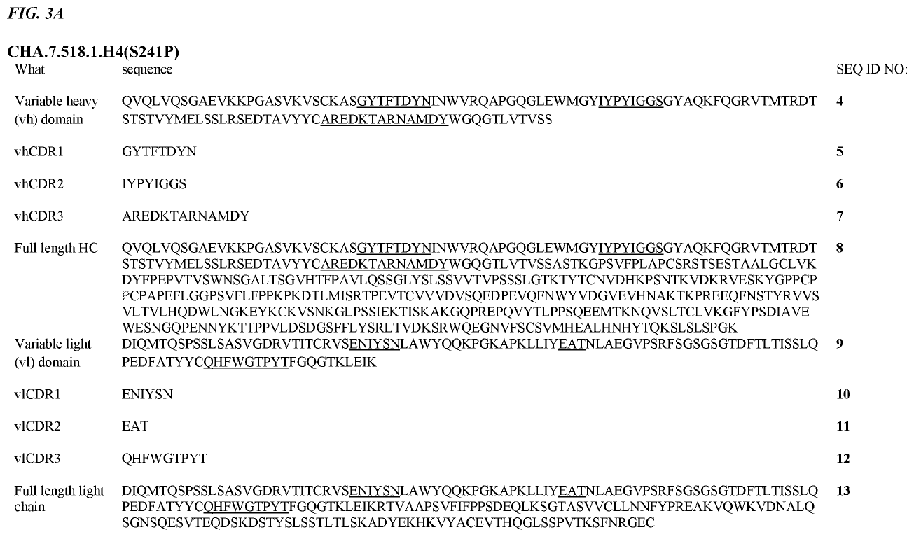

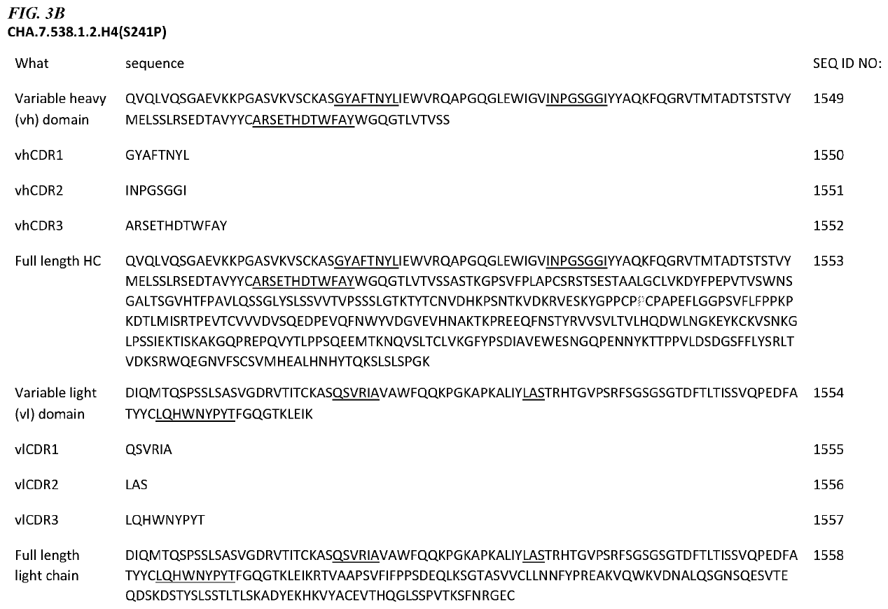

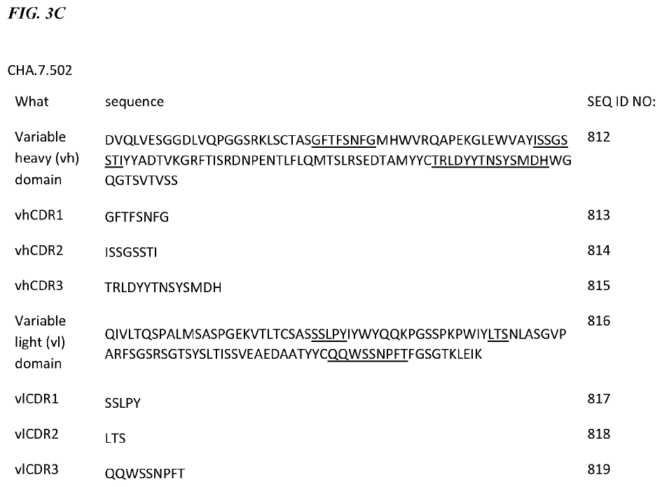

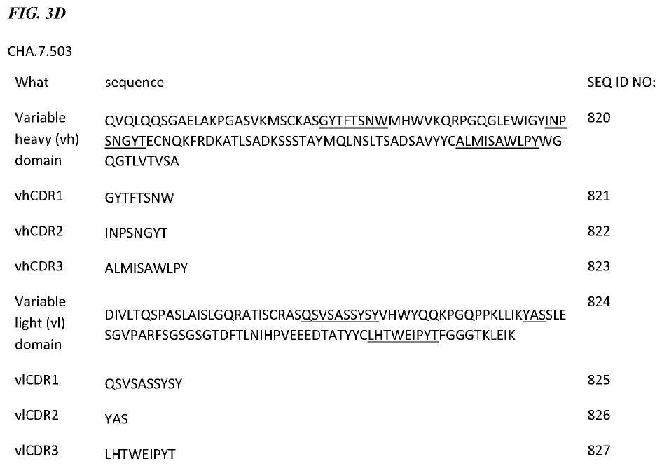

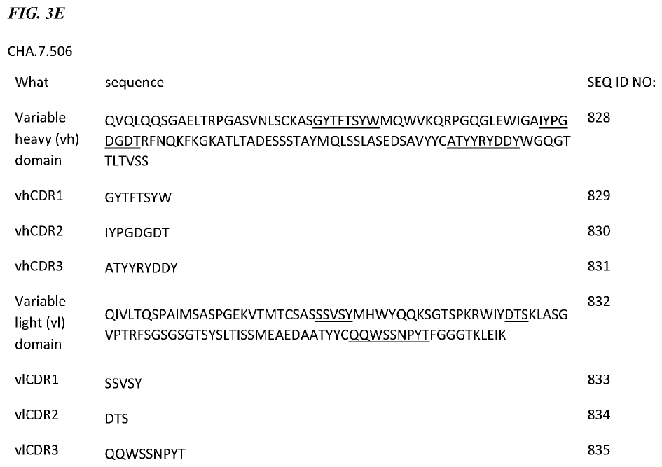

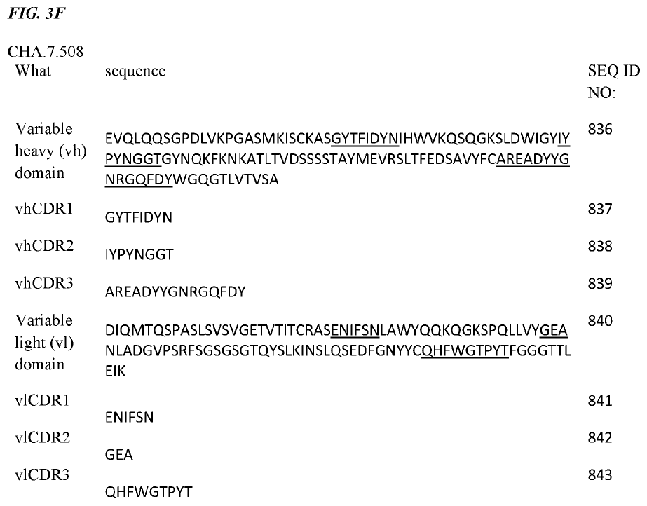

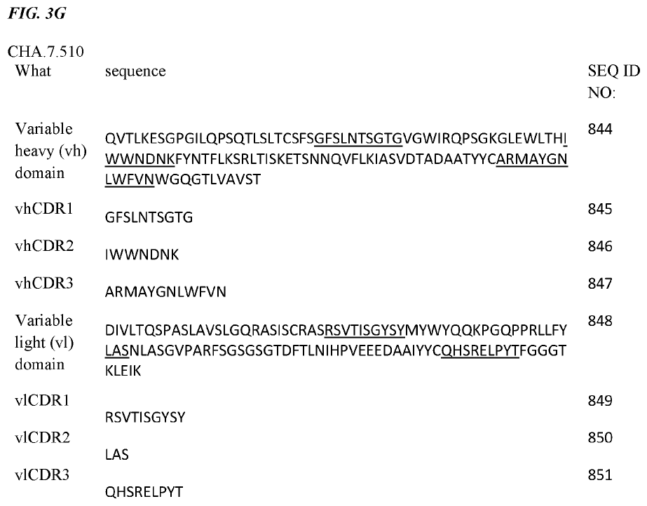

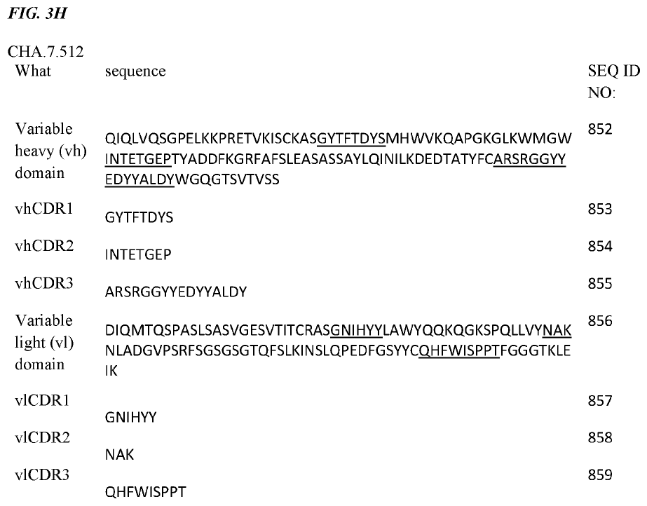

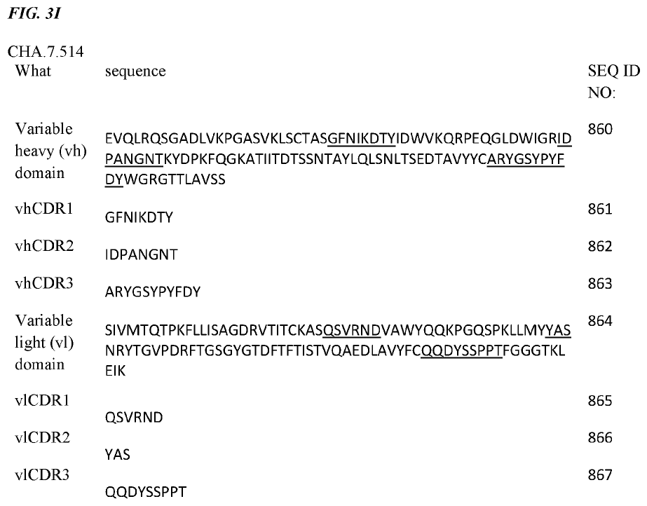

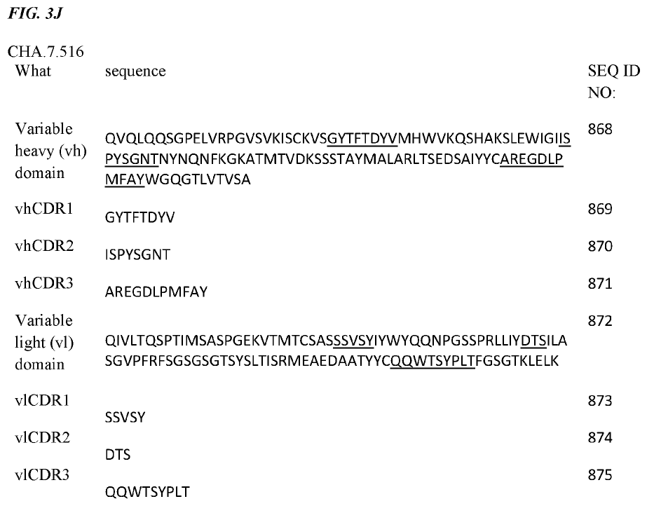

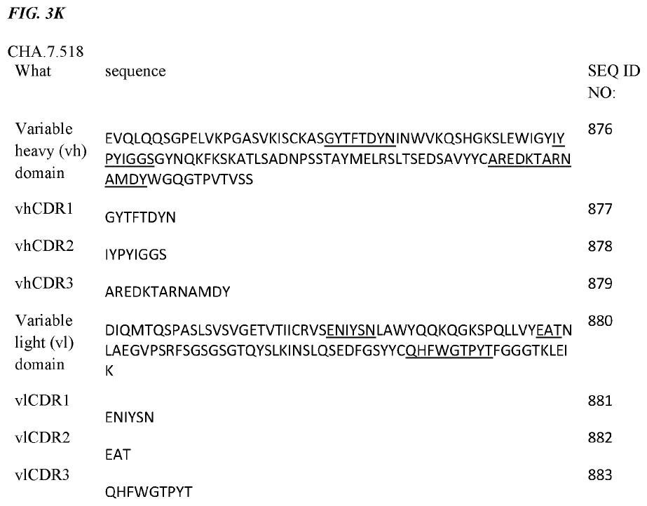

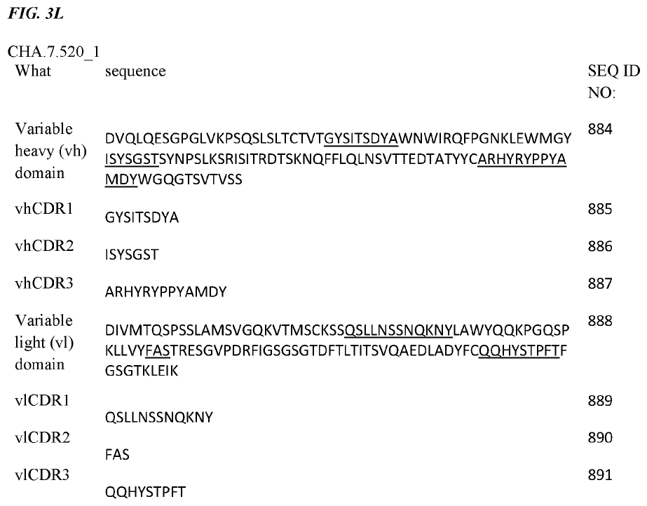

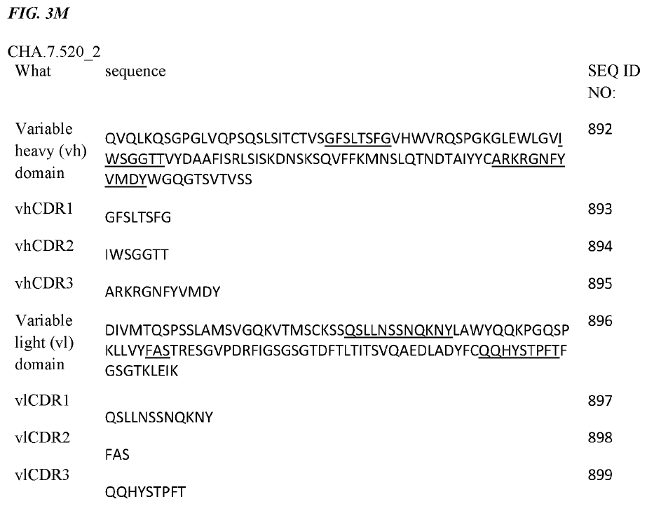

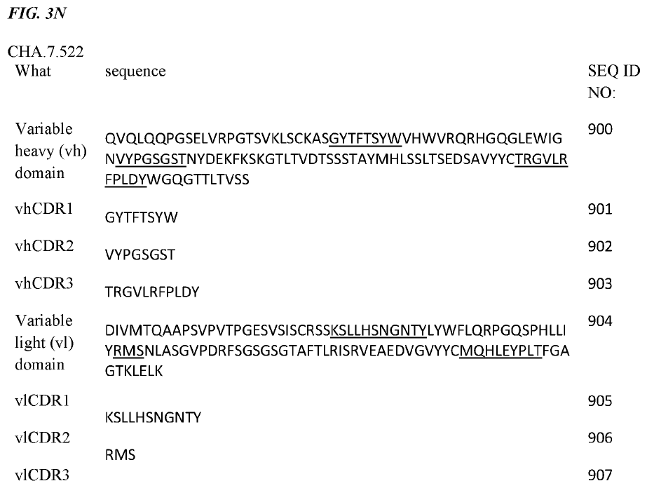

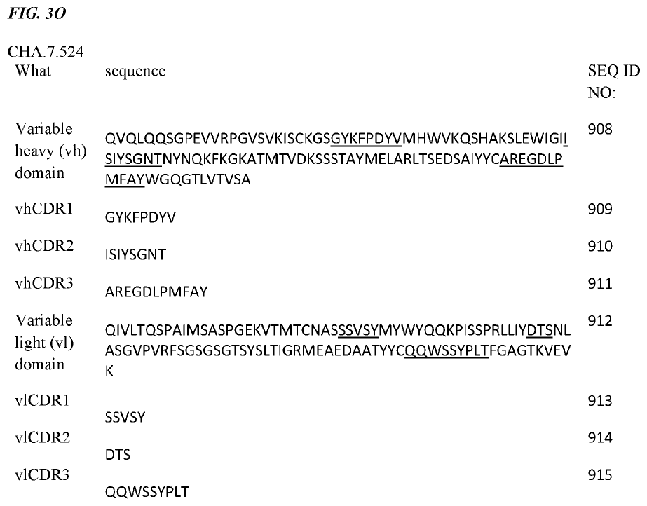

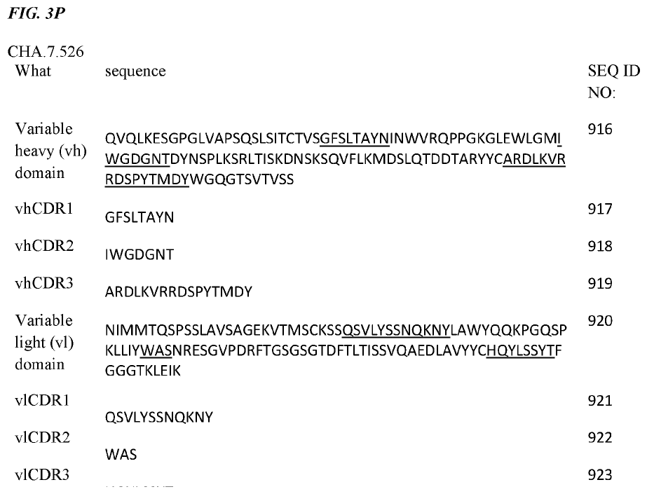

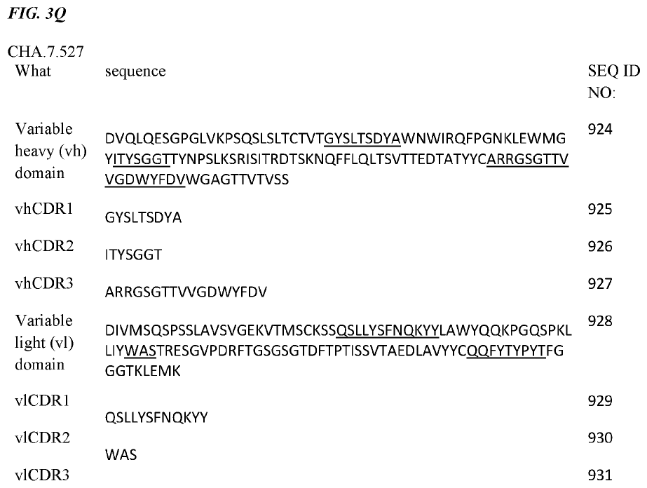

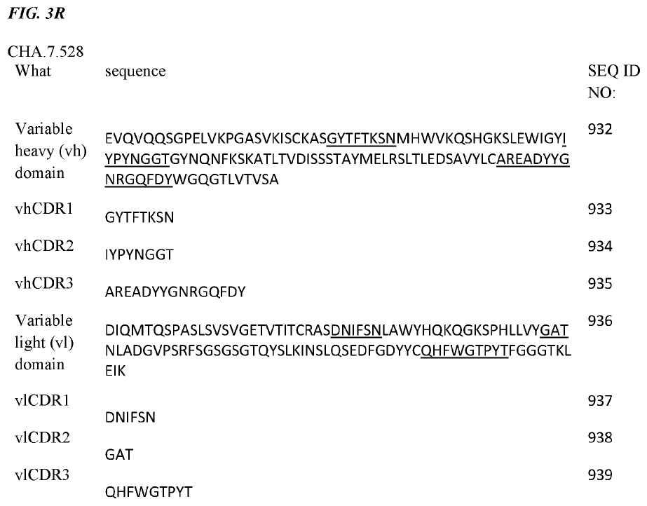

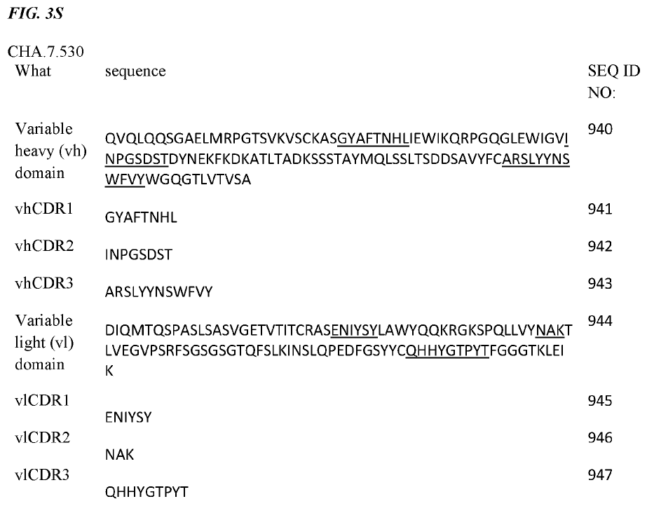

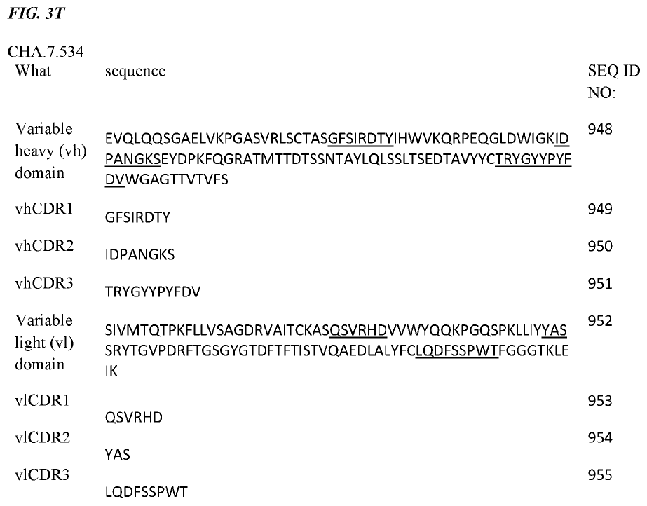

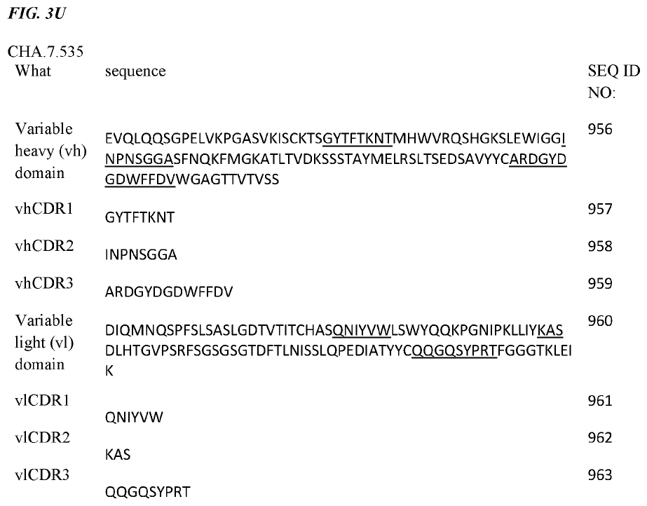

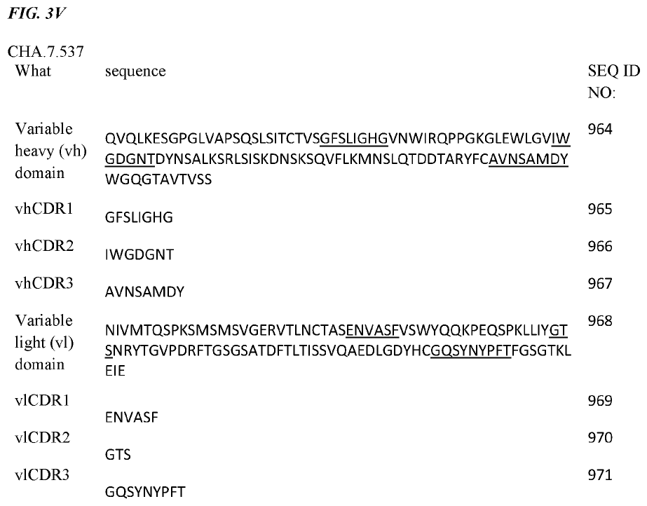

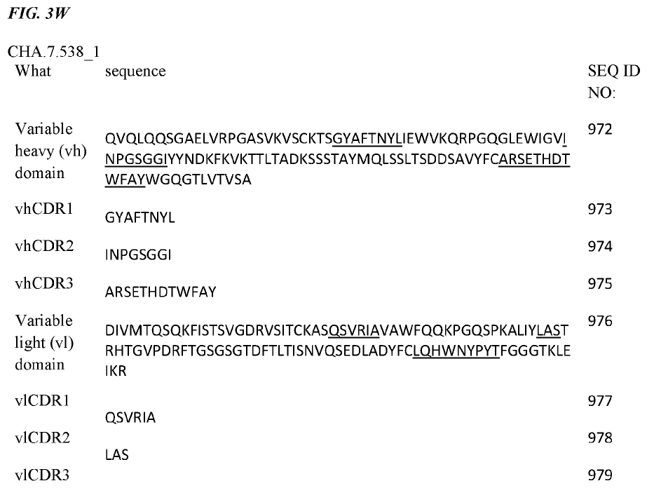

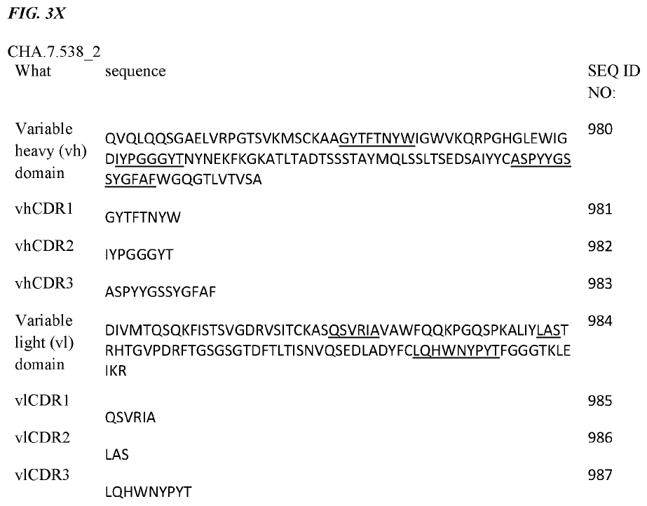

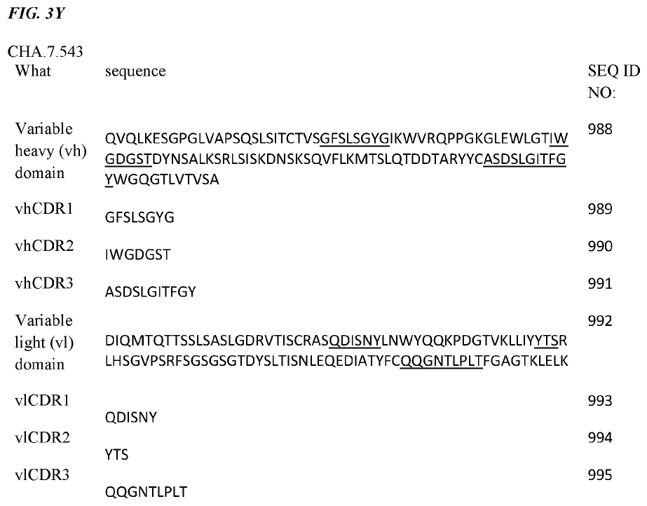

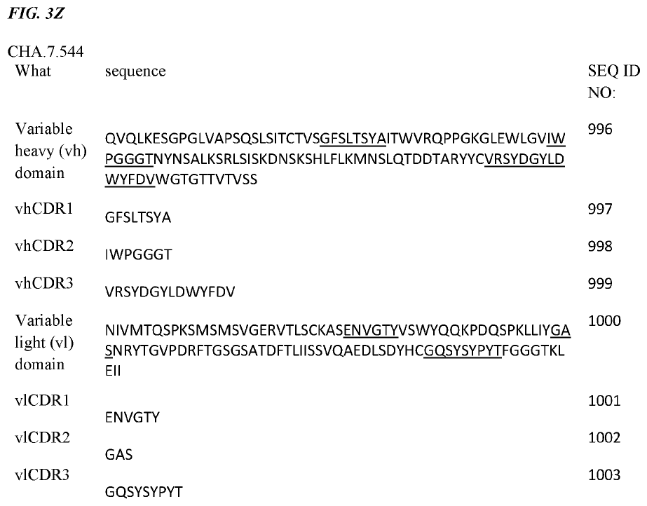

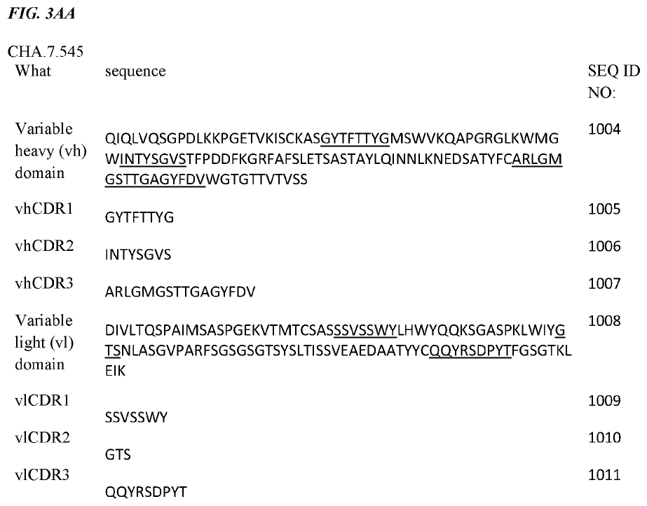

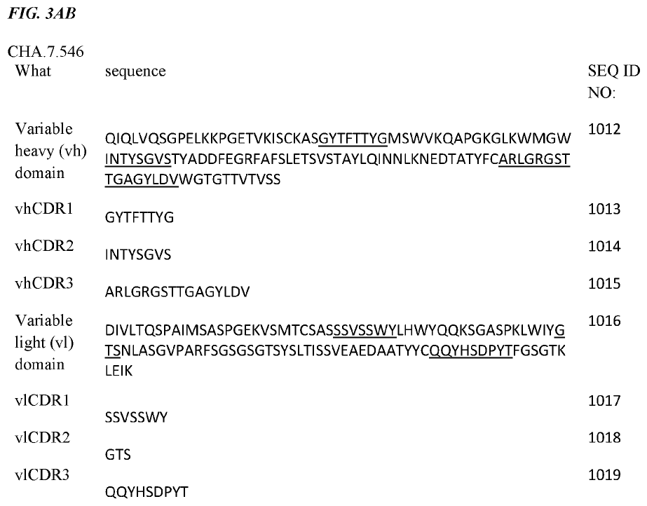

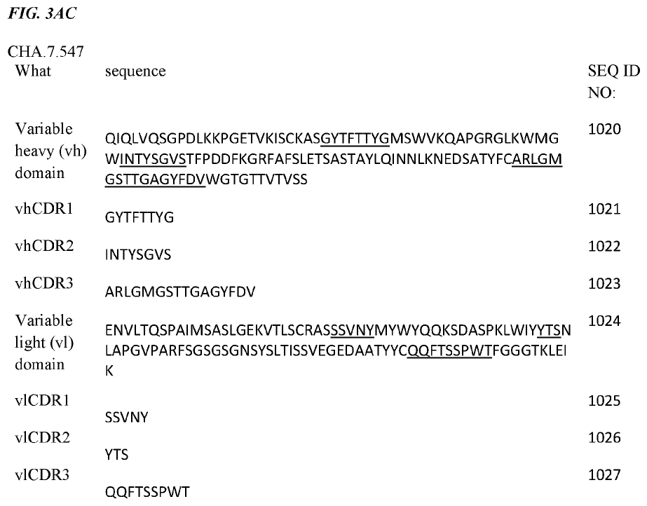

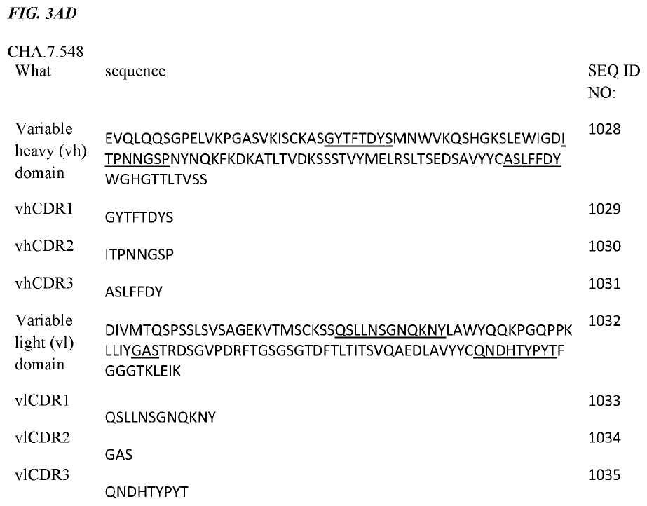

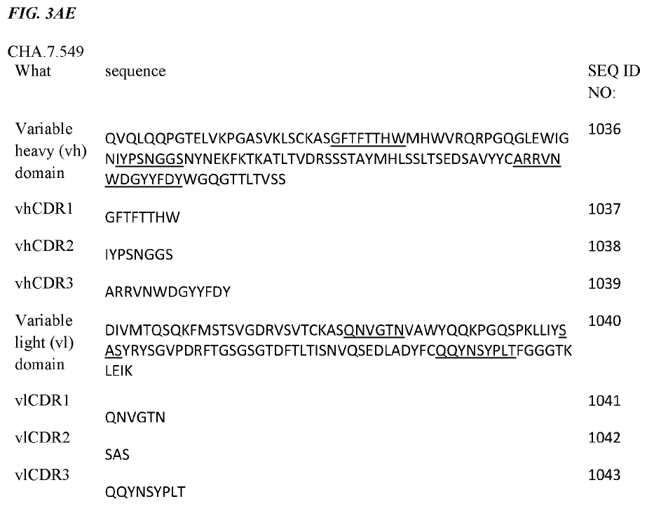

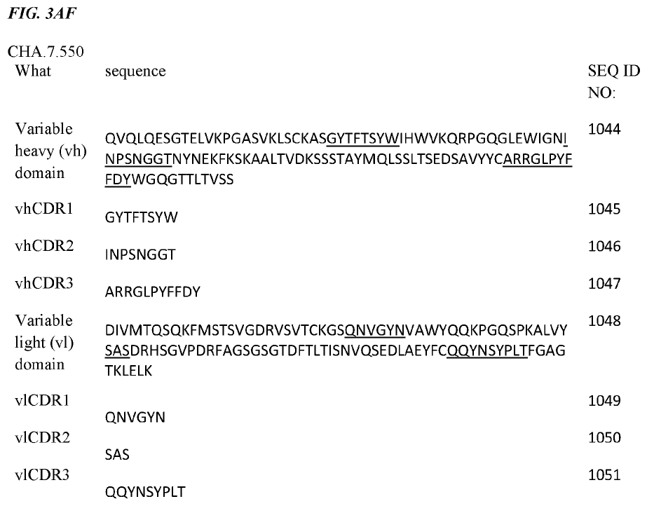

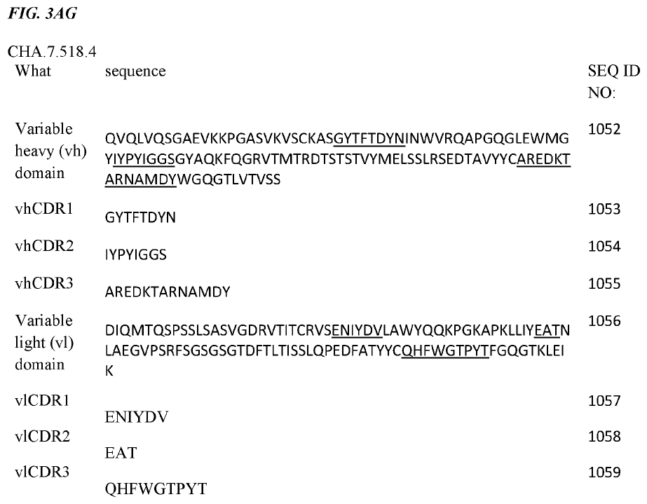

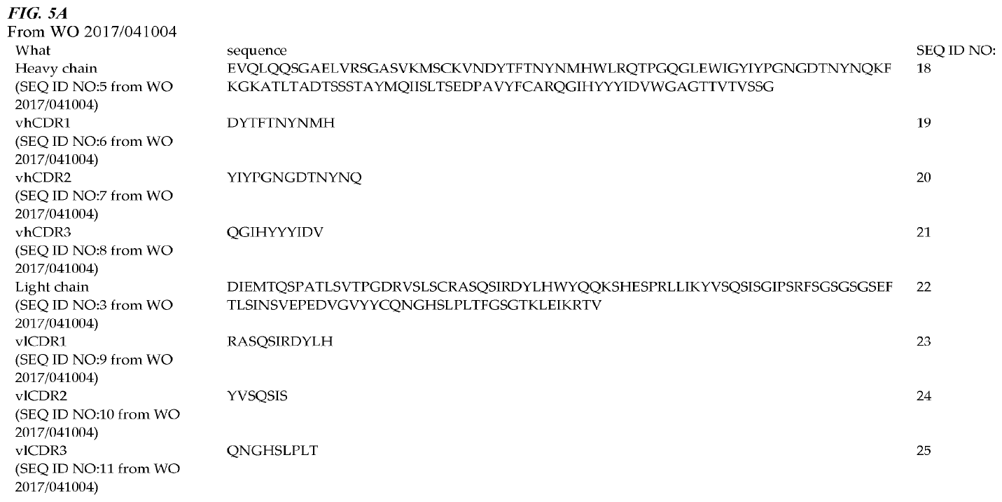

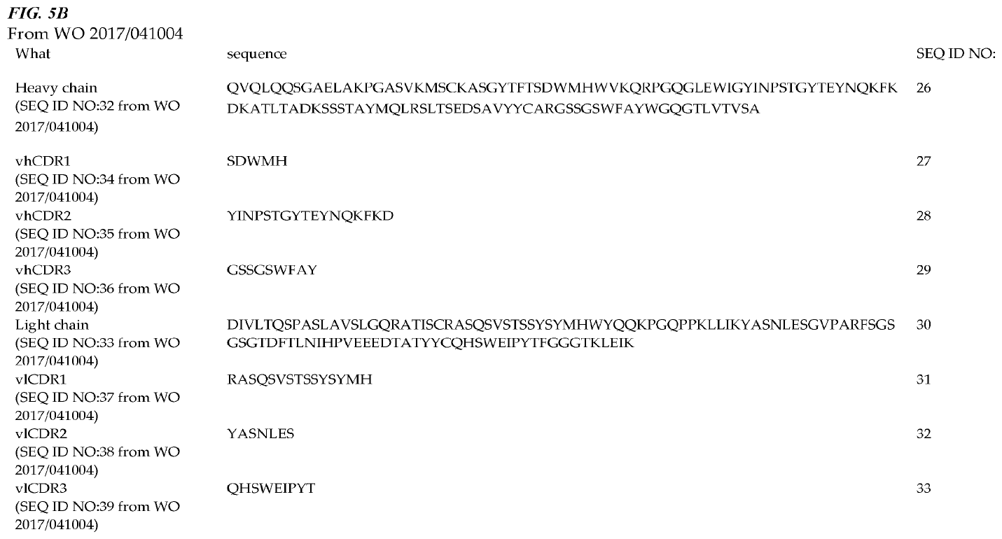

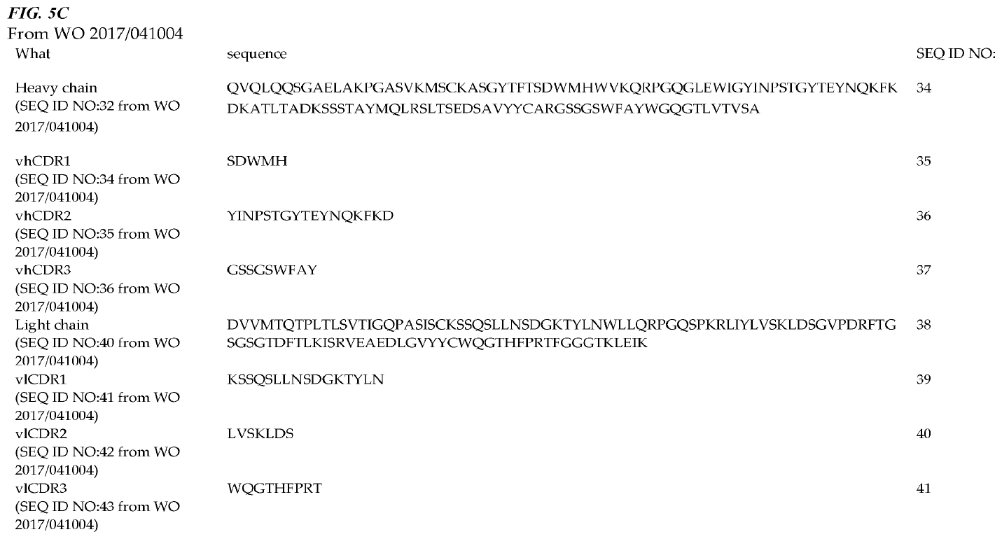

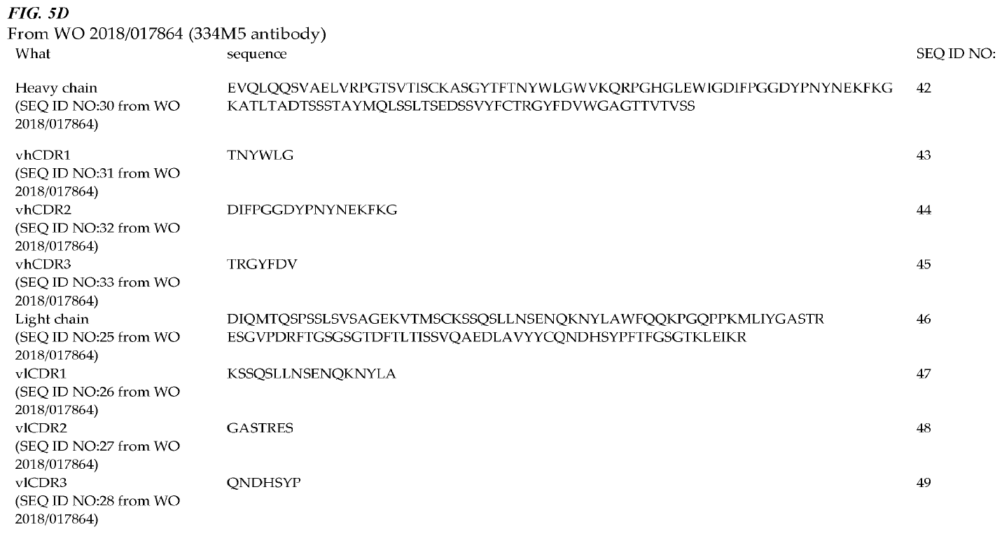

Accordingly, the present invention provides formulations comprising antibodies, including antigen binding domains, that bind to the human PVRIG and peptides thereof and methods of activating T cells and/or NK cells to treat diseases such as cancer and infectious diseases, and other conditions where increased immune activity results in treatment. In particular, the invention provides formulations comprising antibodies comprising heavy and light chains as well as the vhCDR1, vhCDR2, vhCDR3, vlCDR1, vlCDR2 and vlCDR3 sequences from CHA.7.518.1.H4 (S241P). In some embodiments, anti-PVRIG antibodies include those with CDRs identical to those shown in FIG. 3. In some embodiments, anti-PVRIG antibodies include those with CDRs identical to those shown in FIGS. 5A-5D, as well as anti-PVRIG antibodies comprising the heavy and light chains as provided in FIGS. 5A-5D.

### II. PVRIG Proteins

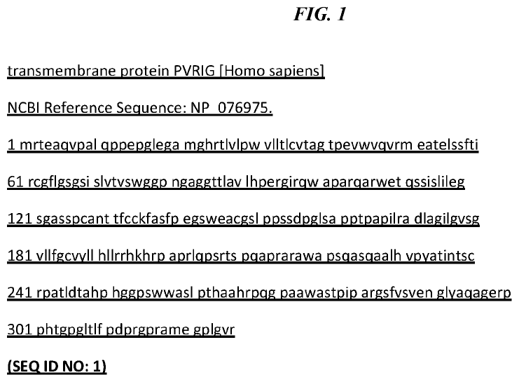

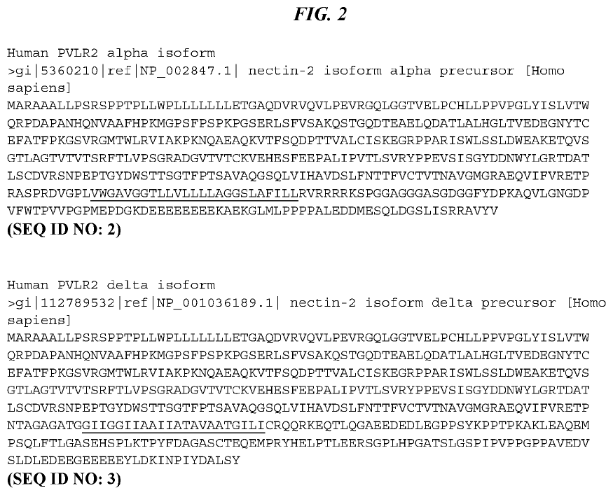

The present invention provides formulations comprising antibodies that specifically bind to PVRIG proteins. “Protein” in this context is used interchangeably with “polypeptide”, and includes peptides as well. The present invention provides antibodies that specifically bind to PVRIG proteins. PVRIG is a transmembrane domain protein of 326 amino acids in length, with a signal peptide (spanning from amino acid 1 to 40), an extracellular domain (spanning from amino acid 41 to 171), a transmembrane domain (spanning from amino acid 172 to 190) and a cytoplasmic domain (spanning from amino acid 191 to 326). The full length human PVRIG protein is shown in FIG. 1. There are two methionines that can be start codons, but the mature proteins are identical.

Accordingly, as used herein, the term “PVRIG” or “PVRIG protein” or “PVRIG polypeptide” may optionally include any such protein, or variants, conjugates, or fragments thereof, including but not limited to known or wild type PVRIG, as described herein, as well as any naturally occurring splice variants, amino acid variants or isoforms, and in particular the ECD fragment of PVRIG. The term “soluble” form of PVRIG is also used interchangeably with the terms “soluble ectodomain (ECD)” or “ectodomain” or “extracellular domain (ECD) as well as “fragments of PVRIG polypeptides”, which may refer broadly to one or more of the following optional polypeptides:

The PVRIG proteins contain an immunoglobulin (Ig) domain within the extracellular domain, which is a PVR-like Ig fold domain. The PVR-like Ig fold domain may be responsible for functional counterpart binding, by analogy to the other B7 family members. The PVR-like Ig fold domain of the extracellular domain includes one disulfide bond formed between intra domain cysteine residues, as is typical for this fold and may be important for structure-function. These cysteines are located at residues 22 and 93 (or 94). In one embodiment, there is provided a soluble fragment of PVRIG that can be used in testing of PVRIG antibodies. Included within the definition of PVRIG proteins are PVRIG ECD fragments, including know ECD fragments such as those described in U.S. Pat. No. 9,714,289, incorporate by reference herein in its entirety for all purposes.

As noted herein and more fully described below, the anti-PVRIG antibodies (including antigen-binding fragments) that both bind to PVRIG and prevent activation by PVRL2 (e.g., most commonly by blocking the interaction of PVRIG and PVLR2), are used to enhance T cell and/or NK cell activation and be used in treating diseases such as cancer and pathogen infection.

### III. TIGIT Proteins

The present invention provides antibodies that specifically bind to TIGIT proteins and prevent activation by its ligand protein, PVR, poliovirus receptor (aka, CD155) a human plasma membrane glycoprotein. TIGIT, or T cell immunoreceptor with Ig and ITIM domains, is a co-inhibitory receptor protein also known as WUCAM, Vstm3 or Vsig9. TIGIT has an immunoglobulin variable domain, a transmembrane domain, and an immunoreceptor tyrosine-based inhibitory motif (ITIM) and contains signature sequence elements of the PVR protein family. The extracellular domain (ECD) sequences of TIGIT and of PVR are shown in FIG. 1B. The antibodies of the invention are specific for the TIGIT ECD such that the binding of TIGIT and PVR is blocked

Accordingly, as used herein, the term “TIGIT” or “TIGIT protein” or “TIGIT polypeptide” may optionally include any such protein, or variants, conjugates, or fragments thereof, including but not limited to known or wild type TIGIT, as described herein, as well as any naturally occurring splice variants, amino acid variants or isoforms, and in particular the ECD fragment of TIGIT.

As noted herein and more fully described below, anti-TIGIT antibodies (including antigen-binding fragments) that both bind to TIGIT and prevent activation by PVR (e.g., most commonly by blocking the interaction of TIGIT and PVR), are used to enhance T cell and/or NK cell activation and be used in treating diseases such as cancer and pathogen infection.

### IV. Antibodies

Accordingly, the invention provides anti-PVRIG antibodies that can be formulated according to the formulations described herein and which are provided in FIG. 3 (e.g., including anti-PVRIG antibodies including those with CDRs identical to those shown in FIG. 3). PVRIG, also called Poliovirus Receptor Related Immunoglobulin Domain Containing Protein, Q6DKI7 or C7orf15, relates to amino acid and nucleic acid sequences shown in RefSeq accession identifier NP_076975, shown in FIG. 1. The antibodies of the invention are specific for the PVRIG extracellular domain.

As is discussed below, the term “antibody” is used generally. Antibodies that find use in the present invention can take on a number of formats as described herein, including traditional antibodies as well as antibody derivatives, fragments and mimetics, described below. In general, the term “antibody” includes any polypeptide that includes at least one antigen binding domain, as more fully described below. Antibodies may be polyclonal, monoclonal, xenogeneic, allogeneic, syngeneic, or modified forms thereof, as described herein, with monoclonal antibodies finding particular use in many embodiments. In some embodiments, antibodies of the invention bind specifically or substantially specifically to PVRIG molecules. The terms “monoclonal antibodies” and “monoclonal antibody composition”, as used herein, refer to a population of antibody molecules that contain only one species of an antigen-binding site capable of immunoreacting with a particular epitope of an antigen, whereas the term “polyclonal antibodies” and “polyclonal antibody composition” refer to a population of antibody molecules that contain multiple species of antigen-binding sites capable of interacting with a particular antigen. A monoclonal antibody composition, typically displays a single binding affinity for a particular antigen with which it immunoreacts.

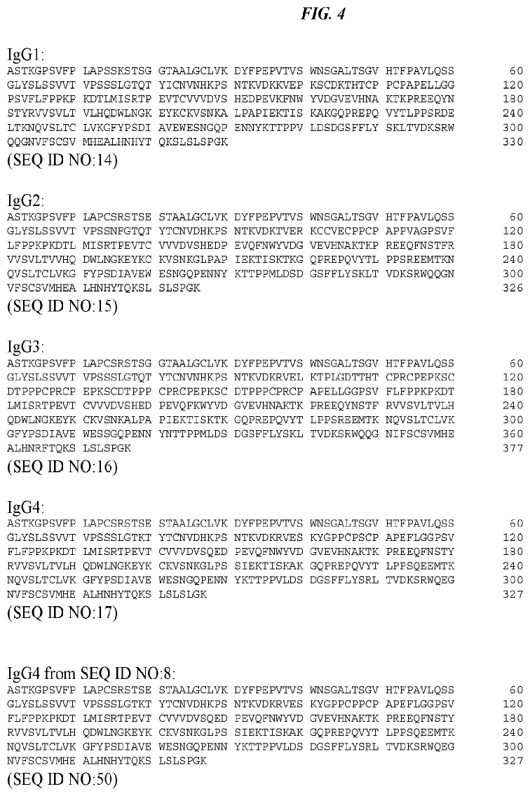

Traditional full length antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. The present invention is directed to the IgG class, which has several subclasses, including, but not limited to IgG1, IgG2, IgG3, and IgG4. Thus, “isotype” as used herein is meant any of the subclasses of immunoglobulins defined by the chemical and antigenic characteristics of their constant regions. While the exemplary antibodies herein designated “CPA” are based on IgG1 heavy constant regions, as shown in FIG. 4, the anti-PVRIG antibodies of the invention include those using IgG2, IgG3 and IgG4 sequences, or combinations thereof. For example, as is known in the art, different IgG isotypes have different effector functions which may or may not be desirable. Accordingly, the CPA antibodies of the invention can also swap out the IgG1 constant domains for IgG2, IgG3 or IgG4 constant domains (depicted in FIG. 4), with IgG2 and IgG4 finding particular use in a number of situations, for example for ease of manufacture or when reduced effector function is desired, the latter being desired in some situations.

The amino-terminal portion of each chain includes a variable region of about 100 to 110 or more amino acids primarily responsible for antigen recognition, generally referred to in the art and herein as the “Fv domain” or “Fv region”. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. “Variable” refers to the fact that certain segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions”.

Each VH and VL is composed of three hypervariable regions (“complementary determining regions,” “CDRs”) and four FRs, arranged from amino-terminus to carboxy-terminus in the following order: FR1-CDR1-FR2-CDR2-FR3-CDR3-FR4.

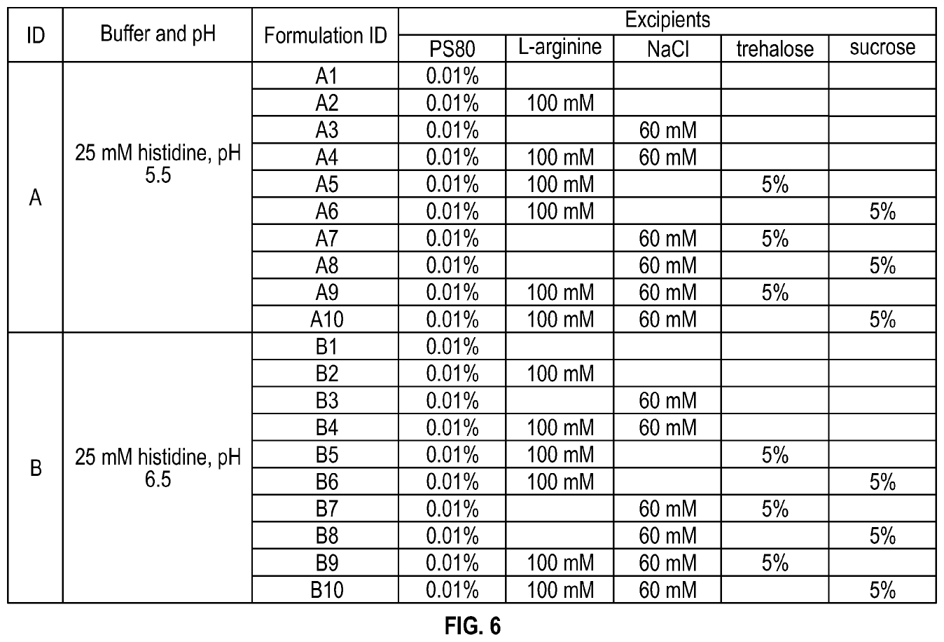

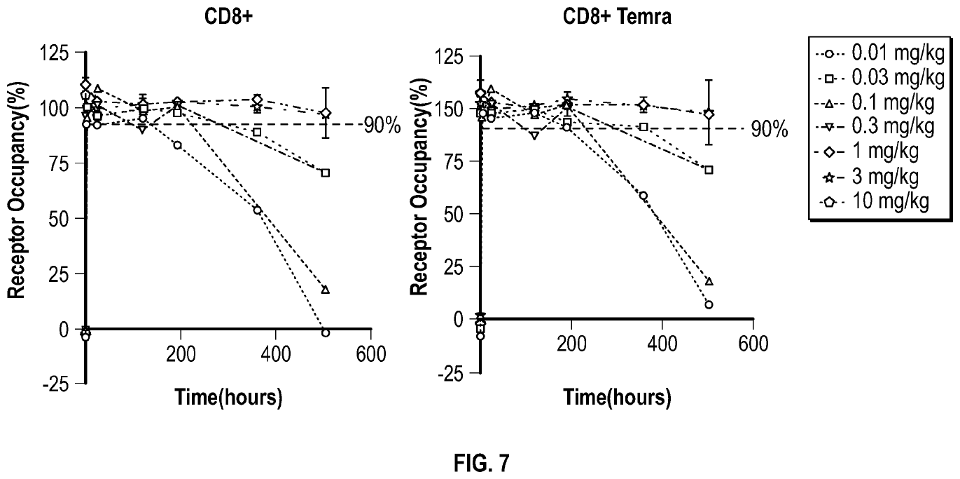

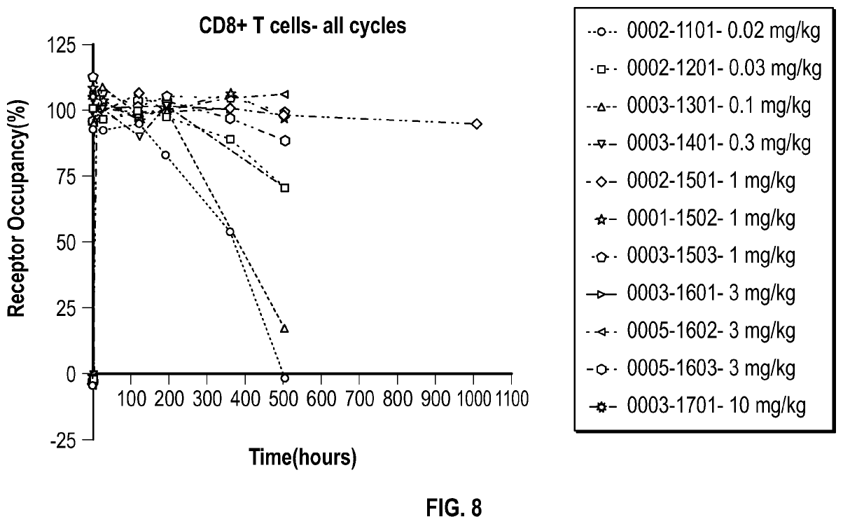

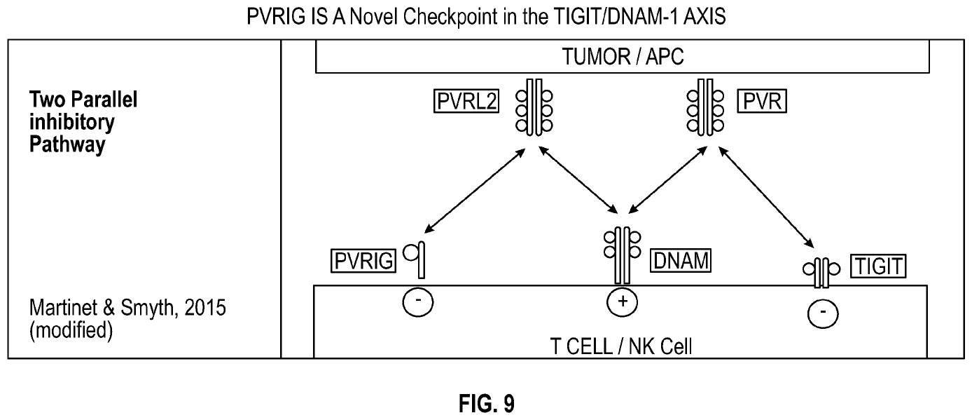

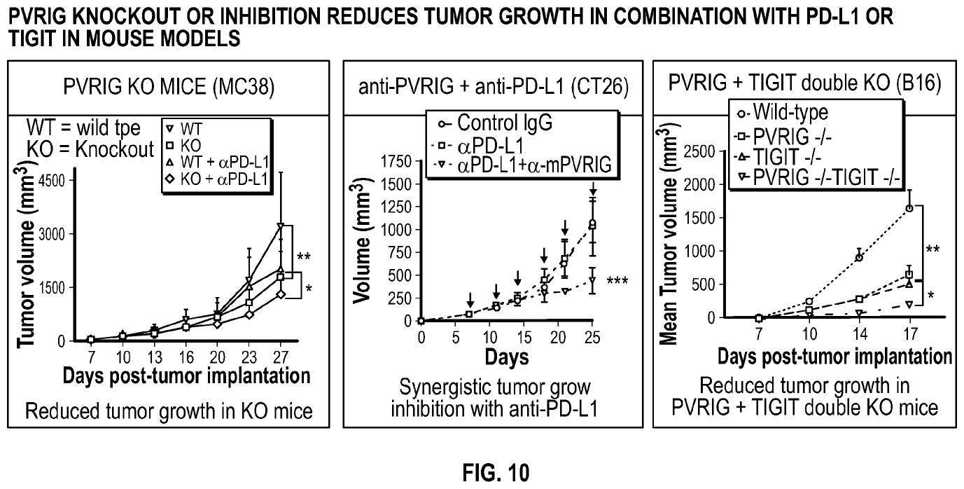

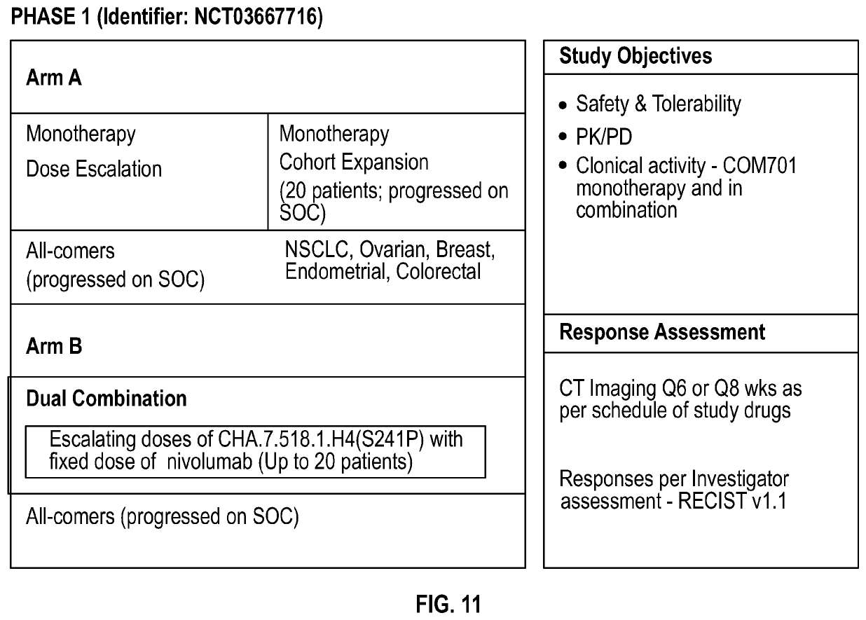

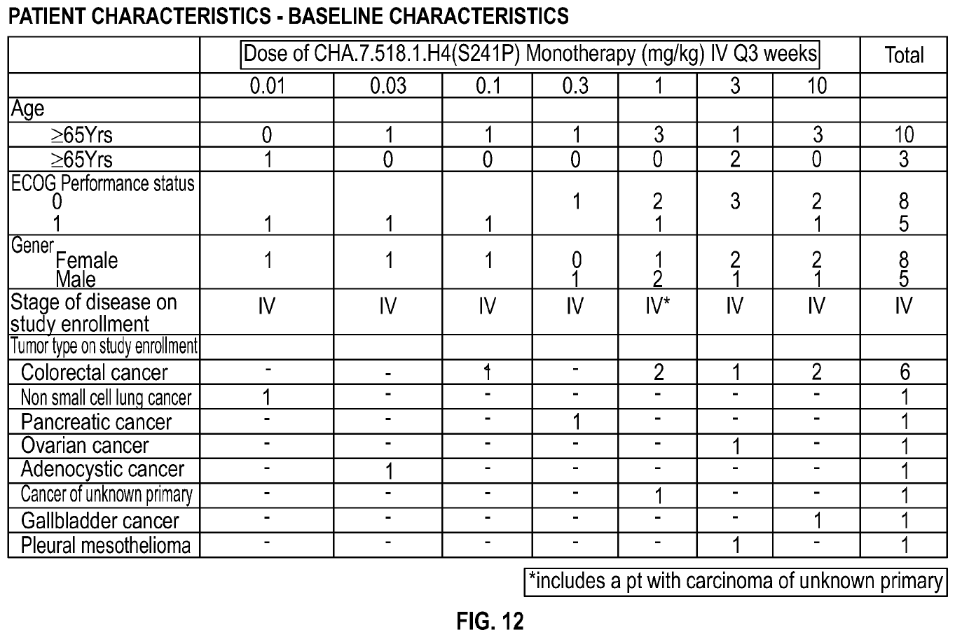

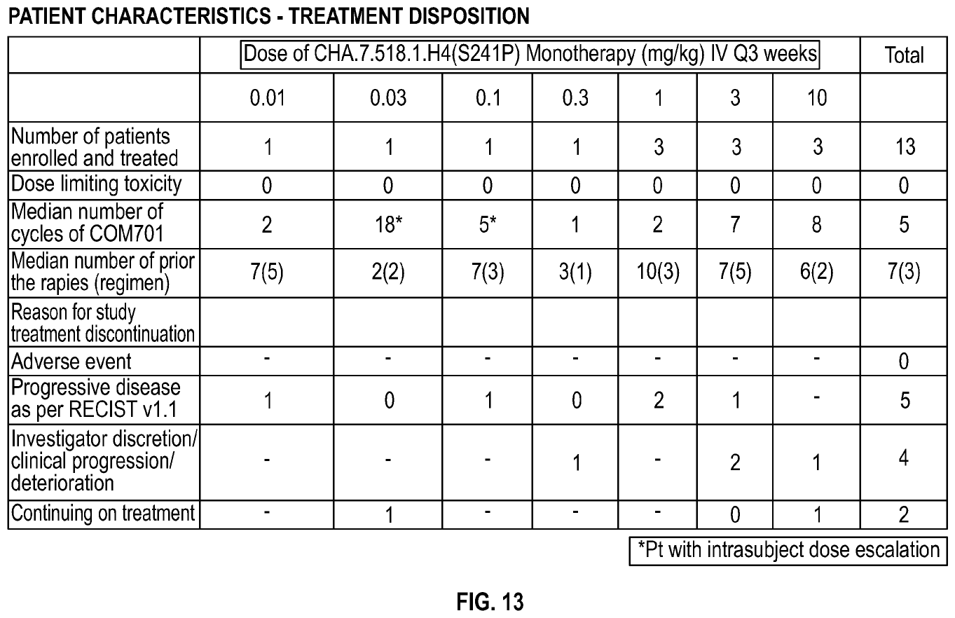

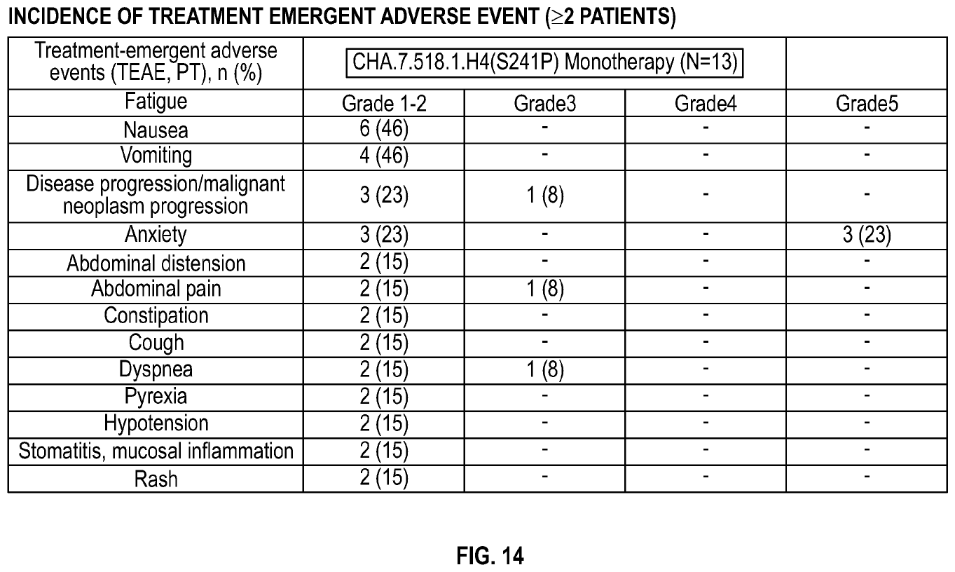

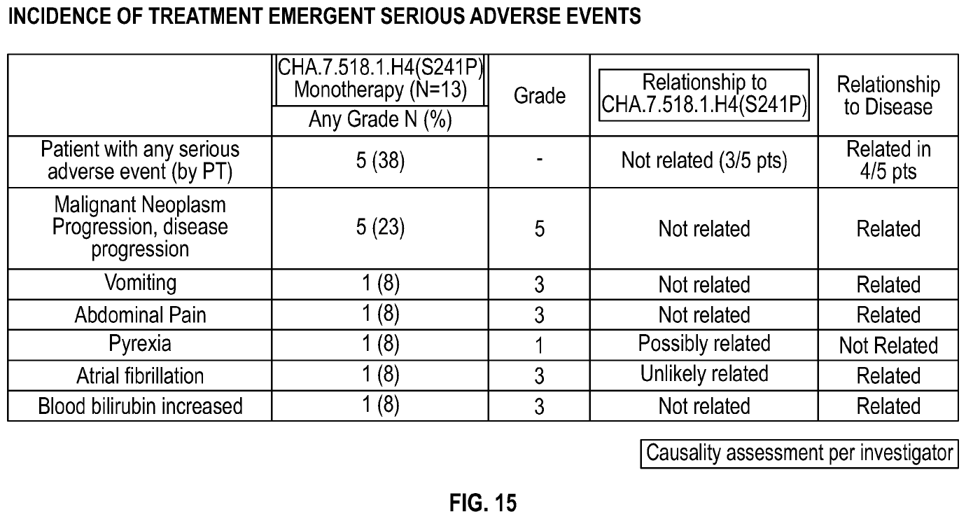

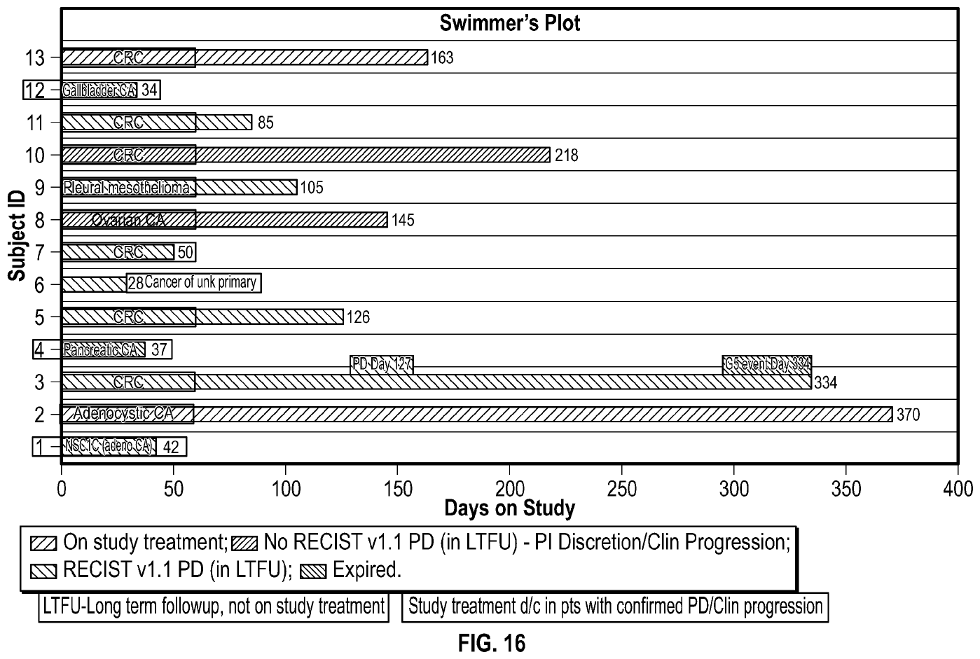

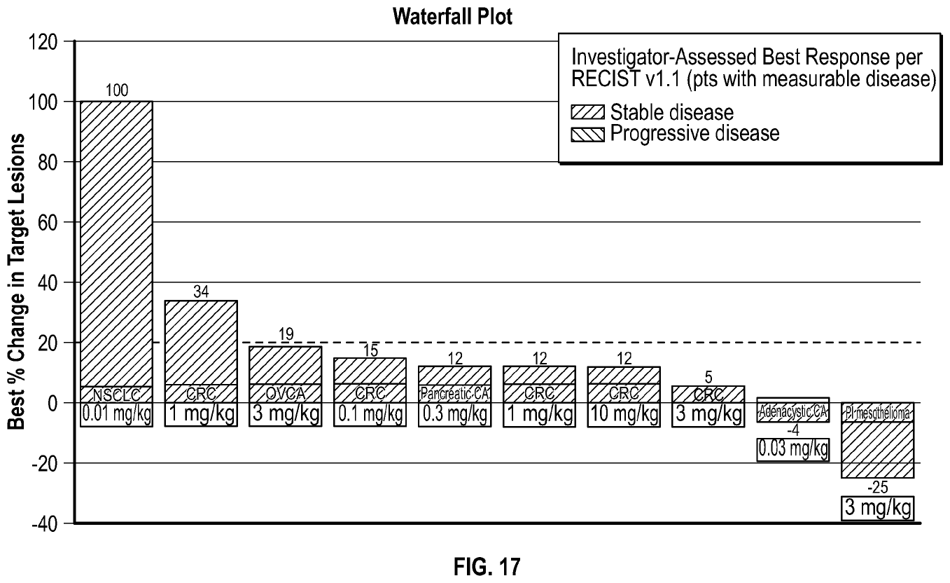

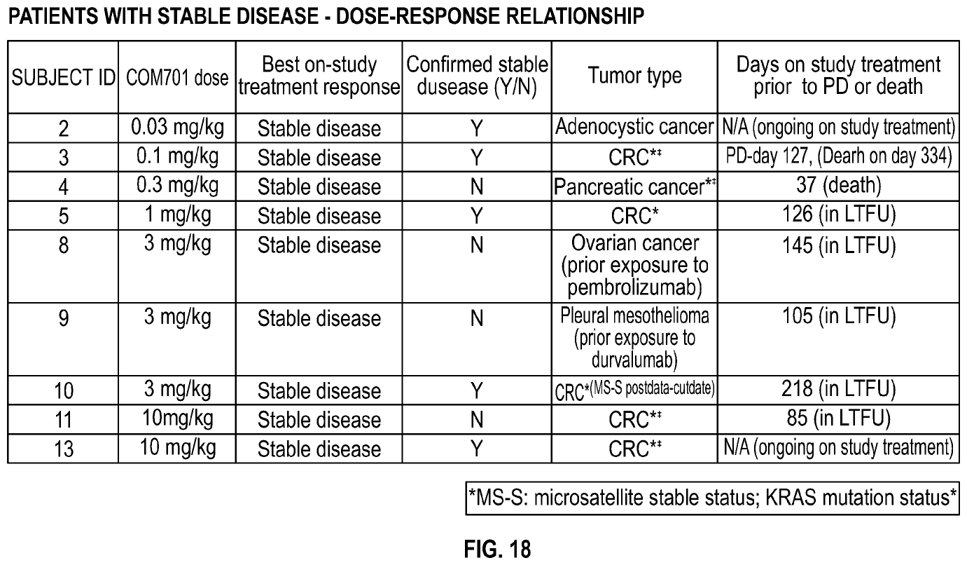

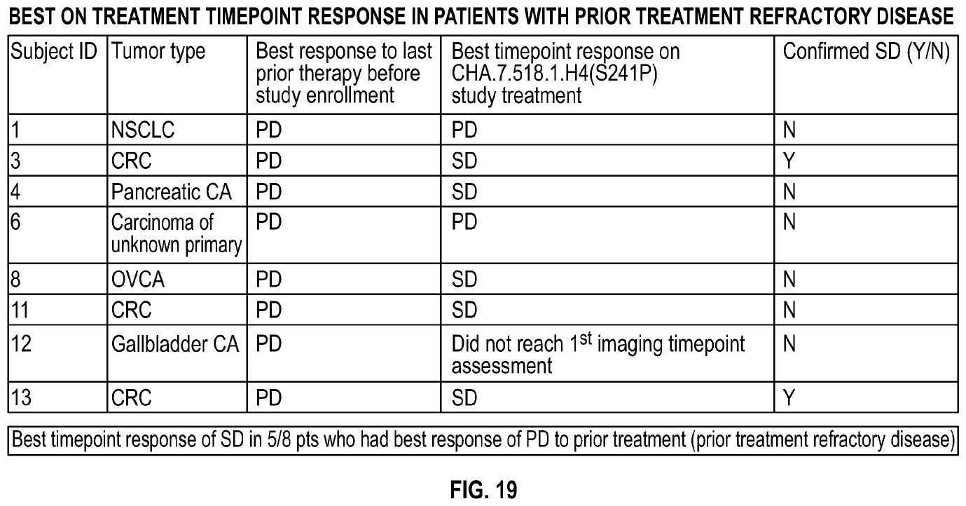

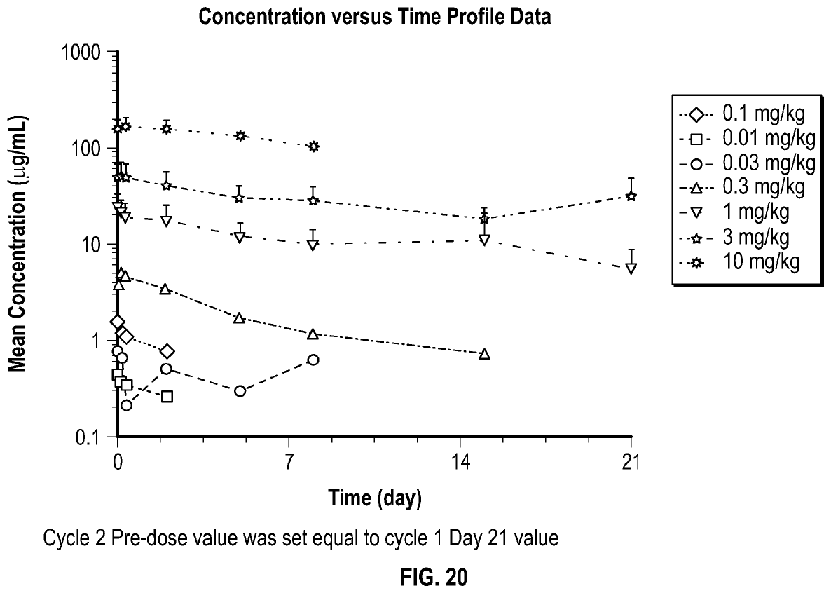

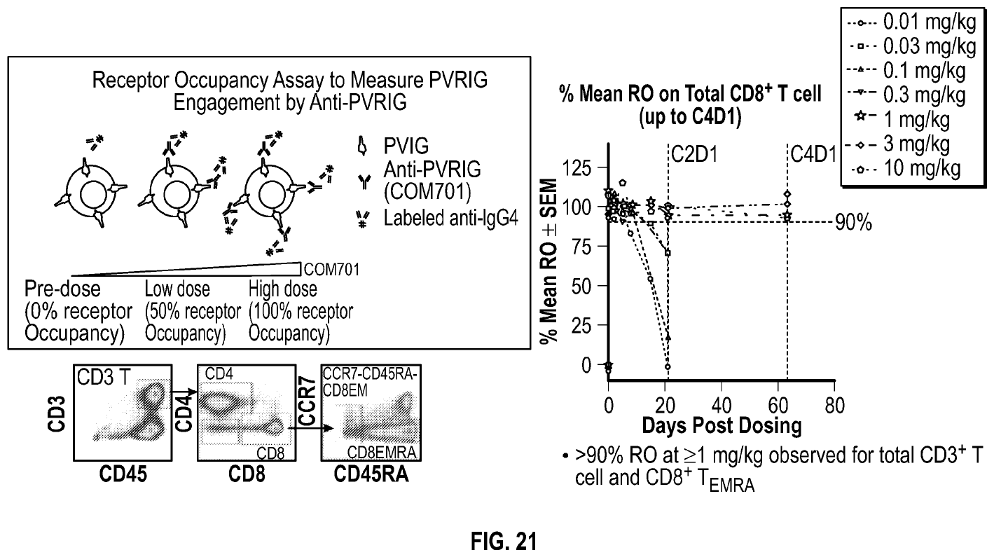

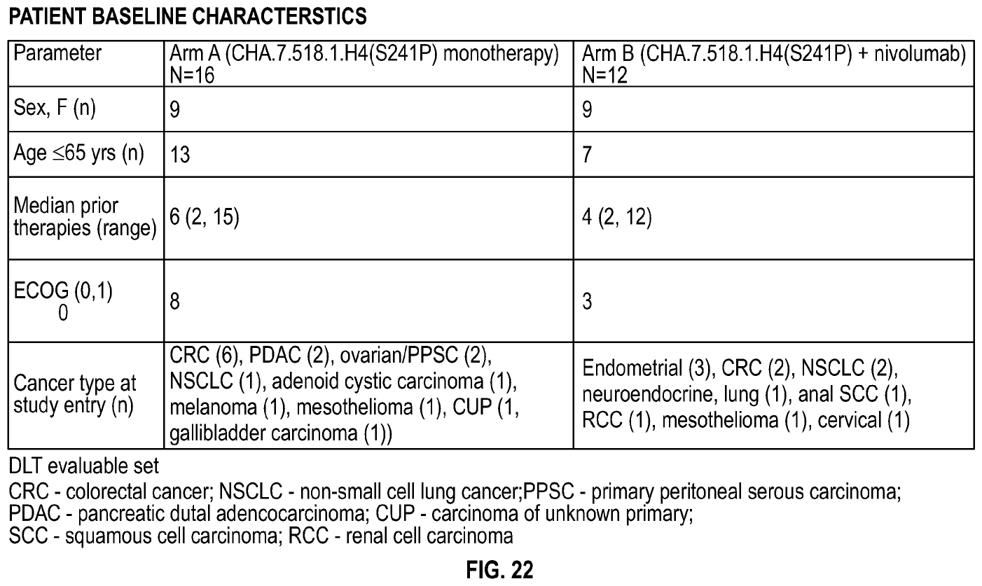

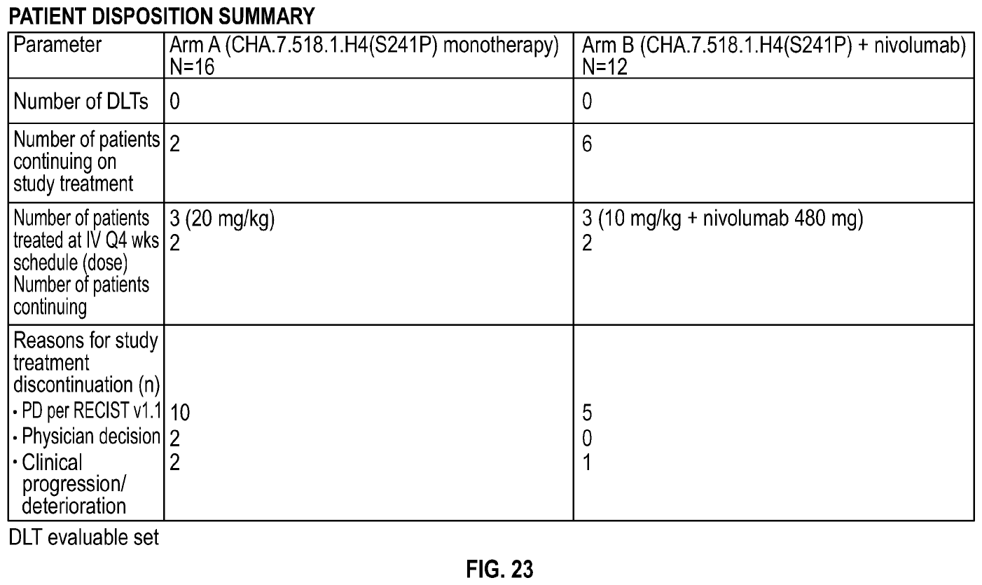

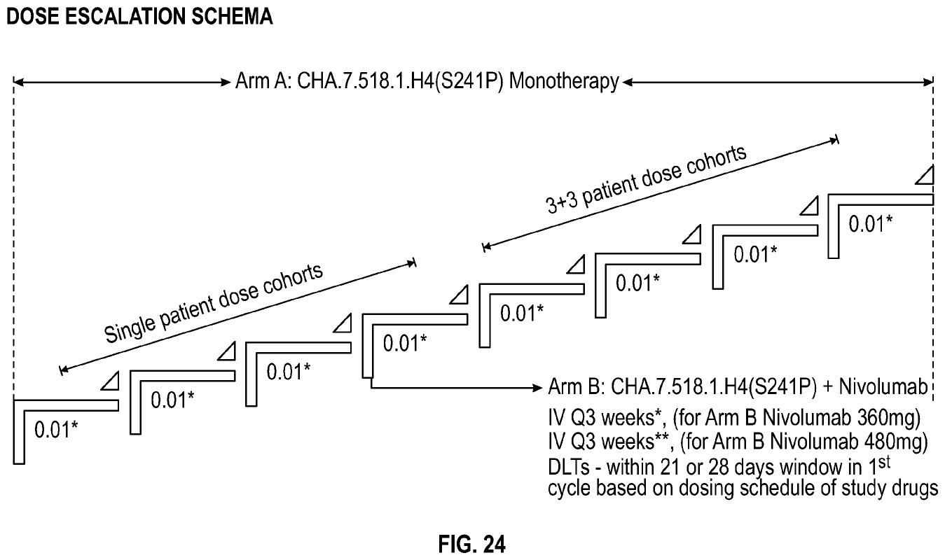

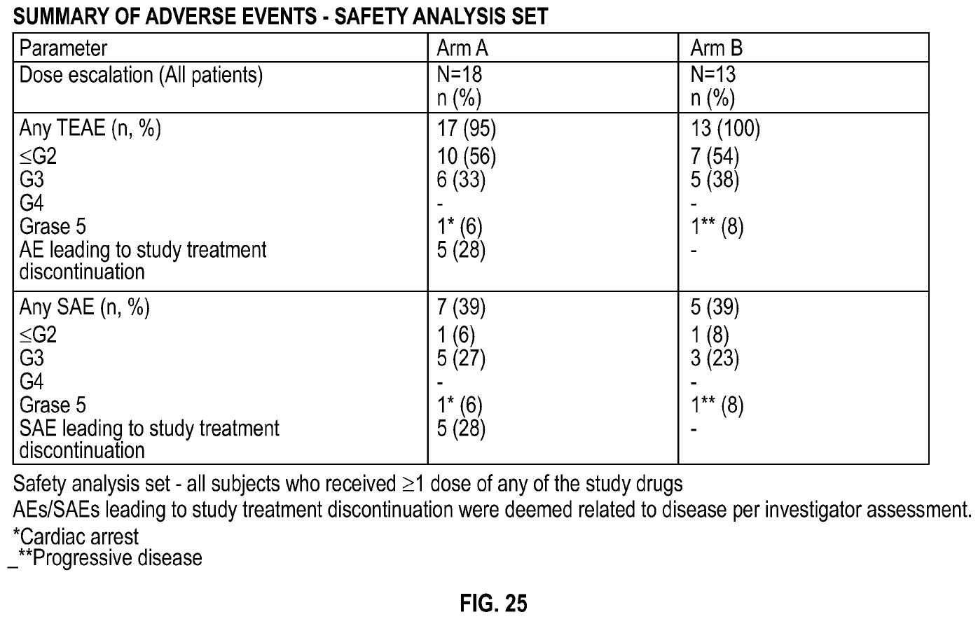

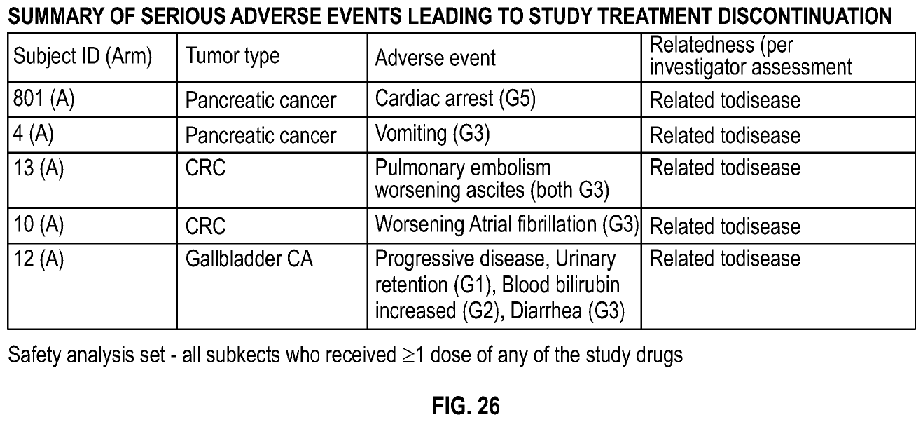

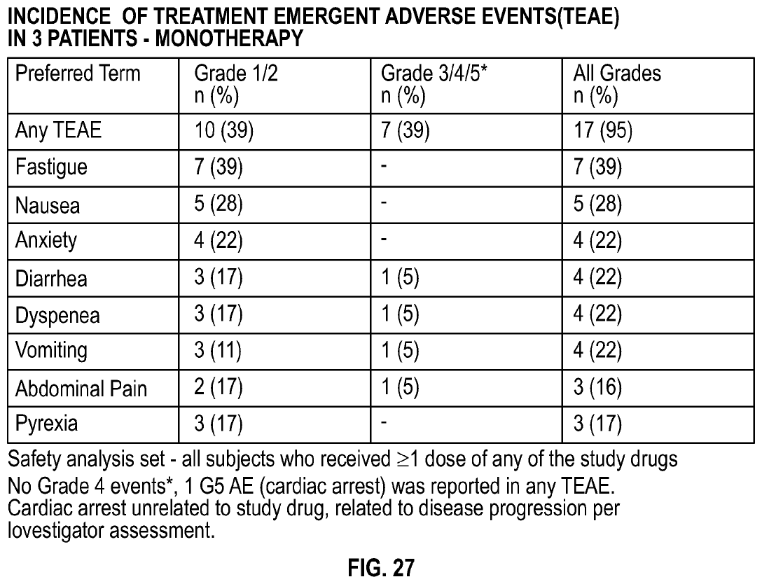

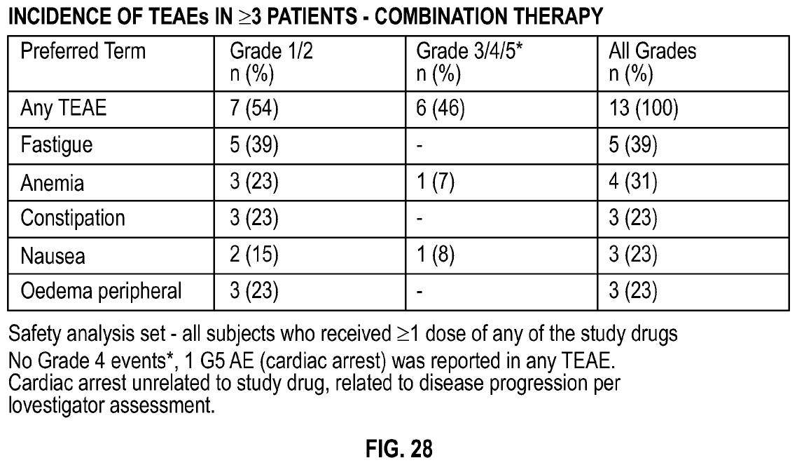

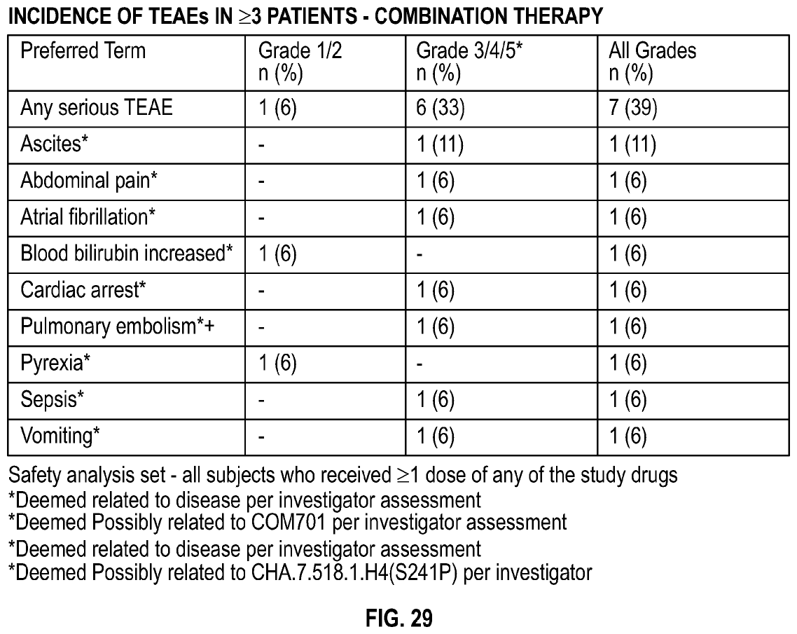

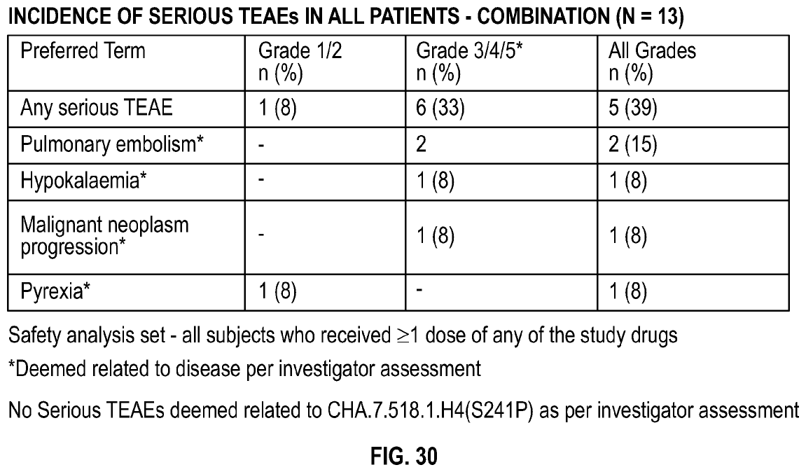

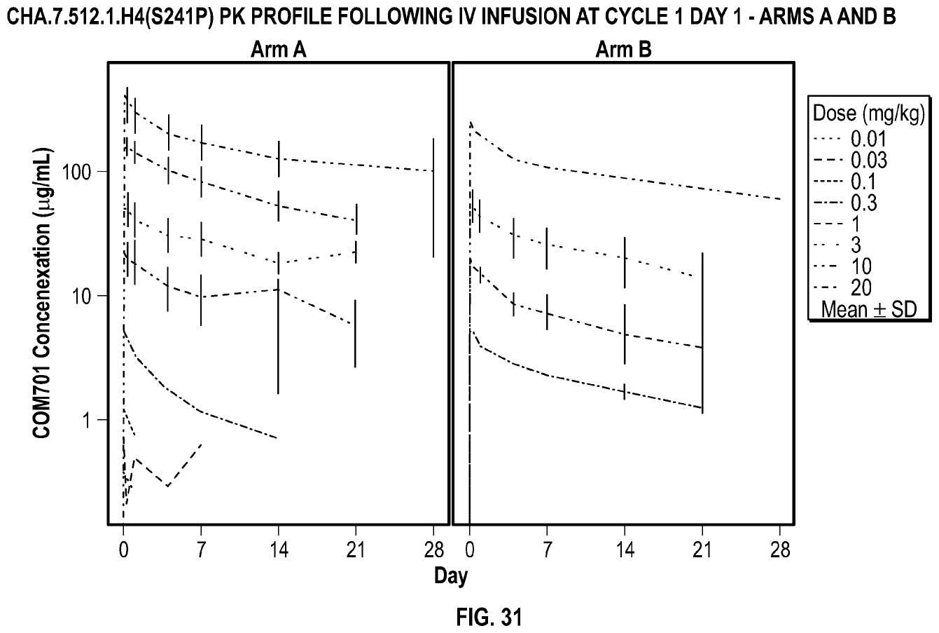

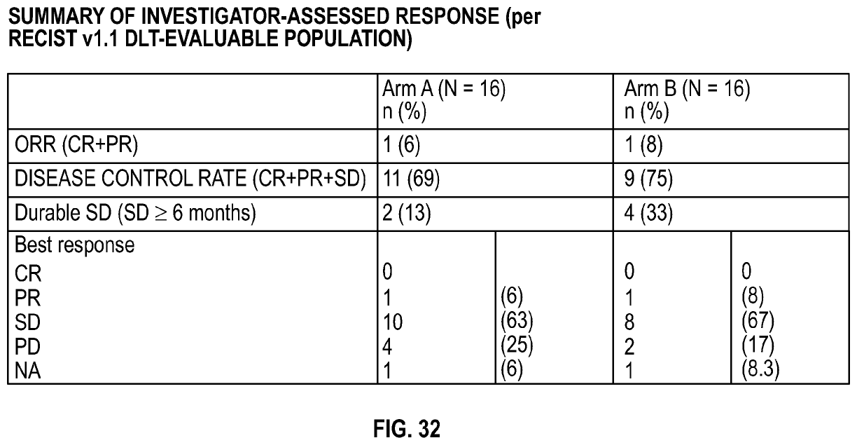

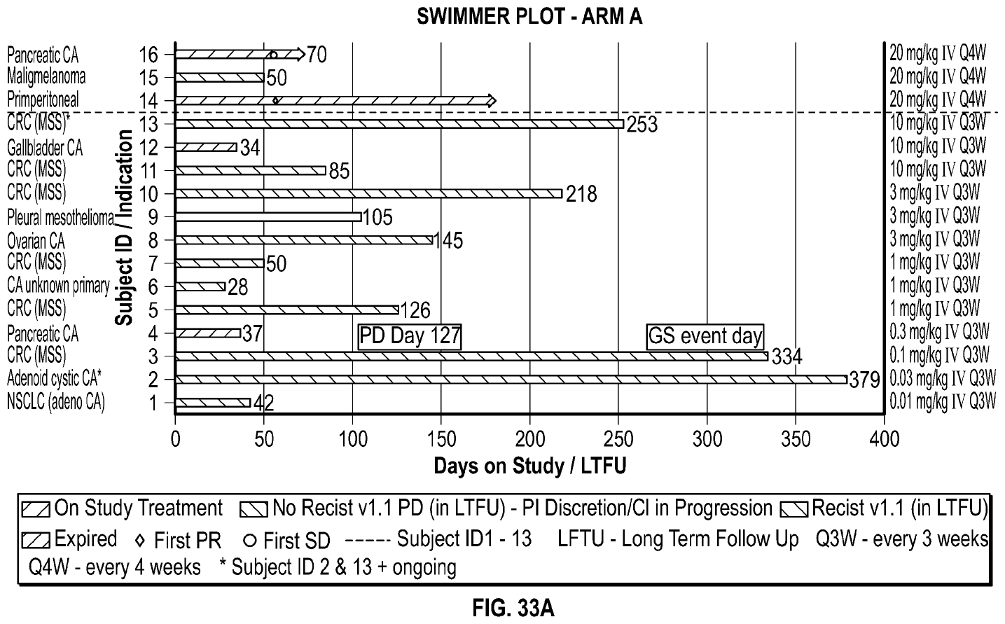

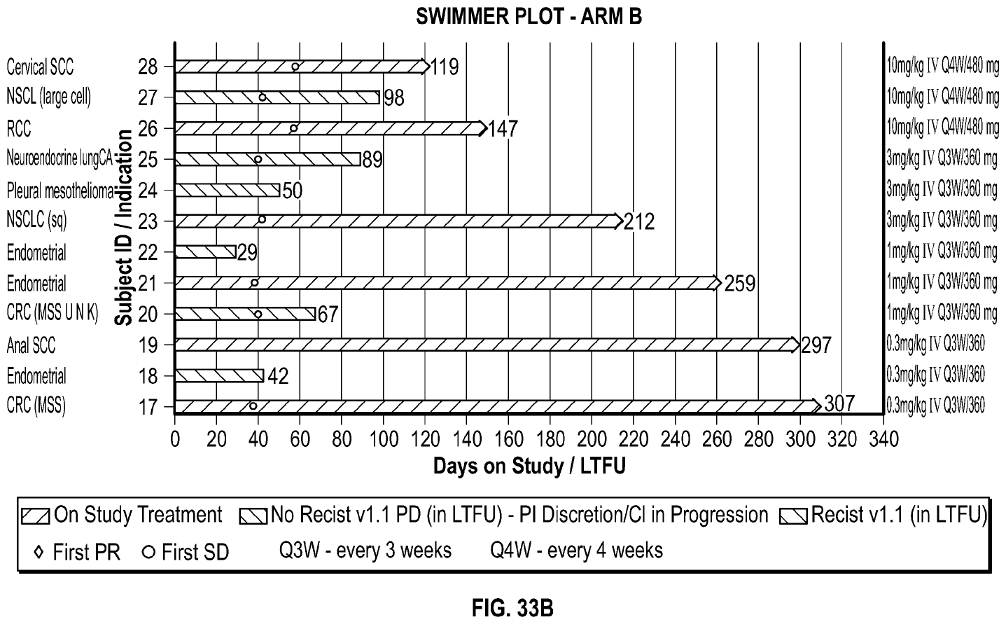

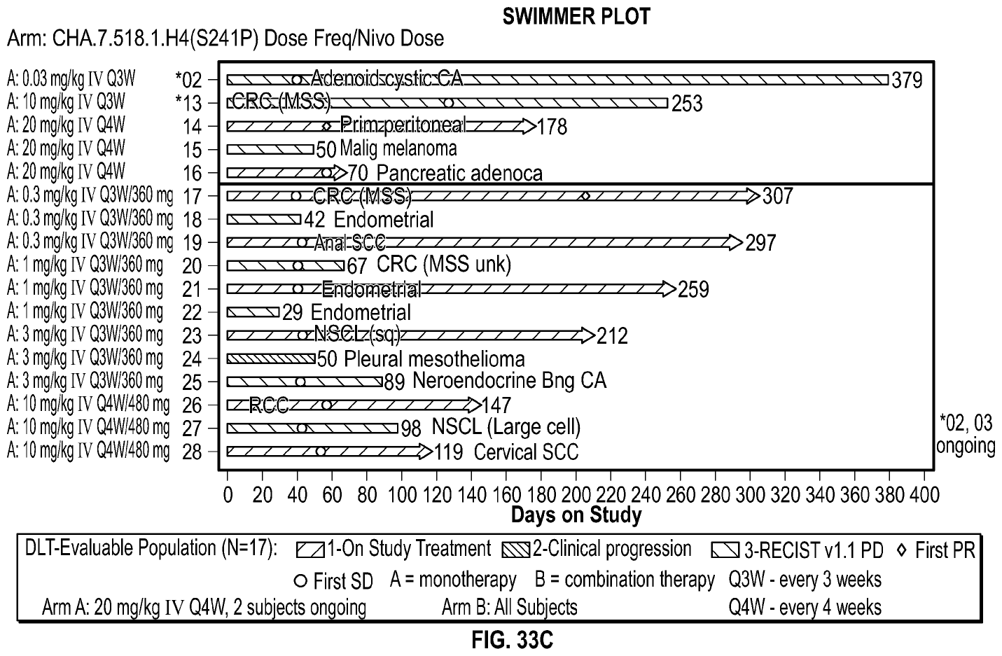

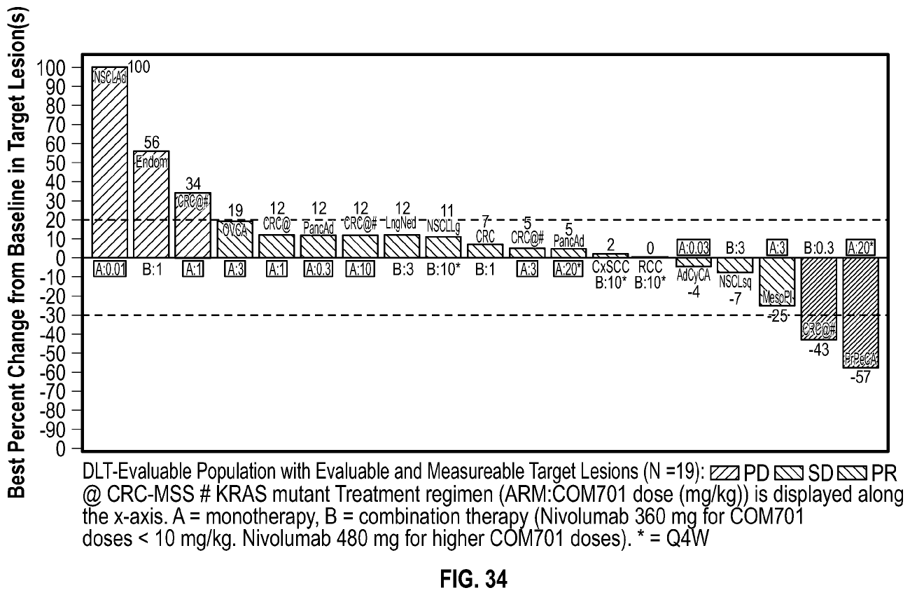

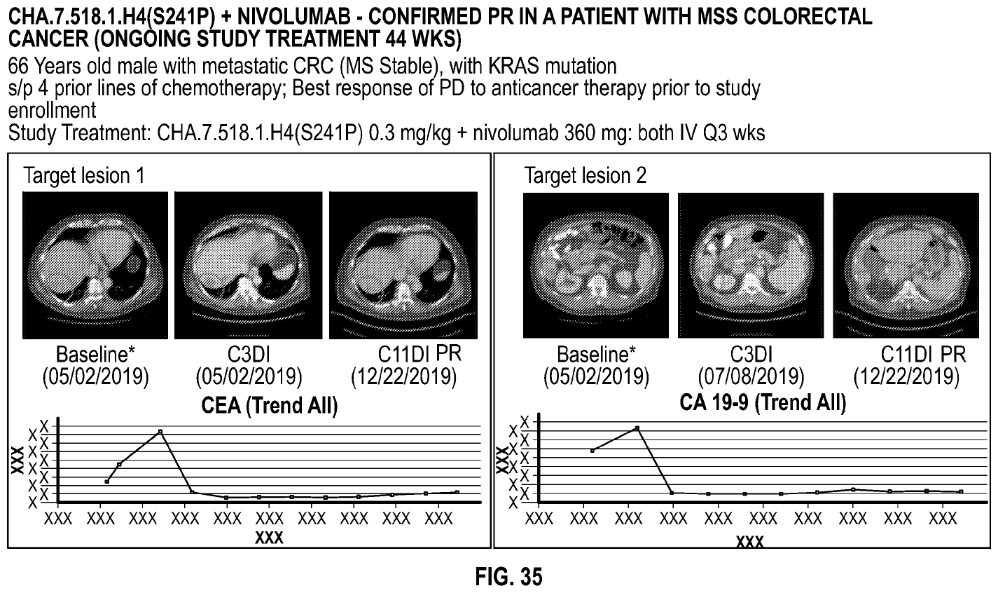

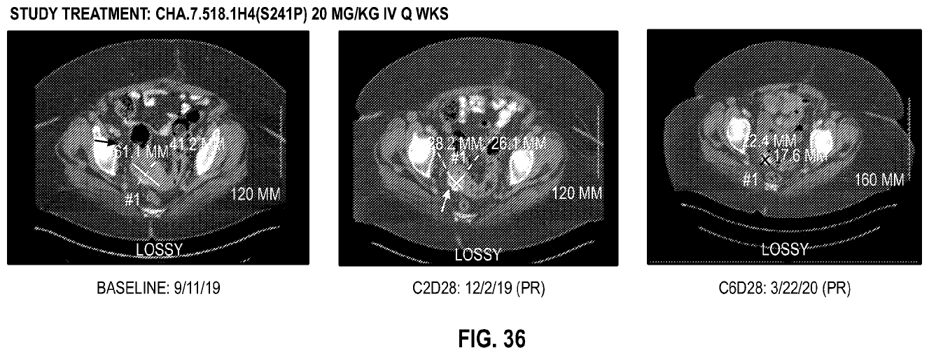

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region, although sometimes the numbering is shifted slightly as will be appreciated by those in the art; Kabat et al., SEQUENCES OF PROTEINS OF IMMUNOLOGICAL INTEREST, 5 th Ed. Public Health Service, National Institutes of Health, Bethesda, Md. (1991) and/or those residues forming a hypervariable loop (e.g., residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below and shown in FIG. 6A-6D.

The carboxy-terminal portion of each chain defines a constant region primarily responsible for effector function. Kabat et al., collected numerous primary sequences of the variable regions of heavy chains and light chains. Based on the degree of conservation of the sequences, they classified individual primary sequences into the CDR and the framework and made a list thereof (see SEQUENCES OF IMMUNOLOGICAL INTEREST, 5 th edition, NIH publication, No. 91-3242, E. A. Kabat et al., entirely incorporated by reference).

In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat.

Accordingly, the invention provides variable heavy domains, variable light domains, heavy constant domains, light constant domains and Fc domains to be used as outlined herein. By “variable region” as used herein is meant the region of an immunoglobulin that comprises one or more Ig domains substantially encoded by any of the Vκ or Vλ, and/or VH genes that make up the kappa, lambda, and heavy chain immunoglobulin genetic loci respectively. Accordingly, the variable heavy domain comprises vhFR1-vhCDR1-vhFR2-vhCDR2-vhFR3-vhCDR3-vhFR4, and the variable light domain comprises vlFR1-vlCDR1-vlFR2-v1CDR2-vlFR3-vlCDR3-vlFR4. By “heavy constant region” herein is meant the CH1-hinge-CH2-CH3 portion of an antibody. By “Fc” or “Fc region” or “Fc domain” as used herein is meant the polypeptide comprising the constant region of an antibody excluding the first constant region immunoglobulin domain and in some cases, part of the hinge. Thus Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cγ2 and Cγ3 (Cγ2 and Cγ3) and the lower hinge region between Cγ1 (Cγ1) and Cγ2 (Cγ2). Although the boundaries of the Fc region may vary, the human IgG heavy chain Fc region is usually defined to include residues C226 or P230 to its carboxyl-terminus, wherein the numbering is according to the EU index as in Kabat. In some embodiments, as is more fully described below, amino acid modifications are made to the Fc region, for example to alter binding to one or more FcγR receptors or to the FcRn receptor.

Thus, “Fc variant” or “variant Fc” as used herein is meant a protein comprising an amino acid modification in an Fc domain. The Fc variants of the present invention are defined according to the amino acid modifications that compose them. Thus, for example, N434S or 434S is an Fc variant with the substitution serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as M428L/N434S, and so on. For all positions discussed in the present invention that relate to antibodies, unless otherwise noted, amino acid position numbering is according to the EU index.

By “Fab” or “Fab region” as used herein is meant the polypeptide that comprises the VH, CH1, VL, and CL immunoglobulin domains. Fab may refer to this region in isolation, or this region in the context of a full length antibody, antibody fragment or Fab fusion protein. By “Fv” or “Fv fragment” or “Fv region” as used herein is meant a polypeptide that comprises the VL and VH domains of a single antibody. As will be appreciated by those in the art, these generally are made up of two chains.

Throughout the present specification, either the IMTG numbering system or the Kabat numbering system is generally used when referring to a residue in the variable domain (approximately, residues 1-107 of the light chain variable region and residues 1-113 of the heavy chain variable region) (e.g, Kabat et al., supra (1991)). EU numbering as in Kabat is generally used for constant domains and/or the Fc domains.

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning”. Specific bins are described below.

Included within the definition of “antibody” is an “antigen-binding portion” of an antibody (also used interchangeably with “antigen-binding fragment”, “antibody fragment” and “antibody derivative”). That is, for the purposes of the invention, an antibody of the invention has a minimum functional requirement that it bind to a PVRIG antigen. As will be appreciated by those in the art, there are a large number of antigen fragments and derivatives that retain the ability to bind an antigen and yet have alternative structures, including, but not limited to, (i) the Fab fragment consisting of VL, VH, CL and CH1 domains, (ii) the Fd fragment consisting of the VH and CH1 domains, (iii) F (ab′) 2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883, entirely incorporated by reference), (iv) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448, all entirely incorporated by reference), (v) “domain antibodies” or “dAb” (sometimes referred to as an “immunoglobulin single variable domain”, including single antibody variable domains from other species such as rodent (for example, as disclosed in WO 00/29004), nurse shark and Camelid V-HH dAbs, (vi) SMIPs (small molecule immunopharmaceuticals), camelbodies, nanobodies and IgNAR.

Still further, an antibody or antigen-binding portion thereof (antigen-binding fragment, antibody fragment, antibody portion) may be part of a larger immunoadhesion molecules (sometimes also referred to as “fusion proteins”), formed by covalent or noncovalent association of the antibody or antibody portion with one or more other proteins or peptides. Examples of immunoadhesion molecules include use of the streptavidin core region to make a tetrameric scFv molecule and use of a cysteine residue, a marker peptide and a C-terminal polyhistidine tag to make bivalent and biotinylated scFv molecules. Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

In general, the anti-PVRIG antibodies of the invention are recombinant. “Recombinant” as used herein, refers broadly with reference to a product, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “recombinant antibody”, as used herein, includes all antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

### A. Optional Antibody Engineering

The anti-PVRIG antibodies (e.g., anti-PVRIG antibodies including those with CDRs identical to those shown in FIG. 3) of the invention can be modified, or engineered, to alter the amino acid sequences by amino acid substitutions.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

As discussed herein, amino acid substitutions can be made to alter the affinity of the CDRs for the PVRIG protein (including both increasing and decreasing binding, as is more fully outlined below), as well as to alter additional functional properties of the antibodies. For example, the antibodies may be engineered to include modifications within the Fc region, typically to alter one or more functional properties of the antibody, such as serum half-life, complement fixation, Fc receptor binding, and/or antigen-dependent cellular cytotoxicity. Furthermore, an antibody according to at least some embodiments of the invention may be chemically modified (e.g., one or more chemical moieties can be attached to the antibody) or be modified to alter its glycosylation, again to alter one or more functional properties of the antibody. Such embodiments are described further below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al., The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococeyl protein A (SpA) binding relative to native Fc-hinge domain SpA binding. This approach is described in further detail in U.S. Pat. No. 6,165,745 by Ward et al.,

In some embodiments, amino acid substitutions can be made in the Fc region, in general for altering binding to FcγR receptors. By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII-1 (CD16), and FcγRIII-2 (CD16-2), as well as any undiscovered mouse FcγRs or FcγR isoforms or allotypes.

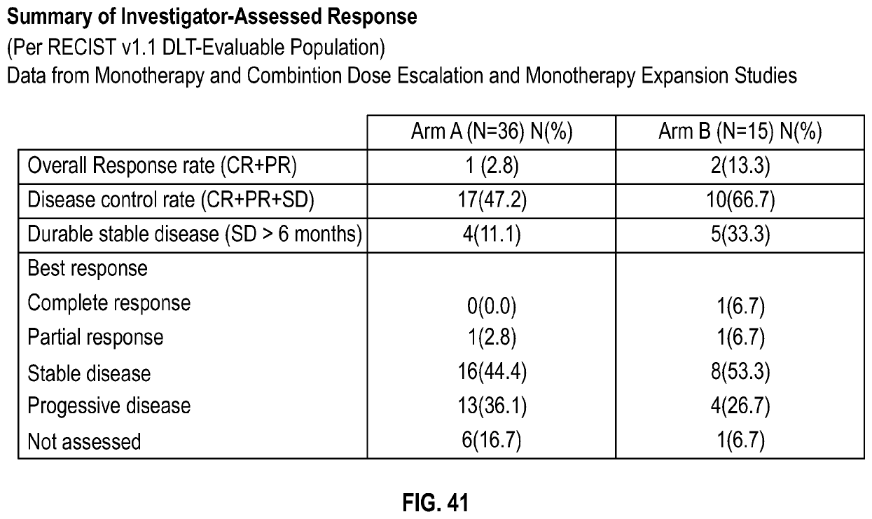

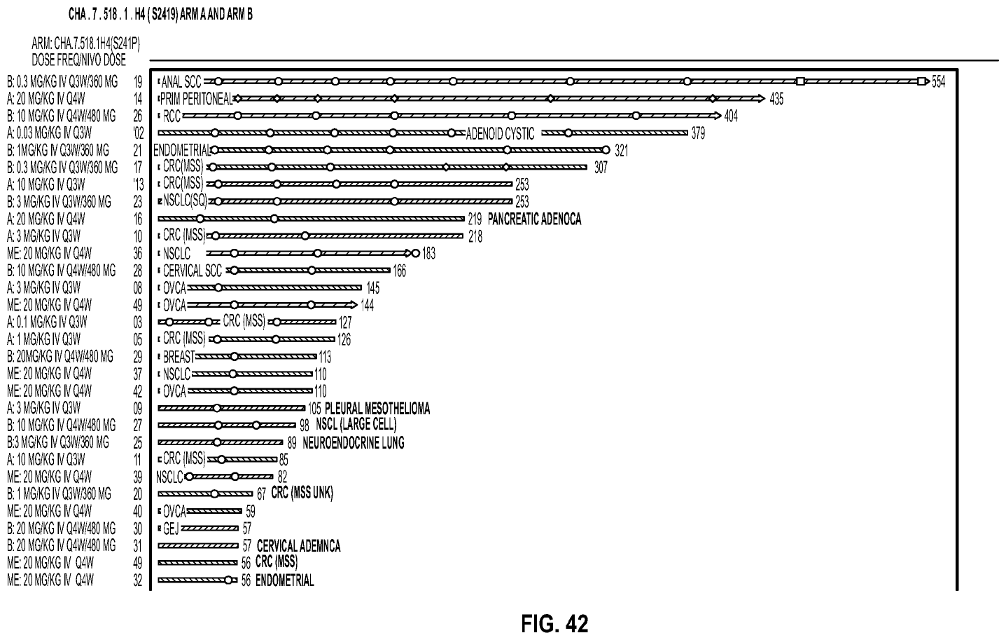

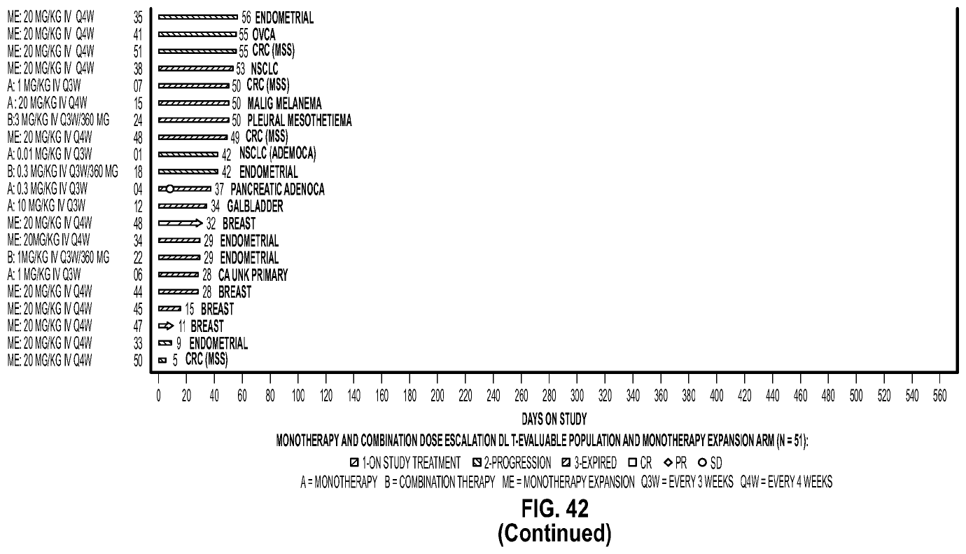

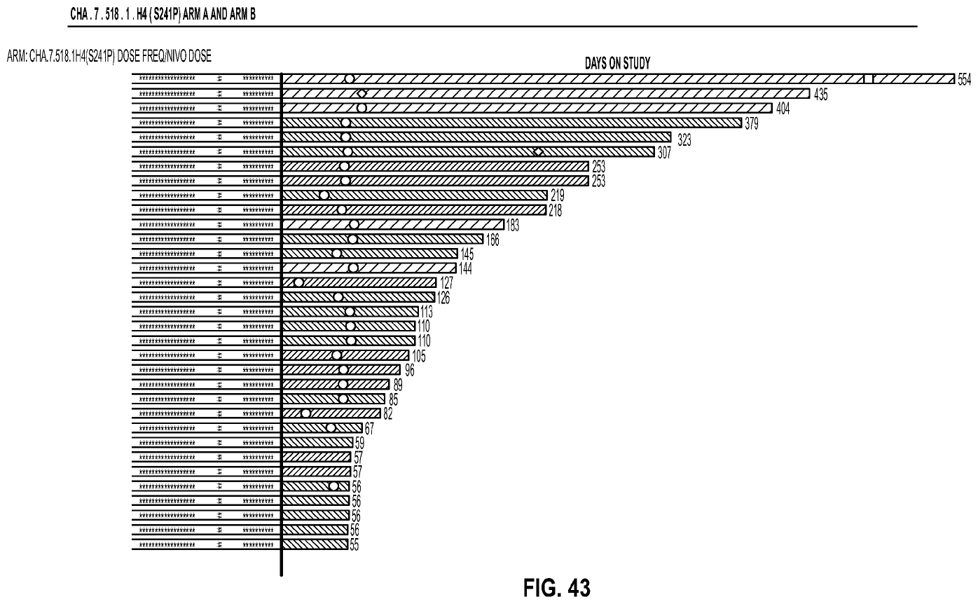

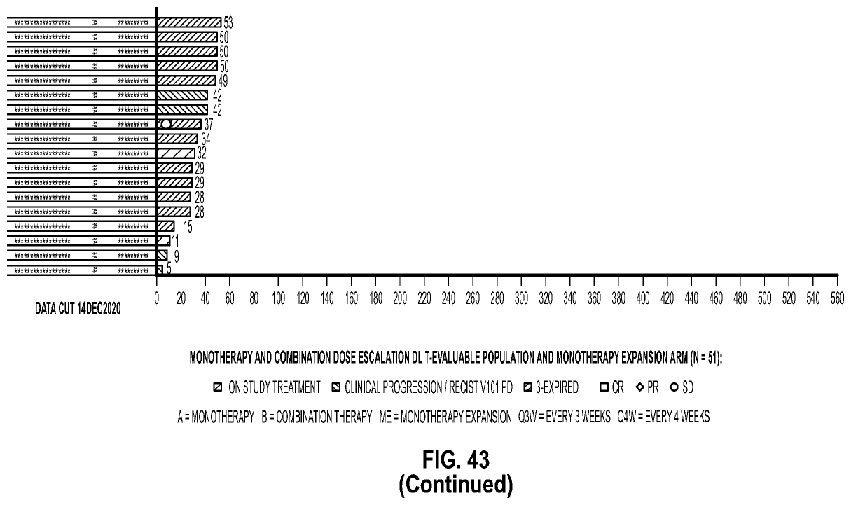

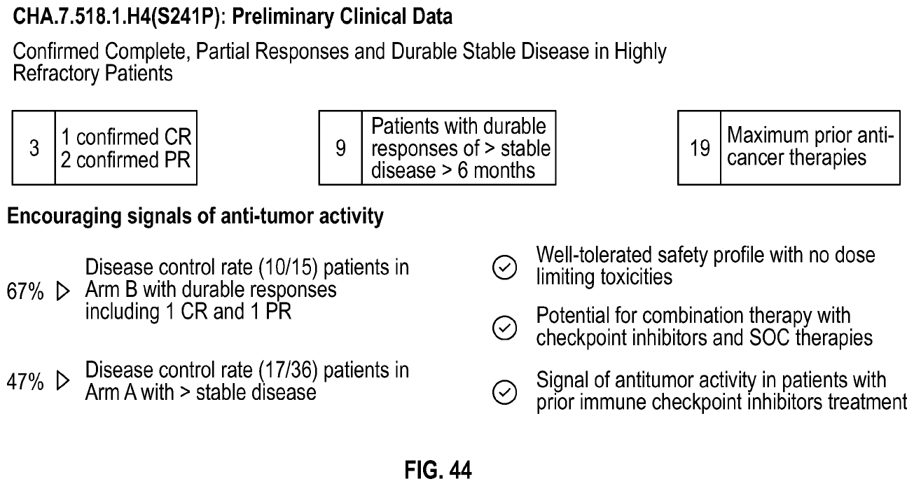

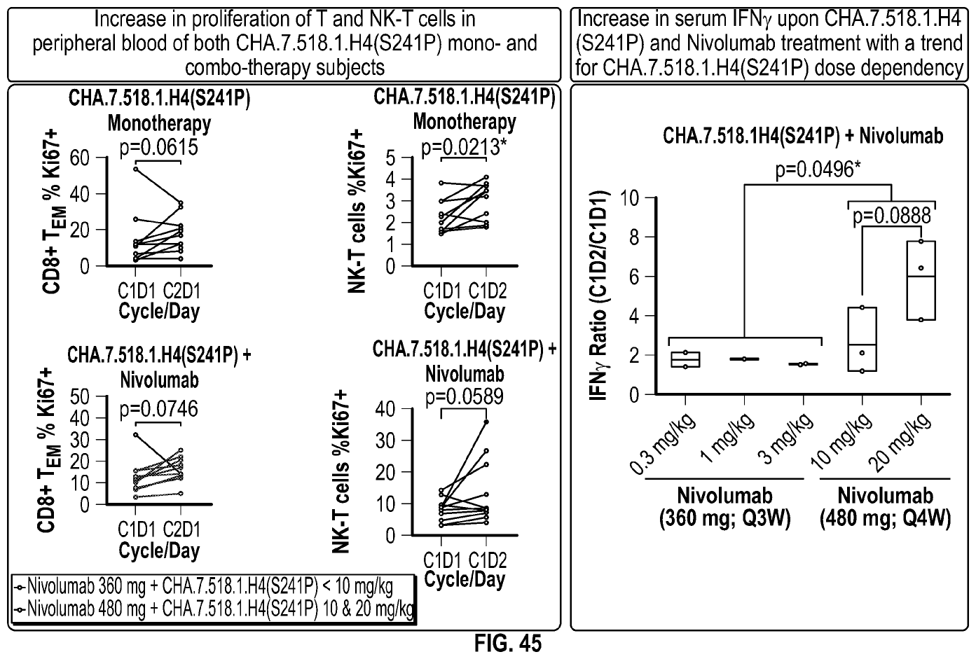

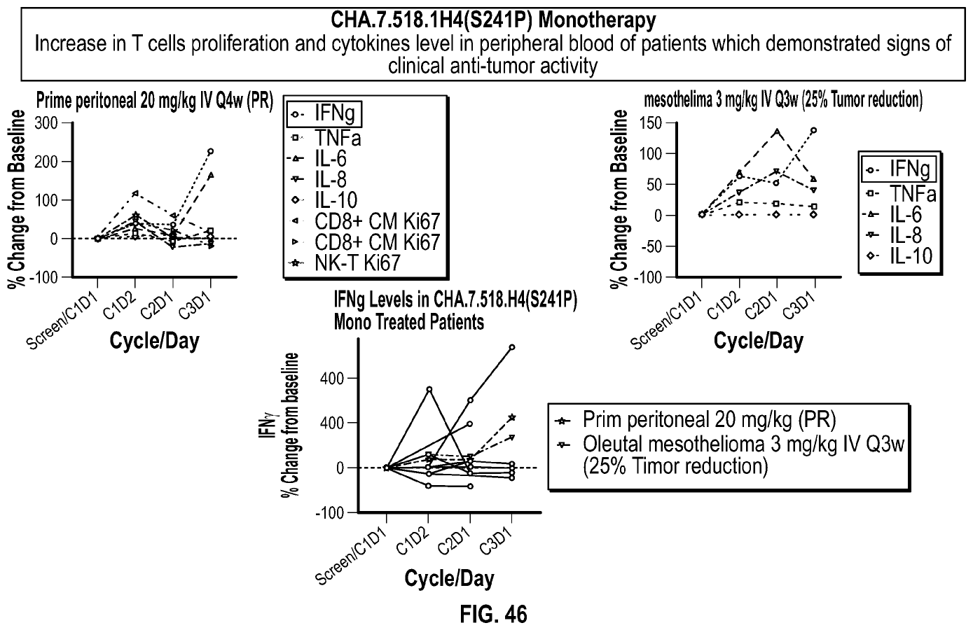

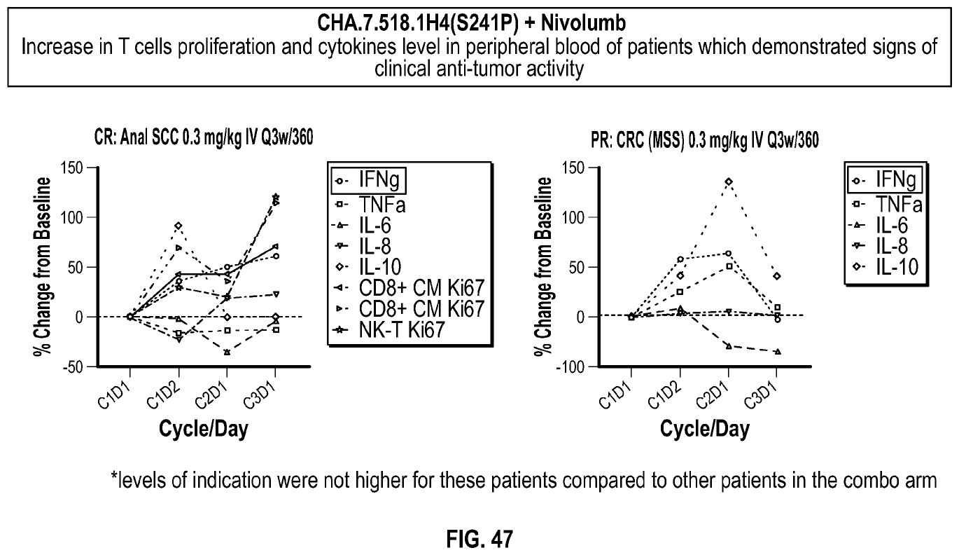

There are a number of useful Fc substitutions that can be made to alter binding to one or more of the FcγR receptors. Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell. Similarly, decreased binding to FcγRIIb (an inhibitory receptor) can be beneficial as well in some circumstances. Amino acid substitutions that find use in the present invention include those listed in U.S. Ser. Nos. 11/124,620 (particularly FIG. 41) and U.S. Pat. No. 6,737,056, both of which are expressly incorporated herein by reference in their entirety and specifically for the variants disclosed therein. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 299T and 297N.

In addition, the antibodies of the invention are modified to increase its biological half-life. Various approaches are possible. For example, one or more of the following mutations can be introduced: T252L, T254S, T256F, as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al., Additional mutations to increase serum half-life are disclosed in U.S. Pat. Nos. 8,883,973, 6,737,056 and 7,371,826, and include 428L, 434A, 434S, and 428L/434S.

In yet other embodiments, the Fc region is altered by replacing at least one amino acid residue with a different amino acid residue to alter the effector functions of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.,

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.,

In another example, one or more amino acid residues within amino acid positions 231 and 239 are altered to thereby alter the ability of the antibody to fix complement. This approach is described further in PCT Publication WO 94/29351 by Bodmer et al.,

In yet another example, the Fc region is modified to increase the ability of the antibody to mediate antibody dependent cellular cytotoxicity (ADCC) and/or to increase the affinity of the antibody for an Fcγ receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgG1 for FcγRI, FcγRII, FcγRIII and FcRn have been mapped and variants with improved binding have been described (see Shields, R. L. et al., (2001) J. Biol. Chem. 276:6591-6604). Specific mutations at positions 256, 290, 298, 333, 334 and 339 are shown to improve binding to FcγRIII. Additionally, the following combination mutants are shown to improve FcγRIII binding: T256A/S298A, S298A/E333A, S298A/K224A and S298A/E333A/K334A. Furthermore, mutations such as M252Y/S254T/T256E or M428L/N434S improve binding to FcRn and increase antibody circulation half-life (see Chan C A and Carter P J (2010) Nature Rev Immunol 10:301-316).

In still another embodiment, the antibody can be modified to abrogate in vivo Fab arm exchange. Specifically, this process involves the exchange of IgG4 half-molecules (one heavy chain plus one light chain) between other IgG4 antibodies that effectively results in bispecific antibodies which are functionally monovalent. Mutations to the hinge region and constant domains of the heavy chain can abrogate this exchange (see Aalberse, RC, Schuurman J., 2002, Immunology 105:9-19).

In still another embodiment, the glycosylation of an antibody is modified. For example, an aglycosylated antibody can be made (i.e., the antibody lacks glycosylation). Glycosylation can be altered to, for example, increase the affinity of the antibody for antigen or reduce effector function such as ADCC. Such carbohydrate modifications can be accomplished by, for example, altering one or more sites of glycosylation within the antibody sequence, for example N297. For example, one or more amino acid substitutions can be made that result in elimination of one or more variable region framework glycosylation sites to thereby eliminate glycosylation at that site.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies according to at least some embodiments of the invention to thereby produce an antibody with altered glycosylation. For example, the cell lines Ms704, Ms705, and Ms709 lack the fucosyltransferase gene, FUT8 (a (1,6) fucosyltransferase), such that antibodies expressed in the Ms704, Ms705, and Ms709 cell lines lack fucose on their carbohydrates. The Ms704, Ms705, and Ms709 FUT8 cell lines are created by the targeted disruption of the FUT8 gene in CHO/DG44 cells using two replacement vectors (see U.S. Patent Publication No. 20040110704 by Yamane et al., and Yamane-Ohnuki et al., (2004) Biotechnol Bioeng 87:614-22). As another example, EP 1,176,195 by Hanai et al., describes a cell line with a functionally disrupted FUT8 gene, which encodes a fucosyl transferase, such that antibodies expressed in such a cell line exhibit hypofucosylation by reducing or eliminating the a 1,6 bond-related enzyme. Hanai et al., also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 by Presta describes a variant CHO cell line, Lec13 cells, with reduced ability to attach fucose to Asn (297)-linked carbohydrates, also resulting in hypofucosylation of antibodies expressed in that host cell (see also Shields, R. L. et al., (2002) J. Biol. Chem. 277:26733-26740). PCT Publication WO 99/54342 by Umana et al., describes cell lines engineered to express glycoprotein-modifying glycosyl transferases (e.g., β (1,4)-N-acetylglucosaminyltransferase III (GnTIII)) such that antibodies expressed in the engineered cell lines exhibit increased bisecting GlcNac structures which results in increased ADCC activity of the antibodies (see also Umana et al., (1999) Nat. Biotech. 17:176-180). Alternatively, the fucose residues of the antibody may be cleaved off using a fucosidase enzyme. For example, the fucosidase α-L-fucosidase removes fucosyl residues from antibodies (Tarentino, A. L. et al., (1975) Biochem. 14:5516-23).

Another modification of the antibodies herein that is contemplated by the invention is pegylation or the addition of other water soluble moieties, typically polymers, e.g., in order to enhance half-life. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies according to at least some embodiments of the invention. See for example, EP 0 154 316 by Nishimura et al., and EP 0 401 384 by Ishikawa et al.

In addition to substitutions made to alter binding affinity to FcγRs and/or FcRn and/or increase in vivo serum half-life, additional antibody modifications can be made, as described in further detail below.

In some cases, affinity maturation is done. Amino acid modifications in the CDRs are sometimes referred to as “affinity maturation”. An “affinity matured” antibody is one having one or more alteration(s) in one or more CDRs which results in an improvement in the affinity of the antibody for antigen, compared to a parent antibody which does not possess those alteration(s). In some cases, although rare, it may be desirable to decrease the affinity of an antibody to its antigen, but this is generally not preferred.

In some embodiments, one or more amino acid modifications are made in one or more of the CDRs of the PVRIG antibodies of the invention. In general, only 1 or 2 or 3-amino acids are substituted in any single CDR, and generally no more than from 1, 2, 3. 4, 5, 6, 7, 8 9 or 10 changes are made within a set of CDRs. However, it should be appreciated that any combination of no substitutions, 1, 2 or 3 substitutions in any CDR can be independently and optionally combined with any other substitution.

Affinity maturation can be done to increase the binding affinity of the antibody for the PVRIG antigen by at least about 10% to 50-100-150% or more, or from 1 to 5 fold as compared to the “parent” antibody. Preferred affinity matured antibodies will have nanomolar or even picomolar affinities for the PVRIG antigen. Affinity matured antibodies are produced by known procedures. See, for example, Marks et al., 1992, Biotechnology 10:779-783 that describes affinity maturation by variable heavy chain (VH) and variable light chain (VL) domain shuffling. Random mutagenesis of CDR and/or framework residues is described in: Barbas, et al., 1994, Proc. Nat. Acad. Sci, USA 91:3809-3813; Shier et al., 1995, Gene 169:147-155; Yelton et al., 1995, J. Immunol. 155:1994-2004; Jackson et al., 1995, J. Immunol. 154 (7): 3310-9; and Hawkins et al., 1992, J. Mol. Biol. 226:889-896, for example.

Alternatively, amino acid modifications can be made in one or more of the CDRs of the antibodies of the invention that are “silent”, e.g., that do not significantly alter the affinity of the antibody for the antigen. These can be made for a number of reasons, including optimizing expression (as can be done for the nucleic acids encoding the antibodies of the invention).

Thus, included within the definition of the CDRs and antibodies of the invention are variant CDRs and antibodies; that is, the antibodies of the invention can include amino acid modifications in one or more of the CDRs of the enumerated antibodies of the invention. In addition, as outlined below, amino acid modifications can also independently and optionally be made in any region outside the CDRs, including framework and constant regions.

### V. PVRIG Antibodies

The present invention provides anti-PVRIG antibodies. (For convenience, “anti-PVRIG antibodies” and “PVRIG antibodies” are used interchangeably). The anti-PVRIG antibodies of the invention specifically bind to human PVRIG, and preferably the ECD of human PVRIG1, as depicted in FIG. 3, including, e.g., anti-PVRIG antibodies including those with CDRs identical to those shown in FIG. 3.

Specific binding for PVRIG or a PVRIG epitope can be exhibited, for example, by an antibody having a KD of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the PVRIG antigen or epitope.

However, as shown in the Examples, for optimal binding to PVRIG expressed on the surface of NK and T-cells, the antibodies preferably have a KD less 50 nM and most preferably less than 1 nM, with less than 0.1 nM and less than 1 pM and 0.1 pM finding use in the methods of the invention.

Also, specific binding for a particular antigen or an epitope can be exhibited, for example, by an antibody having a KA or Ka for a PVRIG antigen or epitope of at least 20-, 50-, 100-, 500-, 1000-, 5,000-, 10,000- or more times greater for the epitope relative to a control, where KA or Ka refers to an association rate of a particular antibody-antigen interaction. s

In some embodiments, the anti-PVRIG antibodies of the invention bind to human PVRIG with a K_{D} of 100 nM or less, 50 nM or less, 10 nM or less, or 1 nM or less (that is, higher binding affinity), or 1 pM or less, wherein K_{D} is determined by known methods, e.g., surface plasmon resonance (SPR, e.g., Biacore assays), ELISA, KINEXA, and most typically SPR at 25° or 37° C.

The invention provides antigen binding domains, including full length antibodies, which contain a number of specific, enumerated sets of 6 CDRs, as provided in FIG. 3. The invention provides antigen binding domains, including full length antibodies, which contain a number of specific, enumerated sets of 6 CDRs, as provided in FIG. 3.

The invention further provides variable heavy and light domains as well as full length heavy and light chains.

As discussed herein, the invention further provides variants of the above components, including variants in the CDRs, as outlined above. In addition, variable heavy chains can be at least 80%, at least 90%, at least 95%, at least 98% or at least 99% identical to the “VH” sequences herein, and/or contain from 1, 2, 3, 4, 5, 6, 7, 8, 9, 10 amino acid changes, or more, when Fc variants are used. Variable light chains are provided that can be at least 80%, at least 90%, at least 95%, at least 98% or at least 99% identical to the “VL” sequences herein, and/or contain from 1, 2, 3, 4, 5, 6, 7, 8, 9, 10 amino acid changes, or more, when Fc variants are used. Similarly, heavy and light chains are provided that are at least 80%, at least 90%, at least 95%, at least 98% or at least 99% identical to the “HC” and “LC” sequences herein, and/or contain from 1, 2, 3, 4, 5, 6, 7, 8, 9, 10 amino acid changes, or more, when Fc variants are used.

Accordingly, the present invention provides antibodies, usually full length or scFv domains, that comprise the following CHA sets of CDRs, the sequences of which are shown in FIG. 3:

CHA.7.518.1.H4 (S241P) vhCDR1, CHA.7.518.1.H4 (S241P) vhCDR2, CHA.7.518.1.H4 (S241P) vhCDR3, CHA.7.518.1.H4 (S241P) vlCDR1, CHA.7.518.1.H4 (S241P) vlCDR2, and CHA.7.518.1.H4 (S241P) vlCDR3.

In addition, the framework regions of the variable heavy and variable light chains can be humanized as is known in the art (with occasional variants generated in the CDRs as needed), and thus humanized variants of the VH and VL chains of FIG. 3 can be generated. Furthermore, the humanized variable heavy and light domains can then be fused with human constant regions, such as the constant regions from IgG1, IgG2, IgG3 and IgG4.

In addition, also included are sequences that may have the identical CDRs but changes in the variable domain (or entire heavy or light chain). For example, PVRIG antibodies include those with CDRs identical to those shown in FIG. 3 or FIGS. 5A-5D but whose identity along the variable region can be lower, for example 95 or 98% percent identical. For example, PVRIG antibodies include those with CDRs identical to those shown in FIG. 3 but whose identity along the variable region can be lower, for example 95 or 98% percent identical, and in some embodiments at least 95% or at least 98%.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available commercially), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al., (1990) J Mol. Biol. 215:403-10. BLAST protein searches can be performed with the XBLAST program, score=50, wordlength=3 to obtain amino acid sequences homologous to the antibody molecules according to at least some embodiments of the invention. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used.

In general, the percentage identity for comparison between PVRIG antibodies is at least 75%, at least 80%, at least 90%, with at least about 95, 96, 97, 98 or 99% percent identity being preferred. The percentage identity may be along the whole amino acid sequence, for example the entire heavy or light chain or along a portion of the chains. For example, included within the definition of the anti-PVRIG antibodies of the invention are those that share identity along the entire variable region (for example, where the identity is 95 or 98% identical along the variable regions, and in some embodiments at least 95% or at least 98%), or along the entire constant region, or along just the Fc domain.

### VI. TIGIT Antibodies

The present invention provides anti-TIGIT antibodies. (For convenience, “anti-TIGIT antibodies” and “TIGIT antibodies” are used interchangeably). The anti-TIGIT antibodies of the invention specifically bind to human TIGIT, and preferably the ECD of human TIGIT. The invention further provides antigen binding domains, including full length antibodies, which contain a number of specific, enumerated sets of 6 CDRs that bind to TIGIT.

Specific binding for TIGIT or a TIGIT epitope can be exhibited, for example, by an antibody having a K_{D} of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, at least about 10⁻¹³ M, at least about 10⁻¹⁴ M, at least about 10⁻¹⁵ M, or greater, where K_{D} refers to the equilibrium dissociation constant of a particular antibody-antigen interaction. Typically, an antibody that specifically binds an antigen will have a K_{D} that is 20-, 50-, 100-, 500-, 1000-, 5,000-, 10,000- or more times greater for a control molecule relative to the TIGIT antigen or epitope.

However, for optimal binding to TIGIT expressed on the surface of NK and T-cells, the antibodies preferably have a K_{D} less 50 nM and most preferably less than 1 nM, with less than 0.1 nM and less than 1 pM finding use in the methods of the invention

Also, specific binding for a particular antigen or an epitope can be exhibited, for example, by an antibody having a ka (referring to the association rate constant) for a TIGIT antigen or epitope of at least 20-, 50-, 100-, 500-, 1000-, 5,000-, 10,000- or more times greater for the epitope relative to a control, where ka refers to the association rate constant of a particular antibody-antigen interaction.

In some embodiments, the anti-TIGIT antibodies of the invention bind to human TIGIT with a K_{D} of 100 nM or less, 50 nM or less, 10 nM or less, or 1 nM or less (that is, higher binding affinity), or 1 pM or less, wherein K_{D} is determined by known methods, e.g., surface plasmon resonance (SPR, e.g., Biacore assays), ELISA, KINEXA, and most typically SPR at 25° or 37° C.

The invention provides antigen binding domains, including full length antibodies, which contain a number of specific, enumerated sets of 6 CDRs, as provided in FIG. 3. The invention provides antigen binding domains, including full length antibodies, which contain a number of specific, enumerated sets of 6 CDRs, as provided in FIG. 3.

The invention further provides variable heavy and light domains as well as full length heavy and light chains.

As discussed herein, the invention further provides variants of the above components, including variants in the CDRs, as outlined above. In addition, variable heavy chains can be at least 80%, at least 90%, at least 95%, at least 98% or at least 99% identical to the “VH” sequences herein, and/or contain from 1, 2, 3, 4, 5, 6, 7, 8, 9, 10 amino acid changes, or more, when Fc variants are used. Variable light chains are provided that can be at least 80%, at least 90%, at least 95%, at least 98% or at least 99% identical to the “VL” sequences herein, and/or contain from 1, 2, 3, 4, 5, 6, 7, 8, 9, 10 amino acid changes, or more, when Fc variants are used. Similarly, heavy and light chains are provided that are at least 80%, at least 90%, at least 95%, at least 98% or at least 99% identical to the “HC” and “LC” sequences herein, and/or contain from 1, 2, 3, 4, 5, 6, 7, 8, 9, 10 amino acid changes, or more, when Fc variants are used.

In addition, the framework regions of the variable heavy and variable light chains can be humanized as is known in the art (with occasional variants generated in the CDRs as needed), and thus humanized variants of the VH and VL chains of FIG. 3 can be generated. Furthermore, the humanized variable heavy and light domains can then be fused with human constant regions, such as the constant regions from IgG1, IgG2, IgG3 and IgG4.

In addition, also included are sequences that may have the identical CDRs but changes in the variable domain (or entire heavy or light chain). For example, TIGIT antibodies include those with CDRs identical to those shown in the present disclosure but whose identity along the variable region can be lower, for example 95 or 98% percent identical. For example, TIGIT antibodies include those with CDRs identical to those shown in the present disclosure but whose identity along the variable region can be lower, for example 95 or 98% percent identical, and in some embodiments at least 95% or at least 98%.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available commercially), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al., (1990) J Mol. Biol. 215:403-10. BLAST protein searches can be performed with the XBLAST program, score=50, wordlength=3 to obtain amino acid sequences homologous to the antibody molecules according to at least some embodiments of the invention. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used.

In general, the percentage identity for comparison between TIGIT antibodies is at least 75%, at least 80%, at least 90%, with at least about 95, 96, 97, 98 or 99% percent identity being preferred. The percentage identity may be along the whole amino acid sequence, for example the entire heavy or light chain or along a portion of the chains. For example, included within the definition of the anti-TIGIT antibodies of the invention are those that share identity along the entire variable region (for example, where the identity is 95 or 98% identical along the variable regions, and in some embodiments at least 95% or at least 98%), or along the entire constant region, or along just the Fc domain.

The present invention provides an anti-TIGIT antibody, BMS-986207. mAb BMS-986207 is disclosed as mAb 22G2 in Int'l Pat. Pub. No. WO 2016/106302 (incorporated herein by reference in its entirety). BMS-986207, the 22G2 mAB, specifically binds to huTIGIT.

BMS-986207, the 22G2 mAB, comprises heavy chain CDRH1, CDRH2, and CDRH3 sequences comprising:
CDRH1: Ser Gly Ile Tyr Tyr Trp Ser (SEQ ID NO: 2000);
CDRH2: Tyr Ile Tyr Tyr Ser Gly Ser Thr Asn Tyr Asn Pro Ser Leu Lys Ser (SEQ ID NO: 2001); and
CDRH3: Asp Tyr Tyr Val Ser Gly Asn Tyr Tyr Asn Val Asp Tyr Tyr Phe Phe Gly Val Asp Val (SEQ ID NO: 2002).

BMS-986207, the 22G2 mAB, comprises light chain CDRL1, CDRL2, and CDRL3 sequences comprising:
CDRL1: Arg Ala Ser Gln Ser Val Ser Ser Tyr Leu Ala (SEQ ID NO: 2003);
CDRL2: Asp Ala Ser Asn Arg Ala Thr (SEQ ID NO: 2004); and
CDRL3: Gln Gln Arg Ser Asn Trp Pro Pro Leu Phe Thr (SEQ ID NO: 2005).

### A. Anti-TIGIT Antibodies in Combination Therapy

The TIGIT and PVRIG antibodies of the invention find particular use in the treatment of cancer when used in combination and for example with a checkpoint inhibitor such as an anti-PD-1 antibody, as described herein. In general, the antibodies of the invention are immunomodulatory, in that rather than directly attack cancerous cells, the anti-TIGIT and anti-PVRIG antibodies of the invention stimulate the immune system, generally by inhibiting the action of TIGIT and PVRIG, respectively. Thus, unlike tumor-targeted therapies, which are aimed at inhibiting molecular pathways that are crucial for tumor growth and development, and/or depleting tumor cells, cancer immunotherapy is aimed to stimulate the patient's own immune system to eliminate cancer cells, providing long-lived tumor destruction. Various approaches can be used in cancer immunotherapy, among them are therapeutic cancer vaccines to induce tumor-specific T cell responses, and immunostimulatory antibodies (i.e. antagonists of inhibitory receptors=immune checkpoints) to remove immunosuppressive pathways.

Clinical responses with targeted therapy or conventional anti-cancer therapies tend to be transient as cancer cells develop resistance, and tumor recurrence takes place. However, the clinical use of cancer immunotherapy in the past few years has shown that this type of therapy can have durable clinical responses, showing dramatic impact on long term survival. However, although responses are long term, only a small number of patients respond (as opposed to conventional or targeted therapy, where a large number of patients respond, but responses are transient).

By the time a tumor is detected clinically, it has already evaded the immune-defense system by acquiring immunoresistant and immunosuppressive properties and creating an immunosuppressive tumor microenvironment through various mechanisms and a variety of immune cells.

Accordingly, the anti-TIGIT and anti-PVRIG combinations of the invention are useful in treating cancer. Due to the nature of an immuno-oncology mechanism of action, TIGIT and or PVRIG do not necessarily need to be overexpressed on or correlated with a particular cancer type; that is, the goal is to have the anti-TIGIT antibodies de-suppress T cell and NK cell activation, such that the immune system will go after the cancers.

### VII. Biomarkers and/or Companion Diagnostic

In some embodiments, the method for treatment includes in part a method for determining or predicting the efficacy of treatment with an anti-PVRIG antibody alone or in combination with an anti-PD-1 antibody in a cancer patient, the method comprising:
(a) measuring the level of one or more cellular components comprising activated DC cells, effector memory CD8 positive T cells, CD8 positive T cells, and NK-T cells, in a biological sample from of a cancer patient;
(b) quantitating the measurement of the level of the one or more cellular components; and
(c) correlating the level of the one or more cellular components with the efficacy of treatment.

In some embodiments, the method for treatment includes in part a method for determining a cancer patient population for treatment with an anti-PVRIG antibody alone or in combination with an anti-PD-1 antibody, the method comprising:
(a) detecting the presence in a biological sample from the cancer patient one or more cellular components comprising activated DC cells, effector memory CD8 positive T cells, CD8 positive T cells, and NK-T cells;
(b) quantitating the measurement of the level of the one or more cellular components;
(c) treating the cancer patient with an anti-PVRIG antibody alone or in combination with an anti-PD-1 antibody when the level of the one or more cellular components are present at an increased level as compared to a control, pre-treatment sample, or a patient that does not have detectable levels of the cells.

In some embodiments, the method for treatment includes in part a method for determining or predicting the efficacy of treatment with an anti-PVRIG antibody alone or in combination with an anti-PD-1 antibody in a cancer patient, the method comprising:
(a) measuring the level of one or more biomarkers in a biological sample from the cancer patient;
(b) quantitating the measurement of the level of the one or more biomarkers; and
(c) correlating the level of the one or more biomarkers with the efficacy of treatment.

In some embodiments, the method for treatment includes in part a method for determining a cancer patient population for treatment with an anti-PVRIG antibody alone or in combination with an anti-PD-1 antibody, the method comprising:
(a) detecting the presence in a biological sample from the cancer patient one or more biomarkers;
(b) quantitating the measurement of the level of the one or more biomarkers;
(c) treating the cancer patient with an anti-PVRIG antibody alone or in combination with an anti-PD-1 antibody when the level of the one or more biomarkers is present at an increased or decreased level as compared to a control, pre-treatment sample, or a patient that does not have detectable levels of the one or more biomarkers

In some embodiments, the biomarkers are proteins or protein levels and/or mRNAs or mRNA levels.

In some embodiments, the biological sample is obtained from a tumor, tumor microenvironment, and/or peripheral blood from the cancer patient.

In some embodiments, the biological sample is analyzed using assays including immunoassays, protein expression assays, and/or cell assays. In some embodiments, high-throughput assays are conducted to analyze the biological sample. In some embodiments, a proximity extension assay is conducted to analyze the biological sample. In some embodiments, the biological sample is analyzed using Olink Explore 1536. In some embodiments, the biological sample is analyzed using Nanostring DSP technology. In some embodiments, the biological sample is analyzed using flow cytometry. In some embodiments, the biological sample is analyzed using histological assays. In some embodiments, the biological sample is analyzed using immunostaining.

In some embodiments, the activated DC cells being present and/or being present at an increased level as compared to a control, pre-treatment sample, or a patient that does not have detectable levels of the activated DC cells, is indicative of treatment efficacy for treatment with an anti-PVRIG antibody alone or in combination with an anti-PD-1 antibody.

In some embodiments, the activated DC cells express one or more biomarkers selected from the group consisting of LAMP3, HLA-DR and CD83.

In some embodiments, the measuring the level of the activated DC cells comprises measuring expression level of one or more biomarkers selected from the group consisting of LAMP3, HLA-DR and CD83.

In some embodiments, the level of the activated DC cells is indicative of treatment efficacy for treatment with an anti-PVRIG antibody alone or in combination with an anti-PD-1 antibody when the expression level of the one or more biomarkers is increased by at least 1-fold, 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.5-fold, 5.6-fold, 5.7-fold, 5.8-fold, 5.9-fold, 6-fold, 6.1-fold, 6.2-fold, 6.3-fold, 6.4-fold, 6.5-fold, 6.6-fold, 6.7-fold, 6.8-fold, 6.9-fold, 7-fold, 7.1-fold, 7.2-fold, 7.3-fold, 7.4-fold, 7.5-fold, 7.6-fold, 7.7-fold, 7.8-fold, 7.9-fold, 8-fold, 8.1-fold, 8.2-fold, 8.3-fold, 8.4-fold, 8.5-fold, 8.6-fold, 8.7-fold, 8.8-fold, 8.9-fold, 9-fold, 9.1-fold, 9.2-fold, 9.3-fold, 9.4-fold, 9.5-fold, 9.6-fold, 9.7-fold, 9.8-fold, 9.9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, or 30-fold as compared to a control, pre-treatment sample, or a patient that does not respond to treatment with an anti-PVRIG antibody alone or in combination with an anti-PD-1 antibody.

In some embodiments, the effector memory CD8 positive T cells, or CD8 positive T cells, being present and/or being present at an increased level in the peripheral blood of the patient as compared to a control, pre-treatment sample, or a patient that does not have detectable levels of the effector memory CD8 positive T cells, or CD8 positive T cells, is indicative of treatment efficacy for treatment with an anti-PVRIG antibody alone or in combination with an anti-PD-1 antibody.

In some embodiments, the one or more biomarkers are selected from the group consisting of LAMP3, HLA-DR and CD83.

In some embodiments, the method comprises measuring the expression level of one or more of LAMP3, HLA-DR and CD83. In some embodiments, the method comprises measuring the expression level of LAMP3. In some embodiments, the method comprises measuring the expression level of HLA-DR. In some embodiments, the method comprises measuring the expression level of CD83.

In some embodiments, the level of the one or more biomarkers selected from the group consisting of LAMP3, HLA-DR and CD83 is indicative of treatment efficacy for treatment with an anti-PVRIG antibody alone or in combination with an anti-PD-1 antibody, when the expression level of the one or more of these biomarkers is increased by at least 1-fold, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, or 30-fold as compared to a control, pre-treatment sample, or a patient that does not respond to treatment with an anti-PVRIG antibody alone or in combination with an anti-PD-1 antibody.

In some embodiments, the level of LAMP3 is indicative of treatment efficacy for treatment with an anti-PVRIG antibody alone or in combination with an anti-PD-1 antibody, when the expression level of LAMP3 is increased by at least 1-fold, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, or 30-fold as compared to a control, pre-treatment sample, or a patient that does not respond to treatment with an anti-PVRIG antibody alone or in combination with an anti-PD-1 antibody.

In some embodiments, the level of HLA-DR is indicative of treatment efficacy for treatment with an anti-PVRIG antibody alone or in combination with an anti-PD-1 antibody, when the expression level of HLA-DR is increased by at least 1-fold, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, or 30-fold as compared to a control, pre-treatment sample, or a patient that does not respond to treatment with an anti-PVRIG antibody alone or in combination with an anti-PD-1 antibody.

In some embodiments, the level of CD83 is indicative of treatment efficacy for treatment with an anti-PVRIG antibody alone or in combination with an anti-PD-1 antibody, when the expression level of CD83 is increased by at least 1-fold, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, or 30-fold as compared to a control, pre-treatment sample, or a patient that does not respond to treatment with an anti-PVRIG antibody alone or in combination with an anti-PD-1 antibody.

In some embodiments, the biomarkers is KI67.

In some embodiments, measuring the level of the biomarker comprises measuring expression level of KI67.

In some embodiments, the level of the effector memory CD8 positive T cells, CD8 positive T cells, is indicative of treatment efficacy for treatment with an anti-PVRIG antibody alone or in combination with an anti-PD-1 antibody when the expression level of KI67 is increased by at least 1-fold, 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.5-fold, 5.6-fold, 5.7-fold, 5.8-fold, 5.9-fold, 6-fold, 6.1-fold, 6.2-fold, 6.3-fold, 6.4-fold, 6.5-fold, 6.6-fold, 6.7-fold, 6.8-fold, 6.9-fold, 7-fold, 7.1-fold, 7.2-fold, 7.3-fold, 7.4-fold, 7.5-fold, 7.6-fold, 7.7-fold, 7.8-fold, 7.9-fold, 8-fold, 8.1-fold, 8.2-fold, 8.3-fold, 8.4-fold, 8.5-fold, 8.6-fold, 8.7-fold, 8.8-fold, 8.9-fold, 9-fold, 9.1-fold, 9.2-fold, 9.3-fold, 9.4-fold, 9.5-fold, 9.6-fold, 9.7-fold, 9.8-fold, 9.9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, or 50-fold, as compared to a control, pre-treatment sample, or a patient that does not respond to treatment with an anti-PVRIG antibody alone or in combination with an anti-PD-1 antibody. In some embodiments, the one or more biomarkers include CD8 and CD4.

In some embodiments, the method comprises measuring the level of CD8. In some embodiments, the method comprises measuring the level of CD8. In some embodiments, the method comprises measuring the level of CD8. In some embodiments, the method comprises measuring the level of CD4. In some embodiments, the method comprises measuring the ratio of CD8/CD4.

In some embodiments, level of the CD8/CD4 ratio is indicative of treatment efficacy for treatment with an anti-PVRIG antibody in combination with nivolumab when the CD8/CD4 ratio is increased by at least about 10%, 20%, 30%, 40%, 50%, 60%, 70%, 80%, 90%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, or 1000%, as compared to a control, pre-treatment sample, or a patient that does not respond to treatment with an anti-PVRIG antibody in combination with nivolumab.

In some embodiments, the level of the CD8/CD4 ratio is indicative of treatment efficacy for treatment with an anti-PVRIG antibody in combination with nivolumab when the CD8/CD4 ratio is increased by about 1.1-fold, 1.2-fold, 1.3-fold, 1.4-fold, 1.5-fold, 1.6-fold, 1.7-fold, 1.8-fold, 1.9-fold, 2-fold, 2.25-fold, 2.5-fold, 2.75-fold, 3-fold, 3.25-fold, 3.5-fold, 3.75-fold, 4-fold, 4.25-fold, 4.5-fold, 4.75-fold, 5-fold, 5.25-fold, 5.5-fold, 5.75-fold, 6-fold, 6.25-fold, 6.5-fold, 6.75-fold, 7-fold, 7.25-fold, 7.5-fold, 7.75-fold, 8-fold, 8.25-fold, 8.5-fold, 8.75-fold, 9-fold, 9.25-fold, 9.5-fold, 9.75-fold, 10-fold, 10.25-fold, 10.5-fold, 10.75-fold or 11-fold, as compared to a control, pre-treatment sample, or a patient that does not respond to treatment with an anti-PVRIG antibody in combination with nivolumab.

In some embodiments, the effector memory CD8 positive T cells are CD8+CD45RA−CCR7−.

In some embodiments, the level of the effector memory CD8 positive T cells is measured by measuring the expression level of one or more of CD8, CD45RA, and CCR7.

In some embodiments, the expression level of CD8 is indicative of treatment efficacy for treatment with an anti-PVRIG antibody in combination with nivolumab, when the expression level of CD8 is increased by at least 1-fold, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, or 30-fold as compared to a control, pre-treatment sample, or a patient that does not respond to treatment with an anti-PVRIG antibody in combination with nivolumab.

In some embodiments, the expression level of CD45RA is indicative of treatment efficacy for treatment with an anti-PVRIG antibody in combination with nivolumab, when the expression level of CD45RA is decreased by at least 5%, 10%, 15%, 20%, 25%, 30%, 35%, 40%, 45%, 50%, 55%, 60%, 65%, 70%, 75%, 80%, 85%, 90%, 95%, or more as compared to a control, pre-treatment sample, or a patient that does not respond to treatment with an anti-PVRIG antibody in combination with nivolumab.

In some embodiments, the expression level of CCR7 is indicative of treatment efficacy for treatment with an anti-PVRIG antibody in combination with nivolumab, when the expression level of CCR7 is decreased by at least 5%, 10%, 15%, 20%, 25%, 30%, 35%, 40%, 45%, 50%, 55%, 60%, 65%, 70%, 75%, 80%, 85%, 90%, 95%, or more as compared to a control, pre-treatment sample, or a patient that does not respond to treatment with an anti-PVRIG antibody in combination with nivolumab.

In some embodiments, the NK-T cells being present and/or being present at an increased level in the peripheral blood of the patient as compared to a control, pre-treatment sample, or a patient that does not have detectable levels of the NK-T cells, is indicative of treatment efficacy for treatment with an anti-PVRIG antibody alone or in combination with an anti-PD-1 antibody.

In some embodiments, the level of the NK-T cells is indicative of treatment efficacy for treatment with the anti-PVRIG antibody alone or in combination with an anti-PD-1 antibody when the level of NK-T cells is increased by at least 1-fold, 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.5-fold, 5.6-fold, 5.7-fold, 5.8-fold, 5.9-fold, 6-fold, 6.1-fold, 6.2-fold, 6.3-fold, 6.4-fold, 6.5-fold, 6.6-fold, 6.7-fold, 6.8-fold, 6.9-fold, 7-fold, 7.1-fold, 7.2-fold, 7.3-fold, 7.4-fold, 7.5-fold, 7.6-fold, 7.7-fold, 7.8-fold, 7.9-fold, 8-fold, 8.1-fold, 8.2-fold, 8.3-fold, 8.4-fold, 8.5-fold, 8.6-fold, 8.7-fold, 8.8-fold, 8.9-fold, 9-fold, 9.1-fold, 9.2-fold, 9.3-fold, 9.4-fold, 9.5-fold, 9.6-fold, 9.7-fold, 9.8-fold, 9.9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, or 50-fold, as compared to a control, pre-treatment sample, or a patient that does not respond to treatment with an anti-PVRIG antibody alone or in combination with an anti-PD-1 antibody.

In some embodiments, the biological sample is obtained from a tumor, tumor microenvironment, and/or peripheral blood from the cancer patient.

In some embodiments, the method of treatment provided herein includes in part a method for determining or predicting the efficacy of treatment with an anti-PVRIG antibody alone or in combination with an anti-PD-1 antibody, the method comprising:
(a) measuring the level of TCR diversity and repertoire value in a biological sample of a cancer patient;
(b) quantitating the measurement of the level of TCR diversity and repertoire value; and
(c) correlating the level of the TCR diversity and repertoire value with the efficacy of treatment.

In some embodiments, the method of treatment provided herein includes in part a method for determining a cancer patient population for treatment with an anti-PVRIG antibody alone or in combination with an anti-PD-1 antibody, the method comprising:
(a) measuring the level of TCR diversity and repertoire value in a biological sample from the cancer patient;
(b) quantitating the measurement of the level of TCR diversity and repertoire value;
(c) treating the cancer patient with the anti-PVRIG antibody alone or in combination with the anti-PD-1 antibody when there is a decrease in the TCR diversity index and increase in the number of expanded TCR repertoire level as compared to a control, pre-treatment sample, or a patient that does not respond to treatment with an anti-PVRIG antibody alone or in combination with an anti-PD-1 antibody.

In some embodiments, the biological sample used for determining the level of TCR diversity and repertoire value is obtained from a tumor, tumor microenvironment, and/or peripheral blood from the cancer patient.

In some embodiments, the TCR diversity and repertoire value is measured before and/or during the treatment with an anti-PVRIG antibody alone or in combination with an anti-PD-1 antibody.

In some embodiments, the TCR diversity and repertoire value is indicated by number of unique clones of TCRα and/or TCRβ.

In some embodiments, measuring the TCR diversity and repertoire value comprises calculating a TCR diversity index from TCR repertoire analysis of T cells in the biological sample.

In some embodiments, the method of measuring the level of TCR diversity and repertoire value comprises calculating the TCR diversity index from TCR repertoire analysis of the T cells in a biopsy taken between cycle 2 and cycle 3 of the treatment.

In some embodiments, the TCR diversity index and/or repertoire is selected from the group consisting of a Shannon index, a Simpson index, an inverse Simpson index, a normalized Shannon index, a Unique50 index, a DE30 index, a DE80 index, Gini Coefficient, proportion of TCR clone reads out of total, and a DE50 index. In some embodiments, the TCR diversity index is a Gini Coefficient. In some embodiments, the TCR diversity index is proportion of TCR clone reads out of total. In some embodiment, the TCR diversity index is characterized by the proportion of top unique TCR clones (by ranking of TCR reads) in total TCR reads.

In some embodiments, a Gini Coefficient of about 0.6 or more is indicative of treatment efficacy for treatment with the anti-PVRIG antibody. In some embodiments, an increase of a Gini Coefficient of about 0.3 or more during the treatment, is indicative of treatment efficacy for treatment with an anti-PVRIG antibody alone or in combination with an anti-PD-1 antibody.

In some embodiments, an increase of top unique TCR clones (by ranking of TCR reads) the top deciles comprises about 50% or more of total TCR reads during the treatment is indicative of treatment efficacy for treatment with an anti-PVRIG antibody alone or in combination with an anti-PD-1 antibody.

In some embodiments, the method of treatment includes in part a method for determining or predicting the efficacy of treatment with an anti-PVRIG antibody alone or in combination with an anti-PD-1 antibody, the method comprising:
(a) measuring the level of TCR diversity and/or level of serum IFNγ of a cancer patient;
(b) quantitating the measurement of the level of TCR diversity and/or level of serum IFNγ; and
(c) correlating the level of the TCR diversity and/or level of serum IFNγ with the efficacy of treatment.

In some embodiments, the method of treatment includes in part a method for a method for determining a cancer patient population for treatment with an anti-PVRIG antibody alone or in combination with an anti-PD-1 antibody, the method comprising:
(a) detecting the level of TCR diversity and/or level of serum IFNγ in a biological sample from the cancer patient;
(b) quantitating the measurement of the level of TCR diversity and/or level of serum IFNγ;
(c) treating the cancer patient with the anti-PVRIG antibody alone or in combination with the anti-PD-1 antibody when the level of serum IFNγ is present at an increased level and/or when there is a decrease in the TCR diversity index and increase in the number of expanded TCR repertoire level as compared to a control, pre-treatment sample, or a patient that does not respond to treatment with the anti-PVRIG antibody alone or in combination with the anti-PD-1 antibody.

In some embodiments, the biological sample is obtained from a tumor, tumor microenvironment, and/or peripheral blood from the cancer patient.

In some embodiments, TCR diversity is indicated by number of unique clones of TCRα and/or TCRβ.

In some embodiments, the level of TCR diversity is indicative of treatment efficacy for treatment with the anti-PVRIG antibody alone or in combination with an anti-PD-1 antibody when the level of TCR diversity is decreased by at least 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.5-fold, 5.6-fold, 5.7-fold, 5.8-fold, 5.9-fold, 6-fold, 6.1-fold, 6.2-fold, 6.3-fold, 6.4-fold, 6.5-fold, 6.6-fold, 6.7-fold, 6.8-fold, 6.9-fold, 7-fold, 7.1-fold, 7.2-fold, 7.3-fold, 7.4-fold, 7.5-fold, 7.6-fold, 7.7-fold, 7.8-fold, 7.9-fold, 8-fold, 8.1-fold, 8.2-fold, 8.3-fold, 8.4-fold, 8.5-fold, 8.6-fold, 8.7-fold, 8.8-fold, 8.9-fold, 9-fold, 9.1-fold, 9.2-fold, 9.3-fold, 9.4-fold, 9.5-fold, 9.6-fold, 9.7-fold, 9.8-fold, 9.9-fold, 10-fold, 20-fold, 30-fold, 40-fold, 50-fold, 60-fold, 70-fold, 80-fold, 90-fold, 100-fold, 110-fold, 120-fold, 130-fold, 140-fold, 150-fold, 160-fold, 170-fold, 180-fold, 190-fold, or 200-fold, as compared to a control, pre-treatment sample, or a patient that does not respond to treatment with an anti-PVRIG antibody alone or in combination with an anti-PD-1 antibody.

In some embodiments, the level of serum IFNγ is indicative of treatment efficacy for treatment with an anti-PVRIG antibody alone or in combination with an anti-PD-1 antibody when the level of serum IFNγ is increased by at least 1-fold, 2-fold, 3-fold, 4-fold, 5-fold, 6-fold, 7-fold, 8-fold, 9-fold, or 10-fold, as compared to a control, pre-treatment sample, or a patient that does not respond to treatment with an anti-PVRIG antibody alone or in combination with an anti-PD-1 antibody.

In some embodiments, the method of treatment provided herein includes in part a method for determining or predicting the efficacy of treatment with an anti-PVRIG antibody alone or in combination with an anti-PD-1 antibody, the method comprising:
(a) measuring the expression level of IFNγ in a biological sample of the cancer patient;
(b) quantitating the measurement of the expression level of IFNγ; and
(c) correlating the expression level of IFNγ with the efficacy of treatment.

In some embodiments, the method of treatment provided herein includes in part a method for determining a cancer patient population for treatment with an anti-PVRIG antibody alone or in combination with an anti-PD-1 antibody, the method comprising:
(a) measuring the expression level of IFNγ in a biological sample from the cancer patient;
(b) quantitating the measurement of the expression level of IFNγ;
(c) treating the cancer patient with an anti-PVRIG antibody alone or in combination with an anti-PD-1 antibody when there is an increase the expression level of IFNγ as compared to a control, pre-treatment sample, or a patient that does not respond to treatment with an anti-PVRIG antibody alone or in combination with an anti-PD-1 antibody.

In some embodiments, measuring the expression level of IFNγ comprises measuring the expression level of IFNγ signature and/or Lymphoid signature.

In some embodiments, the IFNγ signature comprises one or more of CXCL10, CXCL9, IDO1, STAT1, HLA_DRA, and IFNG.

In some embodiments, the Lymphoid signature comprises one of more of PRF1, GZMB, CD8A, CD8B, CD3G, CD4, CD3D, and CD3E.

In some embodiments, measuring the expression level of IFNγ comprises measuring the expression level of one or more of the CXCL10, CXCL9, CD137, HLA_DRA, IFNG, PRF1, GZMB, GZMA, GZMH proteins or protein levels and/or mRNAs or mRNA levels.

In some embodiments, the expression level of the IFNγ, IFNγ signature and/or Lymphoid signature, is indicative of treatment efficacy for treatment with an anti-PVRIG antibody alone or in combination with an anti-PD-1 antibody when the expression level of any of the IFNγ, IFNγ signature and/or Lymphoid signature is increased by at least 1-fold, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, or 10-fold, as compared to a control, pre-treatment sample, or a patient that does not respond to treatment with an anti-PVRIG antibody alone or in combination with an anti-PD-1 antibody.

In some embodiments, the expression level of any one of the CXCL10, CXCL9, CD137, HLA_DRA, IFNG, PRF1, GZMB, GZMA, GZMH proteins or protein levels and/or mRNAs or mRNA levels, is indicative of treatment efficacy for treatment with an anti-PVRIG antibody alone or in combination with an anti-PD-1 antibody when the expression level of any of the of any one of the CXCL10, CXCL9, CD137, HLA_DRA, IFNG, PRF1, GZMB, GZMA, GZMH proteins or protein levels and/or mRNAs or mRNA levels is increased by at least 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, or 10-fold, as compared to a control, pre-treatment sample, or a patient that does not respond to treatment with an anti-PVRIG antibody alone or in combination with an anti-PD-1 antibody.

In some embodiments, the anti-PD-1 antibody is nivolumab.

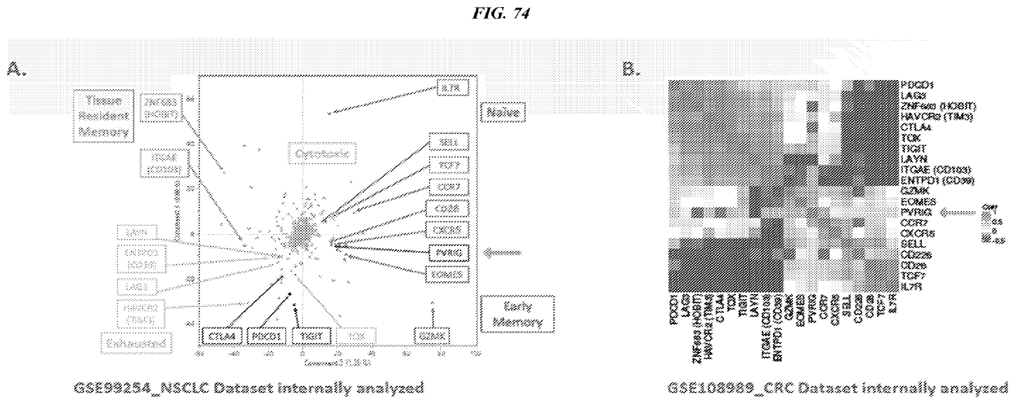

In some embodiments, the anti-PVRIG antibodies for use with the invention comprise the PVRIG binding portion comprising CDRs identical to those shown in FIGS. 3, 5 and 74, as provided in US Patent Publication No. 2020/040081 and PCT Publication Nos. WO 2021/113831, WO 2017/041004 and WO 2018/017864, each of which has been incorporated herein by reference in its entirety.

In some embodiments, nivolumab is used in combination with the anti-PVRIG antibody comprising
i) a heavy chain variable domain comprising the vhCDR1, vhCDR2, and vhCDR3 from the heavy chain of CHA.7.518.1.H4 (S241P) (SEQ ID NO:4), and
ii) a light chain variable domain comprising the vlCDR1, vlCDR2, and vlCDR3 from the light chain of CHA.7.518.1.H4 (S241P) (SEQ ID NO:9).

In some embodiments of the method of determining or predicting the efficacy of treatment with an anti-PVRIG antibody alone or in combination with an anti-PD-1 antibody, the anti-PVRIG antibody comprises:
i) a heavy chain variable domain comprising the vhCDR1, vhCDR2, and vhCDR3 from the heavy chain of CHA.7.518.1.H4 (S241P) (SEQ ID NO:4), and
ii) a light chain variable domain comprising the vlCDR1, vlCDR2, and vlCDR3 from the light chain of CHA.7.518.1.H4 (S241P) (SEQ ID NO:9).

In some embodiments of the method of method for determining a cancer patient population for treatment with an anti-PVRIG antibody alone or in combination with an anti-PD-1 antibody, the anti-PVRIG antibody comprises:
i) a heavy chain variable domain comprising the vhCDR1, vhCDR2, and vhCDR3 from the heavy chain of CHA.7.518.1.H4 (S241P) (SEQ ID NO:4), and
ii) a light chain variable domain comprising the vlCDR1, vlCDR2, and vlCDR3 from the light chain of CHA.7.518.1.H4 (S241P) (SEQ ID NO:9).

In some embodiments, wherein said anti-PVRIG antibody comprises a CH1-hinge-CH2-CH3 sequence of IgG4 (SEQ ID NO:17 or SEQ ID NO:50), wherein said hinge region optionally comprises mutations. In some embodiments, wherein said anti-PVRIG antibody comprises the CH1-hinge-CH2-CH3 region from IgG1, IgG2, IgG3, or IgG4, wherein said hinge region optionally comprises mutations. In some embodiments, wherein said heavy chain variable domain is from the heavy chain of CHA.7.518.1.H4 (S241P) (SEQ ID NO:4) and said light chain variable domain is from the light chain of CHA.7.518.1.H4 (S241P) (SEQ ID NO: 9). In some embodiments, wherein said anti-PVRIG antibody comprises a CL region of human kappa 2 light chain. In some embodiments, wherein the anti-PVRIG antibody is administered as a stable liquid pharmaceutical formulation. In some embodiments, wherein the anti-PVRIG antibody is administered as a stable liquid pharmaceutical formulation as described herein. In some embodiments, the stable liquid pharmaceutical formulation as described herein comprising the anti-PVRIG antibody upon administration to a cancer patient in combination with the anti-PD-1 antibody induces proliferation of one or more cells comprising activated DC cells, effector memory CD8 positive T cells, CD8 positive T cells, and NK-T cells.

In some embodiments, the stable liquid pharmaceutical formulation as described herein comprising the anti-PVRIG antibody upon administration to a cancer patient in combination with the anti-PD-1 antibody increases the level of serum IFNγ and/or there is a decrease in the TCR diversity index and increase in the number of expanded TCR repertoire level as compared to a control, pre-treatment sample, pre treatment sample, or a patient that does not respond to treatment with an anti-PVRIG antibody alone or in combination with an anti-PD-1 antibodyan anti-PVRIG antibody alone or in combination with an anti-PD-1 antibody.

In some embodiments, the early memory CD8 T cells can be employed as a biomarker or companion diagnostic for use in the determining treatment regimens as well as predicting or determining treatment efficacy. In some embodiments, the presence of early memory CD8 T cells can be employed as a biomarker or companion diagnostic for use in the determining treatment regimens as well as predicting or determining treatment efficacy for treatment with an anti-PVRIG antibody alone or in combination with the anti-PD-1 antibody. In some embodiments, the percentage of early memory CD8 T cells can be employed as a biomarker or companion diagnostic for use in the determining treatment regimens as well as predicting or determining treatment efficacy for treatment with an anti-PVRIG antibody alone or in combination with the anti-PD-1 antibody.

In some embodiments, the activated dendritic cells can be employed as a biomarker or companion diagnostic for use in the determining treatment regimens as well as predicting or determining treatment efficacy. In some embodiments, the presence of activated dendritic cells can be employed as a biomarker or companion diagnostic for use in the determining treatment regimens as well as predicting or determining treatment efficacy for treatment with an anti-PVRIG antibody alone or in combination with the anti-PD-1 antibody. In some embodiments, the proliferation of activated dendritic cells can be employed as a biomarker or companion diagnostic for use in the determining treatment regimens as well as predicting or determining treatment efficacy for treatment with an anti-PVRIG antibody alone or in combination with the anti-PD-1 antibody. In some embodiments, the presence of activated dendritic cells can be employed as a biomarker or companion diagnostic for use in the determining treatment regimens as well as predicting or determining treatment efficacy for treatment with an anti-PVRIG antibody alone or in combination with the anti-PD-1 antibody. In some embodiments, the activated dendritic cell percentage of total myeloid cells can be employed as a biomarker or companion diagnostic for use in the determining treatment regimens as well as predicting or determining treatment efficacy for treatment with an anti-PVRIG antibody alone or in combination with the anti-PD-1 antibody. In some embodiments, the presence of at least 1% of activated dendritic cells out of total myeloid cells in tumor, TME, and/or peripheral blood of a patient is indicative for predicting or determining treatment efficacy for treatment with an anti-PVRIG antibody alone or in combination with the anti-PD-1 antibody.

In some embodiments, the NK-T cells can be employed as a biomarker or companion diagnostic for use in the determining treatment regimens as well as predicting or determining treatment efficacy. In some embodiments, the presence of NK-T cells can be employed as a biomarker or companion diagnostic for use in the determining treatment regimens as well as predicting or determining treatment efficacy for treatment with an anti-PVRIG antibody alone or in combination with the anti-PD-1 antibody. In some embodiments, the proliferation of NK-T cells can be employed as a biomarker or companion diagnostic for use in the determining treatment regimens as well as predicting or determining treatment efficacy for treatment with an anti-PVRIG antibody alone or in combination with the anti-PD-1 antibody. In some embodiments, the presence of NK-T cells can be employed as a biomarker or companion diagnostic for use in the determining treatment regimens as well as predicting or determining treatment efficacy for treatment with an anti-PVRIG antibody alone or in combination with the anti-PD-1 antibody.

In some embodiments, the biomarkers provided herein can be used for determining, modifying, altering, and/or predicting the outcome for treatment of tumors. In some embodiments, the effector memory T cell markers, effector memory CD8 positive T cell markers, CD8 positive T cell markers, activated DC markers, and/or NK-T cell markers can be used for determining, modifying, altering, and/or predicting the outcome for treatment of tumors. In some embodiments, one or more biomarkers comprising LAMP3, HLA-DR, and CD83 can be used for determining, modifying, altering, and/or predicting the outcome for treatment of tumors. In some embodiments, KI67 can be used for determining, modifying, altering, and/or predicting the outcome for treatment of tumors. In some embodiments, level of TCR diversity can be used for determining, modifying, altering, and/or predicting the outcome for treatment of tumors. In some embodiments, level of serum IFNγ can be used for determining, modifying, altering, and/or predicting the outcome for treatment of tumors. In some embodiments, TME immune activation can be used for determining, modifying, altering, and/or predicting the outcome for treatment of tumors. In some embodiments, the activation and/or proliferation of effector memory T cell, effector memory CD8 positive T cell, CD8 positive T cell, dendritic cells, and/or NK-T cells can be employed as a biomarker for determining, modifying, altering, and/or predicting the outcome for treatment of tumors.

In some embodiments, the biomarkers of the present invention can be used for determining, modifying, altering, and/or predicting the outcome for treatment of cancer including carcinoma, lymphoma, sarcoma, and/or leukemia. In some embodiments, the biomarkers can be used for determining, modifying, altering, and/or predicting the outcome for cancer including vascularized tumors, melanoma, non-melanoma skin cancer (squamous and basal cell carcinoma), mesothelioma, squamous cell cancer, lung cancer, small-cell lung cancer, non-small cell lung cancer, neuroendocrine lung cancer (including pleural mesothelioma, neuroendocrine lung carcinoma), NSCL (large cell), NSCLC large cell adenocarcinoma, non-small cell lung carcinoma (NSCLC), NSCLC squamous cell, soft-tissue sarcoma, Kaposi's sarcoma, adenocarcinoma of the lung, squamous carcinoma of the lung, NSCLC with PDL1>=50% TPS, neuroendocrine lung carcinoma, atypical carcinoid lung cancer, cancer of the peritoneum, esophageal cancer, hepatocellular cancer, liver cancer (including HCC), gastric cancer, stomach cancer (including gastrointestinal cancer), pancreatic cancer, glioblastoma, cervical cancer, ovarian cancer, urothelial cancer, bladder cancer, hepatoma, glioma, brain cancer (as well as edema, such as that associated with brain tumors), breast cancer (including, for example, triple negative breast cancer), testis cancer, testicular germ cell tumors, colon cancer, colorectal cancer (CRC), colorectal cancer MSS (MSS-CRC); microsatellite stable colorectal carcinoma/carcinoma), metastatic MSS-CRC, refractory metastatic MSS-CRC, refractory MSS colorectal; MSS (microsatellite stable status), primary peritoneal cancer, microsatellite stable primary peritoneal cancer, platinum resistant microsatellite stable primary peritoneal cancer, CRC (MSS unknown), rectal cancer, endometrial cancer (including endometrial carcinoma), uterine carcinoma, salivary gland carcinoma, kidney cancer, renal cell cancer (RCC), renal cell carcinoma (RCC), gastro-esophageal junction cancer, prostate cancer, vulval cancer, thyroid cancer, hepatic carcinoma, carcinoid carcinoma, head and neck cancer, B-cell lymphoma (including non-Hodgkin's lymphoma, as well as low grade/follicular non-Hodgkin's lymphoma (NHL), small lymphocytic (SL) NHL, intermediate grade/follicular NHL, intermediate grade diffuse NHL, Diffuse Large B cell lymphoma, high grade immunoblastic NHL, high grade lymphoblastic NHL, high grade small non-cleaved cell NHL, bulky disease NHL, mantle cell lymphoma, AIDS-related lymphoma, and Waldenström's Macroglobulinemia, Hodgkin's lymphoma (HD), chronic lymphocytic leukemia (CLL), acute lymphoblastic leukemia (ALL), T cell Acute Lymphoblastic Leukemia (T-ALL), Acute myeloid leukemia (AML), Hairy cell leukemia, chronic myeloblastic leukemia, multiple myeloma, post-transplant lymphoproliferative disorder (PTLD), abnormal vascular proliferation associated with phakomatoses, Meigs' syndrome, Merkel Cell cancer, MSI-high cancer, KRAS mutant tumors, adult T-cell leukemia/lymphoma, adenoid cystic cancer (including adenoid cystic carcinoma), malignant melanoma, pancreatic cancer, pancreatic adenocarcinoma, ovarian cancer (including ovarian carcinoma), platinum resistant ovarian cancer, high grade serous adenocarcinoma, pleural mesothelioma, cervical squamous cell carcinoma (cervical SCC), anal squamous cell carcinoma (anal SCC), carcinoma of unknown primary, gallbladder cancer, malignant melanoma, pleural mesothelioma, chordoma, endometrial sarcoma, chondrosarcoma, uterine sarcoma, uveal melanoma, amyloidosis, AL-amyloidosis, astrocytoma, and/or Myelodysplastic syndromes (MDS).

In some embodiments, the biomarkers of the present invention can be used for determining, modifying, altering, and/or predicting the outcome for a cancer selected from the group consisting of prostate cancer, liver cancer (HCC), rectal cancer, colorectal cancer (CRC), colorectal cancer MSS (MSS-CRC; microsatellite stable colorectal carcinoma/carcinoma), metastatic MSS-CRC, refractory metastatic MSS-CRC, refractory MSS colorectal, CRC (MSS unknown), ovarian cancer (including ovarian carcinoma), platinum resistant ovarian cancer, hi grade serous adenocarcinoma, endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), uveal melanoma, glioma, renal cell cancer (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cell cancer, MSI-high cancer, KRAS mutant tumors, adult T-cell leukemia/lymphoma, pleural mesothelioma, anal SCC, neuroendocrine lung cancer (including neuroendocrine lung carcinoma), small cell lung cancer, NSCLC, NSCLC large cell, NSCLC squamous cell, NSCLC adenocarcinoma, atypical carcinoid lung cancer, NSCLC with PDL1>=50% TPS, cervical SCC, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, Myelodysplastic syndromes (MDS), HNSCC, PD1 refractory or relapsing cancer, gastroesophageal junction cancer, gastric cancer, chordoma, sarcoma, endometrial sarcoma, chondrosarcoma, uterine sarcoma, plasma cell disorders, multiple myeloma, amyloidosis, AL-amyloidosis, glioblastoma, astrocytoma and fallopian tube cancer.

In some embodiments, the biomarkers can be used for determining, modifying, altering, and/or predicting the outcome for a cancer selected from the group consisting of prostate cancer, liver cancer (HCC), rectal cancer, colorectal cancer (CRC), colorectal cancer MSS (MSS-CRC; microsatellite stable colorectal carcinoma/carcinoma), metastatic MSS-CRC, refractory metastatic MSS-CRC, refractory MSS colorectal, CRC (MSS unknown), ovarian cancer (including ovarian carcinoma), platinum resistant ovarian cancer, high grade serous adenocarcinoma, endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), uveal melanoma, glioma, renal cell cancer (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cell cancer, MSI-high cancer, KRAS mutant tumors, adult T-cell leukemia/lymphoma, pleural mesothelioma, anal SCC, neuroendocrine lung cancer (including neuroendocrine lung carcinoma), small cell lung cancer, NSCLC, NSCLC large cell, NSCLC squamous cell, NSCLC adenocarcinoma, atypical carcinoid lung cancer, NSCLC with PDL1>=50% TPS, cervical SCC, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, Myelodysplastic syndromes (MDS), HNSCC, PD1 refractory or relapsing cancer, gastroesophageal junction cancer, gastric cancer, chordoma, sarcoma, endometrial sarcoma, chondrosarcoma, uterine sarcoma, plasma cell disorders, multiple myeloma, amyloidosis, AL-amyloidosis, glioblastoma, astrocytoma and fallopian tube cancer.

### VIII. Formulations of Anti-PVRIG Antibodies

The anti-PVRIG antibodies and/or antigen binding portions thereof compositions (e.g., anti-PVRIG antibodies including those with CDRs identical to those shown in FIG. 3) can be formulated into pharmaceutical compositions comprising a carrier suitable for the desired delivery method. Suitable carriers include any material that when combined with the therapeutic composition retains the anti-tumor function of the therapeutic composition and is generally non-reactive with the patient's immune system. Examples include, but are not limited to, any of a number of standard pharmaceutical carriers such as sterile phosphate buffered saline solutions, bacteriostatic water, and the like (see, generally, Remington's Pharmaceutical Sciences 16^{th} Edition, A. Osal., Ed., 1980). Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g., Zn-protein complexes); and/or non-ionic surfactants such as TWEEN™, PLURONICS™, or polyethylene glycol (PEG).

In a preferred embodiment, the pharmaceutical composition that comprises anti-PVRIG antibodies including those with CDRs identical to those shown in FIG. 3) of the invention may be in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine. The formulations to be used for in vivo administration are preferrably sterile. This is readily accomplished by filtration through sterile filtration membranes or other methods.

As used herein, the term “activity” refers to a functional activity or activities of anti-PVRIG antibodies and/or antigen binding portions thereof. Functional activities include, but are not limited to, biological activity and binding affinity.

As used herein, the term “stability” is used in a structural context, e.g., relating to the structural integrity of an anti-PVRIG antibody and/or antigen binding portion thereof, or in a functional context, e.g., relating to a an anti-PVRIG antibody and/or antigen binding portion thereof's ability to retain its function and/or activity over time (e.g., including anti-PVRIG antibody and/or antigen binding portion thereof stability or anti-PVRIG antibody and/or antigen binding portion thereof formulation stability, wherein the anti-PVRIG antibody includes those with CDRs identical to those shown in FIG. 3). As will be appreciated, the anti-PVRIG antibody and/or antigen binding portion thereof under discussion may be contained within a formulation in accordance with the methods and compositions described herein, and the stability of that protein refers to its stability in that formulation. In some embodiments, the stability of an anti-PVRIG antibody and/or antigen binding portion thereof composition is determined by measuring the binding activity of the composition, including for example, using the assays described in the application and figures provided herewith, as well as an other applicable assays known in the art. In some embodiments, the stability of an anti-PVRIG antibody and/or antigen binding portion thereof composition is formulated with sugar, sugar alcohol, and/or non-ionic surfactant, as described herein, is compared to an anti-PVRIG antibody and/or antigen binding portion thereof composition formulated without the at least one amino acid, salt, and/or non-ionic surfactant and/or with a different combination of components. In some embodiments, the formulation does not comprise a sugar and/or sugar alcohol.

As used herein, a “storage stable” aqueous an anti-PVRIG antibody and/or antigen binding portion thereof composition refers to a an anti-PVRIG antibody and/or antigen binding portion thereof comprising solution that has been formulated to increase the stability of the protein in solution, for example by at least 10%, over a given storage time. In the context of the present disclosure, an anti-PVRIG antibody and/or antigen binding portion thereof can be made “storage stable” by the addition of at least one amino acid, salt, or non-ionic surfactant as a stabilizing agent. In some embodiments, the stability of the anti-PVRIG antibody and/or antigen binding portion thereof in any given formulation can be measured, for example, by monitoring the formation of aggregates, loss of bulk binding activity, or formation of degradation products, over a period of time. The absolute stability of a formulation, and the stabilizing effects of the sugar, sugar alcohol, or non-ionic surfactant, will vary dependent upon the particular composition being stabilized. In one embodiment, the stability of an anti-PVRIG antibody and/or antigen binding portion thereof composition is determined by measuring the anti-PVRIG antibody and/or antigen binding portion thereof binding activity of the composition. For example, by using an ELISA or other binding activity assay. In one embodiment, the stability of an anti-PVRIG antibody and/or antigen binding portion thereof composition formulated with sugar, sugar alcohol, and/or non-ionic surfactant, as described herein, is compared to an anti-PVRIG antibody and/or antigen binding portion thereof composition formulated without the a least one amino acid, salt, and/or non-ionic surfactant and/or with a different combination of components. In some embodiments, the formulation does not comprise a sugar and/or sugar alcohol.

As used herein, “shelf-life” refers to the period of time a formulation maintains a predetermined level of stability at a predetermined temperature. In particular embodiments, the predetermined temperature refers to frozen (e.g., −80° C., −25° C., 0° C.), refrigerated (e.g., 0° to 10° C.), or room temperature (e.g., 18° C. to 32° C.) storage.

As used herein, the term “time of stability” refers to the length of time a formulation is considered stable. For example, the time of stability for a formulation may refer to the length of time for which the level of protein aggregation and/or degradation in the formulation remains below a certain threshold (e.g., 1%, 2%, 3%, 4%, 5%, 6%, 7%, 8%, 9%, 10%, 1 1%, 12%, 13%, 14%, 15%, 16%, 17%, 18%, 19%, 20%, etc.), and/or the length of time a formulation maintains biological activity above a certain threshold (e.g., 100%, 95%, 90%, 85%, 80%, 75%, 70%, 65%, 60%, 55%, 50%, etc.) of the amount of activity (including, for example, binding activity) present in the formulation at the start of the storage period.

In the context of the present disclosure, a storage stable aqueous composition of a an anti-PVRIG antibody and/or antigen binding portion thereof formulated with a sugar, sugar alcohol, and/or non-ionic surfactant will have a longer time of stability than a composition of the same an anti-PVRIG antibody and/or antigen binding portion thereof formulated without the at least one amino acid, salt, and/or non-ionic surfactant. In some embodiments, a storage stable aqueous composition of an anti-PVRIG antibody and/or antigen binding portion thereof, will have a time of stability that is, for example, at least 10% greater than the time of stability for an anti-PVRIG antibody and/or antigen binding portion thereof composition formulated in the absence of the at least one amino acid, salt, and/or non-ionic surfactant, or at least 15%, 20%, 25%, 30%, 35%, 40%, 45%, 50%, 55%, 60%, 65%, 70%, 75%, 80%, 85%, 90%, 95%, 100%, 1 10%, 120%, 130%, 140%, 150%, 160%, 170%, 180%, 190% greater, or at least 2 times greater, or at least 2.5 times, 3.0 times, 3.5 times, 4.0 times, 4.5 times, 5.0 times, 5.5 times, 6.0 times, 6.5 times, 7.0 times, 7.5 times, 8.0 times, 8.5 times, 9.0 times, 9.5 times, 10 times, or more times greater than the time of stability for the anti-PVRIG antibody and/or antigen binding portion thereof composition formulated in the absence of the at least amino acid, salt, and/or non-ionic surfactant.

As used herein, “BDS” refers to “Bulk Drug Substance.”

### A. Stabilized Liquid Formulations

In some embodiments, the present disclosure provides stabilized aqueous formulations of an anti-PVRIG antibody and/or antigen binding portion thereof (e.g., anti-PVRIG antibodies including those with CDRs identical to those shown in FIG. 3). The following embodiments are based in part on the discovery that inclusion of at least one amino acid, salt, and/or non-ionic surfactant stabilizes the liquid anti-PVRIG antibody and/or antigen binding portion thereof compositions, as compared to compositions lacking the at least one amino acid, salt, and/or non-ionic surfactant. In some embodiments, the formulation does not comprise a sugar and/or sugar alcohol.

As will be recognized by one of skill in the art, an anti-PVRIG antibody and/or antigen binding portion thereof formulated according to the embodiments provided herein may contain, in addition to the components explicitly disclosed, counter ions contributed by the inclusion of solution components or pH modifying agents, for example, sodium or potassium contributed from an acetate salt, sodium hydroxide, or potassium hydroxide or chloride contributed by calcium chloride or hydrochloric acid. In the context of the present disclosure, a storage stable an anti-PVRIG antibody and/or antigen binding portion thereof composition consisting of or consisting essentially of a given formulation may further comprise one or more counter ion, as necessitated by the formulation process at a particular pH.

In one embodiment, a storage stable anti-PVRIG antibody and/or antigen binding portion provided herein will be stabilized at refrigerated temperature (i.e., between 2° C. and 10° C.) for a period of time. For example, in one embodiment, a stable liquid pharmaceutical formulations comprising an anti-PVRIG antibody or antigen binding fragment thereof will be stable when stored at refrigerated temperature for at least 4 days. In other embodiments, the anti-PVRIG antibody and/or antigen binding portion composition will be stabile at refrigerated temperature for at least 1, 2, 3, 4, 5, 6, 7, 8, 9, 10, 11, 12, 13, 14, 21, 28, or more days. In some embodiments, the anti-PVRIG antibody and/or antigen binding portion composition will be stable for at least 1 week, 2 weeks, 3 weeks, 4 weeks, 5 weeks, 6 weeks, 7 weeks, 8 weeks, 9 weeks, 10 weeks, or more. In some embodiments, the anti-PVRIG antibody and/or antigen binding portion composition will be stable for at least 1 month. In some embodiments, the composition will be stable for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more months. In some embodiments, the anti-PVRIG antibody and/or antigen binding portion composition will be stable for an extended period of time when stored at a temperature between 2° C. and 8° C.

In one embodiment, a stable liquid pharmaceutical formulations comprising an anti-PVRIG antibody or antigen binding fragment thereof provided herein will be stabilized at room temperature (i.e., between 18° C. and 32° C.) for a period of time. For example, in one embodiment, a stable liquid pharmaceutical formulations comprising an anti-PVRIG antibody or antigen binding fragment thereof will be stable when stored at room temperature for at least 4 days. In some embodiments, the anti-PVRIG antibody and/or antigen binding portion composition will be stabile at room temperature for at least 1, 2, 3, 4, 5, 6, 7, 8, 9, 10, 11, 12, 13, 14, 21, 28, or more days. In some embodiments, the anti-PVRIG antibody and/or antigen binding portion composition will be stable for at least 1 week, 2 weeks, 3 weeks, 4 weeks, 5 weeks, 6 weeks, or more. In some embodiments, the anti-PVRIG antibody and/or antigen binding portion composition will be stable for at least 1 month. In yet other embodiments, the composition will be stable for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more months. In some embodiments, room temperature refers to between 20° C. and 30° C., between 21° C. and 29° C., between 22° C. and 28° C., between 23° C. and 27° C., between 24° C. and 26° C., or about 25° C. In some embodiments, the anti-PVRIG antibody and/or antigen binding portion composition will be stable for an extended period of time when stored at a temperature between 20° C. and 25° C. In some embodiments, the anti-PVRIG antibody and/or antigen binding portion composition will be stable for an extended period of time when stored at a temperature of about 25° C.

In one embodiment, a storage stable anti-PVRIG antibody and/or antigen binding portion provided herein will be stabilized at elevated temperature (i.e., between 32° C. and 42° C.) for a period of time. For example, in one embodiment, a stable liquid pharmaceutical formulations comprising an anti-PVRIG antibody or antigen binding fragment thereof will be stable when stored at elevated temperature for at least 4 days. In some embodiments, the anti-PVRIG antibody and/or antigen binding portion composition will be stabile at elevated temperature for at least 1, 2, 3, 4, 5, 6, 7, 8, 9, 10, 11, 12, 13, 14, 21, 28, or more days. In some embodiments, the anti-PVRIG antibody and/or antigen binding portion composition will be stable for at least 1 week, 2 weeks, 3 weeks, 4 weeks, 5 weeks, or more. In some embodiments, the anti-PVRIG antibody and/or antigen binding portion composition will be stable for at least 1 month. In yet other embodiments, the anti-PVRIG antibody and/or antigen binding portion composition will be stable for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more months. In some embodiments, the anti-PVRIG antibody and/or antigen binding portion composition will be stable for an extended period of time when stored at a temperature between 35° C. and 40° C.

In one embodiment, a stored anti-PVRIG antibody and/or antigen binding portion composition is considered storage stable as long as the composition maintains at least 40% of the antibody binding activity present at the start of the storage period (e.g., at time=0). In another embodiment, a stored composition is considered stable as long as the composition maintains at least 45%, 50%, 55%, 60%, 65%, 70%, 75%, 80%, 85%, 90%, 95% or more of the antibody binding activity present at the start of the storage period (e.g., at time=0). In one embodiment, antibody binding activity is measure using any assay known in the art.

In some embodiments, an anti-PVRIG antibody and/or antigen binding portion composition is considered to have been stabilized by the addition of a stabilizing agent (e.g., at least one amino acid, salt, and/or non-ionic surfactant) when the anti-PVRIG antibody and/or antigen binding portion composition contains at least 10% more antibody binding activity after storage for a period of time, as compared to an anti-PVRIG antibody and/or antigen binding portion composition not containing the stabilizing agent or containing a lower amount of the stabilizing agent. In other embodiments, an anti-PVRIG antibody and/or antigen binding portion composition is considered to have been stabilized by the addition of a stabilizing agent (e.g., at least one amino acid, salt, and/or non-ionic surfactant) when the composition contains at least 15%, 20%, 25%, 30%, 35%, 40%, 45%, 50%, 55%, 60%, 65%, 70%, 75%, 80%, 85%, 90%, 95%, 100%, or a greater percentage more anti-PVRIG antibody and/or antigen binding portion activity after storage for a period of time, as compared to an anti-PVRIG antibody and/or antigen binding portion composition not containing the stabilizing agent or containing a lower amount of the stabilizing agent.

In one embodiment, a stored anti-PVRIG antibody and/or antigen binding portion composition is considered stable as long as the percentage of anti-PVRIG antibody and/or antigen binding portion present in an aggregated state remains no more than 50%. In some embodiments, a stored anti-PVRIG antibody and/or antigen binding portion thereof composition is considered stable as long as the percentage of the anti-PVRIG antibody and/or antigen binding portion thereof present in an aggregated state remains no more than 45%, 40%, 35%, 30%, 25%, 24%, 23%, 22%, 21%, 20%, 19%, 18%, 17%, 16%, 15%, 14%, 13%, 12%, 1 1%, 10%, 9%, 8%, 7%, 6%, 5%, 4%, 3%, 2%, 1%, or less.

In some embodiments, an anti-PVRIG antibody and/or antigen binding portion composition is considered to have been stabilized by the addition of a stabilizing agent (anti-PVRIG antibody and/or antigen binding portion composition, at least one amino acid, salt, and/or non-ionic surfactant) when the composition contains at least 10% less anti-PVRIG antibody and/or antigen binding portion present in an aggregated state after storage for a period of time, as compared to an anti-PVRIG antibody and/or antigen binding portion composition not containing the stabilizing agent or containing a lower amount of the stabilizing agent. In other embodiments, an anti-PVRIG antibody and/or antigen binding portion composition is considered to have been stabilized by the addition of a stabilizing agent (e.g., at least one amino acid, salt, and/or non-ionic surfactant) when the composition contains at least 15%, 20%, 25%, 30%, 35%, 40%, 45%, 50%, 55%, 60%, 65%, 70%, 75%, 80%, 85%, 90%, 95%, 100%, or a greater percentage less anti-PVRIG antibody and/or antigen binding portion present in an aggregated state after storage for a period of time, as compared to an anti-PVRIG antibody and/or antigen binding portion composition not containing the stabilizing agent or containing a lower amount of the stabilizing agent

In some embodiments, a stored anti-PVRIG antibody and/or antigen binding portion composition is considered stable as long as the composition maintains at least 40% of the starting binding activity (e.g., at time=0) after being subjected to mechanical stress. In another embodiment, a stored composition is considered stable as long as the composition maintains 45%, 50%, 55%, 60%, 65%, 70%, 75%, 80%, 85%, 90%, 95% or more of the starting binding activity (e.g., at time=0) after being subjected to mechanical stress. In some embodiments, the mechanical stress is agitation (e.g., shaking).

In some embodiments, an anti-PVRIG antibody and/or antigen binding portion composition is considered to have been stabilized by the addition of a stabilizing agent (e.g., at least one amino acid, salt, or non-ionic surfactant) when the anti-PVRIG antibody and/or antigen binding portion composition contains at least 10% more binding activity after being subjected to mechanical stress, as compared to an anti-PVRIG antibody and/or antigen binding portion composition not containing the stabilizing agent or containing a lower amount of the stabilizing agent. In other embodiments, an anti-PVRIG antibody and/or antigen binding portion composition is considered to have been stabilized by the addition of a stabilizing agent (e.g., a sugar, sugar alcohol, or non-ionic surfactant) when the composition contains at least 15%, 20%, 25%, 30%, 35%, 40%, 45%, 50%, 55%, 60%, 65%, 70%, 75%, 80%, 85%, 90%, 95%, 100%, or a greater percentage more furin activity after being subjected to mechanical stress, as compared to an anti-PVRIG antibody and/or antigen binding portion composition not containing the stabilizing agent or containing a lower amount of the stabilizing agent. In a specific embodiment, the mechanical stress is agitation (e.g., shaking).

In some embodiments, a stored anti-PVRIG antibody and/or antigen binding portion composition is considered stable as long as the percentage of anti-PVRIG antibody and/or antigen binding portion present in an aggregated state remains no more than 50% after being subjected to mechanical stress. In other embodiments, a stored anti-PVRIG antibody and/or antigen binding portion composition is considered stable as long as the percentage of anti-PVRIG antibody and/or antigen binding portion present in an aggregated state remains no more than 45%, 40%, 35%, 30%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or less after being subjected to mechanical stress. In some embodiments, the mechanical stress is agitation (e.g., shaking).

In some embodiments, an anti-PVRIG antibody and/or antigen binding portion composition is considered to have been stabilized by the addition of a stabilizing agent (e.g., at least one amino acid, salt, or non-ionic surfactant) when the composition contains at least 10% less anti-PVRIG antibody and/or antigen binding portion present in an aggregated state after being subjected to mechanical stress, as compared to an anti-PVRIG antibody and/or antigen binding portion composition not containing the stabilizing agent or containing a lower amount of the stabilizing agent. In some embodiments, an anti-PVRIG antibody and/or antigen binding portion composition is considered to have been stabilized by the addition of a stabilizing agent (e.g., at least one amino acid, salt, or non-ionic surfactant) when the composition contains at least 15%, 20%, 25%, 30%, 35%, 40%, 45%, 50%, 55%, 60%, 65%, 70%, 75%, 80%, 85%, 90%, 95%, 100%, or a greater percentage less anti-PVRIG antibody and/or antigen binding portion present in an aggregated state after being subjected to mechanical stress, as compared to an anti-PVRIG antibody and/or antigen binding portion composition not containing the stabilizing agent or containing a lower amount of the stabilizing agent. In a specific embodiment, the mechanical stress is agitation (e.g., shaking).

In some embodiments, the highly stabilized formulations of the invention have a shelf life of at least 6 months. As will be appreciated, this shelf life may be at frozen temperatures (i.e., −80° C., −25° C., 0° C.), refrigerated (0° C. to 10° C.), or room temperature (20° C. to 32° C.) in liquid or lyophilized form. In further aspects, the highly stabilized formulations of the invention have a shelf life of at least 12, 18, 24, 30, 36, 42, 48, 54, or 60 months.

In some embodiments, shelf life is determined by a percent activity remaining after storage at any of the above temperatures for any of the above periods of time. In some embodiments, shelf life means that the formulation retains at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, 100% of furin activity as measured by any of the assays described herein or known in the art as compared to activity prior to storage for any of the above amounts of time at any of the above temperatures.

In some embodiments, the present invention provides a stable liquid pharmaceutical formulation of an anti-PVRIG antibody comprising:
(a) an anti-PVRIG antibody, wherein the anti-PVRIG antibody comprises an antibody with CDRs identical to those shown in FIG. 3;
(b) 25 mM histidine;
(c) 60 mM NaCl;
(d) 100 mM L-Arginine, and
(e) 0.01% w/v polysorbate 80,
wherein the composition has a pH from 5.5 to 7.0.

In some embodiments, the anti-PVRIG antibody is at a concentration of from 10 mg/mL to 40 mg/mL, 15 mg/mL to 40 mg/mL, 15 mg/mL to 30 mg/mL, 10 mg/mL to 25 mg/mL, or 15 mg/mL to 25 mg/mL. In some embodiments, the anti-PVRIG antibody is at a concentration of from 10 mg/mL to 40 mg/mL. In some embodiments, the anti-PVRIG antibody is at a concentration of from 15 mg/mL to 40 mg/mL. In some embodiments, the anti-PVRIG antibody is at a concentration of from 15 mg/mL to 30 mg/mL. In some embodiments, the anti-PVRIG antibody is at a concentration of from 10 mg/mL to 25 mg/mL. In some embodiments, the anti-PVRIG antibody is at a concentration of from 15 mg/mL to 25 mg/mL. In some embodiments, the anti-PVRIG antibody is at a concentration of from 10 mg/mL to 25 mg/mL. In some embodiments, the anti-PVRIG antibody is at a concentration of from 15 mg/mL to 25 mg/mL. In some embodiments, the anti-PVRIG antibody is at a concentration of from 20 mg/mL to 25 mg/mL. In some embodiments, the anti-PVRIG antibody is at a concentration of about 20 mg/mL.

In some embodiments, the present invention provides a stable liquid pharmaceutical formulation of an anti-PVRIG antibody comprising:
(a) an anti-PVRIG antibody, wherein the anti-PVRIG antibody comprises an antibody with CDRs identical to those shown in FIG. 3;
(b) from 10 mM to 100 mM histidine;
(c) from 30 mM to 100 mM NaCl;
(d) from 20 mM to 150 mM L-Arginine, and
(e) from 0.005% to 0.1% w/v polysorbate 80
wherein the composition has a pH from 5.5 to 7.0.

### B. Amino Acids

In some embodiments, the present invention provides a stable liquid pharmaceutical formulation of an anti-PVRIG antibody or antigen binding fragment thereof (e.g., anti-PVRIG antibodies including those with CDRs identical to those shown in FIG. 3) comprising at least one amino acid. In some embodiments, the at least one amino acid is histidine. In some embodiments, the at least one amino acid is arginine. In some embodiments, the present invention provides a stable liquid pharmaceutical formulation of an anti-PVRIG antibody or antigen binding fragment thereof comprising at least two amino acids. In some embodiments, the at least two amino acids are histidine and arginine.

In some embodiments, the pharmaceutical formulation comprises from 10 mM to 80 mM histidine, from 15 mM to 70 mM histidine, from 20 mM to 60 mM histidine, from 20 mM to 50 mM histidine, or from 20 mM to 30 mM histidine. In some embodiments, the pharmaceutical formulation comprises from 10 mM to 80 mM histidine. In some embodiments, the pharmaceutical formulation comprises from 15 mM to 70 mM histidine. In some embodiments, the pharmaceutical formulation comprises from 20 mM to 60 mM histidine. In some embodiments, the pharmaceutical formulation comprises from 20 mM to 50 mM histidine. In some embodiments, the pharmaceutical formulation comprises from 20 mM to 30 mM histidine. In some embodiments, the pharmaceutical formulation comprises about 25 mM histidine.

In some embodiments, the pharmaceutical formulation comprises from 10 mM to 80 mM histidine. In some embodiments, the pharmaceutical formulation comprises from 15 mM to 70 mM histidine. In some embodiments, the pharmaceutical formulation comprises from 20 mM to 60 mM histidine. In some embodiments, the pharmaceutical formulation comprises from 20 mM to 50 mM histidine. In some embodiments, the pharmaceutical formulation comprises from 20 mM to 30 mM histidine. In some embodiments, the pharmaceutical formulation comprises about 25 mM histidine.

In some embodiments, the pharmaceutical formulation comprises from 20 mM to 140 mM L-arginine, from 30 mM to 140 mM L-arginine, from 40 mM to 130 mM L-arginine, from 50 mM to 120 mM L-arginine, from 60 mM to 110 mM L-arginine, from 70 mM to 110 mM L-arginine, from 80 mM to 110 mM L-arginine, or from 90 mM to 110 mM L-arginine. In some embodiments, the pharmaceutical formulation comprises from 20 mM to 140 mM L-arginine, from 30 mM to 140 mM L-arginine, from 40 mM to 130 mM L-arginine, from 50 mM to 120 mM L-arginine, from 60 mM to 110 mM L-arginine, from 70 mM to 110 mM L-arginine, from 80 mM to 110 mM L-arginine, or from 90 mM to 110 mM L-arginine.

In some embodiments, the pharmaceutical formulation comprises from 20 mM to 140 mM L-arginine. In some embodiments, the pharmaceutical formulation comprises from 30 mM to 140 mM L-arginine. In some embodiments, the pharmaceutical formulation comprises from 40 mM to 130 mM L-arginine. In some embodiments, the pharmaceutical formulation comprises from 50 mM to 120 mM L-arginine. In some embodiments, the pharmaceutical formulation comprises from 60 mM to 110 mM L-arginine. In some embodiments, the pharmaceutical formulation comprises from 70 mM to 110 mM L-arginine. In some embodiments, the pharmaceutical formulation comprises from 80 mM to 110 mM L-arginine. In some embodiments, the pharmaceutical formulation comprises from 90 mM to 110 mM L-arginine. In some embodiments, the pharmaceutical formulation comprises about 100 mM L-arginine.

C. Sugar and/or Sugar Alcohol

In some embodiments, the present invention provides a stable liquid pharmaceutical formulation of an anti-PVRIG antibody or antigen binding fragment thereof (e.g., anti-PVRIG antibodies including those with CDRs identical to those shown in FIG. 3) comprising no sugar and/or sugar alcohol. In some embodiments, the present invention provides a stable liquid pharmaceutical formulation of an anti-PVRIG antibody or antigen binding fragment thereof (e.g., anti-PVRIG antibodies including those with CDRs identical to those shown in FIG. 3) comprising no sugar. In some embodiments, the present invention provides a stable liquid pharmaceutical formulation of an anti-PVRIG antibody or antigen binding fragment thereof (e.g., anti-PVRIG antibodies including those with CDRs identical to those shown in FIG. 3) comprising no sugar alcohol.

In some embodiments, the present invention provides a stable liquid pharmaceutical formulation of an anti-PVRIG antibody or antigen binding fragment thereof comprising a sugar and/or sugar alcohol. In some embodiments, the sugar is trehalose or sucrose. In some embodiments, the sugar is trehalose. In some embodiments, the sugar is sucrose. In some embodiments, the sugar is only one of trehalose or sucrose but not both.

In some embodiments, the sugar is in an amount of from about 0.5% to 10%, 1% to 9.5%, 1.5% to 9%, 2.0% to 8.5%, 2.5% to 8%, 3.0% to 7.5%, 3.5% to 7%, 4.0% to 6.5%, 4.5% to 6%, and/or 4.5% to 5.5%. In some embodiments, the sugar is in an amount of from about 0.5% to 10%. In some embodiments, the sugar is in an amount of from about 1% to 9.5%. In some embodiments, the sugar is in an amount of from about 1.5% to 9%. In some embodiments, the sugar is in an amount of from about 2.0% to 8.5%. In some embodiments, the sugar is in an amount of from about 2.5% to 8%. In some embodiments, the sugar is in an amount of from about 3.0% to 7.5%. In some embodiments, the sugar is in an amount of from about 3.5% to 7%. In some embodiments, the sugar is in an amount of from about 4.0% to 6.5%. In some embodiments, the sugar is in an amount of from about 4.5% to 6%. In some embodiments, the sugar is in an amount of from about 4.5% to 5.5%. In some embodiments, the sugar is in an amount of about 5%

### D. Non-Ionic Surfactants

In some embodiments, the present invention provides a stable liquid pharmaceutical formulation of an anti-PVRIG antibody or antigen binding fragment thereof (e.g., anti-PVRIG antibodies including those with CDRs identical to those shown in FIG. 3) comprising a non-ionic surfactant. In some embodiments, the storage stable compositions of an anti-PVRIG antibody or antigen binding fragment comprise a non-ionic surfactant selected from a non-ionic water soluble monoglyceride, a non-ionic water soluble diglyceride, a non-ionic water soluble triglyceride, a non-ionic water soluble monofatty acid esters of polyethyelene glycol, a non-ionic water soluble difatty acid esters of polyethyelene glycol, a non-ionic water soluble sorbitan fatty acid ester, a non-ionic polyglycolyzed glyceride, a non-ionic water soluble triblock copolymer, and a combination thereof. In some embodiments, the non-ionic surfactant is polysorbate 80 (polyoxyethylene (20) sorbitan monooleate).

In some embodiments, the stable liquid pharmaceutical formulation comprises from 0.006% to 0.1% w/v polysorbate 80, from 0.007% to 0.09% w/v polysorbate 80, from 0.008% to 0.08% w/v polysorbate 80, from 0.009% to 0.09% w/v polysorbate 80, from 0.01% to 0.08% w/v polysorbate 80, from 0.01% to 0.07% w/v polysorbate 80, from 0.01% to 0.07% w/v polysorbate 80, or from 0.01% to 0.06% w/v polysorbate 80, or from 0.009% to 0.05% w/v polysorbate 80. In some embodiments, the stable liquid pharmaceutical formulation comprises from 0.006% to 0.1% w/v polysorbate 80. In some embodiments, the stable liquid pharmaceutical formulation comprises from 0.007% to 0.09% w/v polysorbate 80. In some embodiments, the stable liquid pharmaceutical formulation comprises from 0.008% to 0.08% w/v polysorbate 80. In some embodiments, the stable liquid pharmaceutical formulation comprises from 0.009% to 0.09% w/v polysorbate 80. In some embodiments, the stable liquid pharmaceutical formulation comprises from 0.01% to 0.08% w/v polysorbate 80. In some embodiments, the stable liquid pharmaceutical formulation comprises from 0.01% to 0.07% w/v polysorbate 80. In some embodiments, the stable liquid pharmaceutical formulation comprises from 0.01% to 0.07% w/v polysorbate 80. In some embodiments, the stable liquid pharmaceutical formulation comprises from 0.01% to 0.06% w/v polysorbate 80. In some embodiments, the stable liquid pharmaceutical formulation comprises from 0.009% to 0.05% w/v polysorbate 80. In some embodiments, the stable liquid pharmaceutical formulation comprises about 0.01% polysorbate 80.

### E. Pharmaceutically Acceptable Salts

In some embodiments, the present invention provides a stable liquid pharmaceutical formulation of an anti-PVRIG antibody or antigen binding fragment thereof (e.g., anti-PVRIG antibodies including those with CDRs identical to those shown in FIG. 3) comprising a salt, for example, a pharmaceutically acceptable salt.

In some embodiments, the stable liquid pharmaceutical formulation comprising an anti-PVRIG antibody or antigen binding fragment thereof provided herein include a pharmaceutically acceptable salt at a concentration tolerated by an anti-PVRIG antibody or antigen binding fragment thereof during storage. In some embodiments, the pharmaceutically acceptable salt is a chloride salt. In some embodiments, the pharmaceutically acceptable salt is a monovalent chloride salt. In a more specific embodiment, the pharmaceutically acceptable salt is sodium chloride, potassium chloride, or a combination thereof.

In some embodiments, the stable liquid pharmaceutical formulation comprises from 30 mM to 100 mM NaCl, from 30 mM to 90 mM NaCl, from 40 mM to 80 mM NaCl, from 30 mM to 70 mM NaCl, or from 45 mM to 70 mM NaCl.

In some embodiments, the stable liquid pharmaceutical formulation comprises from 30 mM to 100 mM NaCl. In some embodiments, the stable liquid pharmaceutical formulation comprises from 30 mM to 90 mM NaCl. In some embodiments, the stable liquid pharmaceutical formulation comprises from 40 mM to 80 mM NaCl. In some embodiments, the stable liquid pharmaceutical formulation comprises from 30 mM to 70 mM histidine. In some embodiments, the stable liquid pharmaceutical formulation comprises or from 45 mM to 70 mM NaCl. In some embodiments, pharmaceutical formulation comprises about 60 mM NaCl.

### F. Buffering Agents

In some embodiments, the present invention provides a stable liquid pharmaceutical formulation of an anti-PVRIG antibody or antigen binding fragment thereof (e.g., anti-PVRIG antibodies including those with CDRs identical to those shown in FIG. 3) that is buffered at a physiologically acceptable pH. In some embodiments, the physiologically acceptable pH is from about 6.0 to about 7.0.

In some embodiments, stable liquid pharmaceutical formulation of an anti-PVRIG antibody or antigen binding fragment thereof has a pH of from 6 to 7.0. In some embodiments, stable liquid pharmaceutical formulation of an anti-PVRIG antibody or antigen binding fragment thereof has a pH of 6, 6.1, 6.2, 6.3, 6.4, 6.5, 6.6, 6.7, 6.8, 6.9, or 7.0. In some embodiments, the pH is from 6.1 to 6.9. In some embodiments, the pH is from 6.2 to 6.9. In some embodiments, the pH is from 6.3 to 6.8. In some embodiments, the pH is from 6.3 to 6.7. In some embodiments, the pH is from 6.4 to 6.8. In some embodiments, the pH is from 6.5 to 6.8. In some embodiments, the pH is from 6.6 to 6.8. In some embodiments, the pH is 6.3, 6.4, 6.5, 6.6, or 6.7. In some embodiments, the pH is 6.5+/−0.2.

### G. Methods for Diluting Aqueous Compositions

[In some embodiments, the method includes adding a dilution buffer, to form a diluted stable liquid pharmaceutical formulation comprising an anti-PVRIG antibody or antigen binding fragment thereof (e.g., anti-PVRIG antibodies including those with CDRs identical to those shown in FIG. 3). In some embodiments, the dilution buffer is added at a ratio of from 1:1 (dilution buffer: formulation) to 1000:1 (dilution buffer: formulation). In another embodiment, the dilution buffer is added at a ratio of from 1:1 dilution buffer: formulation) to 500:1 (dilution buffer: formulation). In another embodiment, the dilution buffer is added at a ratio of from 1:1 (dilution buffer: formulation) to 250:1 (dilution buffer: formulation). In another embodiment, the dilution buffer is added at a ratio of from 1:1 (dilution buffer: formulation) to 200:1 (dilution buffer: formulation). In another embodiment, the dilution buffer is added at a ratio of from 1:1 (dilution buffer: formulation) to 100:1 (dilution buffer: formulation). In another embodiment, the dilution buffer is added at a ratio of from 1:1 (dilution buffer: formulation) to 50:1 (dilution buffer: formulation).

In some embodiments, the stable liquid pharmaceutical formulation comprising an anti-PVRIG antibody or antigen binding fragment thereof is diluted from 1-fold to 1000-fold, from 1-fold to 500-fold, from 1-fold to 250-fold, from 1-fold to 200-fold, from 1-fold to 100-fold, from 1-fold to 50-fold, from 1-fold to 10-fold, from 10-fold to 1000-fold, from 10-fold to 500-fold, from 10-fold to 250-fold, from 10-fold to 200-fold, from 10-fold to 100-fold, from 10-fold to 50-fold, from 50-fold to 1000-fold, from 50-fold to 500-fold, from 50-fold to 250-fold, from 50-fold to 200-fold, from 50-fold to 100-fold, from 100-fold to 1000-fold, from 100-fold to 500-fold, from 100-fold to 250-fold, from 100-fold to 200-fold, from 200-fold to 1,000-fold, from 200-fold to 500-fold, or from 200-fold to 250-fold.

### H. Stability Assays

As discussed herein, the stable liquid pharmaceutical formulations comprising an anti-PVRIG antibody or antigen binding fragment thereof (e.g., anti-PVRIG antibodies including those with CDRs identical to those shown in FIG. 3) show improved stability as compared to control formulations. In one embodiment, improved stability includes retention of a higher percentage of binding activity and/or no reduction in binding activity as compared to control formulations in various stability assays. Such assays can be used to determine if a formulation is a highly stabilized formulation. In some embodiments, the highly stabilized formulation has at least 5%, 10%, 20%, 30%, 40%, 50%, 55%, 60%, 65%, 70%, 75%, 80%, 85%, 90%, 95%, 96%, 97%, 98%, 99% or greater activity than a control formulation when assessed by any of the stability assays discussed herein or known in the art.

In some embodiments, the liquid pharmaceutical formulations comprising an anti-PVRIG antibody or antigen binding fragment thereof are tested under stressor conditions, such as storage at high temperature, agitation, freeze/thaw cycles, or some combination thereof. After such stressors, the formulations are assayed using any of the methods described herein or known in the art to determine the stability under these conditions.

### A280 and Appearance Analysis

In some embodiments, an A280 by SoloVPE assay is used to examine the appearance of the stable liquid pharmaceutical formulations comprising an anti-PVRIG antibody or antigen binding fragment thereof.

In some embodiments, the SoloVPE assay can be employed to examine concentrations for the stable liquid pharmaceutical formulations comprising an anti-PVRIG antibody or antigen binding fragment thereof.

A280: Amino acids containing aromatic side chains exhibit strong UV-light absorption at the wavelength of 280 nm. Once an absorptivity coefficient has been established for a given protein, the protein's concentration in solution can be calculated from its absorbance. The method is designed to determine the protein concentration by measuring its absorbance at 280 nm using the SoloVPE instrument without dilution (https://www.ctechnologiesinc.com/products/solovpe)

Appearance: Sample appearance determination is assessed by holding the sample within a controlled light source and observe the appearance of the material. Gently agitate the solution and determine if the appearance changes when viewed against a black and white background. Use adjectives such as “clear”, “turbid”, or “slightly turbid” to assess clarity. Be specific with regards to the color of the material. If the material is colorless then state that as a result (i.e. clear, colorless solution). specify the physical state of the sample (i.e. liquid or frozen liquid)

### Binding Assay Analysis

In some embodiments, a binding assay can be performed to examine the activity of the stable liquid pharmaceutical formulations comprising an anti-PVRIG antibody or antigen binding fragment thereof.

### LabChip Analysis

In some embodiments, a LabChip analysis is employed to examine purity, including for example, IgG purity as well as HC+LC percentages for the stable liquid pharmaceutical formulations comprising an anti-PVRIG antibody or antigen binding fragment thereof. In some embodiments, the stable liquid pharmaceutical formulations comprising an anti-PVRIG antibody or antigen binding fragment thereof exhibit IgG purity percentages greater than 94%, greater than 95%, greater than 96%, greater than 97%, or greater than 98%. In some embodiments, the stable liquid pharmaceutical formulations comprising an anti-PVRIG antibody or antigen binding fragment thereof exhibit IgG purity percentages were from about 95% to 98%. In some embodiments, the stable liquid pharmaceutical formulations comprising an anti-PVRIG antibody or antigen binding fragment thereof exhibit IgG purity percentages from about 96% to 97%. In some embodiments, the stable liquid pharmaceutical formulations comprising an anti-PVRIG antibody or antigen binding fragment thereof exhibit HC+LC percentages from about 96% to 100%. In some embodiments, the stable liquid pharmaceutical formulations comprising an anti-PVRIG antibody or antigen binding fragment thereof exhibit HC+LC percentages from about 97% to 100%. In some embodiments, the stable liquid pharmaceutical formulations comprising an anti-PVRIG antibody or antigen binding fragment thereof exhibit HC+LC percentages from about 98% to 100%.

cIEF Analysis

In some embodiments, a capillary isoelectric focusing (cIEF) can be employed to analyze the stable liquid pharmaceutical formulations comprising an anti-PVRIG antibody or antigen binding fragment thereof for the presence of additional species, including for example, minor acidic species.

### MFI Analysis

Antibodies can form sub-visible particles in response to stressed conditions, such as heat, freeze/thaw cycles, and agitation. In some embodiments, a microflow imaging (MFI) analysis can be employed to analyze the stable liquid pharmaceutical formulations comprising an anti-PVRIG antibody or antigen binding fragment thereof for the formation of particles in response to stressed conditions. In some embodiments, the stable liquid pharmaceutical formulations of the anti-PVRIG antibody or antigen binding fragment thereof provide for a formulation capable of stabilizing the anti-PVRIG antibody or antigen binding fragment thereof against these stressed conditions and protecting against the formation of particles. MFI can be used to evaluate particle counts at different size ranges (<2 μm, <5 μm, <10 μm, and <25 μm) in different formulations under stressed conditions. Typically, MFI data can be evaluated to choose an appropriate formulation based on generation of the lowest amount of particles/mL for all sizes of particles across all time points, conditions, and formulations.

### SEC Analysis

In some embodiments, size exclusion chromatography (SEC) can be employed to analyze the stable liquid pharmaceutical formulations comprising an anti-PVRIG antibody or antigen binding fragment thereof. The SEC data showed HMW throughout all time points and conditions; however, it remained stable at about 1%. LMW was present in accelerated conditions and 2-8° C. 8 week time point. Within the 40° C. condition, the LMW did increase from about 1% to 3% from Week 1 to Week 2.

### I. Selected Formulation Embodiments

In some embodiments, the present invention provides a stable liquid pharmaceutical formulation of an anti-PVRIG antibody comprising:
(a) an anti-PVRIG antibody comprising:
   i) a heavy chain variable domain comprising the vhCDR1, vhCDR2, and vhCDR3 from the heavy chain of CHA.7.518.1.H4 (S241P) (SEQ ID NO:4), and
   ii) a light chain variable domain comprising the vlCDR1, vlCDR2, and vlCDR3 from the light chain of CHA.7.518.1.H4 (S241P) (SEQ ID NO:9);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides a stable liquid pharmaceutical formulation of an anti-PVRIG antibody comprising:
(a) an anti-PVRIG antibody comprising:
   i) a heavy chain variable domain comprising the vhCDR1, vhCDR2, and vhCDR3 from the heavy chain of CHA.7.518.1.H4 (S241P) (SEQ ID NO:4), and
   ii) a light chain variable domain comprising the vlCDR1, vlCDR2, and vlCDR3 from the light chain of CHA.7.518.1.H4 (S241P) (SEQ ID NO:9).
(a) an anti-PVRIG antibody;
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides a stable liquid pharmaceutical formulation comprising:
(a) an anti-PVRIG antibody comprising:
   i) heavy chain variable domain is from the heavy chain of CHA.7.518.1.H4 (S241P) (SEQ ID NO:4), and
   ii) a light chain variable domain is from the light chain of CHA.7.518.1.H4 (S241P) (SEQ ID NO:9);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides a stable liquid pharmaceutical formulation comprising:
(a) an anti-PVRIG antibody comprising:
   i) heavy chain variable domain is from the heavy chain of CHA.7.518.1.H4 (S241P) (SEQ ID NO:4), and
   ii) a light chain variable domain is from the light chain of CHA.7.518.1.H4 (S241P) (SEQ ID NO:9);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides a stable liquid pharmaceutical formulation comprising:
(a) an anti-PVRIG antibody comprising:
   i) a heavy chain comprising:
      a) a VH-CH1-hinge-CH2-CH3, wherein the VH is from CHA.7.518.1.H4 (S241P) (SEQ ID NO:4) and wherein the CH1-hinge-CH2-CH3 region is from IgG4; and
   ii) a light chain comprising:
      a) a VL-CL, wherein the VL from CHA.7.518.1.H4 (S241P) (SEQ ID NO: 9) and wherein the CL region is from human kappa 2 light chain;
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides a stable liquid pharmaceutical formulation comprising:
(a) an anti-PVRIG antibody comprising:
   i) a heavy chain comprising:
      a) a VH-CH1-hinge-CH2-CH3, wherein the VH is from CHA.7.518.1.H4 (S241P) (SEQ ID NO:4) and wherein the CH1-hinge-CH2-CH3 region is from IgG4; and
   ii) a light chain comprising:
      a) a VL-CL, wherein the VL from CHA.7.518.1.H4 (S241P) (SEQ ID NO: 9) and wherein the CL region is from human kappa 2 light chain;
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides a stable liquid pharmaceutical formulation comprising:
(a) an anti-PVRIG antibody comprising:
   i) a heavy chain comprising the heavy chain from CHA.7.518.1.H4 (S241P) (SEQ ID NO:8); and
   ii) a light chain comprising the light chain from CHA.7.518.1.H4 (S241P) (SEQ ID NO:13);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides a stable liquid pharmaceutical formulation comprising:
(a) an anti-PVRIG antibody comprising:
   i) a heavy chain comprising the heavy chain from CHA.7.518.1.H4 (S241P) (SEQ ID NO:8); and
   ii) a light chain comprising the light chain from CHA.7.518.1.H4 (S241P) (SEQ ID NO:13);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

### IX. Administration of Formulations of Anti-PVRIG Antibodies

Administration of the pharmaceutical composition comprising anti-PVRIG antibodies of the present invention (e.g., anti-PVRIG antibodies including those with CDRs identical to those shown in FIG. 3), preferably in the form of a sterile aqueous solution, may be done in a variety of ways, As is known in the art, protein therapeutics are often delivered by IV infusion. The antibodies of the present invention may also be delivered using such methods. For example, administration may venious be by intravenous infusion with 0.9% sodium chloride as an infusion vehicle. Such techniques are disclosed in Remington's Pharmaceutical Sciences 16th edition, Osol, A. Ed., 1980.

The dosing amounts and frequencies of administration are, in some embodiments, selected to be therapeutically or prophylactically effective. As is known in the art, adjustments for protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art. In order to treat a patient, a therapeutically effective dose of the Fc variant of the present invention may be administered. By “therapeutically effective dose” herein is meant a dose that produces the effects for which it is administered.

### X. Dosing

In some embodiments, the anti-PVRIG antibody and/or antigen binding portion thereof formulations of the present invention can be formulated for administration, including as a unit dosage formulation. In some embodiments, the anti-PVRIG antibody and/or antigen binding portion thereof formulations are administered at a dosage of 0.01 mg/kg of the anti-PVRIG antibody and/or antigen binding portion thereof. In some embodiments, the anti-PVRIG antibody and/or antigen binding portion thereof formulations are administered at a dosage of 0.02 mg/kg of the anti-PVRIG antibody and/or antigen binding portion thereof. In some embodiments, the anti-PVRIG antibody and/or antigen binding portion thereof formulations are administered at a dosage of 0.03 mg/kg of the anti-PVRIG antibody and/or antigen binding portion thereof. In some embodiments, the anti-PVRIG antibody and/or antigen binding portion thereof formulations are administered at a dosage of 0.04 mg/kg of the anti-PVRIG antibody and/or antigen binding portion thereof. In some embodiments, the anti-PVRIG antibody and/or antigen binding portion thereof formulations are administered at a dosage of 0.05 mg/kg of the anti-PVRIG antibody and/or antigen binding portion thereof. In some embodiments, the anti-PVRIG antibody and/or antigen binding portion thereof formulations are administered at a dosage of 0.06 mg/kg of the anti-PVRIG antibody and/or antigen binding portion thereof. In some embodiments, the anti-PVRIG antibody and/or antigen binding portion thereof formulations are administered at a dosage of 0.07 mg/kg of the anti-PVRIG antibody and/or antigen binding portion thereof. In some embodiments, the anti-PVRIG antibody and/or antigen binding portion thereof formulations are administered at a dosage of 0.08 mg/kg of the anti-PVRIG antibody and/or antigen binding portion thereof. In some embodiments, the anti-PVRIG antibody and/or antigen binding portion thereof formulations are administered at a dosage of 0.09 mg/kg of the anti-PVRIG antibody and/or antigen binding portion thereof. In some embodiments, the anti-PVRIG antibody and/or antigen binding portion thereof formulations are administered at a dosage of 0.1 mg/kg of the anti-PVRIG antibody and/or antigen binding portion thereof. In some embodiments, the anti-PVRIG antibody and/or antigen binding portion thereof formulations are administered at a dosage of 0.2 mg/kg of the anti-PVRIG antibody and/or antigen binding portion thereof. In some embodiments, the anti-PVRIG antibody and/or antigen binding portion thereof formulations are administered at a dosage of 0.3 mg/kg of the anti-PVRIG antibody and/or antigen binding portion thereof. In some embodiments, the anti-PVRIG antibody and/or antigen binding portion thereof formulations are administered at a dosage of 0.5 mg/kg of the anti-PVRIG antibody and/or antigen binding portion thereof. In some embodiments, the anti-PVRIG antibody and/or antigen binding portion thereof formulations are administered at a dosage of 0.8 mg/kg of the anti-PVRIG antibody and/or antigen binding portion thereof. In some embodiments, the anti-PVRIG antibody and/or antigen binding portion thereof formulations are administered at a dosage of 1 mg/kg of the anti-PVRIG antibody and/or antigen binding portion thereof. In some embodiments, the anti-PVRIG antibody and/or antigen binding portion thereof formulations are administered at a dosage of 2 mg/kg of the anti-PVRIG antibody and/or antigen binding portion thereof. In some embodiments, the anti-PVRIG antibody and/or antigen binding portion thereof formulations are administered at a dosage of 3 mg/kg of the anti-PVRIG antibody and/or antigen binding portion thereof. In some embodiments, the anti-PVRIG antibody and/or antigen binding portion thereof formulations are administered at a dosage of 4 mg/kg of the anti-PVRIG antibody and/or antigen binding portion thereof. In some embodiments, the anti-PVRIG antibody and/or antigen binding portion thereof formulations are administered at a dosage of 5 mg/kg of the anti-PVRIG antibody and/or antigen binding portion thereof. In some embodiments, the anti-PVRIG antibody and/or antigen binding portion thereof formulations are administered at a dosage of 6 mg/kg of the anti-PVRIG antibody and/or antigen binding portion thereof. In some embodiments, the anti-PVRIG antibody and/or antigen binding portion thereof formulations are administered at a dosage of 7 mg/kg of the anti-PVRIG antibody and/or antigen binding portion thereof. In some embodiments, the anti-PVRIG antibody and/or antigen binding portion thereof formulations are administered at a dosage of 8 mg/kg of the anti-PVRIG antibody and/or antigen binding portion thereof. In some embodiments, the anti-PVRIG antibody and/or antigen binding portion thereof formulations are administered at a dosage of 9 mg/kg of the anti-PVRIG antibody and/or antigen binding portion thereof. In some embodiments, the anti-PVRIG antibody and/or antigen binding portion thereof formulations are administered at a dosage of 10 mg/kg of the anti-PVRIG antibody and/or antigen binding portion thereof. In some embodiments, the anti-PVRIG antibody and/or antigen binding portion thereof formulations are administered at a dosage of 20 mg/kg of the anti-PVRIG antibody and/or antigen binding portion thereof.

In some embodiments, the anti-PVRIG antibody and/or antigen binding portion thereof formulations is administered at a dosage of about 0.01 mg/kg to about 20 mg/kg of the anti-PVRIG antibody. In some embodiments, the anti-PVRIG antibody and/or antigen binding portion thereof formulations is administered at a dosage of about 0.01 mg/kg to about 10 mg/kg of the anti-PVRIG antibody. In some embodiments, the anti-PVRIG antibody and/or antigen binding portion thereof formulations is administered at a dosage of about 20 mg/kg. In some embodiments, the anti-PVRIG antibody and/or antigen binding portion thereof formulations is administered at a dosage of about 20 mg/kg each 4 weeks. In some embodiments, the anti-PVRIG antibody and/or antigen binding portion thereof formulations is administered at a dosage of about 20 mg/kg IV each 4 weeks. In some embodiments, formulation is administered at a dosage of about 0.1 mg/kg to about 10 mg/kg of the anti-PVRIG antibody. In some embodiments, formulation is administered at a dosage of about 1 mg/kg to about 10 mg/kg of the anti-PVRIG antibody. In some embodiments, formulation is administered at a dosage of about 2 mg/kg to about 10 mg/kg of the anti-PVRIG antibody. In some embodiments, formulation is administered at a dosage of about 3 mg/kg to about 10 mg/kg of the anti-PVRIG antibody. In some embodiments, formulation is administered at a dosage of about 4 mg/kg to about 10 mg/kg of the anti-PVRIG antibody. In some embodiments, formulation is administered at a dosage of about 5 mg/kg to about 10 mg/kg of the anti-PVRIG antibody. In some embodiments, formulation is administered at a dosage of about 5 mg/kg to about 10 mg/kg of the anti-PVRIG antibody. In some embodiments, formulation is administered at a dosage of about 7 mg/kg to about 10 mg/kg of the anti-PVRIG antibody. In some embodiments, formulation is administered at a dosage of about 8 mg/kg to about 10 mg/kg of the anti-PVRIG antibody. In some embodiments, formulation is administered at a dosage of about 9 mg/kg to about 10 mg/kg of the anti-PVRIG antibody. In some embodiments, formulation is administered at a dosage of about 20 mg/kg of the anti-PVRIG antibody. In some embodiments, formulation is administered at a dosage of about 0.01 mg/kg, 0.03 mg/kg, 0.1 mg/kg, 0.3 mg/kg, 1 mg/kg, 3 mg/kg, 10 mg/kg or 20 mg/kg of the anti-PVRIG antibody.

A. Selected Dosing with Formulation Embodiments

In some embodiments, the present invention provides for administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg and wherein the stable liquid formulation of the anti-PVRIG antibody comprises:
(a) an anti-PVRIG antibody comprising:
   i) a heavy chain variable domain comprising the vhCDR1, vhCDR2, and vhCDR3 from the heavy chain of CHA.7.518.1.H4 (S241P) (SEQ ID NO:4), and
   ii) a light chain variable domain comprising the vlCDR1, vlCDR2, and vlCDR3 from the light chain of CHA.7.518.1.H4 (S241P) (SEQ ID NO:9);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain variable domain comprising the vhCDR1, vhCDR2, and vhCDR3 from the heavy chain of CHA.7.518.1.H4 (S241P) (SEQ ID NO:4), and
   ii) a light chain variable domain comprising the vlCDR1, vlCDR2, and vlCDR3 from the light chain of CHA.7.518.1.H4 (S241P) (SEQ ID NO:9).
(a) an anti-PVRIG antibody;
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) heavy chain variable domain is from the heavy chain of CHA.7.518.1.H4 (S241P) (SEQ ID NO:4), and
   ii) a light chain variable domain is from the light chain of CHA.7.518.1.H4 (S241P) (SEQ ID NO:9);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) heavy chain variable domain is from the heavy chain of CHA.7.518.1.H4 (S241P) (SEQ ID NO:4), and
   ii) a light chain variable domain is from the light chain of CHA.7.518.1.H4 (S241P) (SEQ ID NO:9);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising:
      a) a VH-CH1-hinge-CH2-CH3, wherein the VH is from CHA.7.518.1.H4 (S241P) (SEQ ID NO:4) and wherein the CH1-hinge-CH2-CH3 region is from IgG4; and
   ii) a light chain comprising:
      a) a VL-CL, wherein the VL from CHA.7.518.1.H4 (S241P) (SEQ ID NO: 9) and wherein the CL region is from human kappa 2 light chain;
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising:
      a) a VH-CH1-hinge-CH2-CH3, wherein the VH is from CHA.7.518.1.H4 (S241P) (SEQ ID NO:4) and wherein the CH1-hinge-CH2-CH3 region is from IgG4; and
   ii) a light chain comprising:
      a) a VL-CL, wherein the VL from CHA.7.518.1.H4 (S241P) (SEQ ID NO: 9) and wherein the CL region is from human kappa 2 light chain;
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising the heavy chain from CHA.7.518.1.H4 (S241P) (SEQ ID NO:8); and
   ii) a light chain comprising the light chain from CHA.7.518.1.H4 (S241P) (SEQ ID NO:13);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising the heavy chain from CHA.7.518.1.H4 (S241P) (SEQ ID NO:8); and
   ii) a light chain comprising the light chain from CHA.7.518.1.H4 (S241P) (SEQ ID NO:13);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments of the stable liquid pharmaceutical formulation, the formulation is administered with an anti-PD-1 antibody.

In some embodiments of the stable liquid pharmaceutical formulation, the anti-PD-1 antibody is nivolumab. In some embodiments of the stable liquid pharmaceutical formulation, the anti-PD-1 antibody is nivolumab is administered at a dosage of about 360 mg or 480 mg. In some embodiments of the stable liquid pharmaceutical formulation, the anti-PD-1 antibody is nivolumab is administered at a dosage of about 360 mg. In some embodiments of the stable liquid pharmaceutical formulation, the anti-PD-1 antibody is nivolumab is administered at a dosage of about 480 mg.

### XI. Methods of Using the Anti-PVRIG Antibody Formulations

### A. Therapeutic Uses

The anti-PVRIG antibodies (e.g., anti-PVRIG antibodies including those with CDRs identical to those shown in FIG. 3) find use in treating patients, such as human subjects, generally with a condition associated with PVRIG. The term “treatment” as used herein, refers to both therapeutic treatment and prophylactic or preventative measures, which in this example relates to treatment of cancer; however, also as described below, uses of antibodies and pharmaceutical compositions are also provided for treatment of infectious disease, sepsis, and/or autoimmune conditions, and/or for inhibiting an undesirable immune activation that follows gene therapy. Those in need of treatment include those already with cancer as well as those in which the cancer is to be prevented. Hence, the mammal to be treated herein may have been diagnosed as having the cancer or may be predisposed or susceptible to the cancer. As used herein the term “treating” refers to preventing, delaying the onset of, curing, reversing, attenuating, alleviating, minimizing, suppressing, halting the deleterious effects or stabilizing of discernible symptoms of the above-described cancerous diseases, disorders or conditions. It also includes managing the cancer as described above. By “manage” it is meant reducing the severity of the disease, reducing the frequency of episodes of the disease, reducing the duration of such episodes, reducing the severity of such episodes, slowing/reducing cancer cell growth or proliferation, slowing progression of at least one symptom, amelioration of at least one measurable physical parameter and the like. For example, immunostimulatory anti-PVRIG immune molecules should promote T cell or NK or cytokine immunity against target cells, e.g., cancer, infected or pathogen cells and thereby treat cancer or infectious diseases by depleting the cells involved in the disease condition. Conversely, immunoinhibitory anti-PVRIG immune molecules should reduce T cell or NK activity and/or or the secretion of proinflammatory cytokines which are involved in the disease pathology of some immune disease such as autoimmune, inflammatory or allergic conditions and thereby treat or ameliorate the disease pathology and tissue destruction that may be associated with such conditions (e.g., joint destruction associated with rheumatoid arthritis conditions).

The PVRIG antibodies of the invention are provided in therapeutically effective dosages. A “therapeutically effective dosage” of an anti-PVRIG immune molecule according to at least some embodiments of the present invention preferably results in a decrease in severity of disease symptoms, an increase in frequency and duration of disease symptom-free periods, an increase in lifespan, disease remission, or a prevention or reduction of impairment or disability due to the disease affliction. For example, for the treatment of PVRIG positive tumors, a “therapeutically effective dosage” preferably inhibits cell growth or tumor growth by at least about 20%, more preferably by at least about 40%, even more preferably by at least about 60%, and still more preferably by at least about 80% relative to untreated subjects. The ability of a compound to inhibit tumor growth can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, or otherwise ameliorate symptoms in a subject.

One of ordinary skill in the art would be able to determine a therapeutically effective amount based on such factors as the subject's size, the severity of the subject's symptoms, and the particular composition or route of administration selected.

### 1. Cancer Treatment

The PVRIG antibody formulations of the invention find particular use in the treatment of cancer. In general, the antibodies of the invention are immunomodulatory, in that rather than directly attack cancerous cells, the anti-PVRIG antibodies of the invention stimulate the immune system, generally by inhibiting the action of PVRIG. Thus, unlike tumor-targeted therapies, which are aimed at inhibiting molecular pathways that are crucial for tumor growth and development, and/or depleting tumor cells, cancer immunotherapy is aimed to stimulate the patient's own immune system to eliminate cancer cells, providing long-lived tumor destruction. Various approaches can be used in cancer immunotherapy, among them are therapeutic cancer vaccines to induce tumor-specific T cell responses, and immunostimulatory antibodies (i.e. antagonists of inhibitory receptors=immune checkpoints) to remove immunosuppressive pathways.

Clinical responses with targeted therapy or conventional anti-cancer therapies tend to be transient as cancer cells develop resistance, and tumor recurrence takes place. However, the clinical use of cancer immunotherapy in the past few years has shown that this type of therapy can have durable clinical responses, showing dramatic impact on long term survival. However, although responses are long term, only a small number of patients respond (as opposed to conventional or targeted therapy, where a large number of patients respond, but responses are transient).

By the time a tumor is detected clinically, it has already evaded the immune-defense system by acquiring immunoresistant and immunosuppressive properties and creating an immunosuppressive tumor microenvironment through various mechanisms and a variety of immune cells.

Accordingly, the anti-PVRIG antibodies of the invention are useful in treating cancer. Due to the nature of an immuno-oncology mechanism of action, PVRIG does not necessarily need to be overexpressed on or correlated with a particular cancer type; that is, the goal is to have the anti-PVRIG antibodies de-suppress T cell and NK cell activation, such that the immune system will go after the cancers.

“Cancer,” as used herein, refers broadly to any neoplastic disease (whether invasive or metastatic) characterized by abnormal and uncontrolled cell division causing malignant growth or tumor (e.g., unregulated cell growth). The term “cancer” or “cancerous” as used herein should be understood to encompass any neoplastic disease (whether invasive, non-invasive or metastatic) which is characterized by abnormal and uncontrolled cell division causing malignant growth or tumor, non-limiting examples of which are described herein. This includes any physiological condition in mammals that is typically characterized by unregulated cell growth.

In some embodiments, the anti-PVRIG formulations of the present invention can be used in the treatment of solid tumors (including, for example, cancers of the lung, liver, breast, brain, GI tract) and blood cancers (including for example, leukemia and preleukemic disorders, lymphoma, plasma cell disorders) carcinoma, lymphoma, blastoma, sarcoma, and leukemia or lymphoid malignancies. In some embodiments, the cancer is early. In some embodiments, the cancer is advanced (including metastatic). In some embodiments, the cancers amenable for treatment of the invention include cancers that express or do not express PVRIG and further include non-metastatic or non-invasive, as well as invasive or metastatic cancers, including cancers where PVRIG expression by immune, stromal, or diseased cells suppresses antitumor responses and anti-invasive immune responses. In some embodiments, the anti-PVRIG formulations can be used for the treatment of vascularized tumors. In some embodiments, the cancer for treatment using the anti-PVRIG formulations of the present invention includes carcinoma, lymphoma, sarcoma, and/or leukemia. In some embodiments, the cancer for treatment using the anti-PVRIG formulations of the present invention includes vascularized tumors, melanoma, non-melanoma skin cancer (squamous and basal cell carcinoma), mesothelioma, squamous cell cancer, lung cancer, small-cell lung cancer, non-small cell lung cancer, neuroendocrine lung cancer (including pleural mesothelioma, neuroendocrine lung carcinoma), NSCL (large cell), NSCLC large cell adenocarcinoma, non-small cell lung carcinoma (NSCLC), NSCLC squamous cell, soft-tissue sarcoma, Kaposi's sarcoma, adenocarcinoma of the lung, squamous carcinoma of the lung, NSCLC with PDL1>=50% TPS, neuroendocrine lung carcinoma, atypical carcinoid lung cancer, cancer of the peritoneum, esophageal cancer, hepatocellular cancer, liver cancer (including HCC), gastric cancer, stomach cancer (including gastrointestinal cancer), pancreatic cancer, glioblastoma, cervical cancer, ovarian cancer, urothelial cancer, bladder cancer, hepatoma, glioma, brain cancer (as well as edema, such as that associated with brain tumors), breast cancer (including, for example, triple negative breast cancer), testis cancer, testicular germ cell tumors, colon cancer, colorectal cancer (CRC), colorectal cancer MSS (MSS-CRC), microsatellite stable colorectal carcinoma/carcinoma), metastatic MSS-CRC, refractory metastatic MSS-CRC, refractory MSS colorectal MSS (microsatellite stable status), primary peritoneal cancer, microsatellite stable primary peritoneal cancer, platinum resistant microsatellite stable primary peritoneal cancer, CRC (MSS unknown), rectal cancer, endometrial cancer (including endometrial carcinoma), uterine carcinoma, salivary gland carcinoma, kidney cancer, renal cell cancer (RCC), renal cell carcinoma (RCC), gastro-esophageal junction cancer, prostate cancer, vulval cancer, thyroid cancer, hepatic carcinoma, carcinoid carcinoma, head and neck cancer, B-cell lymphoma (including non-Hodgkin's lymphoma, as well as low grade/follicular non-Hodgkin's lymphoma (NHL), small lymphocytic (SL) NHL, intermediate grade/follicular NHL, intermediate grade diffuse NHL, Diffuse Large B cell lymphoma, high grade immunoblastic NHL, high grade lymphoblastic NHL, high grade small non-cleaved cell NHL, bulky disease NHL, mantle cell lymphoma, AIDS-related lymphoma, and Waldenström's Macroglobulinemia, Hodgkin's lymphoma (HD), chronic lymphocytic leukemia (CLL), acute lymphoblastic leukemia (ALL), T cell Acute Lymphoblastic Leukemia (T-ALL), Acute myeloid leukemia (AML), Hairy cell leukemia, chronic myeloblastic leukemia, multiple myeloma, post-transplant lymphoproliferative disorder (PTLD), abnormal vascular proliferation associated with phakomatoses, Meigs' syndrome, Merkel Cell cancer, MSI-high cancer, KRAS mutant tumors, adult T-cell leukemia/lymphoma, adenoid cystic cancer (including adenoid cystic carcinoma), malignant melanoma, pancreatic cancer, pancreatic adenocarcinoma, ovarian cancer (including ovarian carcinoma), platinum resistant ovarian cancer, high grade serous adenocarcinoma, pleural mesothelioma, cervical squamous cell carcinoma (cervical SCC), anal squamous cell carcinoma (anal SCC), carcinoma of unknown primary, gallbladder cancer, malignant melanoma, pleural mesothelioma, chordoma, endometrial sarcoma, chondrosarcoma, uterine sarcoma, uveal melanoma, amyloidosis, AL-amyloidosis, astrocytoma, and/or Myelodysplastic syndromes (MDS).

In some embodiments, the cancer for treatment using the anti-PVRIG formulations of the present invention includes a cancer selected from the group consisting of prostate cancer, liver cancer (HCC), rectal cancer, colorectal cancer (CRC), colorectal cancer MSS (MSS-CRC; microsatellite stable colorectal carcinoma/carcinoma), metastatic MSS-CRC, refractory metastatic MSS-CRC, refractory MSS colorectal, CRC (MSS unknown), ovarian cancer (including ovarian carcinoma), platinum resistant ovarian cancer, high grade serous adenocarcinoma, endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), uveal melanoma, glioma, renal cell cancer (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cell cancer, MSI-high cancer, KRAS mutant tumors, adult T-cell leukemia/lymphoma, pleural mesothelioma, anal SCC, neuroendocrine lung cancer (including neuroendocrine lung carcinoma), small cell lung cancer, NSCLC, NSCLC large cell, NSCLC squamous cell, NSCLC adenocarcinoma, atypical carcinoid lung cancer, NSCLC with PDL1>=50% TPS, cervical SCC, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, Myelodysplastic syndromes (MDS), HNSCC, PD1 refractory or relapsing cancer, gastroesophageal junction cancer, gastric cancer, chordoma, sarcoma, endometrial sarcoma, chondrosarcoma, uterine sarcoma, plasma cell disorders, multiple myeloma, amyloidosis, AL-amyloidosis, glioblastoma, astrocytoma and fallopian tube cancer.

In some embodiments, prior to being treated in accordance with the embodiments disclosed herein, the cancer patient has received at least one line of prior treatment. In some embodiments, the patient has received at least 1, 2, 3, 4, 5, 6, 7, 8, 9, or 10 lines of prior treatment. In some embodiments, the patient has received at least 1 line of prior treatment. In some embodiments, the patient In some embodiments, the patient has received at least 2 lines of prior treatment. In some embodiments, the patient has received at least 2 lines of prior treatment. In some embodiments, the patient has received at least 3 lines of prior treatment. In some embodiments, the patient has received at least 4 lines of prior treatment. In some embodiments, the patient has received at least 5 lines of prior treatment. In some embodiments, the patient has received at least 6 lines of prior treatment. In some embodiments, the patient has received at least 7 lines of prior treatment. In some embodiments, the patient has received at least 8 lines of prior treatment. In some embodiments, the patient has received at least 9 lines of prior treatment. In some embodiments, the patient has received at least 10 lines of prior treatment.

In some embodiments, the cancer patient has received prior treatment with lenvatinib, pembrolizumab, or a combination treatment comprising lenvatinib and pembrolizumab.

In some embodiments the cancer patient has received prior treatment with one or more of capecitabine, irinotecan, abemaciclib, fulvestrant, tamoxifen, taxotere, adriamycin, cyclophosphamide, carboplatin, gemcitabine, etoposide, cisplatin, atezolizumab, doxil, bevacizumab, everolimus, abraxane, anastrozole, trodelvy, 5FU (fluorouracil), and any combination thereof.

“Cancer therapy” herein refers to any method that prevents or treats cancer or ameliorates one or more of the symptoms of cancer. Typically such therapies will comprises administration of immunostimulatory anti-PVRIG antibodies (including antigen-binding fragments) either alone or in combination with chemotherapy or radiotherapy or other biologics and for enhancing the activity thereof, i.e., in individuals wherein expression of PVRIG suppresses antitumor responses and the efficacy of chemotherapy or radiotherapy or biologic efficacy. In some embodiments, the cancer therapy is a receptor tyrosine kinase inhibitor therapy. In some embodiments, the receptor tyrosine kinase inhibitor therapy comprises lenvatinib.

In some embodiments, the anti-PD-1 antibody is nivolumab. In some embodiments, the anti-PD-1 antibody is nivolumab is administered at 360 mg. In some embodiments, the anti-PD-1 antibody is nivolumab is administered at 360 mg IV. In some embodiments, the anti-PD-1 antibody is nivolumab is administered at 360 mg IV 3 weeks (e.g., 360 mg IV Q-3 weeks). In some embodiments, the anti-PD-1 antibody is nivolumab is administered at 480 mg. In some embodiments, the anti-PD-1 antibody is nivolumab is administered at 480 mg IV. In some embodiments, the anti-PD-1 antibody is nivolumab is administered at 480 mg IV 3 weeks (e.g., 480 mg IV Q-3 weeks). In some embodiments, the anti-PD-1 antibody nivolumab is administered at 360 mg and the anti-PVRIG is administered at 20 mg/kg. In some embodiments, the anti-PD-1 antibody nivolumab is administered at 360 mg IV and the anti-PVRIG is administered at 20 mg/kg IV. In some embodiments, the anti-PD-1 antibody nivolumab is administered at 480 mg and the anti-PVRIG is administered at 20 mg/kg. In some embodiments, the anti-PD-1 antibody nivolumab is administered at 480 mg IV and the anti-PVRIG is administered at 20 mg/kg IV. In some embodiments, the anti-PD-1 antibody nivolumab is administered at 360 mg IV for 3 weeks (e.g., 360 mg IV Q-3 weeks) and the anti-PVRIG is administered at 20 mg/kg IV for 3 weeks. In some embodiments, the anti-PD-1 antibody nivolumab is administered at 480 mg IV for 3 weeks (e.g., 480 mg IV Q-3 weeks) and the anti-PVRIG is administered at 20 mg/kg for 3 weeks. In some embodiments, the anti-PD-1 antibody nivolumab is administered at 360 mg IV for 4 weeks (e.g., 360 mg IV Q-4 weeks) and the anti-PVRIG is administered at 20 mg/kg IV for 4 weeks. In some embodiments, the anti-PD-1 antibody nivolumab is administered at 480 mg IV for 4 weeks (e.g., 480 mg IV Q-4 weeks) and the anti-PVRIG is administered at 20 mg/kg for 4 weeks. In some embodiments the anti-PVRIG is CHA.7.518.1.H4 (S241P). In some embodiments, the subject administered the anti-PVRIG antibody in combination with the anti-PD-1 antibody has exhausted all available standard therapy, including for example, but not limited to ECOG 0-1, prior anti-PD-1, prior anti-PD-L1, prior anti-CTLA-4, prior OX-40, and/or prior CD137 therapies.

In some aspects, the disclosure herein provides methods for increasing infiltration of T cells in a tumor in a patient with an anti-PVRIG antibody in combination with nivolumab as disclosed herein. In some embodiments, the methods increase the infiltration of T cells in a tumor in a patient with PD-L1^{low} cancer. In some embodiments, the methods disclosed herein increase the infiltration in a tumor in a patient with PVRL2+PD-L1^{low} cancer.

In some embodiments, the tumor has a PD-L1 CPS of less than 1, 2, or 3. In some embodiments, the tumor has a PD-L1 CPS of less than 1. In some embodiments, the tumor has a PD-L1 CPS of less than 2. In some embodiments, the tumor has a PD-L1 CPS of less than 3.

In some embodiments, the tumor prior to the treatment and/or during treatment has a PVRL2 H-score of at least 250, 255, 260, 265, 270, 275, 280, 285, 290, 295, 300, or more. In some embodiments, the tumor prior to the treatment and/or during treatment has a PVRL2 H-score of at least 250.

In some embodiments, the T cells comprise effector memory CD8 positive T cells and/or CD8 positive T cells.

In some embodiments, the increased infiltration of T cells in a tumor is characterized by one or more selected from the group consisting of
(a) presence of and/or presence of an increased level of effector memory CD8 positive T cells, or CD8 positive T cells in the tumor;
(b) an increase in TCR diversity index in the tumor; and
(c) an increase in the expression level of IFNγ signature in the tumor; as compared to a control, pre-treatment sample, or a patient that does not respond to treatment with an anti-PVRIG antibody in combination with nivolumab.
   2. Selected Monotherapy Treatment with Formulation Embodiments

In some embodiments, the present invention provides for treatment of cancer in a subject in need thereof by administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain variable domain comprising the vhCDR1, vhCDR2, and vhCDR3 from the heavy chain of CHA.7.518.1.H4 (S241P) (SEQ ID NO:4), and
   ii) a light chain variable domain comprising the vlCDR1, vlCDR2, and vlCDR3 from the light chain of CHA.7.518.1.H4 (S241P) (SEQ ID NO:9);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain variable domain comprising the vhCDR1, vhCDR2, and vhCDR3 from the heavy chain of CHA.7.518.1.H4 (S241P) (SEQ ID NO:4), and
   ii) a light chain variable domain comprising the vlCDR1, vlCDR2, and vlCDR3 from the light chain of CHA.7.518.1.H4 (S241P) (SEQ ID NO:9).
(a) an anti-PVRIG antibody;
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) heavy chain variable domain is from the heavy chain of CHA.7.518.1.H4 (S241P) (SEQ ID NO:4), and
   ii) a light chain variable domain is from the light chain of CHA.7.518.1.H4 (S241P) (SEQ ID NO:9);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) heavy chain variable domain is from the heavy chain of CHA.7.518.1.H4 (S241P) (SEQ ID NO:4), and
   ii) a light chain variable domain is from the light chain of CHA.7.518.1.H4 (S241P) (SEQ ID NO:9);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising:
      a) a VH-CH1-hinge-CH2-CH3, wherein the VH is from CHA.7.518.1.H4 (S241P) (SEQ ID NO:4) and wherein the CH1-hinge-CH2-CH3 region is from IgG4; and
   ii) a light chain comprising:
      a) a VL-CL, wherein the VL from CHA.7.518.1.H4 (S241P) (SEQ ID NO: 9) and wherein the CL region is from human kappa 2 light chain;
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising:
      a) a VH-CH1-hinge-CH2-CH3, wherein the VH is from CHA.7.518.1.H4 (S241P) (SEQ ID NO:4) and wherein the CH1-hinge-CH2-CH3 region is from IgG4; and
   ii) a light chain comprising:
      a) a VL-CL, wherein the VL from CHA.7.518.1.H4 (S241P) (SEQ ID NO: 9) and wherein the CL region is from human kappa 2 light chain;
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising the heavy chain from CHA.7.518.1.H4 (S241P) (SEQ ID NO:8); and
   ii) a light chain comprising the light chain from CHA.7.518.1.H4 (S241P) (SEQ ID NO:13);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising the heavy chain from CHA.7.518.1.H4 (S241P) (SEQ ID NO:8); and
   ii) a light chain comprising the light chain from CHA.7.518.1.H4 (S241P) (SEQ ID NO:13);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.
   3. Selected Combination Treatment with Formulation Embodiments

In some embodiments, the present invention provides for treatment of cancer in a subject in need thereof by administration of nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain variable domain comprising the vhCDR1, vhCDR2, and vhCDR3 from the heavy chain of CHA.7.518.1.H4 (S241P) (SEQ ID NO:4), and
   ii) a light chain variable domain comprising the vlCDR1, vlCDR2, and vlCDR3 from the light chain of CHA.7.518.1.H4 (S241P) (SEQ ID NO:9);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain variable domain comprising the vhCDR1, vhCDR2, and vhCDR3 from the heavy chain of CHA.7.518.1.H4 (S241P) (SEQ ID NO:4), and
   ii) a light chain variable domain comprising the vlCDR1, vlCDR2, and vlCDR3 from the light chain of CHA.7.518.1.H4 (S241P) (SEQ ID NO:9).
(a) an anti-PVRIG antibody;
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) heavy chain variable domain is from the heavy chain of CHA.7.518.1.H4 (S241P) (SEQ ID NO:4), and
   ii) a light chain variable domain is from the light chain of CHA.7.518.1.H4 (S241P) (SEQ ID NO:9);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) heavy chain variable domain is from the heavy chain of CHA.7.518.1.H4 (S241P) (SEQ ID NO:4), and
   ii) a light chain variable domain is from the light chain of CHA.7.518.1.H4 (S241P) (SEQ ID NO:9);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising:
      a) a VH-CH1-hinge-CH2-CH3, wherein the VH is from CHA.7.518.1.H4 (S241P) (SEQ ID NO:4) and wherein the CH1-hinge-CH2-CH3 region is from IgG4; and
   ii) a light chain comprising:
      a) a VL-CL, wherein the VL from CHA.7.518.1.H4 (S241P) (SEQ ID NO: 9) and wherein the CL region is from human kappa 2 light chain;
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising:
      a) a VH-CH1-hinge-CH2-CH3, wherein the VH is from CHA.7.518.1.H4 (S241P) (SEQ ID NO:4) and wherein the CH1-hinge-CH2-CH3 region is from IgG4; and
   ii) a light chain comprising:
      a) a VL-CL, wherein the VL from CHA.7.518.1.H4 (S241P) (SEQ ID NO: 9) and wherein the CL region is from human kappa 2 light chain;
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising the heavy chain from CHA.7.518.1.H4 (S241P) (SEQ ID NO:8); and
   ii) a light chain comprising the light chain from CHA.7.518.1.H4 (S241P) (SEQ ID NO:13);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising the heavy chain from CHA.7.518.1.H4 (S241P) (SEQ ID NO:8); and
   ii) a light chain comprising the light chain from CHA.7.518.1.H4 (S241P) (SEQ ID NO:13);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of 360 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain variable domain comprising the vhCDR1, vhCDR2, and vhCDR3 from the heavy chain of CHA.7.518.1.H4 (S241P) (SEQ ID NO:4), and
   ii) a light chain variable domain comprising the vlCDR1, vlCDR2, and vlCDR3 from the light chain of CHA. 7.518.1.H4 (S241P) (SEQ ID NO:9);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of 360 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain variable domain comprising the vhCDR1, vhCDR2, and vhCDR3 from the heavy chain of CHA.7.518.1.H4 (S241P) (SEQ ID NO:4), and
   ii) a light chain variable domain comprising the vlCDR1, vlCDR2, and vlCDR3 from the light chain of CHA. 7.518.1.H4 (S241P) (SEQ ID NO:9).
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of 360 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) heavy chain variable domain is from the heavy chain of CHA.7.518.1.H4 (S241P) (SEQ ID NO:4), and
   ii) a light chain variable domain is from the light chain of CHA.7.518.1.H4 (S241P) (SEQ ID NO:9);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of 360 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) heavy chain variable domain is from the heavy chain of CHA.7.518.1.H4 (S241P) (SEQ ID NO:4), and
   ii) a light chain variable domain is from the light chain of CHA.7.518.1.H4 (S241P) (SEQ ID NO:9);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of 360 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising:
      a) a VH-CH1-hinge-CH2-CH3, wherein the VH is from CHA.7.518.1.H4 (S241P) (SEQ ID NO:4) and wherein the CH1-hinge-CH2-CH3 region is from IgG4; and
   ii) a light chain comprising:
      a) a VL-CL, wherein the VL from CHA.7.518.1.H4 (S241P) (SEQ ID NO: 9) and wherein the CL region is from human kappa 2 light chain;
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of 360 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising:
      a) a VH-CH1-hinge-CH2-CH3, wherein the VH is from CHA.7.518.1.H4 (S241P) (SEQ ID NO:4) and wherein the CH1-hinge-CH2-CH3 region is from IgG4; and
   ii) a light chain comprising:
      a) a VL-CL, wherein the VL from CHA.7.518.1.H4 (S241P) (SEQ ID NO: 9) and wherein the CL region is from human kappa 2 light chain;
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of 360 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising the heavy chain from CHA.7.518.1.H4 (S241P) (SEQ ID NO:8); and
   ii) a light chain comprising the light chain from CHA.7.518.1.H4 (S241P) (SEQ ID NO:13);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of 360 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising the heavy chain from CHA.7.518.1.H4 (S241P) (SEQ ID NO:8); and
   ii) a light chain comprising the light chain from CHA.7.518.1.H4 (S241P) (SEQ ID NO:13);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of 480 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain variable domain comprising the vhCDR1, vhCDR2, and vhCDR3 from the heavy chain of CHA.7.518.1.H4 (S241P) (SEQ ID NO:4), and
   ii) a light chain variable domain comprising the vlCDR1, vlCDR2, and vlCDR3 from the light chain of CHA.7.518.1.H4 (S241P) (SEQ ID NO:9);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of 480 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain variable domain comprising the vhCDR1, vhCDR2, and vhCDR3 from the heavy chain of CHA.7.518.1.H4 (S241P) (SEQ ID NO:4), and
   ii) a light chain variable domain comprising the vlCDR1, vlCDR2, and vlCDR3 from the light chain of CHA.7.518.1.H4 (S241P) (SEQ ID NO:9).
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of 480 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) heavy chain variable domain is from the heavy chain of CHA.7.518.1.H4 (S241P) (SEQ ID NO:4), and
   ii) a light chain variable domain is from the light chain of CHA.7.518.1.H4 (S241P) (SEQ ID NO:9);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of 480 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) heavy chain variable domain is from the heavy chain of CHA.7.518.1.H4 (S241P) (SEQ ID NO:4), and
   ii) a light chain variable domain is from the light chain of CHA.7.518.1.H4 (S241P) (SEQ ID NO:9);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of 480 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising:
      a) a VH-CH1-hinge-CH2-CH3, wherein the VH is from CHA.7.518.1.H4 (S241P) (SEQ ID NO:4) and wherein the CH1-hinge-CH2-CH3 region is from IgG4; and
   ii) a light chain comprising:
      a) a VL-CL, wherein the VL from CHA.7.518.1.H4 (S241P) (SEQ ID NO: 9) and wherein the CL region is from human kappa 2 light chain;
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of 480 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising:
      a) a VH-CH1-hinge-CH2-CH3, wherein the VH is from CHA.7.518.1.H4 (S241P) (SEQ ID NO:4) and wherein the CH1-hinge-CH2-CH3 region is from IgG4; and
   ii) a light chain comprising:
      a) a VL-CL, wherein the VL from CHA.7.518.1.H4 (S241P) (SEQ ID NO: 9) and wherein the CL region is from human kappa 2 light chain;
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of 480 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising the heavy chain from CHA.7.518.1.H4 (S241P) (SEQ ID NO:8); and
   ii) a light chain comprising the light chain from CHA.7.518.1.H4 (S241P) (SEQ ID NO:13);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of 480 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising the heavy chain from CHA.7.518.1.H4 (S241P) (SEQ ID NO:8); and
   ii) a light chain comprising the light chain from CHA.7.518.1.H4 (S241P) (SEQ ID NO:13);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of BMS-986207, nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain variable domain comprising the vhCDR1, vhCDR2, and vhCDR3 from the heavy chain of CHA.7.518.1.H4 (S241P) (SEQ ID NO:4), and
   ii) a light chain variable domain comprising the vlCDR1, vlCDR2, and vlCDR3 from the light chain of CHA.7.518.1.H4 (S241P) (SEQ ID NO:9);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of BMS-986207, nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain variable domain comprising the vhCDR1, vhCDR2, and vhCDR3 from the heavy chain of CHA.7.518.1.H4 (S241P) (SEQ ID NO:4), and
   ii) a light chain variable domain comprising the vlCDR1, vlCDR2, and vlCDR3 from the light chain of CHA.7.518.1.H4 (S241P) (SEQ ID NO:9).
(a) an anti-PVRIG antibody;
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of BMS-986207, nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) heavy chain variable domain is from the heavy chain of CHA.7.518.1.H4 (S241P) (SEQ ID NO:4), and
   ii) a light chain variable domain is from the light chain of CHA.7.518.1.H4 (S241P) (SEQ ID NO:9);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of BMS-986207, nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) heavy chain variable domain is from the heavy chain of CHA.7.518.1.H4 (S241P) (SEQ ID NO:4), and
   ii) a light chain variable domain is from the light chain of CHA.7.518.1.H4 (S241P) (SEQ ID NO:9);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of BMS-986207, nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising:
      a) a VH-CH1-hinge-CH2-CH3, wherein the VH is from CHA.7.518.1.H4 (S241P) (SEQ ID NO:4) and wherein the CH1-hinge-CH2-CH3 region is from IgG4; and
   ii) a light chain comprising:
      a) a VL-CL, wherein the VL from CHA.7.518.1.H4 (S241P) (SEQ ID NO: 9) and wherein the CL region is from human kappa 2 light chain;
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of BMS-986207, nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising:
      a) a VH-CH1-hinge-CH2-CH3, wherein the VH is from CHA.7.518.1.H4 (S241P) (SEQ ID NO:4) and wherein the CH1-hinge-CH2-CH3 region is from IgG4; and
   ii) a light chain comprising:
      a) a VL-CL, wherein the VL from CHA.7.518.1.H4 (S241P) (SEQ ID NO: 9) and wherein the CL region is from human kappa 2 light chain;
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of BMS-986207, nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising the heavy chain from CHA.7.518.1.H4 (S241P) (SEQ ID NO:8); and
   ii) a light chain comprising the light chain from CHA.7.518.1.H4 (S241P) (SEQ ID NO:13);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of BMS-986207, nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising the heavy chain from CHA.7.518.1.H4 (S241P) (SEQ ID NO:8); and
   ii) a light chain comprising the light chain from CHA.7.518.1.H4 (S241P) (SEQ ID NO:13);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of BMS-986207, 360 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain variable domain comprising the vhCDR1, vhCDR2, and vhCDR3 from the heavy chain of CHA.7.518.1.H4 (S241P) (SEQ ID NO:4), and
   ii) a light chain variable domain comprising the vlCDR1, vlCDR2, and vlCDR3 from the light chain of CHA.7.518.1.H4 (S241P) (SEQ ID NO:9);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of BMS-986207, 360 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain variable domain comprising the vhCDR1, vhCDR2, and vhCDR3 from the heavy chain of CHA.7.518.1.H4 (S241P) (SEQ ID NO:4), and
   ii) a light chain variable domain comprising the vlCDR1, vlCDR2, and vlCDR3 from the light chain of CHA.7.518.1.H4 (S241P) (SEQ ID NO:9).
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of BMS-986207, 360 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) heavy chain variable domain is from the heavy chain of CHA.7.518.1.H4 (S241P) (SEQ ID NO:4), and
   ii) a light chain variable domain is from the light chain of CHA.7.518.1.H4 (S241P) (SEQ ID NO:9);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of BMS-986207, 360 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) heavy chain variable domain is from the heavy chain of CHA.7.518.1.H4 (S241P) (SEQ ID NO:4), and
   ii) a light chain variable domain is from the light chain of CHA.7.518.1.H4 (S241P) (SEQ ID NO:9);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of BMS-986207, 360 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising:
      a) a VH-CH1-hinge-CH2-CH3, wherein the VH is from CHA.7.518.1.H4 (S241P) (SEQ ID NO:4) and wherein the CH1-hinge-CH2-CH3 region is from IgG4; and
   ii) a light chain comprising:
      a) a VL-CL, wherein the VL from CHA.7.518.1.H4 (S241P) (SEQ ID NO: 9) and wherein the CL region is from human kappa 2 light chain;
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of BMS-986207, 360 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising:
      a) a VH-CH1-hinge-CH2-CH3, wherein the VH is from CHA.7.518.1.H4 (S241P) (SEQ ID NO:4) and wherein the CH1-hinge-CH2-CH3 region is from IgG4; and
   ii) a light chain comprising:
      a) a VL-CL, wherein the VL from CHA.7.518.1.H4 (S241P) (SEQ ID NO: 9) and wherein the CL region is from human kappa 2 light chain;
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of BMS-986207, 360 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising the heavy chain from CHA.7.518.1.H4 (S241P) (SEQ ID NO:8); and
   ii) a light chain comprising the light chain from CHA.7.518.1.H4 (S241P) (SEQ ID NO:13);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of BMS-986207, 360 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising the heavy chain from CHA.7.518.1.H4 (S241P) (SEQ ID NO:8); and
   ii) a light chain comprising the light chain from CHA.7.518.1.H4 (S241P) (SEQ ID NO:13);
(b) about 25 mM histidine;
(c) about 60 mM NaCl;
(d) about 100 mM L-Arginine; and
(e) about 0.01% % w/v polysorbate 80,
   wherein the composition has a pH from 6.5+/−0.2.

In some embodiments, the present invention provides for treatment of cancer in a subject in need thereof by administration of BMS-986207, 480 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain variable domain comprising the vhCDR1, vhCDR2, and vhCDR3 from the heavy chain of CHA.7.518.1.H4 (S241P) (SEQ ID NO:4), and
   ii) a light chain variable domain comprising the vlCDR1, vlCDR2, and vlCDR3 from the light chain of CHA.7.518.1.H4 (S241P) (SEQ ID NO:9);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of BMS-986207, 480 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain variable domain comprising the vhCDR1, vhCDR2, and vhCDR3 from the heavy chain of CHA.7.518.1.H4 (S241P) (SEQ ID NO:4), and
   ii) a light chain variable domain comprising the vlCDR1, vlCDR2, and vlCDR3 from the light chain of CHA.7.518.1.H4 (S241P) (SEQ ID NO:9).
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of BMS-986207, 480 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) heavy chain variable domain is from the heavy chain of CHA.7.518.1.H4 (S241P) (SEQ ID NO:4), and
   ii) a light chain variable domain is from the light chain of CHA.7.518.1.H4 (S241P) (SEQ ID NO:9);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of BMS-986207, 480 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) heavy chain variable domain is from the heavy chain of CHA.7.518.1.H4 (S241P) (SEQ ID NO:4), and
   ii) a light chain variable domain is from the light chain of CHA.7.518.1.H4 (S241P) (SEQ ID NO:9);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of BMS-986207, 480 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising:
      a) a VH-CH1-hinge-CH2-CH3, wherein the VH is from CHA.7.518.1.H4 (S241P) (SEQ ID NO:4) and wherein the CH1-hinge-CH2-CH3 region is from IgG4; and
   ii) a light chain comprising:
      a) a VL-CL, wherein the VL from CHA.7.518.1.H4 (S241P) (SEQ ID NO: 9) and wherein the CL region is from human kappa 2 light chain;
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of BMS-986207, 480 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising:
      a) a VH-CH1-hinge-CH2-CH3, wherein the VH is from CHA.7.518.1.H4 (S241P) (SEQ ID NO:4) and wherein the CH1-hinge-CH2-CH3 region is from IgG4; and
   ii) a light chain comprising:
      a) a VL-CL, wherein the VL from CHA.7.518.1.H4 (S241P) (SEQ ID NO: 9) and wherein the CL region is from human kappa 2 light chain;
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for treatment of cancer in a subject in need thereof by administration of BMS-986207, 480 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising the heavy chain from CHA.7.518.1.H4 (S241P) (SEQ ID NO:8); and
   ii) a light chain comprising the light chain from CHA.7.518.1.H4 (S241P) (SEQ ID NO:13);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for treatment of cancer in a subject in need thereof by administration of BMS-986207, 480 mg nivolumab and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg, and wherein the stable liquid formulation of the anti-PVRIG antibody comprises:
(a) an anti-PVRIG antibody comprising:
   i) a heavy chain comprising the heavy chain from CHA.7.518.1.H4 (S241P) (SEQ ID NO:8); and
   ii) a light chain comprising the light chain from CHA.7.518.1.H4 (S241P) (SEQ ID NO:13);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

### EXAMPLES

### Example 1: Phase 1 Study of CHA.7.518.1.H4 (S241P) Monotherapy and in Combination with Nivolumab in Patients with Advanced Solid Tumors

### Background

CHA.7.518.1.H4 (S241P) is a novel first-in-class humanized IgG4 monoclonal antibody that binds with high affinity to poliovirus receptor related immunoglobulin domain containing (PVRIG) blocking its interaction with its ligand, PVRL2 [1]

Nivolumab is an anti-PD-1 antibody approved in patients with several malignancies [2].

PD-1 inhibitors play an important role in this axis by modulating DNAM activation [3]

In preclinical experiments it has been demonstrated that PVRIG inhibition alone and in combination with anti-PD-1 leads to activation of T cells in the tumor microenvironment generating an anti-tumor immune response and tumor growth inhibition [1]

Although ICI revolutionized cancer treatment there is an urgent need to develop treatments for patients who are refractory or relapse after treatment with ICI.

While not being bound by theory, it is hypothesized that CHA.7.518.1.H4 (S241P) will be safe and tolerable and demonstrate antitumor activity in pts with R/R solid tumors

### Methods

NCT03667716 is an ongoing open-label first-in-human phase 1 study in pts with R/R solid tumors

The initial part of this study evaluating the safety and tolerability of escalating doses of CHA.7.518.1.H4 (S241P) monotherapy IV Q3 weeks and in combination with nivolumab 360 mg IV Q3 weeks is reported in this example.

### Primary Outcome Measures

To evaluate the safety profile of CHA.7.518.1.H4 (S241P) as monotherapy and in combination with nivolumab in patients with advanced solid tumors

The incidence of adverse events and dose-limiting toxicities (21-day DLT window) graded as per CTCAE v4.03

To identify the maximum tolerated dose and/or the recommended dose for expansion

To characterize the PK profile of CHA.7.518.1.H4 (S241P) as monotherapy and in combination with nivolumab

### Secondary Outcome Measures

To characterize the immunogenicity of CHA.7.518.1.H4 (S241P) alone and in combination with nivolumab

To evaluate preliminary antitumor activity of CHA.7.518.1.H4 (S241P) in combination with nivolumab (Phase 1b only) responses as per RECIST v1.1

### Exploratory Outcome Measures

To evaluate preliminary antitumor activity of CHA.7.518.1.H4 (S241P) as monotherapy

To assess any association of DNAM axis members with clinical outcome

To explore evidence of CHA.7.518.1.H4 (S241P)-mediated PD effect in blood as monotherapy as well as in combination with nivolumab

### Key Inclusion Criteria

Age≥18 yrs
Histologically or cytologically confirmed, locally advanced or metastatic solid malignancy and has exhausted all the available standard therapy or is not a candidate for the available standard therapy
ECOG performance status 0-1
Prior anti-PD-1, anti-PD-L1, anti-CTLA-4, OX-40, CD137 permissible Adequate hematological, hepatic and renal function

### Key Exclusion Criteria

Active autoimmune disease requiring systemic therapy in the last 2 years prior to the first dose of CHA.7.518.1.H4 (S241P)
Symptomatic interstitial lung disease or inflammatory pneumonitis
Untreated or symptomatic central nervous system metastases
History of immune-related events that lead to immunotherapy treatment discontinuation

### Accrual Information

No dose-limiting toxicities have been observed in the 7th CHA. 7.518.1.H4 (S241P) monotherapy dose level and earlier dose levels (red box)
No dose-limiting toxicities have been observed in the 3rd CHA. 7.518.1.H4 (S241P)+nivolumab dose level and earlier dose levels (green box)

As of the date of this presentation the 8th CHA.7.518.1.H4 (S241P) mono dose and 4th CHA.7.518.1.H4 (S241P)+nivolumab dose levels are open to enrollment at IV Q4 weeks schedule

Study NCT03667716 is in collaboration with Bristol-Myers Squibb

### REFERENCE

Spencer L, Ofer L et al., Discovery of COM701, a therapeutic antibody targeting the novel immune checkpoint PVRIG, for the treatment of cancer. J Clin Oncol. 2017; (suppl; abstr 3074)
Nivolumab package insert. http://packageinserts.bms.com/pi/pi_opdivo.pdf. Accessed Jul. 22, 2019.
Wang B, Zhang W et al., Combination cancer immunotherapy targeting PD-1 and GITR can rescue CD8+ T cell dysfunction and maintain memory phenotype. Sci. Immunol. 2018; November 2: 3 (29).

### Example 2: CHA.7.518.1.H4 (S241P) Demonstrates Antitumor Activity as Monotherapy and in Combination with Nivolumab in Patients with Advanced Malignancies

### Background

### Introduction

CHA.7.518.1.H4 (S241P) is a novel first-in-class Immune checkpoint inhibitor (ICI) that binds with high affinity to poliovirus receptor related immunoglobulin domain containing (PVRIG) blocking its interaction with its ligand, PVRL2 and regulating the activity of T/NK cells through the DNAM/TIGIT axis. In preclinical experiments inhibition of PVRIG alone and in combination with anti-PD1 and/or TIGIT leads to tumor growth inhibition and activation of T-cells in the microenvironment generating an antitumor response.

### Methods

A total of 28 pts (Arm A/B 16/12) with a variety of cancer types were enrolled (including patients with a variety of tumor types who had failed all available standard therapies). 16 patients in Arm A (CHA.7.518.1.H4 (S241P) monotherapy dose escalation) and 12 patients in Arm B (CHA.7.518.1.H4 (S241P) dose escalation with nivolumab). Hybrid accelerated (1st 4 dose cohorts in Arm A) and 3+3 study design (cohorts 5-8 in Arm A and all cohorts in Arm B). Patients with performance status ECOG 0-1 and advanced or metastatic solid tumors who failed standard of care treatment were eligible. Prior ICIs were permissible. In Arm A pts received CHA.7.518.1.H4 (S241P) monotherapy 0.01, 0.03, 0.1, 0.3, 1, 3, 10 mg/kg (all IV Q3 weeks (wks)) and 20 mg/kg (IV Q4 wks). In Arm B, pts received CHA.7.518.1.H4 (S241P) at 0.3, 1 or 3 mg/kg plus nivolumab 360 mg IV q3 weeks (3 pts/dose cohort) and 3 pts received 10 mg/kg plus nivolumab 480 mg IV q4 weeks. Treatment emergent adverse events (TEAEs) were reported per CTCAE v4.03 and responses per RECIST v1.1. Dose-limiting toxicities (DLTs) were evaluated within a 21-day or 28-day window (for 3- or 4-weeks dosing schedule respectively). Data cutoff date was Jan. 23, 2020.

### Results

The median number of prior anticancer therapies were: Arm A, 7 (range 2-15), Arm B, 5 (range 2-9). No DLTs have been reported in any of the dose cohorts. Treatment was well tolerated with no subjects discontinuing treatment due to toxicity, the most frequent TEAEs in Arm A were fatigue (46%), nausea (31%) and anxiety (23%)-all G1-2. In Arm B≥4 pts-anemia, lower extremity edema, rash and fatigue the majority being grade 1-2 (88%). In Arms A+B: partial response (PR)+stable disease (SD) was 57% (16/28). Of note: Arm A (CHA.7.518.1.H4 (S241P) 20 mg/kg IV q4 weeks): confirmed PR in a pt with primary peritoneal cancer ongoing on treatment>15 weeks. Arm B: unconfirmed PR in a pt with MSS-CRC on CHA.7.518.1.H4 (S241P) 0.3 mg/kg plus nivolumab. A confirmed partial response in a patient with microsatellite stable primary peritoneal cancer enrolled in the eighth and last dose cohort in Arm A; the patient is continuing on study treatment (more than 15 weeks).

360 mg IV q3 weeks, ongoing on treatment>34 weeks.

Overall 11/28 patients remain on study treatment including 3 patients who have not reached first imaging assessment. For both treatment arms, the timepoint response of partial response and stable disease/disease control rate were reported in 16 of 28 patients (57%).

### Conclusion

CHA.7.518.1.H4 (S241P) is well tolerated as monotherapy and in combination with nivolumab in a variety of heavily pretreated pts with advanced or metastatic solid tumors. CHA.7.518.1.H4 (S241P) demonstrates encouraging preliminary antitumor activity with objective responses as monotherapy and in combination with nivolumab in hard to treat tumor types (primary peritoneal, microsatellite stable primary peritoneal cancer (MSS primary peritoneal cancer or MSS-PPC), and microsatellite stable colorectal cancer (MSS-CRC)).

### Example 3: CHA.7.518.1.H4 (S241P) Demonstrates Antitumor Activity as Monotherapy and in Combination with Nivolumab in Patients with Advanced Malignancies

### Introduction

There is a high unmet medical need for the treatment of patients who are refractory to or relapse following treatment with checkpoint inhibitors.

Inhibition of poliovirus receptor related immunoglobulin domain containing (PVRIG) leads to enhanced activation of T and NK cells, and results in tumor growth inhibition in mouse tumor models (Spencer L, Ofer L et al., Discovery of COM701, a therapeutic antibody targeting the novel. immune checkpoint PVRIG, for the treatment of cancer. J Clin Oncol. 2017; (suppl; abstr 3074)).

CHA.7.518.1.H4 (S241P) is a novel first-in-class humanized IgG4 monoclonal antibody that binds with high affinity PVRIG blocking its interaction with its ligand, PVRL2.

Previous data supported the preliminary antitumor activity of CHA.7.518.1.H4 (S241P) monotherapy (Dumbrava E, Fleming G, Hamilton E et al., Journal for ImmunoTherapy of Cancer 2019, 7 (Suppl 1): P421. SITC November 2019.)

The present data provides data related to the preliminary safety and antitumor activity of CHA.7.518.1.H4 (S241P) in combination with nivolumab (Arm B) and provides a data update in CHA.7.518.1.H4 (S241P) monotherapy dose cohorts (Arm A).

CHA.7.518.1.H4 (S241P) well tolerated and with a manageable safety profile as monotherapy and in combination with nivolumab:
a. No increase in toxicity in combination with nivolumab.
b. No subjects discontinued study treatment due to toxicity of any study drug.

Single-agent MTD CHA.7.518.1.H4 (S241P) 20 mg/kg IV Q4 weeks; combination dose escalation continues.

Confirmed partial responses in 2 patients.

CHA.7.518.1.H4 (S241P) monotherapy 20 mg/kg IV Q4 weeks-primary peritoneal cancer (ongoing on study treatment 25 weeks).

CHA.7.518.1.H4 (S241P), (CHA.7.518.1.H4 (S241P) 0.3 mg/kg IV Q3 weeks)+Nivolumab (480 mg IV Q3 weeks)-MSS-CRC (ongoing on study treatment 44 weeks).

Disease control rate for CHA.7.518.1.H4 (S241P) monotherapy was 11/16 [69%] in diverse tumor types.

Disease control rate for CHA.7.518.1.H4 (S241P)+nivolumab was 9/12 [75%] in diverse tumor types.

Durable stable disease (SD>6 months) in 6/28 pts and diverse tumor types.

Arm A (CHA.7.518.1.H4 (S241P) monotherapy): Adenoid cystic CA, CRC-MSS.

Arm B (CHA.7.518.1.H4 (S241P)+nivolumab): Anal SCC, CRC-MSS, Endometrial, NSCLC (squamous).

Preliminary CHA.7.518.1.H4 (S241P) PK profile supports Q4 weeks dosing.

CHA.7.518.1.H4 (S241P) monotherapy dose expansion at RDFE planned (NSCLC, OVCA, Breast, Endometrial, MSS-CRC).

### Example 4: CHA.7.518.1.H4 (S241P) with or without Nivolumab-Results of an Ongoing Phase 1 Study of Safety, Tolerability and Preliminary Antitumor Activity in Pts with Advanced Solid Malignancies (NCT0366716)

### Introduction

CHA.7.518.1.H4 (S241P) has been examined with or without nivolumab-results of an ongoing phase 1 study of safety, tolerability and preliminary antitumor activity in patients with advanced solid malignancies (NCT0366716).

### Background

CHA.7.518.1.H4 (S241P) is a novel first in class humanized IgG4 monoclonal antibody that binds with high affinity to poliovirus receptor related immunoglobulin domain containing (PVRIG), blocking its interaction with its ligand, PVRL2. Inhibition of PVRIG leads to enhanced activation of T/NK cells and in mouse models results in tumor growth inhibition. As described herein, CHA.7.518.1.H4 (S241P)+nivolumab has antitumor activity and an acceptable safety/tolerability profile.¹ In this example, new data on safety/tolerability/pharmacokinetics in the final dose escalation combination cohort, mono expansion cohort (MEC) and followup data in prior cohorts is provided.

### Methods

In the study, 51 patients were enrolled: Arm A (CHA.7.518.1.H4 (S241P) mono dose escalation), 16 pts in 8 cohorts (0.01-20 mg/kg IV Q3/4 weeks); Arm B (CHA.7.518.1.H4 (S241P) 0.3-20 mg/kg+nivolumab 360 mg or 480 mg IV Q3/Q4 weeks), 15 pts in 5 cohorts; 20 pts in MEC (NSCLC, ovarian, breast, endometrial and colorectal cancer at the recommended dose for expansion (RDFE), 20 mg/kg IV Q4 weeks). Key inclusion criteria: Age≥18 yrs, histologically confirmed metastatic solid malignancy and has exhausted available standard treatment, ECOG 0-1, prior ICI permissible (except prior treatment with CHA.7.518.1.H4 (S241P) or PVRIG inhibitor). Key exclusion criteria: active autoimmune disease requiring systemic treatment, history of inflammatory lung disease. Primary objectives-safety and tolerability of CHA.7.518.1.H4 (S241P)+nivolumab (AEs, CTCAE v4.03), PK of CHA.7.518.1.H4 (S241P) and RDFE. Key secondary/exploratory objectives-antitumor activity of CHA.7.518.1.H4 (S241P)+nivolumab (RECIST v1.1). Correlative studies with PVRL2 expression in tumor biopsy, blood cytokine and immunophenotyping.

Details regarding the Meso Scale Discovery (MSD) assay can be found on the World Wide Web at mesoscale.com/˜/media/files/product % 20inserts/proinflammatory %20panel %201%20human % 20insert.pdf. As stated in the assay materials, MSD provides a plate pre-coated with capture antibodies on independent and well-definte spots. Multiplex assays and individual assays, such as for IFNγ, are commercially available. Sample and solution containing detection antibodies conjugated with electrochemiluminescent labels (MSD SULFO-TAG™) are added over the course of the incubation period. Analytes in the sample bind to capture antibodies immobilized on the working electrode surface and recruitment of the detection antibodies by the bound analytes completes the sandwich. MSD buffer that allows for the appropriate chemical environment for electrochemiluminescence (ECL) and loads the plate into an MSD instrument where a voltage applied to the plate electrodes causes captured labels to meit light, the intensity of which is proportional to the amount of analyte present and provides a quantitative measure of analyte in the sample (such methods have been described and validated in Lee, J. W., et al., Pharm. Res. 23:312-328 (2006)).

### Results

No DLT (dose-limiting toxicity) in Arms A or in B up to cohort 5 (CHA.7.518.1.H4 (S241P) 20 mg/kg+nivolumab 480 mg; all IV Q4 weeks), CHA.7.518.1.H4 (S241P) PK profile cohort 5 similar to CHA.7.518.1.H4 (S241P) mono 20 mg/kg IV Q4 weeks (cohort 8). Grade≤2 AEs 21/38 pts (55%), 8/16 pts (50%) respectively in the mono and combination arms. Most frequent AEs (adverse events) mono Grade≤2 fatigue 12/38 pts (31%), nausea 9/38 (23%); combination arm: fatigue 7/16 pts (44%) and AST increased 4/16 pts (25%). Arm A (cohort 8)-pt with platinum resistant primary peritoneal cancer confirmed PR ongoing at 62 weeks. Arm B (CHA.7.518.1.H4 (S241P) 10 mg/kg+nivolumab 480 mg, all IV Q4 weeks), a pt with renal cell CA with confirmed SD [ongoing at 58 weeks, CHA.7.518.1.H4 (S241P) 0.3 mg/kg+nivolumab 360 mg; IV Q3 weeks] a pt with anal SCCA with confirmed CR, ongoing at 79 weeks, last treatment with prior PD on nivolumab. Best response of SD in 6 patients in MEC [1-endometrial, 3 NSCLC, 2 OVCA], 2 patients [NSCLC, OVCA] ongoing at 26, 20 weeks.

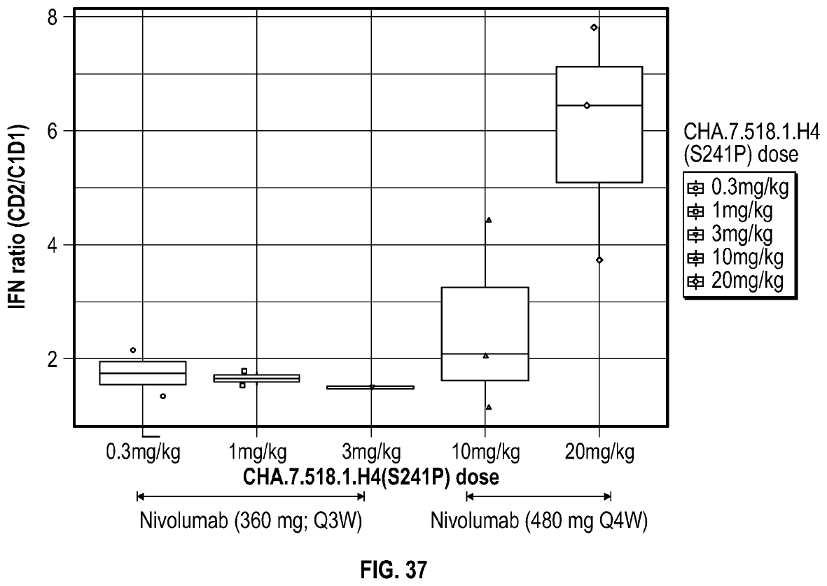

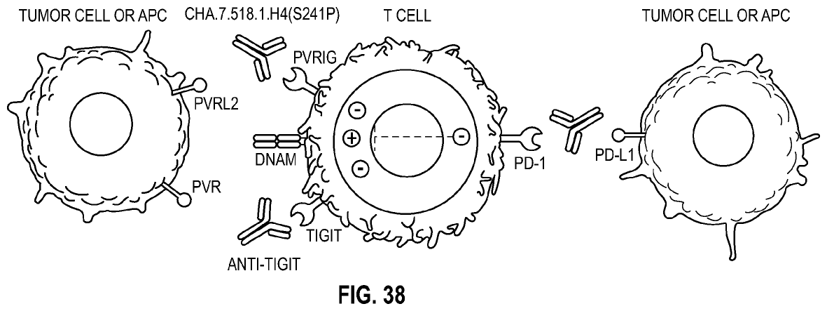

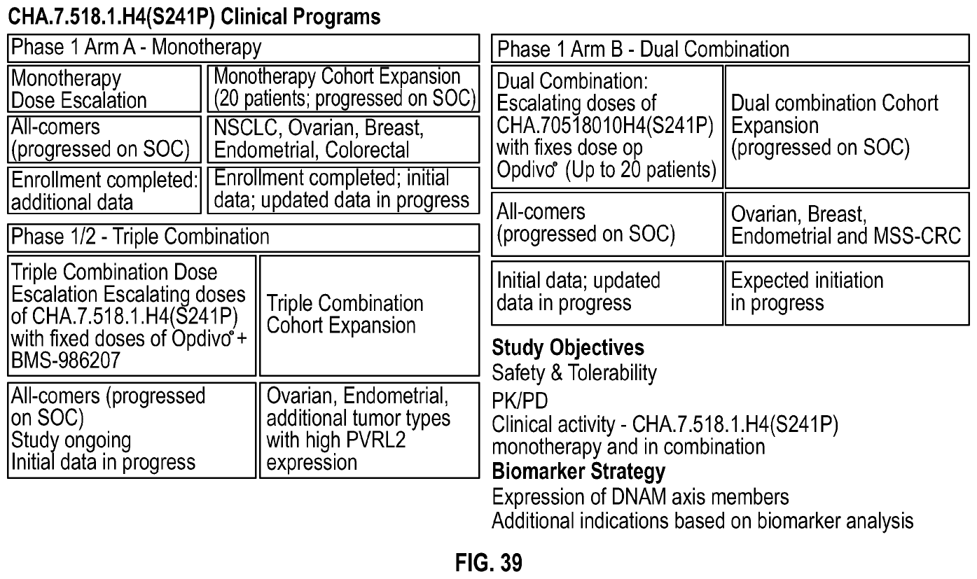

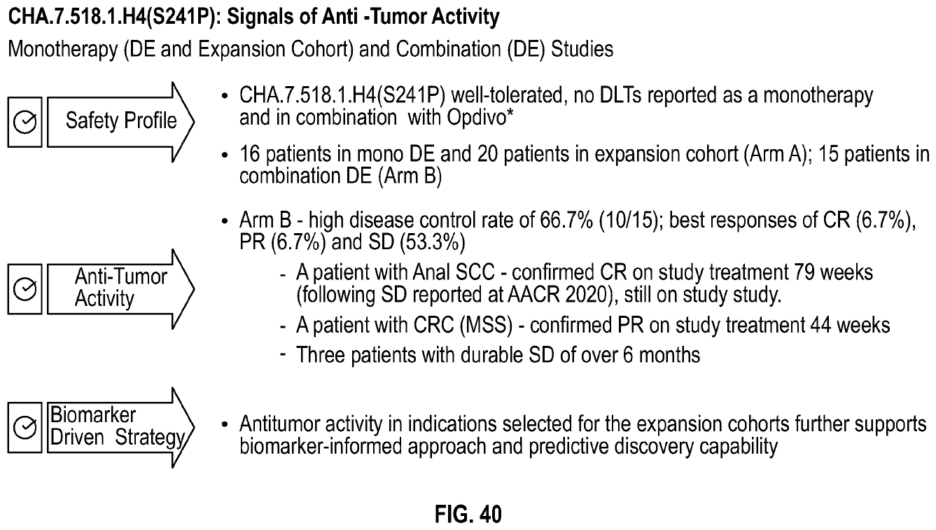

As provided in FIG. 37 data showing changes in Serum IFNγ among treatment groups from P1 arm B combination therapy treated with Nivolumab 360 mg (Q3W; every 3 weeks) for CHA.7.518.1.H4 (S241P) doses <10 mg/kg, nivolumab 480 mg (Q4W; every 4 weeks) for higher CHA.7.518.1.H4 (S241P) doses (10 and 20 mg/kg). Shown are the fold change in the serum levels of IFNγ at day 2 (C1D2) post-treatment compare to baseline (C1D1). Samples were assessed for levels of the cytokine IFNγ using a pro-inflammatory human cytokine 10-plex assay kit and MSD reader (Meso Scale Discovery). Sample signals were compared to calibration curves to determine the concentration of each analyte in serum samples.

### Conclusion

CHA.7.518.1.H4 (S241P)±nivolumab well tolerated with no new safety signals. Encouraging signal of antitumor activity including in patients with prior treatment with ICI. Updated data is in progress.

### REFERENCE

1. COM701 demonstrates preliminary antitumor activity as monotx and in combination with nivolumab in pts with advanced solid tumors. Abstract CT031 Sullivan R, Rasco D et al., AACR Annual Meeting Apr. 27-28, 2020.

### Example 5: CHA.7.518.1.H4 (S241P) with or without Nivolumab-Results of an Ongoing Phase 1 Study of Safety, Tolerability and Preliminary Antitumor Activity in Pts with Advanced Solid Malignancies (NCT03667716)

### Introduction

CHA.7.518.1.H4 (S241P) is a novel 1^{st} in class humanized IgG4 monoclonal antibody that binds with high affinity to poliovirus receptor related immunoglobulin domain containing (PVRIG), blocking its interaction with its ligand, PVRL2. Blocking of PVRIG leads to enhanced activation of T/NK cells and in mouse models inhibits tumor growth. The example provides additional and updated results on safety/tolerability/pharmacokinetics and antitumor activity from the ongoing study including additional results in dose escalation combination cohort, monotherapy expansion cohort (MEC).

### Methods

The study enrolled a total of 51 DLT-evaluable pts: Arm A (CHA.7.518.1.H4 (S241P) mono dose escalation), 16 pts in 8 cohorts (0.01-20 mg/kg IV Q3/4 wks); Arm B (CHA.7.518.1.H4 (S241P) 0.3-20 mg/kg+nivolumab (NIVO) 360 mg/480 mg IV Q3/Q4 wks), 15 pts in 5 cohorts; 20 pts in MEC (NSCLC, OVCA, breast, endometrial and CRC) at the recommended dose for expansion (RDFE), 20 mg/kg IV Q4 wks. Key inclusion criteria: Age≥18 yrs, histologically confirmed metastatic solid malignancy, has exhausted available standard tx, ECOG 0-1, prior ICI permissible (except prior tx with a PVRIG inhibitor). Key exclusion criteria: active autoimmune disease requiring systemic tx, hx inflammatory lung disease. Primary objectives-safety/tolerability of CHA.7.518.1.H4 (S241P)=NIVO (AEs, CTCAE v4.03), PK, RDFE. Key secondary/exploratory objectives-antitumor activity of CHA.7.518.1.H4 (S241P)+NIVO (RECIST v1.1), evaluation of PVRL2 expression in tumor biopsy, blood cytokines and immunophenotyping.

### Results

No DLTs were reported in Arms A or B. CHA.7.518.1.H4 (S241P) PK profile similar in Arm A, 20 mg/kg IV Q4 wks (cohort 8) and Arm B cohort 5 (CHA.7.518.1.H4 (S241P) 20 mg/kg+NIVO 480 mg; all IV Q4 wks). Frequency of TEAEs in safety population (N=54 pts): pts on CHA.7.518.1.H4 (S241P) mono (N=38)-No AE (4), Grade≤2 (21), G3 (11), G4 (1), G5 (1, PD), pts on combo (N=16)-Grade≤2 (8), G3 (7), G5 (1, PD). Serious TEAE: pts on CHA.7.518.1.H4 (S241P) mono 11/38, pts on combo 6/16. Most frequent AEs in Arm A: Grade≤2 fatigue 12/38 pts (31%), nausea 9/38 (23%); Arm B: fatigue 7/16 pts (44%) and AST increased 4/16 pts (25%). Antitumor activity—in Arm A (cohort 8), a pt with platinum resistant primary peritoneal cancer had confirmed PR ongoing 14 months. In Arm B (CHA.7.518.1.H4 (S241P) 10 mg/kg+NIVO 480 mg, all IV Q4 wks), a pt with anal SCCA; confirmed CR, ongoing 18 months, last tx with prior PD on NIVO. In addition, a pt with renal cell CA had confirmed SD [ongoing 13 months, CHA.7.518.1.H4 (S241P) 0.3 mg/kg+NIVO 360 mg; IV Q3 wks] In MEC, 30% (6/20 pts) had best response of SD [1-endometrial, 3 NSCLC, 2 OVCA], 2 pts [NSCLC, OVCA] ongoing at 6/4 months. Overall 16 pts had prior tx-refractory disease, 9 (56%) had best response of >SD. Of 18 pts with prior tx with ICI, 13 (72%) had best response of ≥SD.

Combination arm dose-escalation data. The updated data disclosed include complete data from all 5 dose levels in the study. Overall, disease control rate of about 67% was shown even given the highly refractory nature of this population. Durable responses were observed including the partial response with 2 patients still remaining on study (approximately an additional 10 months). One of these two patients, who progressed on an immune checkpoint inhibitor prior to enrolling to our study has converted to a confirmed complete response.

Based on this data, initiated a Phase 1b cohort expansion combination study evaluating CHA.7.518.1.H4 (S241P) with nivolumab, in parallel to our on-going triplet study and will enroll patients with ovarian, breast, endometrial and microsatellite-stable colorectal cancers. Our recent results, including the complete response in a patient who relapsed on PD-1 therapy, increases our confidence that there are certain patient subpopulations which are likely to respond to PVRIG/PD-1 dual blockade in a clinical setting, including those progressed on immune checkpoint blockers. This study will also provide additional information related to the contribution of the different components of the DNAM axis across our ongoing and future CHA.7.518.1.H4 (S241P) studies, and specifically the ongoing triplet study.

Data from o CHA.7.518.1.H4 (S241P) monotherapy cohort expansion. Study was designed as a safety and tolerability study and has used a biomarker informed strategy to select tumor types likely to respond to treatment alone or in combination based on preclinical expression data and clinical results from the dose escalation arm. These indications are-endometrial, breast, ovarian, colorectal and non-small cell lung cancer.

In this study of 20 patients, for which enrollment was completed in Q4 2020, 6 patients with a best response of stable disease across endometrial, non-small cell lung and ovarian cancer and durable anti-tumor activity of two-three patients (including NSCLC and ovarian) on treatment at 20 and 26 weeks. The combined data of this cohort expansion study with data from the monotherapy dose escalation study that includes a patient with durable confirmed partial response still on study treatment for more than a year, demonstrate signals of antitumor activity of CHA.7.518.1.H4 (S241P) treatment in patients with highly refractory disease in tumor types typically unresponsive to immune checkpoint inhibitors.

Correlative assessments are being performed based on data from patient samples to gain insights relating to CHA.7.518.1.H4 (S241P) and PVRIG/PVRL2 pathway biology, particularly in indications that are typically not responsive to PD-1 to further understand CHA.7.518.1.H4 (S241P) activity, as well as to inform CHA. 7.518.1.H4 (S241P) and TIGIT clinical program development path and additional studies to be conducted in indications with preliminary encouraging signals of antitumor activity.

Initial assessments of patient's peripheral blood samples from our small monotherapy expansion cohorts study suggest that CHA.7.518.1.H4 (S241P) may enhance immune activation in cancer patients, alone or in combination with Nivolumab. Data analysis is still on-going.

Data in patients enrolled in the dose escalation arms of the ongoing Phase 1 study of CHA.7.518.1.H4 (S241P) monotherapy in combination with nivolumab in patients with advanced solid tumors are provided here. This included data from all monotherapy dose escalation cohorts through 20 mg/kg IV Q4 wks and 4 of the 5 dose levels from the dual combination dose escalation arm. Safety and tolerability, and preliminary antitumor activity of CHA.7.518.1.H4 (S241P) monotherapy and in combination with nivolumab are provided.

Notably there were 2 durable partial responses (PR). One PR in the combination arm of a patient with MSS colorectal cancer, with treatment ongoing for more than 10 months. Typically patients with CRC MSS are unresponsive to immune checkpoint inhibitors and published data demonstrates a median PFS of approximately 8 weeks in this patient population. Another PR in the monotherapy arm of a patient with primary peritoneal cancer that is also platinum resistant and with MSS status with treatment ongoing at 24 weeks at the time of the presentation. The study showed durability of antitumor activity and responses with a number of patients in the combination arm remaining on study for over 200 days.

No reported DLTs in dose level 5 of the dual combination dose escalation arm of CHA.7.518.1.H4 (S241P) 20 mg/kg+nivolumab 480 mg, IV Q4 weeks and there were no new safety findings at this dose. Additionally, the PK profile of CHA. 7.518.1.H4 (S241P) was similar to previously reported at the CHA.7.518.1.H4 (S241P) 20 mg/kg IV Q4 wks dose in the monotherapy dose escalation cohort.

The results of all 15 patients who were enrolled in the combination dose escalation cohorts. Two of the patients had a clinical response of CR or PR, and the disease control rate ie best timepoint assessment of SD or better was reported in 10 of 15 patients (67%). This preliminary antitumor activity is encouraging considering this is an all corner heavily pretreated patient population with median of 5 prior therapies. In addition, 5/15 pts (33%) had disease control rate of ≥6 months.

Additional findings from individual patients in the combination dose escalation arm are provided.

Starting first with a patient with anal squamous cell carcinoma with initial assessments of confirmed stable disease for over a year on 0.3 mg/kg IV Q3 wks+nivo 360 mg IV Q 3 wks. This patient now has confirmed complete response on imaging and is continuing on study treatment at more than 18 months. Before enrolling in the ongoing study, this patient received last prior treatment with nivolumab, achieving a confirmed complete response, and then progressed while on nivolumab. This confirmed complete response, following progression on an immune checkpoint inhibitor, suggests that treatment with CHA.7.518.1.H4 (S241P) may broaden the target patient population for CHA.7.518.1.H4 (S241P). In addition, this complete response, along with the rest of this combination study data, also supports that dual inhibition with CHA.7.518.1.H4 (S241P) and a PD1 inhibitor may offer increased clinical benefit vs. monotherapy with an immune checkpoint inhibitor in certain tumor types by inhibiting two parallel non-redundant checkpoint pathways. Anal squamous cell carcinoma is an uncommon and/or orphan cancer and an indication of high unmet medical need for new treatment options.

In addition to the complete response, and a previously reported confirmed partial response in a patient with colorectal cancer (MSS), stable disease is reported in 8/15 patients (53%). Of note are 3 patients with durable stable disease-1 patient with renal cell carcinoma who remains on combination treatment at over 13 months and a patient with squamous cell lung cancer with prior treatment with PD1, PDL1 and CTLA-4 inhibitors who was on study treatment with CHA.7.518.1.H4 (S241P)+nivolumab for over 8 months and a patient with endometrial cancer who was on study treatment for over 10 months.

Taken together these data suggest that CHA.7.518.1.H4 (S241P) in combination with nivolumab may provide meaningful and durable clinical benefit to patients, including in tumor types typically unresponsive to immune checkpoint inhibitors such as ovarian cancer and colorectal cancer (MSS) and in pts who have had prior treatment with immune checkpoint inhibitors. Additional clinical studies are needed to confirm these findings.

These results further support our ongoing combination strategy for the triple combination study and our recently announced dual (CHA.7.518.1.H4 (S241P)+nivolumab) cohort expansion.

Additional data from the monotherapy dose escalation cohorts is included below.

The patient with primary peritoneal cancer—a type of ovarian cancer (platinum resistant, MSS) remains on study treatment with confirmed partial response ongoing for more than 14 months.

A patient with pancreatic adenocarcinoma with confirmed stable disease who was on study treatment for 7 months. This patient was refractory to all 3 prior lines of standard of care therapies. This data are encouraging considering that the antitumor activity reported with CHA.7.518.1.H4 (S241P) monotherapy is in a tumor type that is typically unresponsive to immune checkpoint inhibitors, which represents a high unmet medical need for new treatment options, and in a patient refractory to multiple lines of prior therapy.

Preliminary results from the CHA. 7.518.1.H4 (S241P) monotherapy cohort expansion which enrolled a total of 20 patients with advanced solid tumors including advanced non-small cell lung, ovarian, breast, endometrial and colorectal cancer, who have exhausted all available standard therapies. The key objective of the study was to evaluate the safety and tolerability of CHA.7.518.1.H4 (S241P) at the recommended dose for expansion of CHA.7.518.1.H4 (S241P) (20 mg/kg IV Q 4 wks). An additional exploratory objective is the preliminary evaluation of the antitumor activity of CHA.7.518.1.H4 (S241P) monotherapy at the recommended dose for expansion.

The example reports that 6/20 pts, or 30% of patients had best assessment of stable disease with 1 patient with endometrial cancer, 3 patients with NSCLC and 2 patients with ovarian cancer. Two patients with stable disease remain on study as of the data cut of Dec. 14, 2020-one patient with NSCLC, with treatment ongoing at 6 months and a patient with ovarian cancer with treatment ongoing at 20 weeks. Particularly noteworthy is the patient with NSCLC who remains on treatment and had received>3 prior lines of therapy including prior immune checkpoint inhibitors.

Two additional patients who are still on the study have not reached their first assessment. Twelve patients have stopped study treatment with the majority due to progressive disease. There were no new safety findings at this dose.

Overall, the disease control rate in the monotherapy dose escalation and expansion cohort was 47% with best responses of durable anti-tumor activity, including partial response.

Further analysis of this study data is ongoing. Corelative studies are being performed with regard to patient samples, including preliminary assessment of blood cytokines and immunophenotyping with the objective of complementing work related to CHA.7.518.1.H4 (S241P) modulation of the tumor microenvironment, particularly in indications that are typically not responsive to PD-1 to further understand

CHA.7.518.1.H4 (S241P) activity, which will inform on additional studies to be conducted in indications with preliminary encouraging signals of antitumor activity.

Initial assessments of patient's peripheral blood samples from our small monotherapy expansion cohorts study suggest that CHA.7.518.1.H4 (S241P) may enhance immune activation in cancer patients, alone or in combination with Nivolumab. Initial data from these assessments.

A total of 51 patients who were enrolled to the dose escalation and expansion arms of the monotherapy and the dose escalation arm of the combination study, 16/51 pts (31%) had prior treatment-refractory disease, out of which 9/16 (56%) had best assessment of ≥SD on this ongoing study. Also, 18/51 pts (35%) with prior treatment with immune checkpoint inhibitors, of which 13/18 (72%) had best assessment of >SD. This data suggests that treatment with CHA.7.518.1.H4 (S241P) may address patient's refractory or unresponsive to currently available treatments as well as checkpoint inhibitors. In addition, the overall signals of antitumor activity reported in several tumor types across the CHA.7.518.1.H4 (S241P) clinical program suggest that inhibition of PVRIG blockade may be key to driving immune responses in certain patient populations.

### Conclusion

CHA.7.518.1.H4 (S241P)+NIVO well tolerated with no new safety signals. Encouraging signal of antitumor activity including in pts with prior tx with ICI or prior tx-refractory disease.

In summary, the data derived from the various studies demonstrated that CHA.7.518.1.H4 (S241P), and thus PVRIG blockade, is presenting signals of anti-tumor activity in a clinical setting that can serve as a basis for our next studies. The patients enrolled in these studies had diverse tumor types and were treated with various doses of CHA.7.518.1.H4 (S241P) with or without Nivolumab, but clearly the indications in which the observed the responses and, as well as the number of highly durable stable diseases, are indications which are typically PD-L1^{low} or negative and generally have low response rates to available immune checkpoint inhibitors. Since immunotherapy has been most transformative in PD-L1 positive patients, these first signals were seen in indications with such high unmet need that generally do not respond to immune checkpoint inhibitors.

In conclusion, in the ongoing phase 1 study evaluating CHA.7.518.1.H4 (S241P) monotherapy and in combination with nivolumab have now reported encouraging signals of antitumor activity in patients that are heavily pretreated, in patients who have received prior immune checkpoint treatment, in patients refractory to prior treatment and in tumor types typically unresponsive to immune checkpoint inhibitors. These signals of antitumor activity across our studies include SD (including durable SD), confirmed partial responses, confirmed CR in diverse tumor types such as primary peritoneal cancer/OVCA, anal SCC, endometrial CA, NSCLC, pancreatic cancer, CRC (MSS), RCC, adenoid cystic cancer, cervical cancer. For the most part this should answer the question that at this stage of the study it has been demonstrated that CHA. 7.518.1.H4 (S241P) monotherapy and in combination with nivolumab has an acceptable safety and tolerability profile. Additionally, at this stage there is preliminary evidence of antitumor activity. Studies will continue to examine the hypothesis that driving robust immune responses will require combinatory blockade of PVRIG with PD-L1, TIGIT, or all three together, and each of these combinations are being further explored. Following the evaluation of correlative assessments based on data from patient samples collected in these studies' expansion cohorts the next steps may include the selection of target indications for additional studies including combinations with SOC agents.

Further studies focus on an expanded combination strategy, which includes our ongoing triplet study of CHA.7.518.1.H4 (S241P) with nivolumab and BMS's TIGIT inhibitor, as well as initiating both the doublet expansion study of CHA.7.518.1.H4 (S241P) with nivolumab and a doublet study of CHA.7.518.1.H4 (S241P) and TIGIT. Comprehensive biomarker assessment is planned to be obtained from our cohort expansion studies to gain insights into the PVRIG/PVRL2 pathway biology and shed light on specific contributors to antitumor activity and will guide expanded tumor indications. This next phase will allow generation of multiple data readouts and development of DNAM-axis based new cancer immunotherapy treatments.

Phase 1b cohort expansion combination study evaluating CHA.7.518.1.H4 (S241P) with nivolumab is planned to begin.

The Phase I/II triple combination study will evaluate the safety, tolerability and preliminary anti-tumor activity of CHA.7.518.1.H4 (S241P) in combination with BMS-986207, BMS's anti-TIGIT antibody, and nivolumab.

Progress is ongoing with the triple combination Phase 1/2 open-label study which is evaluating the safety, tolerability and preliminary anti-tumor activity of CHA.7.518.1.H4 (S241P) in combination with nivolumab and Bristol Myers Squibb's anti-TIGIT antibody in selected tumor types, namely ovarian cancer, endometrial cancer as well as a biomarker-driven arm of tumor types with high expression of PVRL2.

### Example 6: CHA.7.518.1.H4 (S241P) Phase 1 Clinical Trial

This example provides data from the Phase 1 dose escalation and expansion study of CHA.7.518.1.H4 (S241P) as a monotherapy, and in a dose escalation combination study with Opdivo® (nivolumab). CHA.7.518.1.H4 (S241P) is a first-in-class investigational therapeutic antibody targeting PVRIG, a novel immune checkpoint.

The data generated to date across the CHA.7.518.1.H4 (S241P) clinical program suggest that PVRIG may be an important immune checkpoint in patients who are unresponsive or refractory to currently available immunotherapies. Results from the CHA.7.518.1.H4 (S241P) plus Opdivo® combination dose escalation, which now include a confirmed complete response in a patient with prior progression on Opdivo® and a previously reported patient with a durable confirmed partial response for almost a year. Combined with a disease control rate of 66.7% and ongoing durable signals of activity beyond or approaching one year in multiple patients and across indications, these results leave us increasingly confident that dual blockade of PVRIG and PD-1 may be a key to driving durable immune responses in certain patient populations. Based on these results, further evaluation of this dual combination regimen in patients with ovarian, breast, endometrial and microsatellite-stable colorectal cancers with the initiation of the CHA.7.518.1.H4 (S241P) and Opdivo® cohort expansion study is ongoing.

The monotherapy cohort expansion study was an important milestone in the CHA.7.518.1.H4 (S241P) monotherapy evaluation. This data, together with data from the previously reported dose escalation study, which includes a confirmed partial response with treatment ongoing for over one year, demonstrate durable signals of antitumor activity in tumor types typically unresponsive to immune checkpoint inhibitors, including patients with prior progression on these treatments. These data, along with future data from ongoing correlative assessments of biological samples from patients will inform the clinical approach and next steps for the broad combination strategy, which includes dual and triple blockade regimens of CHA.7.518.1.H4 (S241P) with TIGIT and PD-1. These studies will allow for comprehensive evaluation of the synergistic blockade of the DNAM axis with PD-1 and continued progress in potentially expanding the reach of immunotherapy.

This example provides data highlights from the Phase 1 dose escalation studies. CHA.7.518.1.H4 (S241P) and Opdivo® combination dose escalation arm:
In 15 patients with a median of five prior anticancer therapies (range of 2-10), CHA.7.518.1.H4 (S241P) in combination with Opdivo® was well-tolerated with no reported dose-limiting toxicities up to the fifth and final dose cohort of CHA.7.518.1.H4 (S241P) 20 mg/kg and Opdivo® 480 mg, both IV Q4 weeks.
The disease control rate (DCR) was 66.7% (N=10) with best responses of complete response (CR) 6.7% (N=1), partial response (PR) 6.7% (N=1) and stable disease (SD) 53.3% (N=8).
A patient with anal squamous cell carcinoma with confirmed SD and now with confirmed CR and remains on treatment at 79 weeks. This patient progressed on Opdivo® prior to enrolling in our study.
A patient with colorectal cancer (MSS) with durable confirmed partial response and remained on study treatment at 44 weeks.
Durable responses of confirmed SD of six months or more in three patients. One patient with renal cell carcinoma remains on treatment at 58 weeks, and one patient with non-small cell lung cancer (NSCLC) (squamous) who failed prior treatment with immune checkpoint inhibitors remained on treatment at 36 weeks, and one patient with endometrial cancer remained on treatment at 46 weeks.

CHA.7.518.1.H4 (S241P) monotherapy arm dose escalation data showed:
The patient with primary peritoneal cancer (platinum resistant, MSS) with durable confirmed partial response remained on study treatment at 62 weeks.
The patient with pancreatic cancer, refractory to all three prior lines of standard of care (SOC) therapy with durable confirmed SD was on study treatment for 31 weeks.

Data from the monotherapy expansion cohort showed:
20 patients enrolled in biomarker and data informed indications; four patients of each: endometrial cancer, NSCLC, ovarian cancer, breast cancer and colorectal cancer.
Six of the 20 patients (30%) had best responses of SD, one patient with endometrial cancer, three patients with NSCLC and two patients with ovarian cancer.
Two patients with SD remain on treatment as of the data cutoff date; one patient with NSCLC who had >3 prior lines of SOC therapy; including prior treatment with immune checkpoint inhibitors with treatment ongoing at 26 weeks, and one patient with ovarian cancer with treatment ongoing at 20 weeks
Two additional patients remain on treatment as of the data cut date
No new safety findings were observed.

Early data shows increased induction of activated DC markers, potentially following efficient T-DC interaction, in serum of two patients responding to CHA.7.518.1.H4 (S241P)+nivolumab. Additional clinical data and initial correlative assessments of biological samples from patients are ongoing.

### Example 7: A Phase 1 Dose Escalation and Expansion Study of the Safety and Tolerability of CHA.7.518.1.H4 (S241P) with or without Nivolumab in Patients with Advanced Solid Tumors

### Background and Objectives

There is a high unmet medical need for the treatment of patients with advanced tumors who are unresponsive to or relapse following treatment with checkpoint inhibitors. Novel immune checkpoint inhibitors with antitumor activity as monotherapy or in combination with standard of care agents are urgently needed. CHA.7.518.1.H4 (S241P) is a novel first-in-class monoclonal antibody that binds with high affinity to PVRIG (poliovirus receptor related immunoglobulin domain containing) blocking its interaction with its ligand, PVRL2. Inhibition of poliovirus receptor related immunoglobulin domain containing (PVRIG) leads to enhanced activation of T and NK cells, and results in tumor growth inhibition in mouse tumor models¹. Both PVRIG and PVRL2 are part of the DNAM axis. PVRL2 is highly expressed in many solid tumors (eg OVCA, endometrial, breast, NSCLC) and commonly expressed in both PD-L1 positive and negative tumors. PVRIG may serve as a dominant inhibitory checkpoint in PD-L1 negative tumors. There is a potential to address patient populations non-responsive to PD 1 therapies and improve outcomes in PD 1 responsive patient populations

### Methods

NCT03667716 is an ongoing open-label first-in-human phase 1 study in pts with advanced solid tumors. Overall study design: During dose escalation the study utilized a hybrid single subject dose cohort and 3+3 study design. Adverse events graded per CTCAE v4.03, investigator assessment of responses per RECIST v1.1.

### Primary Objectives:

Incidence of adverse events and dose-limiting toxicities (21/28-day DLT window) PK profiles of CHA.7.518.1.H4 (S241P) with and without nivolumab

Identify the maximum tolerated dose and/or the recommended dose for expansion of CHA.7.518.1.H4 (S241P) monotherapy.

### Secondary Objectives:

Characterize the immunogenicity and preliminary antitumor activity of CHA.7.518.1.H4 (S241P) in combination with nivolumab.

### Key Eligibility Criteria:

Inclusion criteria: Age≥18 yrs, histologically or cytologically confirmed, locally advanced or metastatic solid malignancy and has exhausted or not eligible for standard therapy, ECOG 0-1, prior anti-PD-1, anti-PD-L1, anti-CTLA-4, OX-40, CD137.

Exclusion criteria: Active autoimmune disease requiring systemic therapy in the last 2 years prior to the first dose of CHA.7.518.1.H4 (S241P), symptomatic interstitial lung disease or inflammatory pneumonitis, untreated or symptomatic CNS metastases

### Enrollment Summary and Conclusions:

Enrollment is complete in the CHA. 7.518.1.H4 (S241P) monotherapy expansion cohort consisting of these select tumor types ovarian, endometrial, breast, lung, CRC (microsatellite stable). Confirmed PR with CHA.7.518.1.H4 (S241P) monotherapy 20 mg/kg IV Q4 wks-primary peritoneal cancer (platinum resistant, MSS).

Enrollment complete into the 5th dose level of CHA.7.518.1.H4 (S241P) (20 mg/kg IV Q4 wks) in combination with nivolumab 480 mg IV Q4 wks. At 1^{st} dose level (CHA.7.518.1.H4 (S241P) 0.3 mg/kg+nivolumab 360 mg both IV Q3 wks), a confirmed partial response in CRC (MSS). Confirmed stable disease in endometrial cancer and other solid tumors at higher combination dose cohorts.

Previous data indicated there were no DLTs in CHA.7.518.1.H4 (S241P) monotherapy doses tested (0.01-20 mg/kg) or in the combination doses ranges tested with nivolumab (360/480 mg IV Q3/4 wks) plus CHA.7.518.1.H4 (S241P) (0.3-10 mg/kg; IV Q3/4 wks) 2.

### Example 8: CHA.7.518.1.H4 (S241P) with or without Nivolumab: Results of an Ongoing Phase 1 Study of Safety, Tolerability and Preliminary Antitumor Activity in Patients with Advanced Solid Malignancies

### Background and Objectives

PVRIG (poliovirus receptor related immunoglobulin domain containing) is a coinhibitory receptor expressed on T and NK cells and is part of an immune checkpoint pathway which is parallel to the TIGIT pathway and part of the DNAM-1 Axis

CHA.7.518.1.H4 (S241P) is a novel first in class humanized IgG4 monoclonal antibody that binds with high affinity to PVRIG, blocking its interaction with its natural ligand PVRL2 expressed in tumor cells and antigen presenting cells and increases T and NK cell activation

In pre-clinical models, inhibition of PVRIG leads to enhanced activation of T and NK cells, and results in tumor growth inhibition, in combination but also independently of anti-PD-1 treatment.

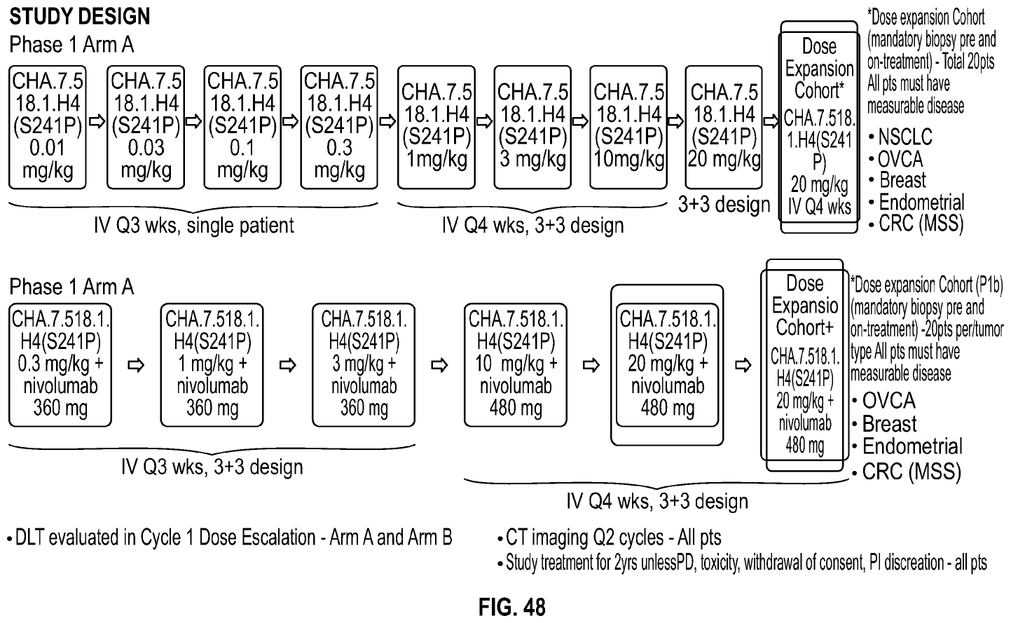

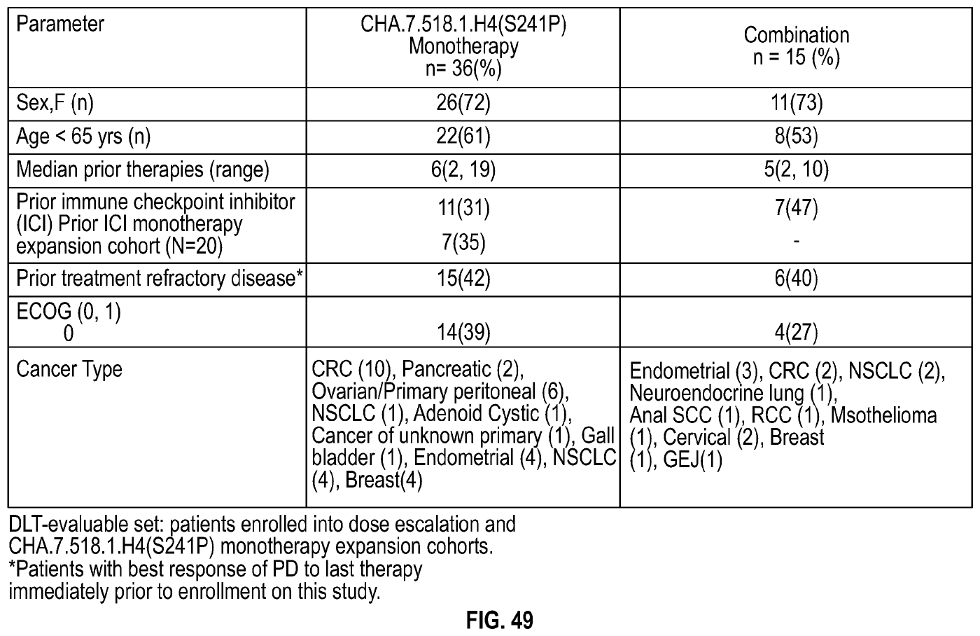

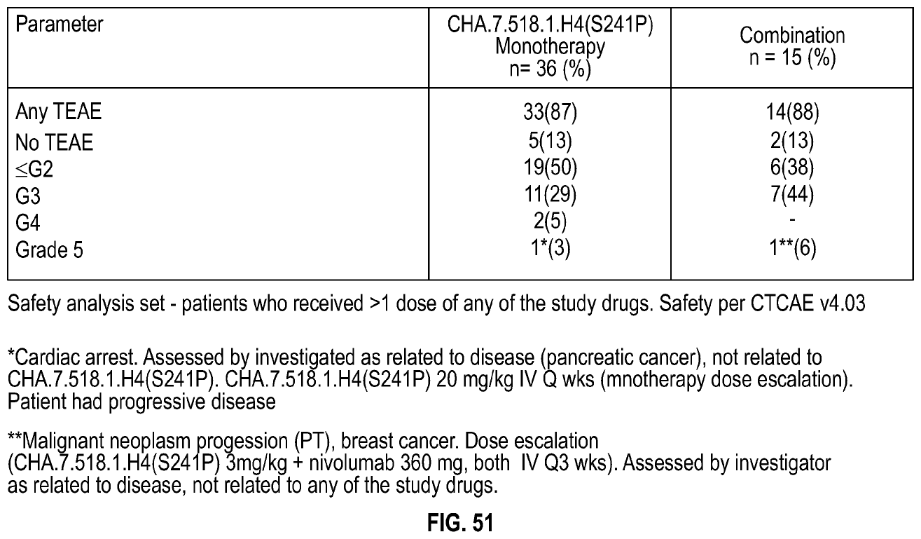

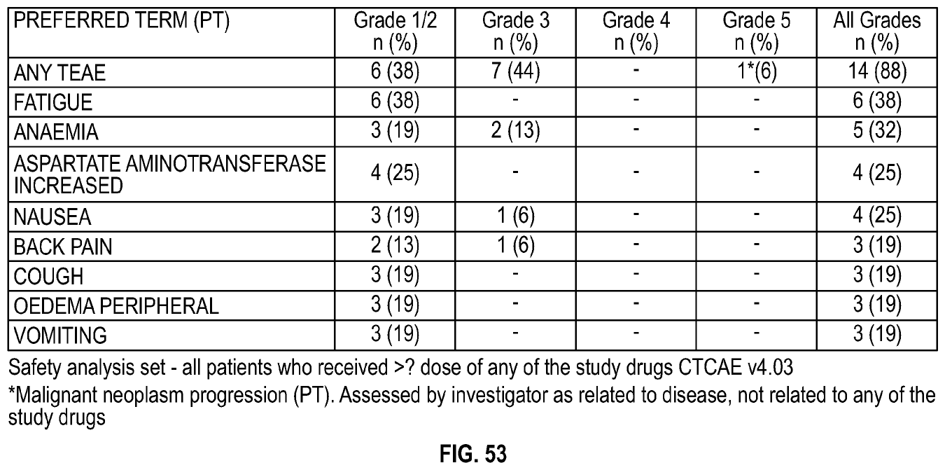

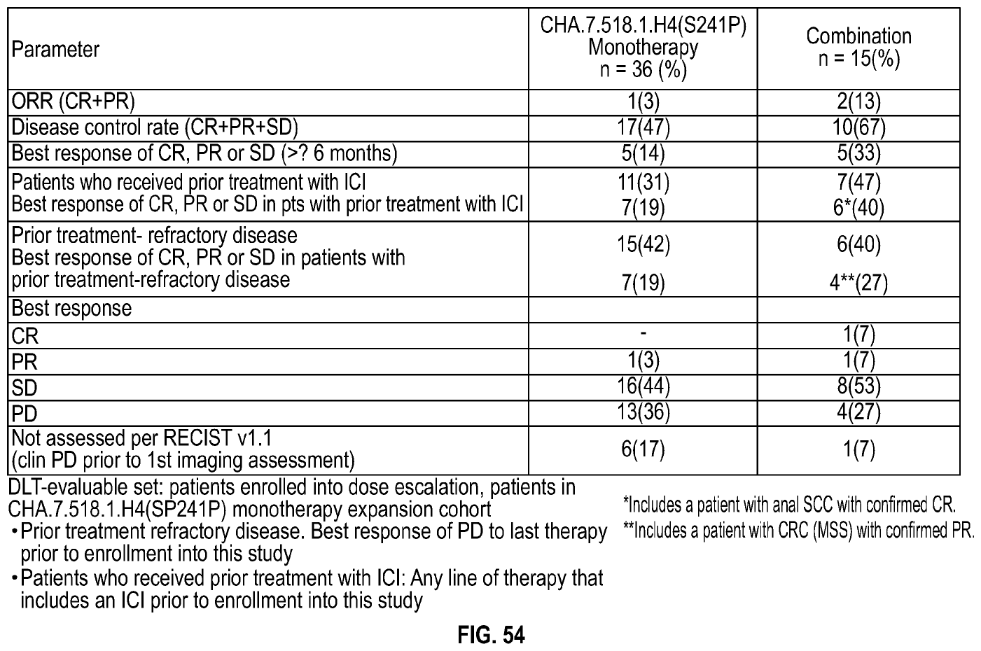

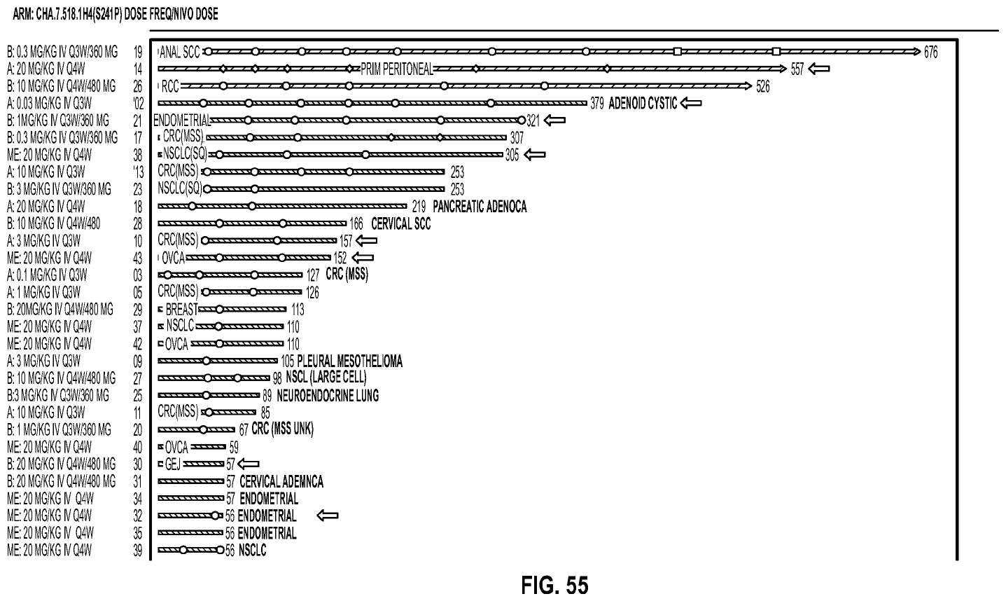

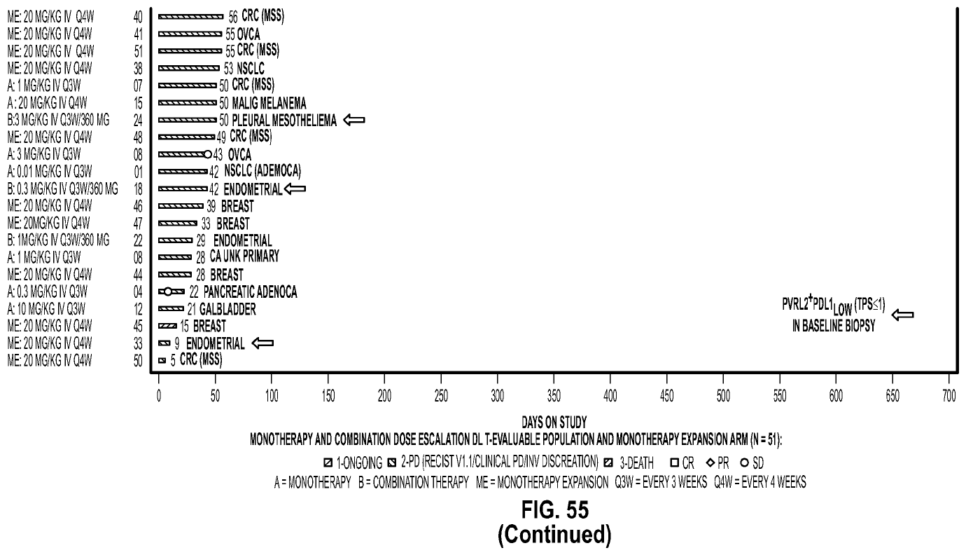

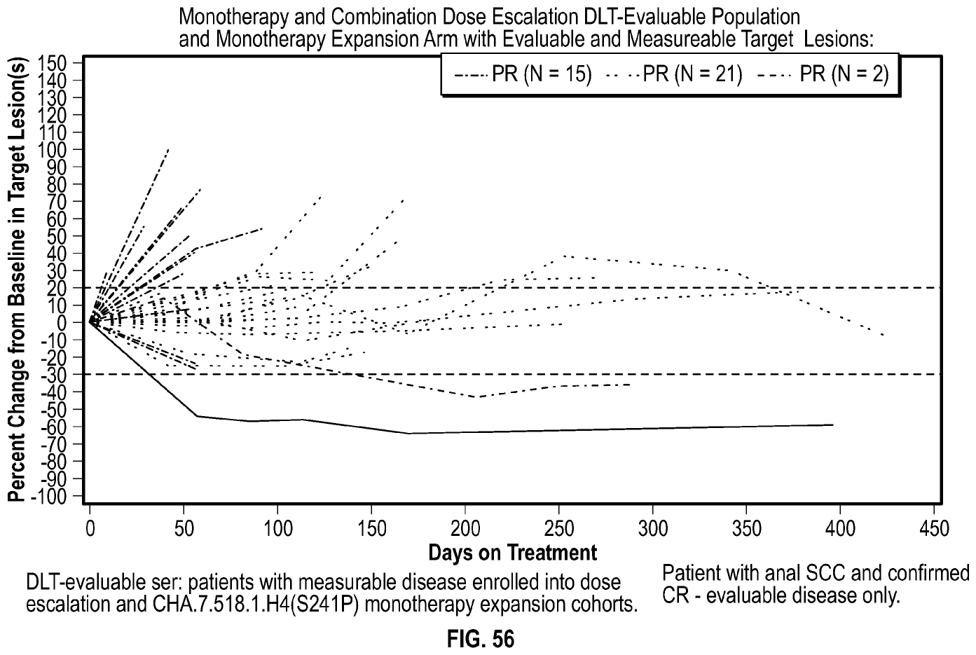

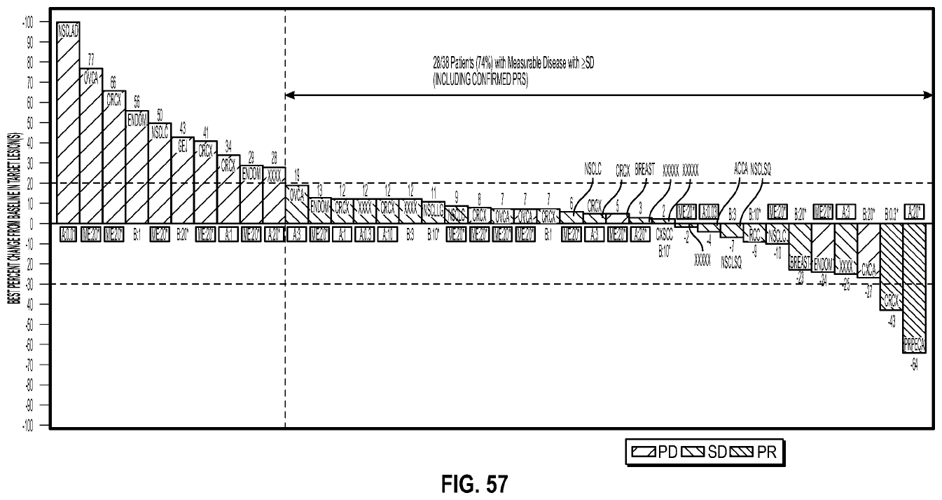

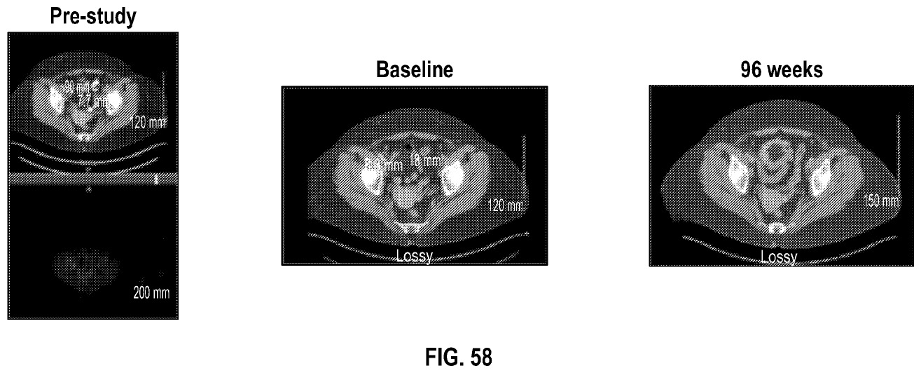

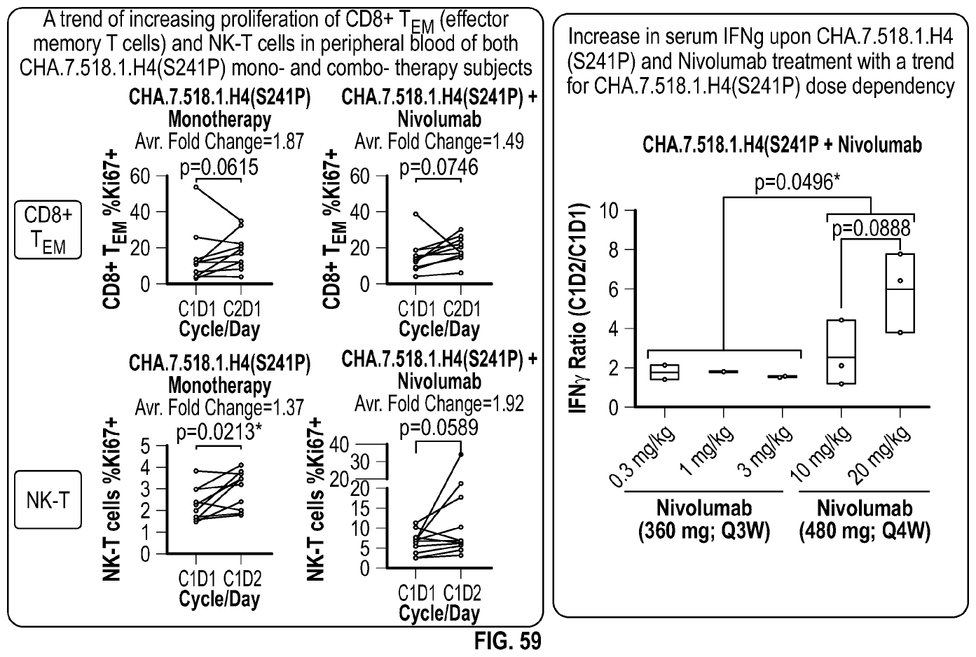

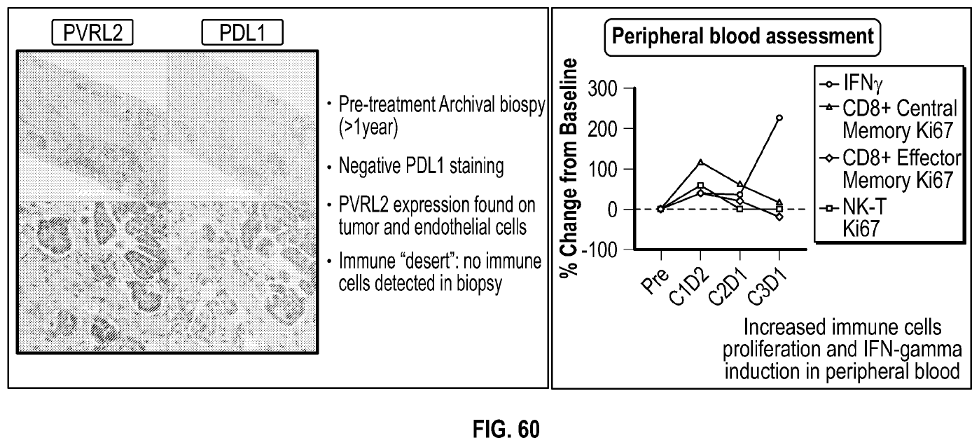

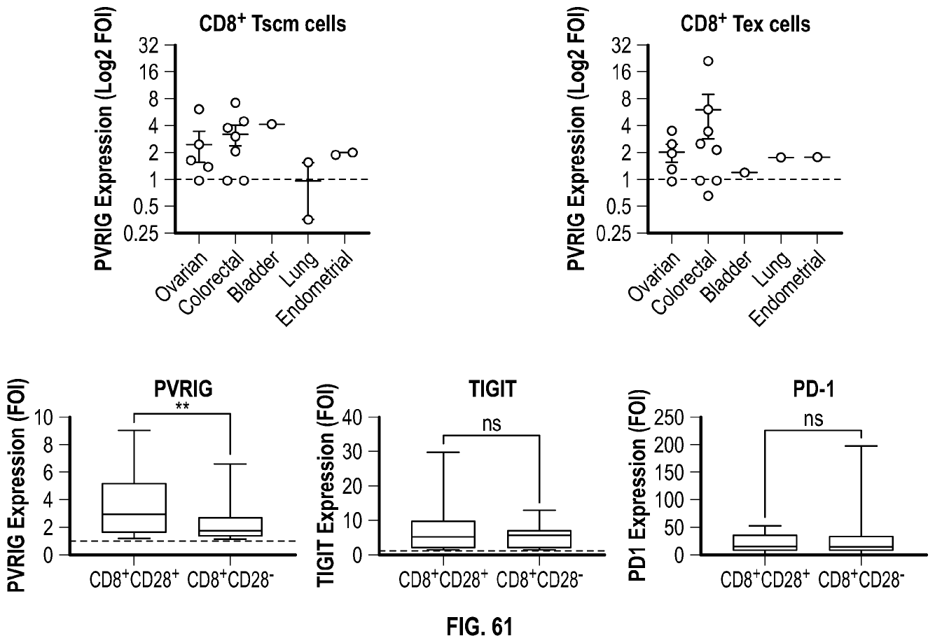

This example and the accompanying FIGS. 48-63 provide updated data on the ongoing phase 1 study.

### Key Inclusion Criteria:

Age≥18 yrs
Histologically confirmed locally advanced or metastatic solid malignancy
Has exhausted all available standard treatment or is not a candidate for available standard therapy
ECOG 0-1
Measurable disease not required in dose escalation

### Key Exclusion Criteria:

Active autoimmune disease requiring systemic treatment
History of inflammatory pneumonitis or interstitial lung disease
History of immune-related toxicities on prior immunotherapy treatment leading to discontinuation

### Key Primary Objectives:

Safety and tolerability of profile of CHA.7.518.1.H4 (S241P)+nivolumab
To identify the MTD and/or recommended dose for expansion of CHA.7.518.1.H4 (S241P)+nivolumab
PK profile of CHA.7.518.1.H4 (S241P)+nivolumab

### Secondary Objectives:

Immunogenicity of CHA.7.518.1.H4 (S241P)+nivolumab
Antitumor activity of CHA.7.518.1.H4 (S241P)+nivolumab (Phase 1b only) Exploratory Objectives:
Antitumor activity of CHA.7.518.1.H4 (S241P)+nivolumab (Phase 1a only)
CHA.7.518.1.H4 (S241P)-mediated pharmacodynamic effect in blood (CHA.7.518.1.H4 (S241P)+nivolumab), immune-related changes (cytokines, immunophenotyping)

### Conclusions

CHA.7.518.1.H4 (S241P)+nivolumab has an acceptable safety profile and was well tolerated with no DLTs at the maximum administered doses evaluated (CHA.7.518.1.H4 (S241P) 20 mg/kg IV Q4 wks monotherapy, CHA.7.518.1.H4 (S241P) 20 mg/kg+nivolumab 480 mg both IV Q4 wks).

CHA.7.518.1.H4 (S241P)+nivolumab demonstrated durable antitumor activity in extensively pretreated patient population.
CR, PR or SD≥6 months in 10/51 (19%) patients
Best response of CR, PR or SD in 11/21 (52%) patients with prior treatment refractory disease
Best response of CR, PR or SD in 13/18 (72%) patients with prior treatment with immune checkpoint inhibitor

### Summary of Responding Patients:

Confirmed CR—ongoing study treatment 22 months in a patient with anal SCC (non-measurable disease at study entry) and prior progression on nivolumab
Confirmed PR (ongoing study treatment 18 months in a patient with primary peritoneal cancer (platinum resistant, MSS)
Confirmed PR in a patient with CRC (MSS) (was on study treatment for 44 wks)

### Evidence of Immune Activation

Demonstrated antitumor activity in patient with PVRL2+PD-L1^{low} tumor
A trend of increasing peripheral immune cell proliferation and in serum IFN-γ, suggesting immune activation by CHA.7.518.1.H4 (S241P)

The results of this phase 1 trial support further clinical development of CHA.7.518.1.H4 (S241P).

### Example 9: PVRIG Monotherapy and Combination Therapy in Anti-Cancer Immunity

This example provides the first demonstration of immune activation in the tumor microenvironment (TME) after PVRIG blockade with CHA.7.518.1.H4 (S241P) monotherapy and in combination with nivolumab, built upon prior data showing peripheral induction of activated dendritic cell markers in patients who responded to treatment of CHA.7.518.1.H4 (S241P) in combination with nivolumab. These data support the distinct role of PVRIG in the DNAM-1 axis which may be critical for driving immune activation in indications unlikely to respond to PD-1 blockade.

Of particular significance is the observed increase in clonal expansion of T cells, immune infiltration and immune activation in an CRC (MSS) patient with a confirmed response after CHA.7.518.1.H4 (S241P) and nivolumab combination therapy given this is an indication that is unlikely to respond to PD-(L) 1 blockade. These results build upon and are consistent with the translational results from the periphery which demonstrate robust immune activation in patients responding to CHA.7.518.1.H4 (S241P) and its combinations and provide evidence that this peripheral immune activation is translating also to immune modulation at location critical for efficacy, the tumor site.

These data, combined with previous findings which showed that PVRIG and its ligand PVRL2 have distinct expression patterns across multiple key immune cells, provide evidence that PVRIG plays a distinct role within the DNAM-1 axis.

Key new findings for PVRIG monotherapy and combination therapy including:
Increased TME immune activation and TCR clonality was observed in a patient with PVRL2high, PD-L1^{low} MSS-CRC with a partial response following treatment of CHA.7.518.1.H4 (S241P) in combination with nivolumab;
CHA.7.518.1.H4 (S241P) induced immune activation in the TME of a non-responding patient with ovarian cancer, showing increased CD8 distribution and a shift from stromal markers toward immune activation;
CHA.7.518.1.H4 (S241P) in combination with nivolumab induced markers of activated DCs in the serum of 2 responding patients.

### Results

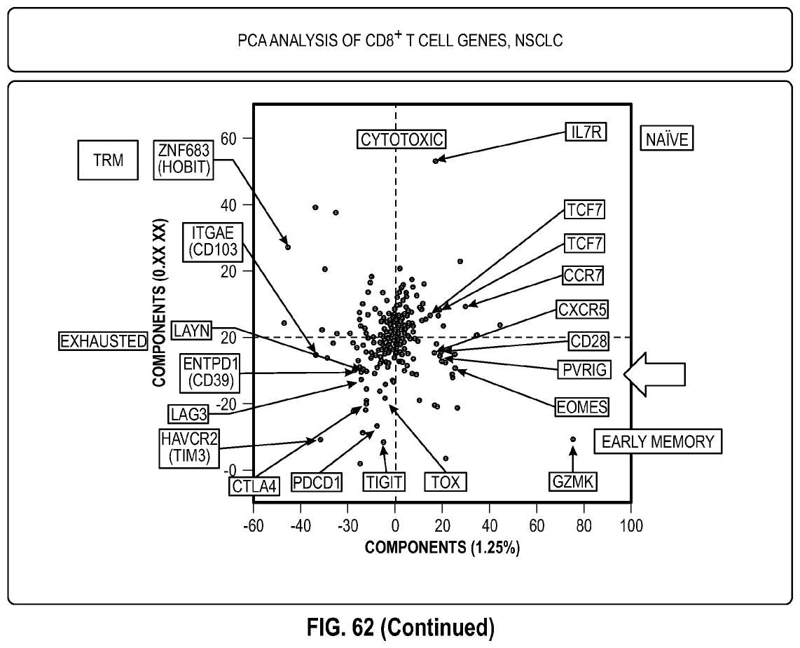

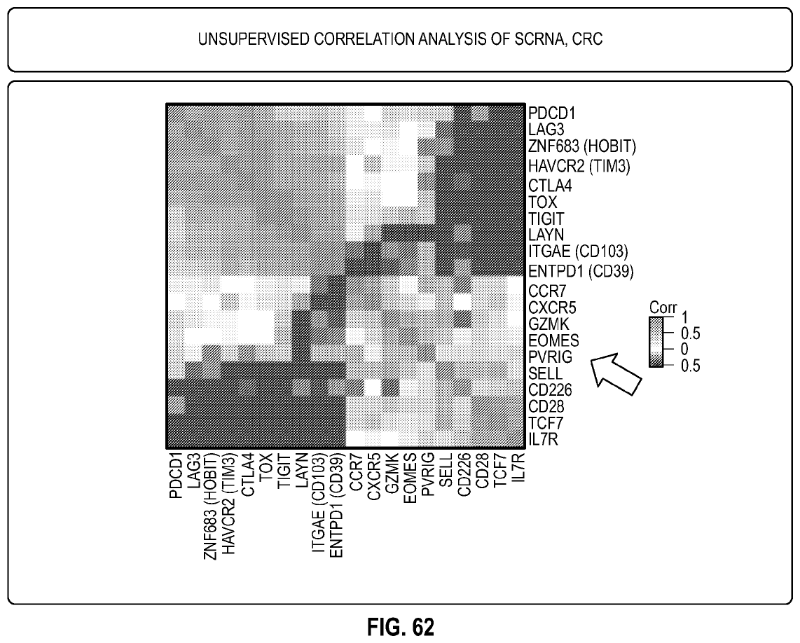

PVRIG expression was found in early differentiated T stem cell-like memory cell (Tscm) has higher expression on early differentiated CD8+ T cells. FIG. 61 illustrates flow cytometry results of PVRIG expression in CD8+Tscm cells and CD8+Tex cells in patients with various cancers. Analysis results of gene expression in T cells including CD8+ T cells from cancer patients using PCA analysis and unsupervised correlation analysis of scRNA are shown in FIG. 62. The analysis results demonstrate unique clustering of PVRIG expression with genes expressed in early differentiated/Tscm cells such as CXCR5, CD28, EOMES, GZMK. These results suggest the potential of PVRIG blockade for optimal Tscm activation, expansion and generation of effector T cells.

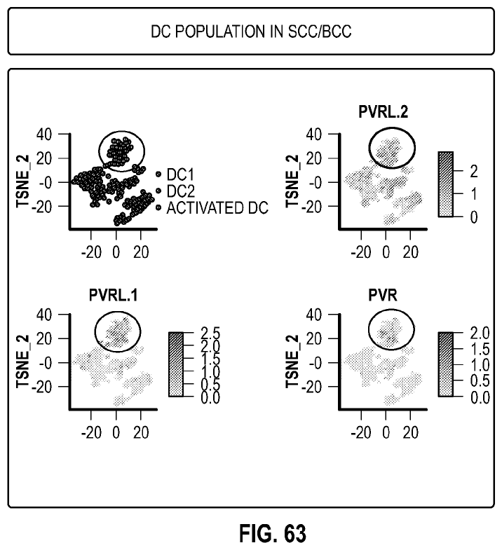

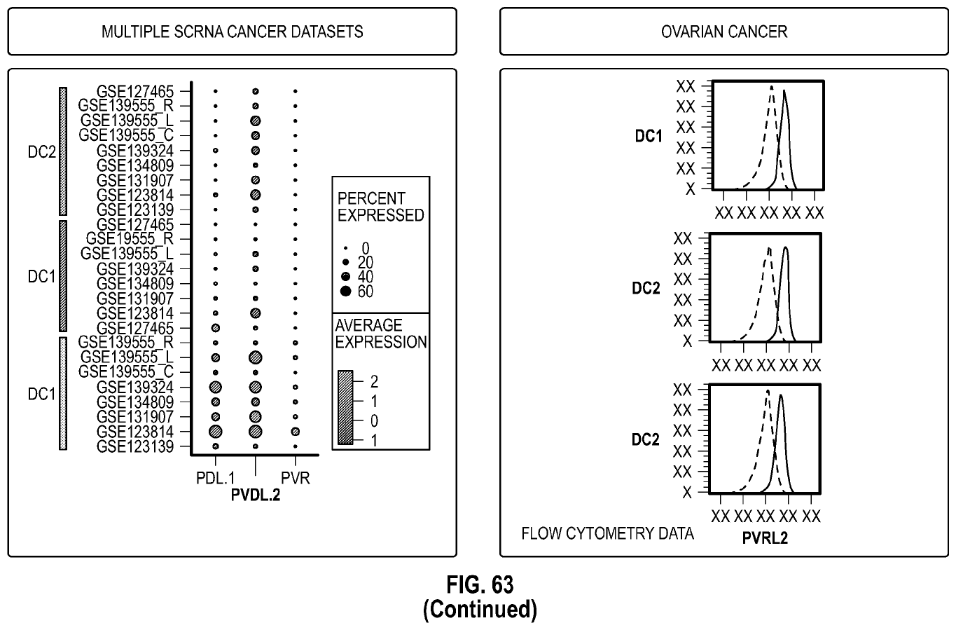

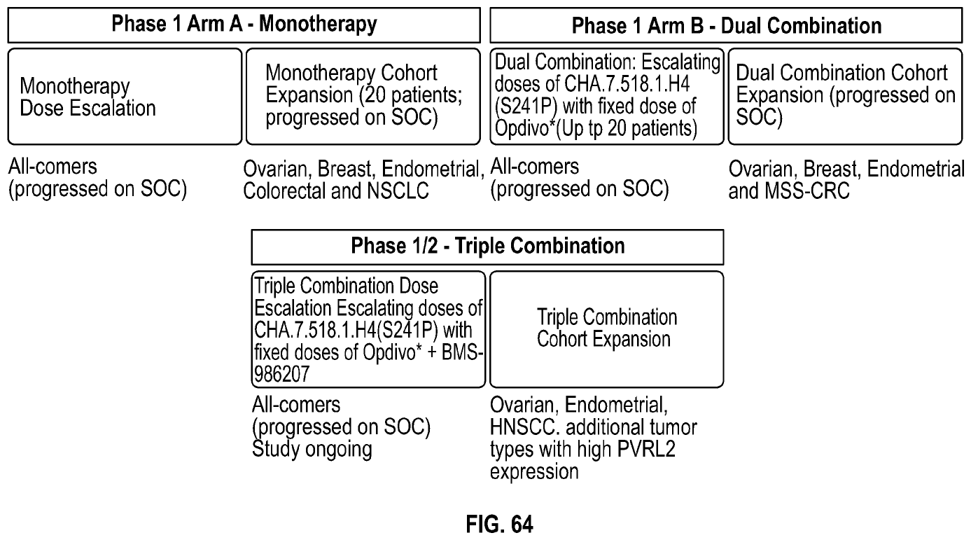

PVRL2 was also found to have a dominant expression on dendritic cells (DCs). FIG. 63 provides data on PVRL2 expression in dendritic cells. PVRIG blockade may enhance interaction and activation of Tscm by DCs in PD-L1^{low} non-inflamed indications.

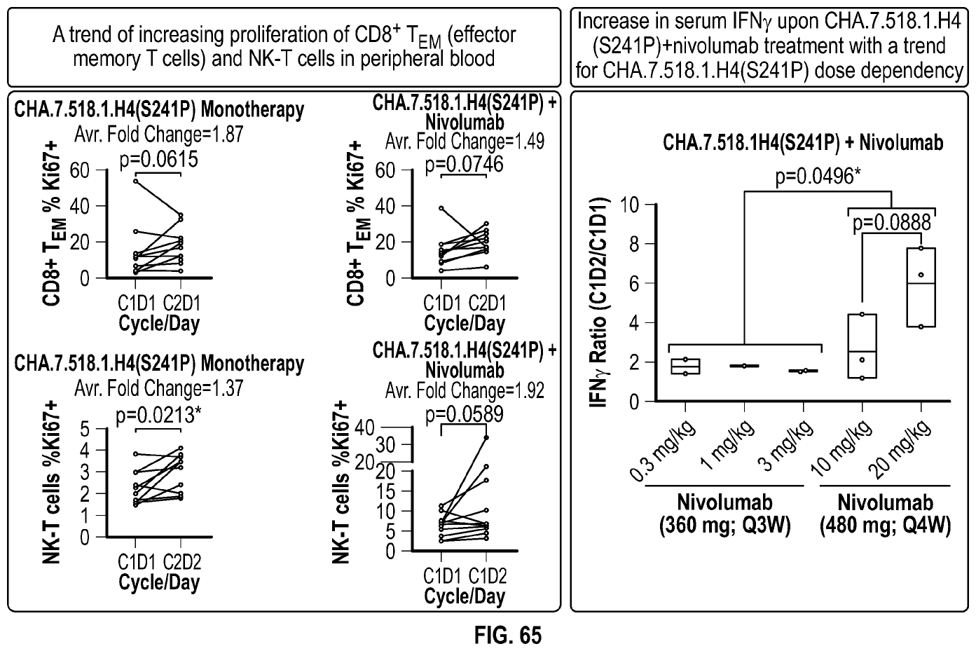

FIG. 65 provides data demonstrating increased immune activation in peripheral blood of patients treated with CHA.7.518.1.H4 (S241P) monotherapy and nivolumab combination therapy. As shown by the results of DSP NanoString measurement, a trend of increasing proliferation of CD8+TEM (effector memory T cells) and NK-T cells in peripheral blood of patients with CHA.7.518.1.H4 (S241P) monotherapy and nivolumab combination therapy was observed. Also observed was an increase in serum IFNγ upon CHA.7.518.1.H4 (S241P)+nivolumab treatment with a trend for CHA.7.518.1.H4 (S241P) dose dependency.

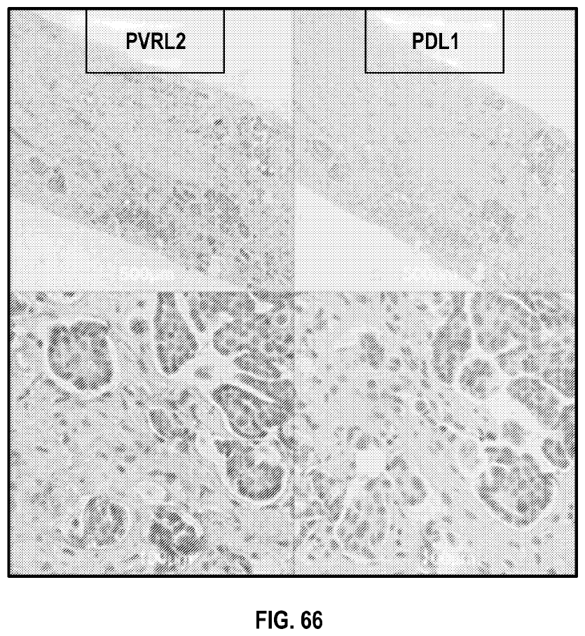

FIG. 66 shows histological data of patient who received 3 prior lines of anti-cancer therapies including:
pre-treatment archival biopsy (>1 year), negative PD-L1 staining,
PVRL2 expression found on tumor and endothelial cells, and
Immune “desert”: no immune cells detected in biopsy

Following CHA.7.518.1.H4 (S241P) monotherapy with the patient, partial response (PR) in patient with primary peritoneal PD-L1^{neg} cancer was observed. Partial response in patient with non-inflamed TME demonstrated immune activation in peripheral blood following CHA.7.518.1.H4 (S241P) monotherapy.

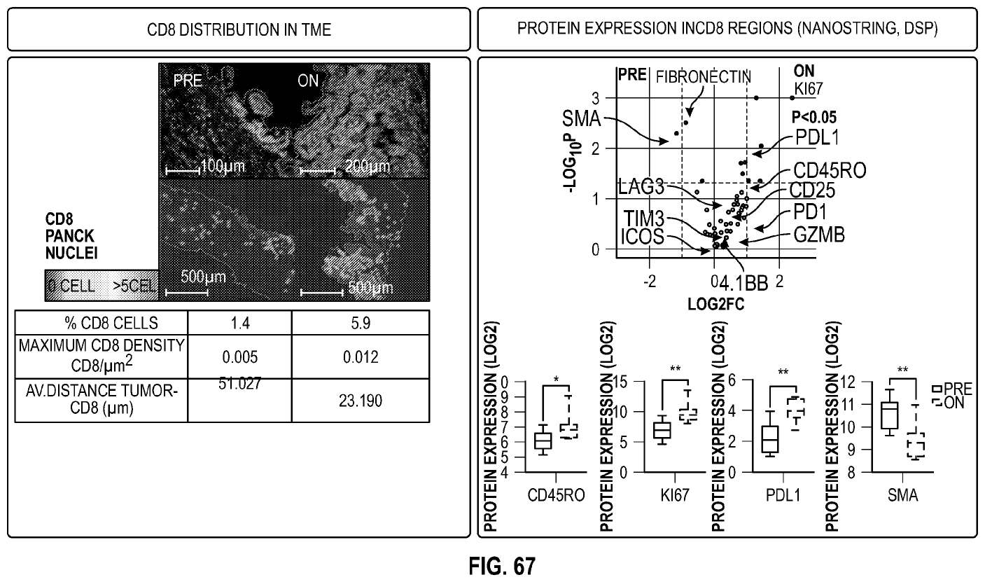

FIG. 67 provides clinical data showing that monotherapy with CHA.7.518.1.H4 (S241P) induced immune activation in TME in ovarian cancer patient (radiologically PD). CD8 expression was visualized and measured in patient sample before and after the monotherapy. Gene expression was also measured across of a range of genes, for example, CD45RO, KI67, PDL1, and SMA, in CD8 enriched regions in patient sample using NanoString, DSP. Overall, patient with PVRL2high (H-score=250) PDL1 (8% TPS) ovarian cancer demonstrated shift from stromal markers towards immune activation in TME following CHA.7.518.1.H4 (S241P) monotherapy.

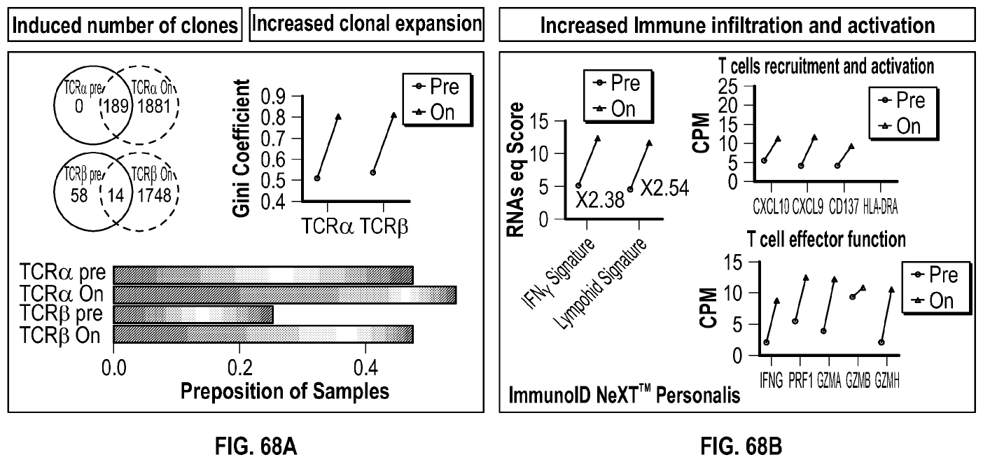

As shown in FIG. 68, following combination therapy with CHA.7.518.1.H4 (S241P) and nivolumab, patients with CRC (MSS) demonstrated increased TME immune activation and TCR clonality. Patients tested received 4 prior lines of chemotherapies. Patients with PVRL2+ (H-score=25) PDL 1^{low} (1% TPS) CRC (MSS) showed increase in TCR numbers and clonality and T cell infiltration and activation in TME following CHA.7.518.1.H4 (S241P)+nivolumab combination therapy.

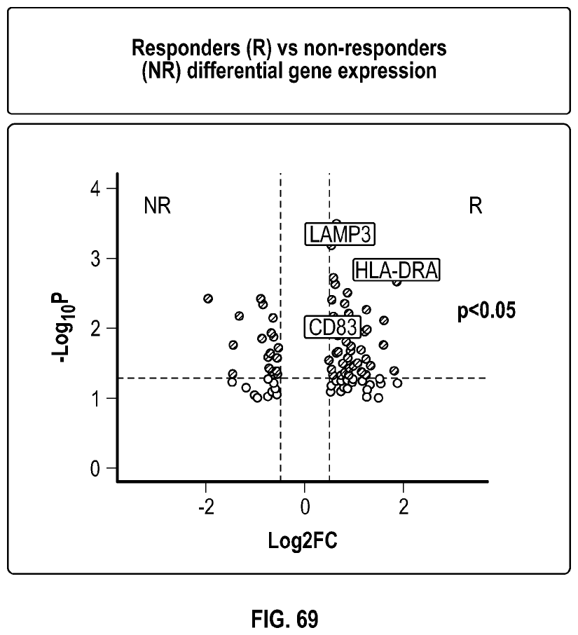

Furthermore, FIG. 69 shows that induced expression of gene markers, such as LAMP3, HLA-DRA, and CD83, in activated DCs in serum of patients responding to the CHA.7.518.1.H4 (S241P)+nivolumab combination therapy as compared to non-responding patients.

### Summary

PVRIG, a novel checkpoint in the DNAM-1 axis, is expressed on stem-like and exhausted T cells but has a unique dominant expression on early differentiated Tscm.

PVRL2 and PVR are expressed in PD-L1^{low} and PD-L1high tumor types.

PVRL2 is dominantly expressed across DC types and in Tertiary Lymphoid Structures.

PVRIG blockade may enhance Tscm activation by DCs, resulting in their increased expansion and differentiation. A potential mechanism which could lead to increased T cell expansion and infiltration into less ‘inflamed’ tumors.

Preliminary data showed that CHA.7.518.1.H4 (S241P) (anti-PVRIG) monotherapy induced immune activation in periphery and signs of anti-tumor activity in patients with PVRL2+PD-L1^{low} tumors.

Increased infiltration and activation of T cells in TME of patients treated with CHA.7.518.1.H4 (S241P) monotherapy was observed.

Dual (PVRIG & PD-1) blockade resulted in increased T cell clonality and activation and increased induction of activated DC markers in serum of 2 patients responding to this therapy.

### Example 10: Novel DNAM-1 Axis Member, PVRIG, is Potentially a Dominant Checkpoint Involved in Stem-Like Memory T Cells-Dendritic Cell Interaction

### Background

T cell accumulation in tumors is a prerequisite for response to cancer immunotherapy. Recent studies highlighted the importance of an early-memory (stem-like) T cell sub-population, that can self-renew and differentiate into effector cells, and of DCs, which are essential for T-cell expansion following checkpoint blockade. PVRIG is a novel inhibitory receptor that competes with the co-activating receptor DNAM-1, for the binding of a shared ligand, PVRL2.

T-cell infiltration and persistence in tumors is a prerequisite for response to cancer immunotherapy. Recent studies highlighted the importance of an early-memory (stem-like) T-cell sub-population, that can self-renew and differentiate into effector cells, and of dendritic cells (DCs), which are essential for T-cell priming and expansion following checkpoint blockade [1, 2]. PVRIG is a novel inhibitory receptor that competes with the co-activating receptor DNAM-1, for the binding of a shared ligand, PVRL2. PVRIG expression is induced on T and NK tumor infiltrating cells, whereas PVRL2 is expressed on tumor, endothelial and myeloid cells in the tumor micro-environment (TME) [3]. The expression of PVRIG and PVRL2 across TME immune subpopulations has been investigated.

### Methods

Publicly available TME scRNA sequencing datasets were analyzed for the expression of PVRIG and PVRL2 across immune subsets. Unsupervised principal component analysis with genes used as entries, and hierarchical co-expression pattern among genes known to be expressed on naïve, memory, and exhausted CD8+ T-cells was performed. Observations were validated by evaluating PVRIG and PVRL2 expression by flow-cytometry and immunohistochemistry across a variety of tumor indications. Proximity Extension Assay (PEA, Olink) was conducted using serums collected at several time-points from CHA.7.518.1.H4 (S241P) (anti-PVRIG antibody) and nivolumab treated patients in a Phase-1 study (NCT03667716).

### Results

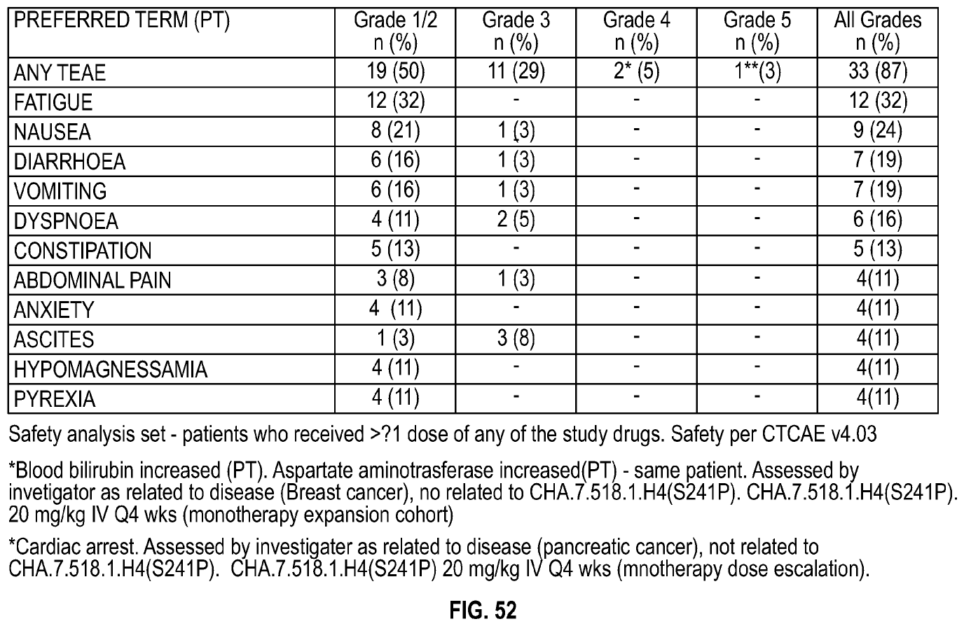

Across scRNA datasets, PVRIG, like TIGIT and PD-1, was expressed by both stem-like (TCF1+PD1+) and exhausted (TIM3+CD39+) CD8+ T-cells. High resolution unsupervised scRNA gene co-expression analysis revealed that while TIGIT is strongly correlated with PD-1, CTLA-4, and other markers of exhausted T-cells, PVRIG uniquely clusters with markers of early memory T-cells. Accordingly, PVRIG protein expression was increased on CD28+early-memory T-cells across indications (FIG. 52).

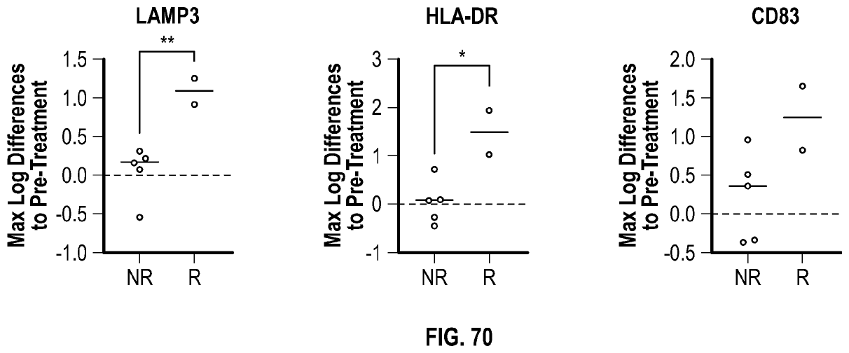

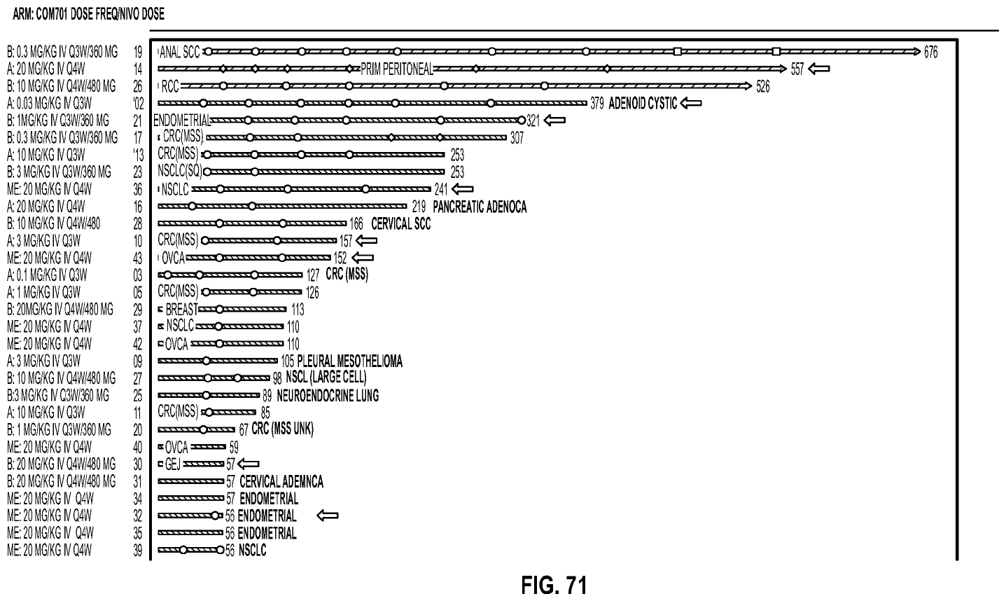

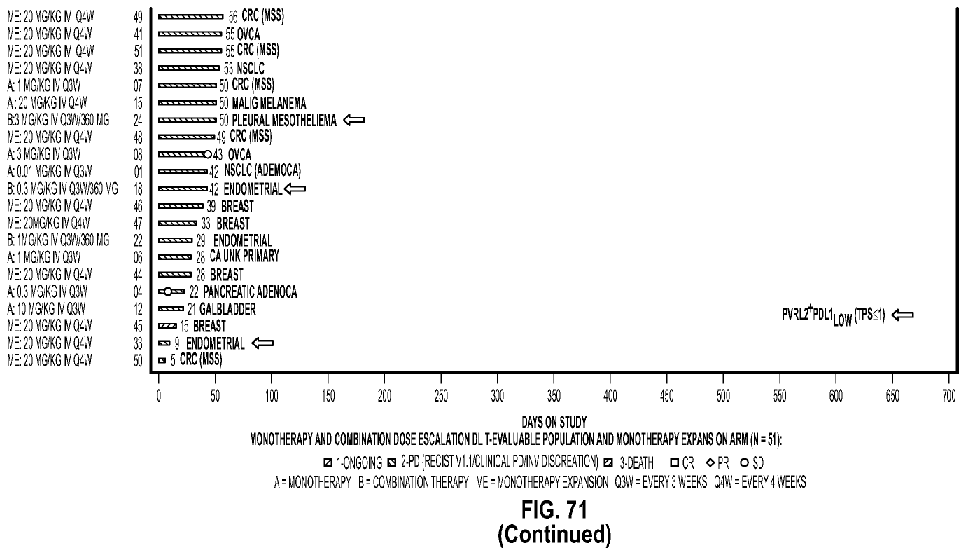

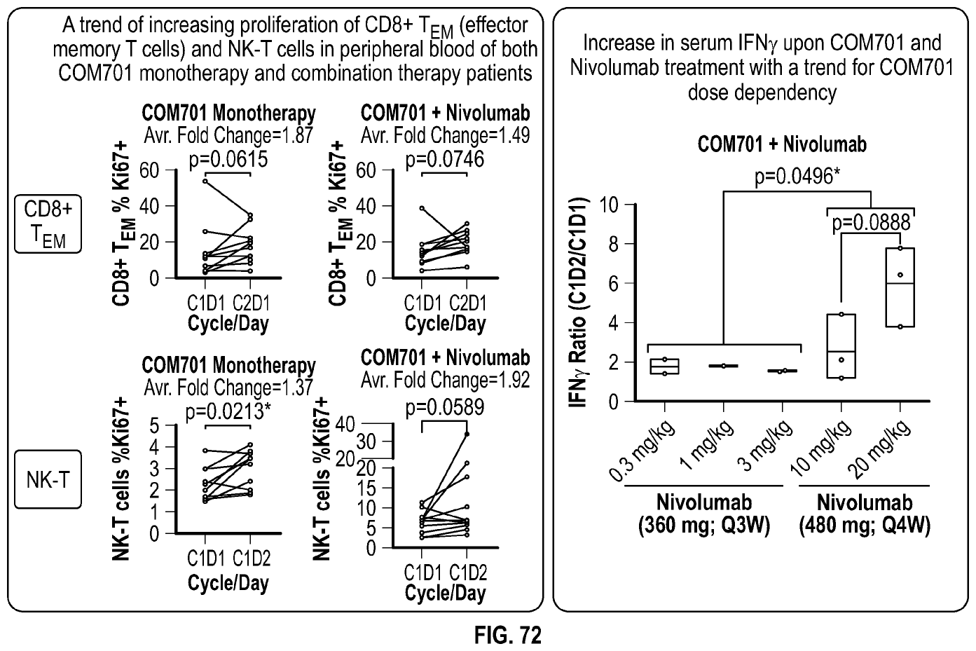

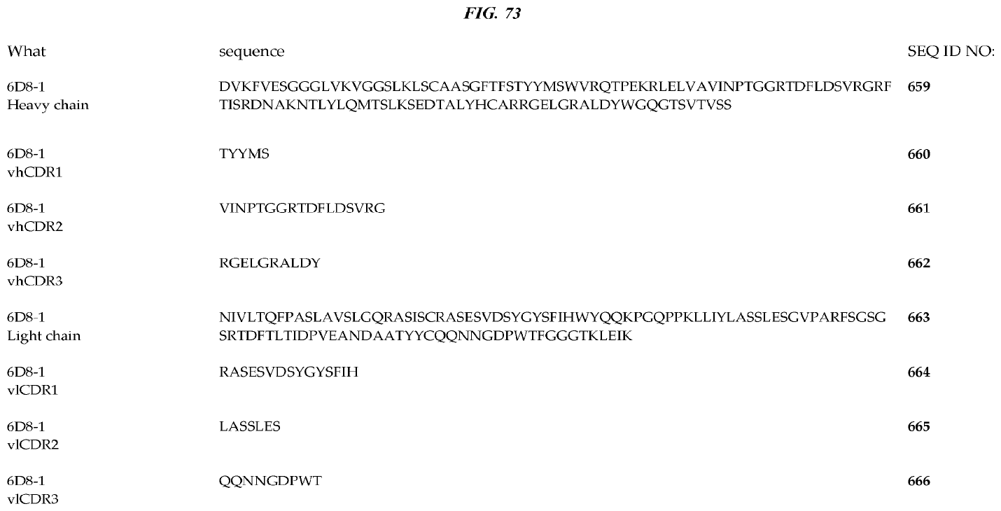

RNA expression data also revealed that PVRL2 is more abundantly expressed across DC-subtypes compared to PD-L1 and PVR (ligand of TIGIT). Flow cytometry confirmed dominant PVRL2 expression on DC subtypes across tumor indications. Immunohistochemistry analysis identified PVRL2 expression in Tertiary Lymphoid Structures. Finally, preliminary analysis of serum from CHA.7.518.1.H4 (S241P)+nivolumab treated patients revealed elevated induction of activated-DCs markers in two patients that responded clinically (RECIST criteria), compared to non-responders (FIG. 70).

### Conclusions

PVRIG is co-expressed with PD-1 and TIGIT on stem-like and exhausted T cells but has a unique dominant expression on early memory cells, while PVRL2 is abundantly expressed across DC-types. PVRIG is also co-expressed with PD-1 and TIGIT on stem-like and exhausted T cells but has a unique dominant expression on early memory cells. PVRIG blockade could therefore enhance memory T-cells activation by DCs, resulting in their increased expansion and differentiation. Accordingly, early data shows increased induction of activated DC markers, potentially following efficient T-DC interaction, in serum of two patients responding to CHA.7.518.1.H4 (S241P)+nivolumab.

### REFERENCES

1. Jansen, C. S., Prokhnevska, N., Master, V. A. et al., An intra-tumoral niche maintains and differentiates stem-like CD8 T cells. Nature 2019; 576; 465-470.
2. Held W, et al., Intratumoral CD8+ T cells with stem cell-like properties: implications for cancer immunotherapy. Sci Transl Med. 2019; 11 (515): eaay6863
3. Whelan S, Ophir E, Kotturi M F, Levy O, Ganguly S, Leung L, et al., PVRIG and PVRL2 are induced in cancer and inhibit CD8+ T-cell function. Cancer Immunol Res. 2019; 7:257-68.

### Example 11: CHA.7.518.1.H4 (S241P) Plus Nivolumab Demonstrates Preliminary Antitumor Activity and Immune Modulation of Tumor Microenvironment in Patients with Metastatic MSS-CRC and Liver Metastases

Background: CHA.7.518.1.H4 (S241P) a novel, 1st in-class, humanized IgG4 monoclonal antibody binds with high affinity to PVRIG, blocking its interaction with its natural ligand PVRL2 expressed in tumor cells and antigen-presenting-cells. Antitumor and pharmacodynamic activity of CHA.7.518.1.H4 (S241P) has been reported [1]. Anti-PD1/L1 therapies have limited to no activity in MSS-CRC. Therefore, novel ICI are urgently needed for the treatment of pts with MSS-CRC particularly pts with liver metastasis. Presented here are preliminary clinical and translational results of the combination in pts with MSS-CRC.

Methods: This is a phase I clinical trial of CHA.7.518.1.H4 (S241P) and nivolumab (NCT03667716). Key objectives were safety/tolerability [primary], preliminary antitumor activity, immune-related changes [secondary/exploratory]. Key inclusion criteria: Age≥18 yrs, histologically/cytologically confirmed advanced malignancy who have exhausted all available standard therapy or not a candidate for standard therapy, MSS-CRC determination per local testing. Pre- and on-treatment biopsies were obtained and analyzed by IHC for PDL1, CD8 expression and omics profiling.

Results: A total of 22 pts were enrolled: 2 pts combination dose-escalation [CHA.7.518.1.H4 (S241P) 0.3, 1 mg/kg+nivolumab 360 mg] both IV Q3W and 20 pts dose-expansion cohort [CHA.7.518.1.H4 (S241P) 20 mg/kg+nivolumab 480 mg IV Q4W]. Age≤65 17/22, [77%], male 16/22 [73%], median [Min, Max] of 3 (2, 10) prior lines of therapy, 17/22 [77%] had liver metastases. Overall, ORR 9% (2/22 pts, PRs]; ORR 12% [2/17] in pts with liver metastases [1 PR, PFS 44 weeks; 1 PR, PFS 16 weeks due to brain metastasis, however, response of target and non-target lesions still maintained]; DCR (CR+PR+SD) 27% (6/22). No new safety findings are reported. In 13 paired biopsy samples, 9 demonstrated induction in PD-L1 expression (mean 16.3+/−7% PD-L1 CPS-score increase, p<0.05), suggesting TME immune-modulation following treatment. In pts with PR or SD>6months greater induction in PD-L1 expression was seen (49.7+/−14.9%). CD8 T-cell quantification was available in 12 paired biopsies with increase>1% in 8 pts (mean % CD8 increase of 9.1+/−4.4%, p=0.08), with substantial increases in responders (36.5% and 44.7% % CD8 increase). In responding pts IFNγ signature up-regulation, increased T-cell clonality and specific clonal expansion, were demonstrated between baseline and on-treatment biopsies.

Conclusion: CHA.7.518.1.H4 (S241P)+nivolumab demonstrates preliminary antitumor activity in pts with heavily pretreated metastatic MSS-CRC with 12% ORR in pts with liver metastases [typically unresponsive to ICI]. TME immune modulation observed in the majority of pts, substantial in responders, suggests unique potential of CHA.7.518.1.H4 (S241P) in less inflamed tumors such as MSS-CRC. The combination warrants further development.

Reference: Vaena, DA, Fleming, GF, Chmielowski B et al., CHA.7.518.1.H4 (S241P) with or without nivolumab: Results of an ongoing phase 1 study of safety, tolerability, and preliminary antitumor activity in patients with advanced solid malignancies (NCT03667716). Journal of Clinical Oncology 2021 39: 15_suppl, 2504-2504.

### Example 12: CHA.7.518.1.H4 (S241P) with or without Nivolumab Demonstrates Preliminary Antitumor Activity in Patients with Metastatic NSCLC Previously Treated with ICI

Background: CHA.7.518.1.H4 (S241P) is a novel, 1st in-class, humanized IgG4 monoclonal antibody that binds with high affinity to PVRIG, blocking its interaction with its natural ligand PVRL2 expressed in tumor cells and antigen-presenting-cells. There is an urgent medical need for the treatment of pts with metastatic NSCLC who have exhausted available standard therapies including treatment with approved ICI. While not being bound by theory, it is hypothesized that CHA.7.518.1.H4 (S241P)+nivolumab will demonstrate clinical activity in this patient population. Presented here are results of preliminary antitumor activity including overall survival.

Methods: The data presented are from an ongoing phase I clinical trial of CHA.7.518.1.H4 (S241P) and nivolumab (NCT03667716). Key objectives were safety/tolerability [primary], preliminary antitumor activity e.g., PFS, OS [secondary/exploratory]. Key inclusion criteria: Age≥18 yrs, histologically/cytologically confirmed advanced malignancy who have exhausted all available standard therapy.

Results: Seven patients were enrolled with NSCLC: 5 pts CHA.7.518.1.H4 (S241P) monotherapy [4 pts-CHA.7.518.1.H4 (S241P) 20 mg/kg IV Q4W monotherapy dose expansion, 1 pt CHA.7.518.1.H4 (S241P) 0.01 mg/kg IV Q3W], 2 pts CHA.7.518.1.H4 (S241P)+nivolumab [CHA.7.518.1.H4 (S241P)/nivolumab 3 mg/kg/360 mg both IV Q3W, CHA.7.518.1.H4 (S241P)/nivolumab 10 mg/kg/480 mg both IV Q4W]. Age>65 4/7 [57%], female 6/7 [86%], prior lines median [Min, Max] 4 [3, 6], all pts had received prior ICI, 4/7 [57%] had received>2 prior lines with ICI. Overall, DCR (CR+PR+SD) 5/7 [71%] pts with SD [no pts with CR/PR], median OS in all patients 9.5 months [95% CI, 2.7-11.6]-[CHA.7.518.1.H4 (S241P) monotherapy 9.5 months [2.7, NE]; combination 10.1 months [95% CI, 8.6, NE]. Historical data metastatic NSCLC data 2nd line after failure of platinum containing regimen [REVEL P3 trial, Docetaxel±ramucirumab-median OS combination 10.5 months vs 9.1 months with placebo+docetaxel]; [CheckMate057, Nivolumab vs docetaxel; median OS 12.2 vs 9.4 months respectively], Post ICI [Lung-MAP S1800A with median OS was 14.5 months for ramucirumab+pembrolizumab and 11.6 months for SOC [predominantly docetaxel+ramucirumab]. No new safety findings are reported.

Conclusion: CHA.7.518.1.H4 (S241P)±nivolumab demonstrates encouraging preliminary antitumor activity with better than predicted OS in pts with heavily pretreated metastatic NSCLC [median 4 prior lines] after prior exposure to ICI. In this small dataset the OS compares favorably to historical data in 2nd line setting prior to ICI. Additional clinical evaluation of CHA.7.518.1.H4 (S241P)±nivolumab in this patient population is warranted.

### Example 13: CHA.7.518.1.H4 (S241P) in Combination with Nivolumab Demonstrates Preliminary Antitumor Activity in Patients with Platinum Resistant Epithelial Ovarian Cancer

Background: There is a high unmet medical need for the treatment of patients [pts] with platinum resistant epithelial ovarian cancer [PROC]. Immune checkpoint inhibitors (ICI) have limited activity in this pt population. CHA.7.518.1.H4 (S241P) is a novel, 1^{st}-in-class ICI that binds to PVRIG, a DNAM-1 axis member, leading to activation of T- and NK-cells. While not being bound by theory, it is hypothesized that in pts with PROC, dual blockade of PVRIG and PD1 would demonstrate antitumor activity with a favorable safety and tolerability profile. This example presents preliminary results.

Methods: A total of 20 patients [pts] with PROC were enrolled. All pts received CHA.7.518.1.H4 (S241P) 20 mg/kg+nivolumab 480 mg both IV Q4W. Primary objectives were safety/tolerability, with secondary objective of preliminary antitumor activity. Key inclusion criteria: Age≥18 yrs, histologically confirmed locally advanced or metastatic solid malignancy and has exhausted all available standard therapy. Key exclusion criteria: prior receipt of anti-PVRIG, anti-TIGIT, no limitation on the number of prior lines of therapy or prior PD-1/PD-L1 inhibitor. Investigator assessed responses were per RECIST v1.1, safety per CTCAE v4.03.

Results: Median age 61.5 yrs, median of 6 prior lines of therapy [Min, Max: 2,9]. Objective response rate (ORR) of 2/20 [10%] pts: 1 pt with fallopian tube CA, 6 prior lines of therapy, clear cell histology; 1 pt with OVCA, serous adenoCA, 7 prior lines of therapy [including prior nivolumab with best response of progressive disease]; both subjects with PR have an ongoing response to therapy, no complete responses (CR); 5 pts with stable disease (SD). Disease control rate [CR+PR+SD] 7/20 [35%]. Most frequent AEs were G1/2 nausea 11 pts, fatigue 11 pts [all G1/2]. Increase of IFNγ was observed, confirming the expected immune activation induced by CHA.7.518.1.H4 (S241P) given in combination with nivolumab.

Conclusion: CHA.7.518.1.H4 (S241P)±nivolumab demonstrates encouraging preliminary signal of antitumor activity and immune activation in pts with heavily pre-treated PROC with a favorable safety/tolerability profile. Additional data analyses and pt follow up are ongoing.

### Example 14: CHA.7.518.1.H4 (S241P)±Nivolumab-Preliminary Results of Antitumor Activity from a Phase 1 Study in Patients with Metastatic NSCLC Who have Received Prior PD-1/PD-L1 Inhibitor

Background: Novel agents are urgently needed for the treatment [tx] of patients [pts] with metastatic NSCLC including post immune checkpoint inhibitors (ICI). CHA.7.518.1.H4 (S241P) [anti-PVRIG] is a novel 1st in class ICI. While not being bound by theory, it is hypothesized that CHA.7.518.1.H4 (S241P)±nivolumab will demonstrate antitumor activity with a favorable safety and tolerability profile in pts with heavily pretreated NSCLC. In this example preliminary results on antitumor activity are provided.

Methods: In total 7 pts with NSCLC were enrolled: 5 CHA.7.518.1.H4 (S241P) monotherapy [mtx] [4-CHA.7.518.1.H4 (S241P) 20 mg/kg IV Q4W mtx expansion, 1 pt CHA.7.518.1.H4 (S241P) 0.01 mg/kg IV Q3W], 2 pts CHA.7.518.1.H4 (S241P)±nivolumab [CHA.7.518.1.H4 (S241P)/nivolumab 3 mg/kg/360 mg both IV Q3W, CHA.7.518.1.H4 (S241P)/nivolumab 10 mg/kg/480 mg both IV Q4W]. Key study objectives were safety/tolerability and preliminary antitumor activity; OS was an exploratory endpoint. Key inclusion criteria: Age≥18 yrs, histologically confirmed metastatic solid malignancy and has exhausted all standard tx. Key exclusion criteria: history of immune toxicities on prior ICI tx leading to discontinuation. Safety per CTCAE v4.03, investigator-assessed response per RECIST v1.1.

Results: Age>65 4/7 [57%], female 6/7 [86%], prior lines median [Min, Max] 4 [3, 6], all pts received prior ICI, 4/7 [57%] received>2 prior lines with ICI. Disease control rate [CR+PR+SD) 5/7 [71%], no CR or PR. Median [m] PFS [all pts] 84 days 95% CI [22, 231], mOS in all pts 9.5 months [mos] [95% CI, 2.7-11.6]-[CHA.7.518.1.H4 (S241P) mtx 9.5 mos [2.7, NE]; combination 10.1 mos [95% CI, 8.6, NE]. The most frequent AE was GI nausea in 2 pts. Post ICI NSCLC data-1 prior line of ICI in metastatic setting, mOS 14.5 mos for ramucirumab+pembrolizumab (Reckamp K L, et al., Phase 2 Randomized Study of Ramucirumab and Pembrolizumab vs SOC in Advanced NSCLC Previously Treated With Immunotherapy-Lung-MAP S1800A. J Clin Oncol. 2022 Jul. 20; 40 (21): 2295-2306).

Conclusion: CHA.7.518.1.H4 (S241P)±nivolumab has preliminary encouraging signal of antitumor activity in a heavily pretreated population of patients with NSCLC with prior ICI tx comparable to historical data. A further study in post IO NSCLC evaluating CPA.9.086.H4 (S241P))+CHA.7.518.1.H4 (S241P)+PD-1 inhibitor and CPA.9.086.H4 (S241P))+CHA.7.518.1.H4 (S241P)+chemotherapy is planned.

### Example 15: PVRIG, a Novel T Cell Checkpoint, is Preferentially Expressed in TLS on Stem-Like Memory T Cells, Potentially Inhibiting their Expansion

### Background

Tertiary lymphoid structures (TLS) recently emerged as an intra-tumoral niche with a predictive value for cancer immunotherapy responses. LAMP3+DCs in the TLS were shown to interact with and support the differentiation of stem-like CD8+ (Tscm) into effector-like cells, that then expand in the tumor micro-environment (TME) and may exert anti-tumor responses. It has been previously shown that PVRIG is expressed on Tscm and exhausted T cells, but has a unique dominant expression on Tscm cells, while PVRL2 is abundantly expressed across DC types (Alteber et al., P252, SITC 2021, http://dx.doi.org/10.1136/jitc-2021-SITC2021.252). In this example, data on the expression of DNAM-1 axis genes in the TME and the immune response in the blood and TME of cancer patients treated with CHA.7.518.1.H4 (S241P) (anti-PVRIG antibody) monotherapy or CHA.7.518.1.H4 (S241P)±nivolumab combination therapy is provided.

### Method

MERFISH technology was employed to detect the expression of 350 distinct mRNA transcripts at subcellular resolution in CRC sections. Extensive omics profiling was performed on pre- and on-treatment biopsies from patients in the CHA.7.518.1.H4 (S241P) and CHA.7.518.1.H4 (S241P)±nivolumab Phase-1 clinical study (NCT03667716).

PVRIG Uniquely Clusters with Early Differentiated/Tscm Genes

Unsupervised PCA was performed on the scRNA expression matrix of TME CD8+ T cells, which includes all variable genes. Using cells as features and genes as entries, co-expression pattern among genes known to be expressed on naïve, memory, and exhausted CD8+ T cells is shown. Nine datasets were analyzed, representative NSCLC dataset is shown. ScRNA-seq datasets (CRC, NSCLC, HNSCC, Melanoma, Liver cancer, n=13) were analyzed for the co-expression pattern of 19 genes, including genes known to be expressed on naïve (TCF7, IL7R, SELL), memory (GZMK, EOMES), and exhausted (PDCD1, LAG3, HAVCR2) CD8+ T cells. Representative dataset of CRC is presented. The analysis results are illustrated in FIG. 76.

Spatial Transcriptomic Analysis of TLS Regions Shows Enrichment of Tscm and Dendritic Cells, while Exhausted Cells Localize to the Tumor

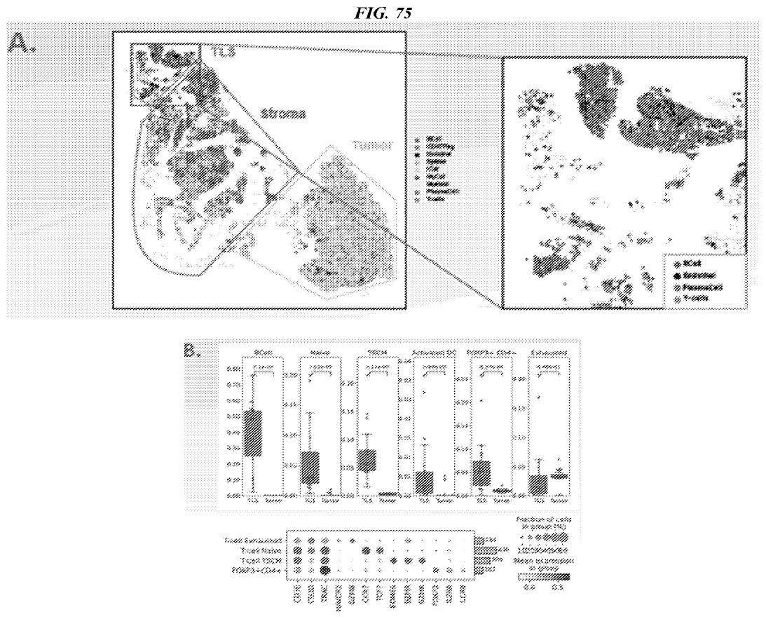

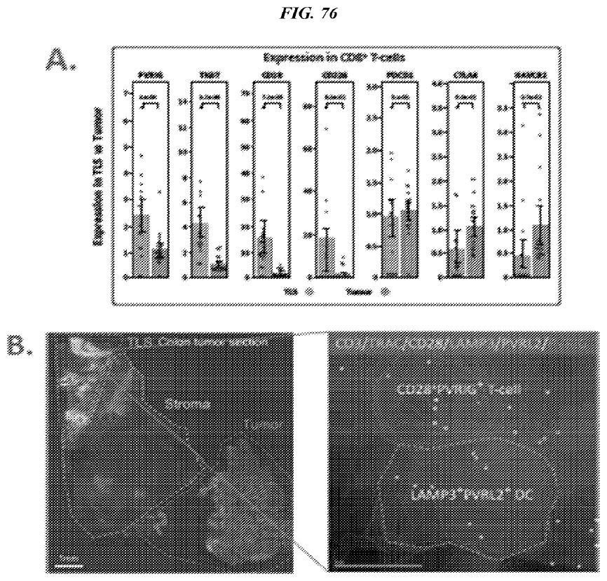

As provided in FIG. 75, analysis of CRC samples by MERFISH allowed identification of cellular composition in Tertiary Lymphoid Structures. TLS region harbored a unique co-localization of B cells, T cells, plasma and endothelial cells, that is unique to these structures. Boxplot shows the cell composition in individual TLS structures or randomly sampled tumor regions. Tscm & naïve T cells localized to TLS, while exhausted cells localized to the tumor region. Dotplot shows the gene expression of selected cell-state markers in a single TLS.

### PVRIG and Other Genes of the DNAM-1 Axis are Dominantly Expressed in TLS Region

DNAM-1 axis is dominantly expressed in TLS region. As shown in FIG. 76, genes of the DNAM-1 axis, PVRIG, TIGIT, and CD226, showed dominant expression in TLS region, whereas other immune checkpoints, such as PD-1, CTLA4, and TIM3 were expressed more in the tumor bed. CD28+CD8+ T cell expressing PVRIG intimately interacted with LAMP3+PVRL2+DC in the TLS of a CRC patient.

CHA.7.518.1.H4 (S241P) Monotherapy Induces TME Immune Modulation in Patients with Ovarian Cancer

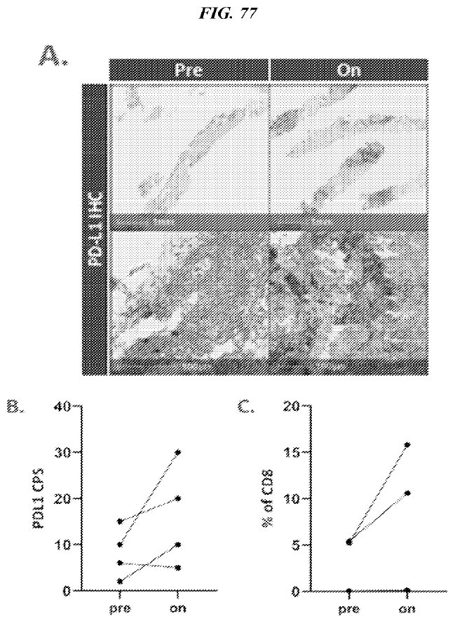

Increased TME immune activation following CHA. 7.518.1.H4 (S241P) monotherapy in ovarian cancer patients. As shown in FIG. 77, monotherapy with CHA.7.518.1.H4 (S241P) increased PD-L1 expression (IHC, SP263) in ovarian cancer. An increase in PD-L1 CPS score (SP263) was observed in 3 out of 4 patients, and in CD8+ cells percentages (IF, C8/144B, quantification with HALO) in 2 out of 3 patients. All panels compare on-treatment (between cycle 2 and 3) to pre-treatment biopsies.

CHA.7.518.1.H4 (S241P)±Nivolumab Combination Induces TME Immune Modulation in Patients with MSS-CRC

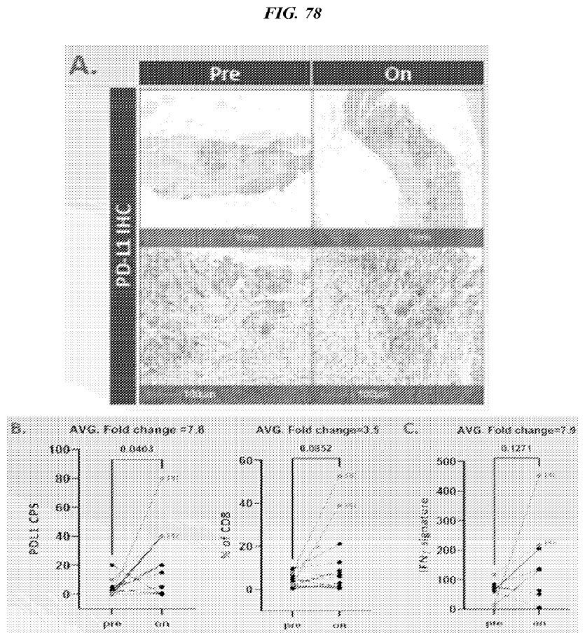

Increased TME Immune activation was induced by CHA.7.518.1.H4 (S241P) and nivolumab combo in MSS CRC patients. FIG. 78 demonstrates PD-L1 expression (IHC, 28-8) was induced by combination therapy. An increase in PD-L1 CPS score (28-8) was observed in 9 out of 13 patients, and CD8 quantification (IHC, C8/144B) observed in 7 out of 11 patients biopsies treated with CHA.7.518.1.H4 (S241P)±nivolumab. An increase in IFNg mRNA signature was observed in 5 out of 8 patients in on-treatment biopsies compared to pre-treatment. All panels in FIG. 78 compare on-treatment (between cycle 2 and 3) to pre-treatment biopsies. PR: patient with partial response per RECIST.

### Extensive TME Modulation in MSS-CRC Patients Partially Responding to CHA.7.518.1.H4 (S241P)±Nivolumab

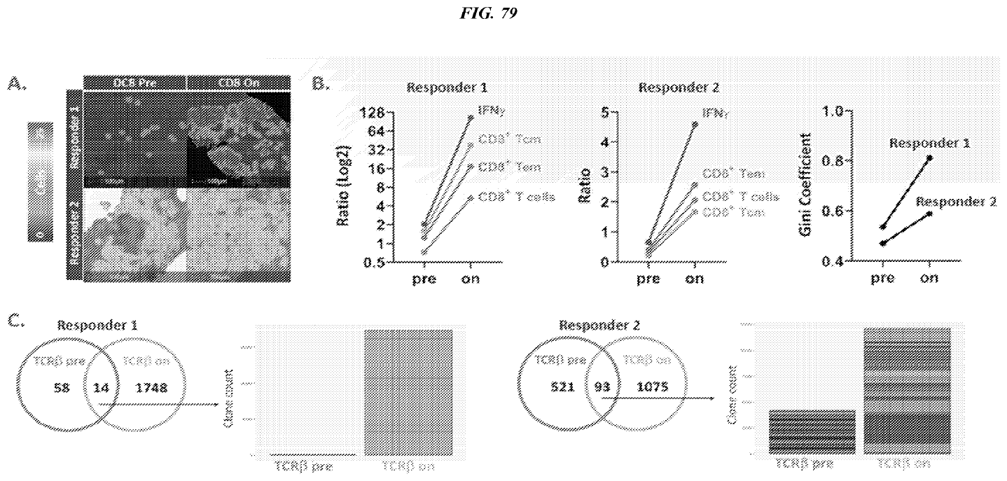

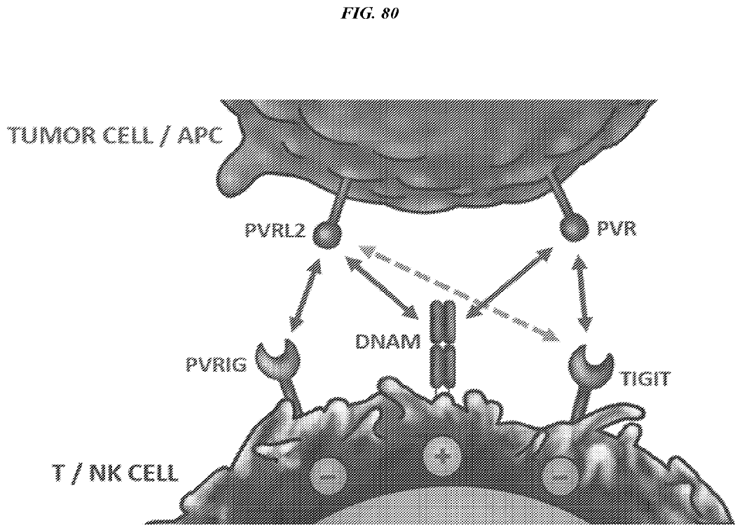

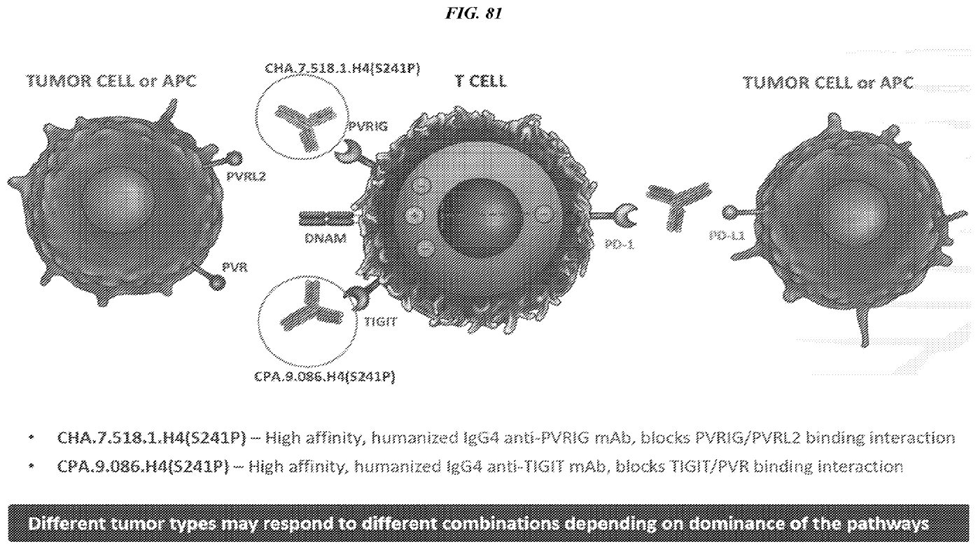

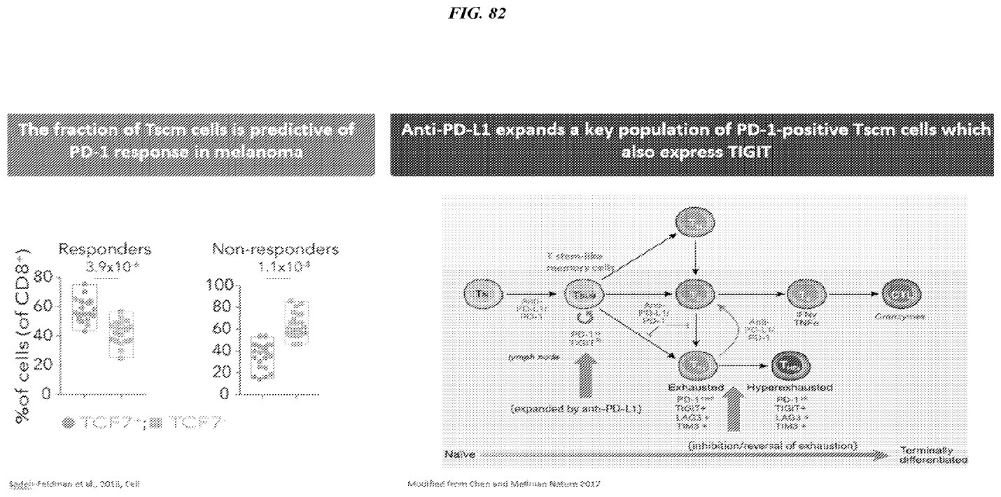

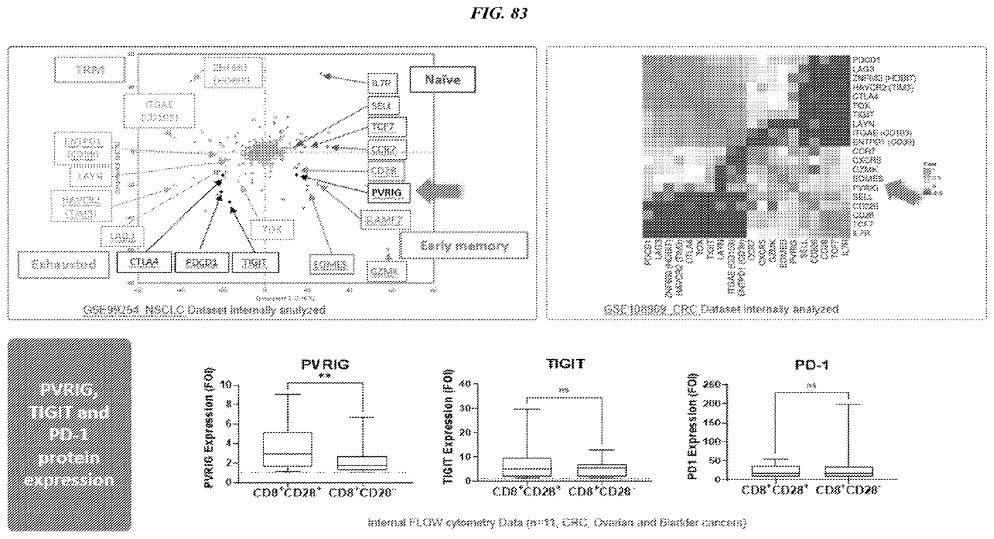

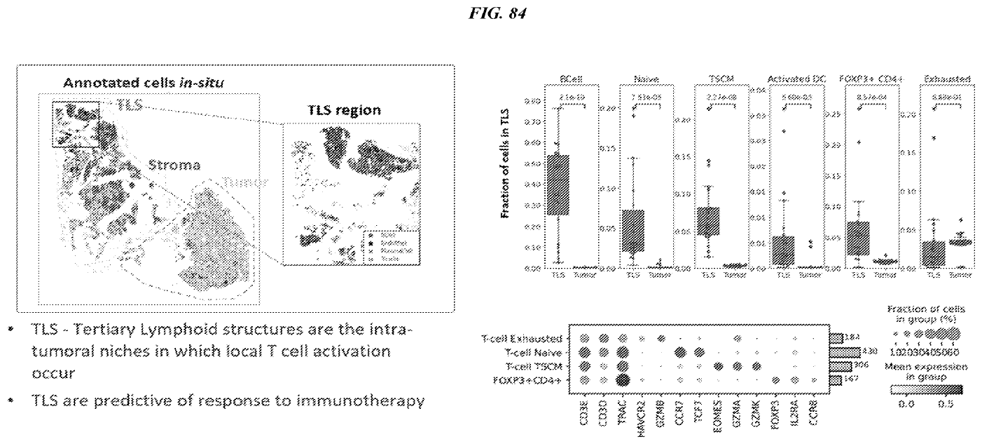

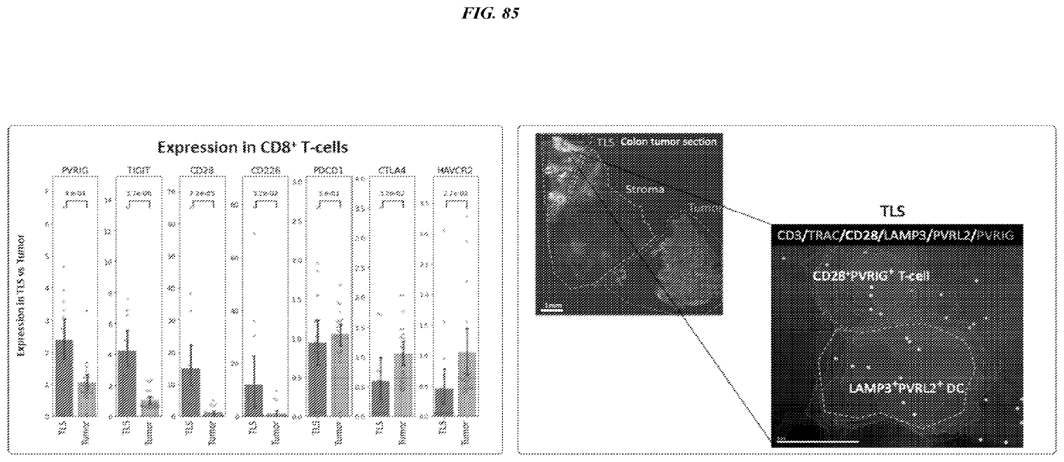

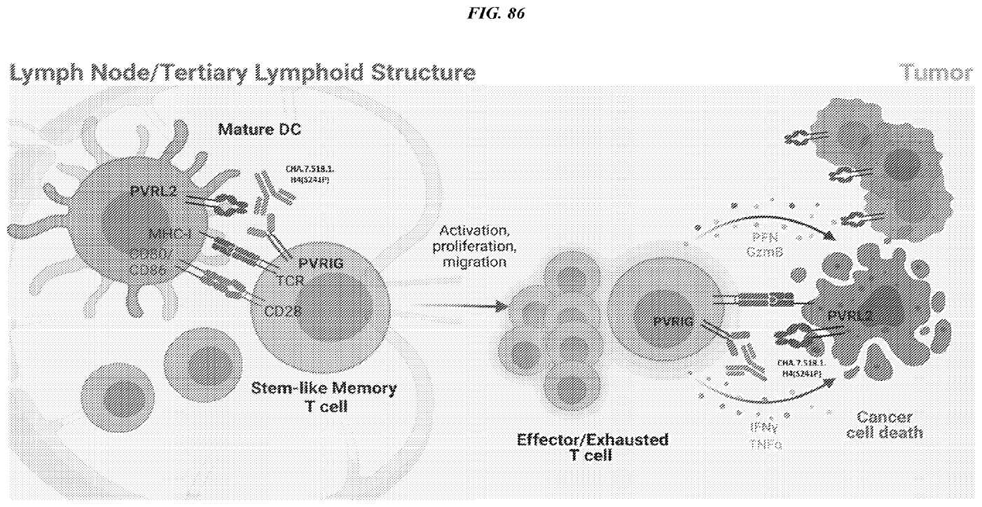

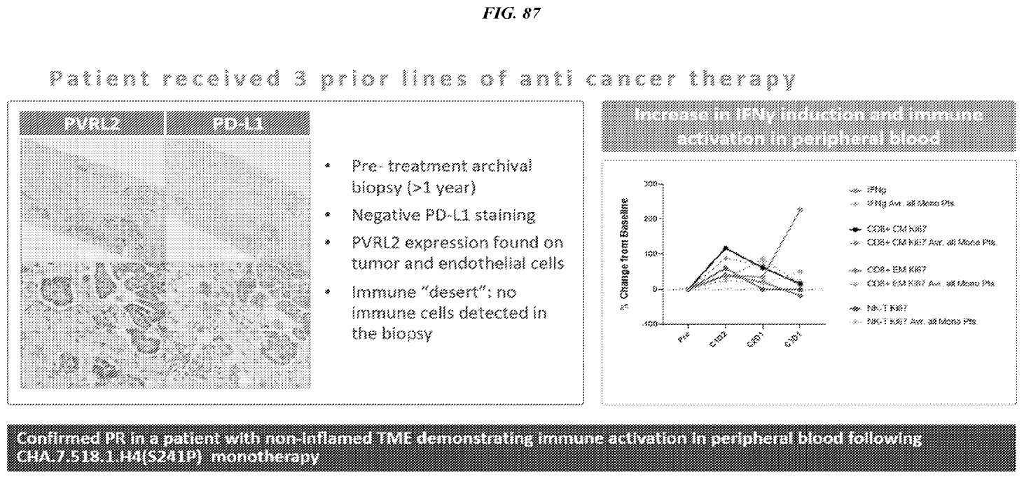

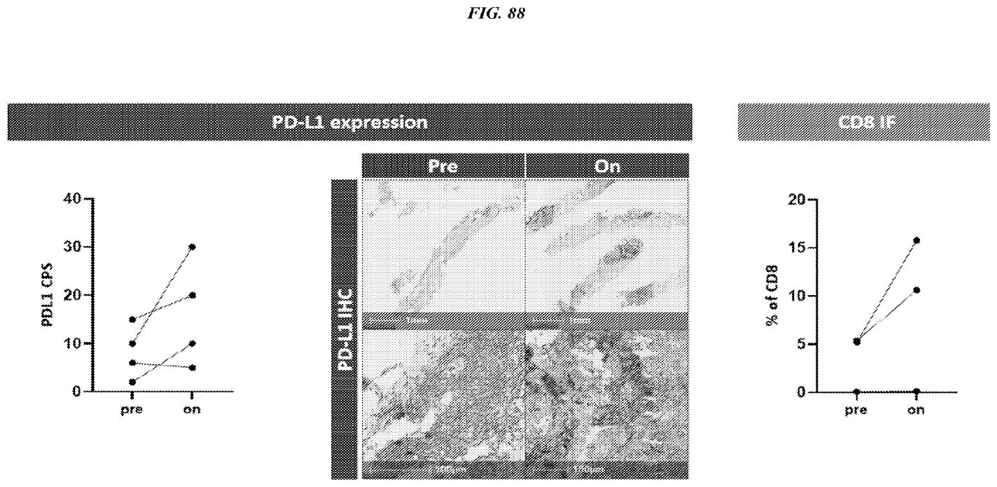

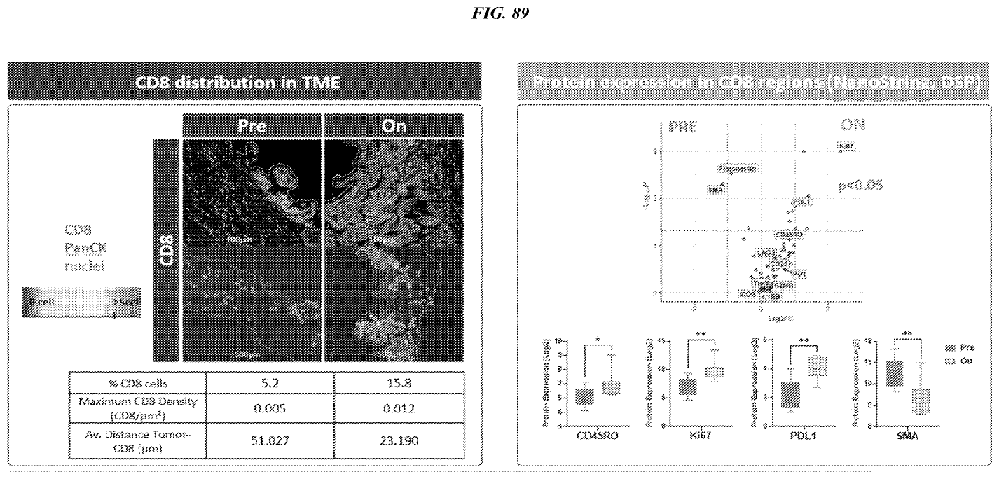

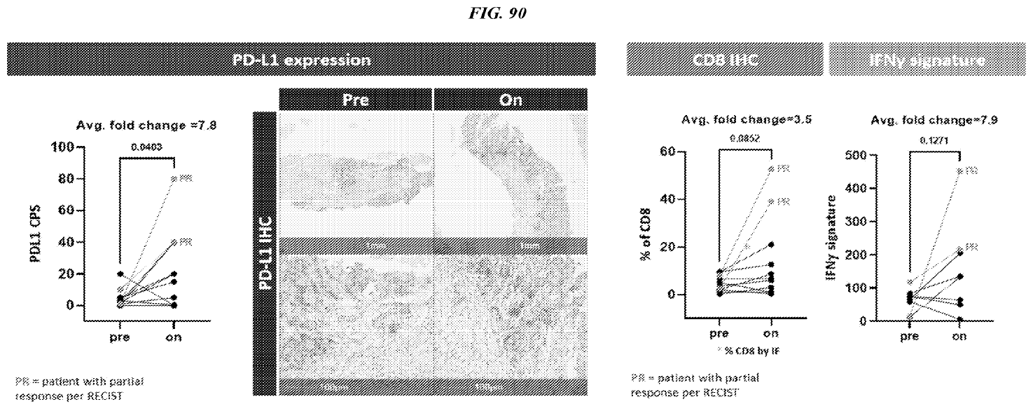

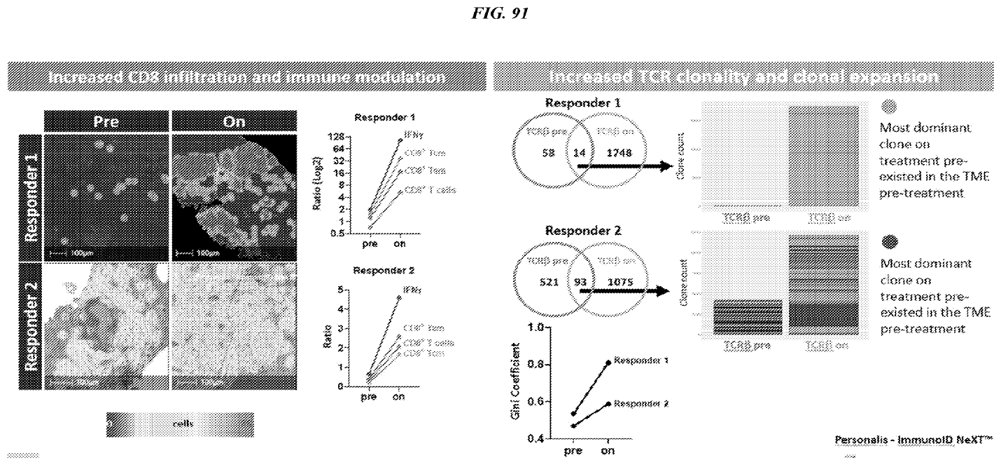

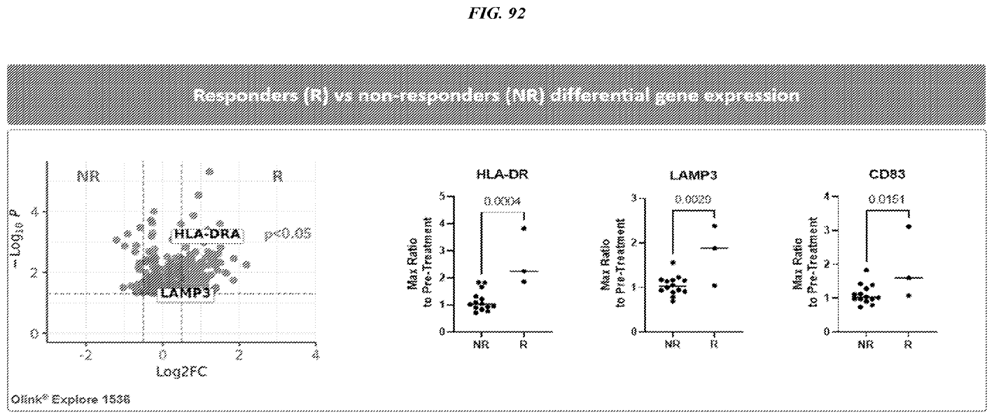

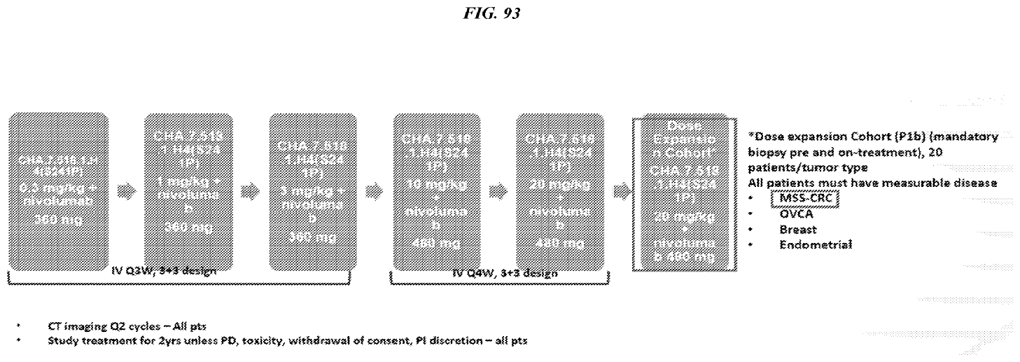

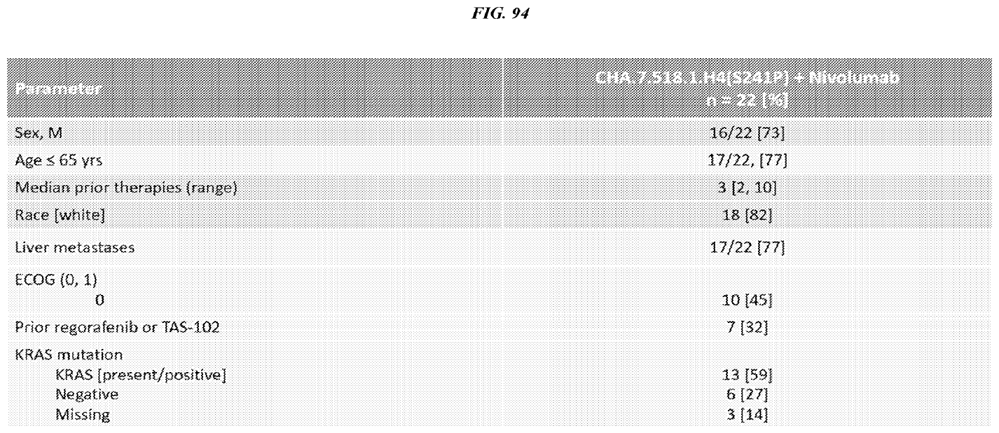

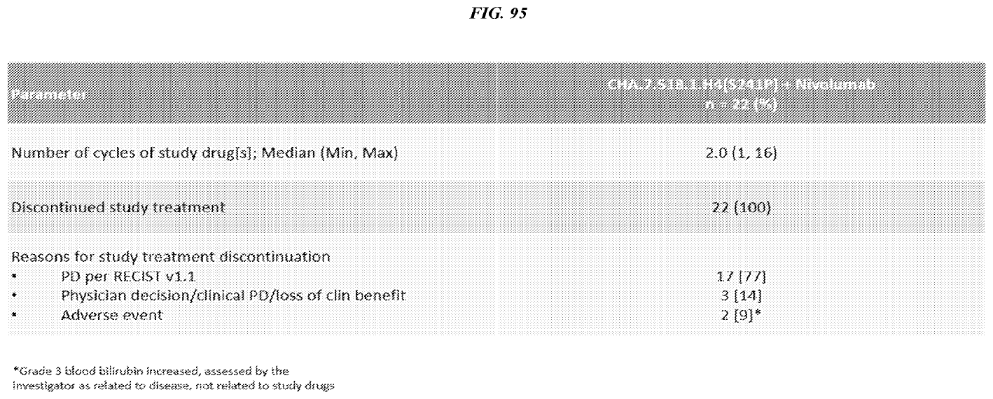

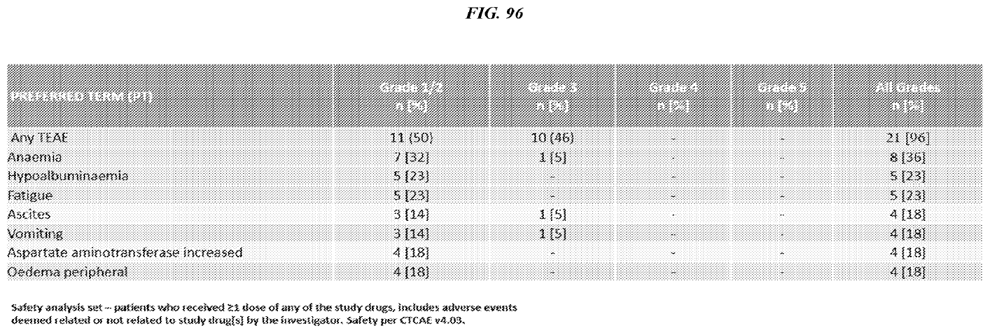

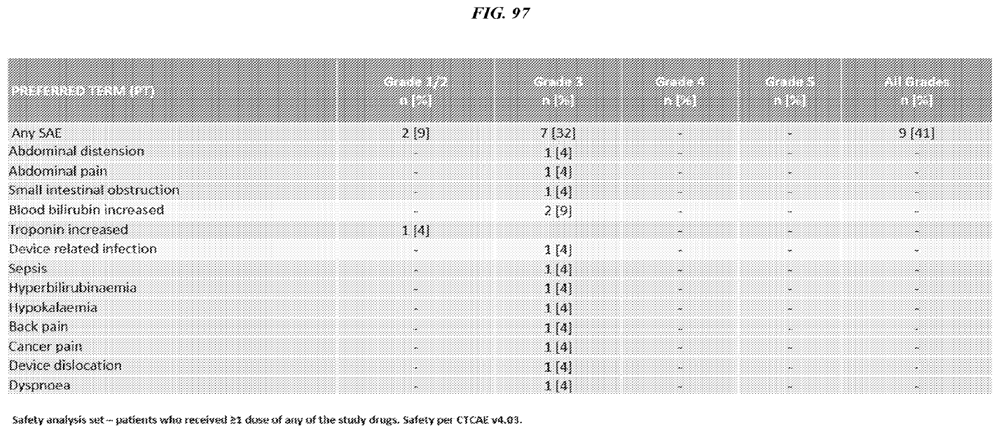

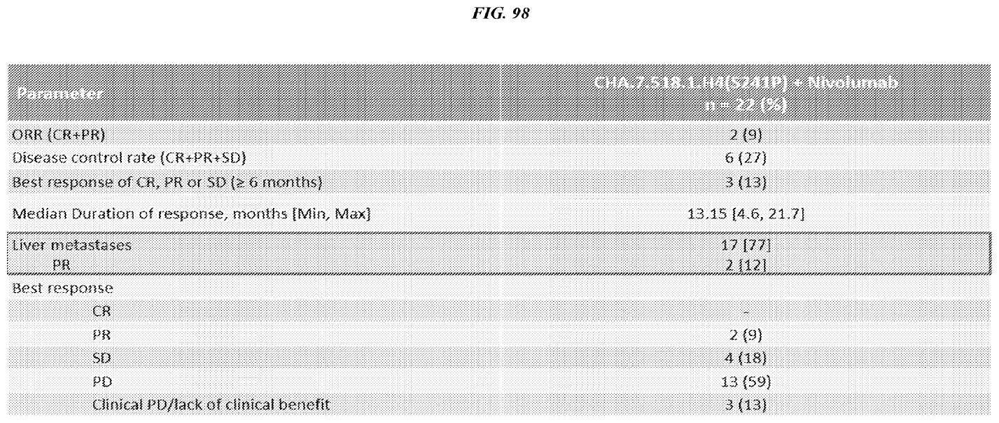

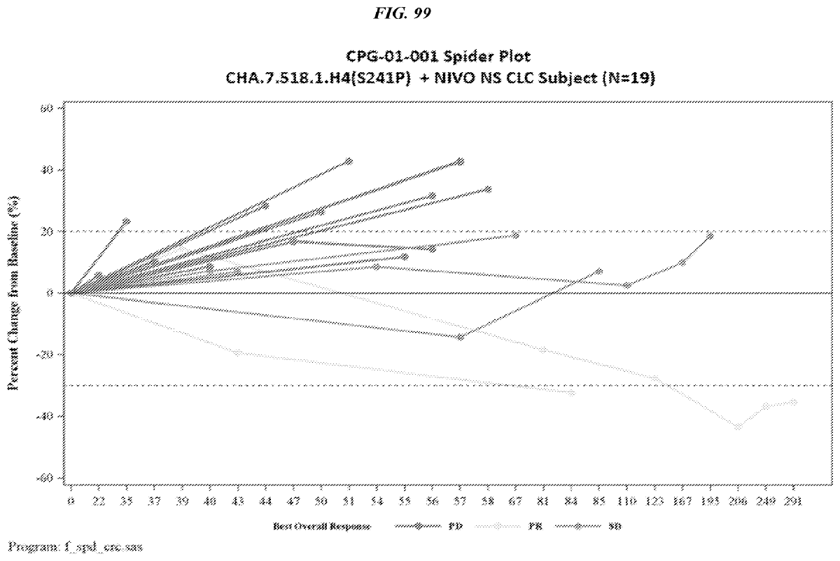

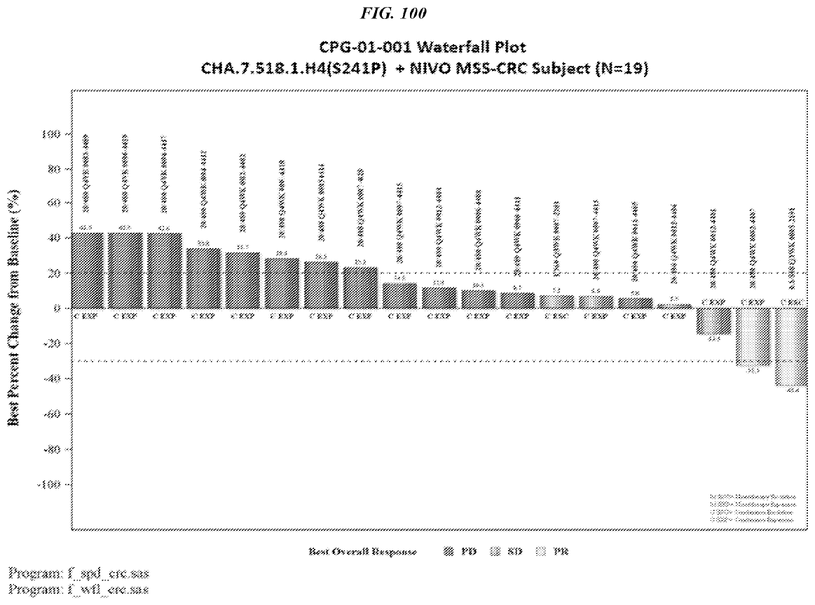

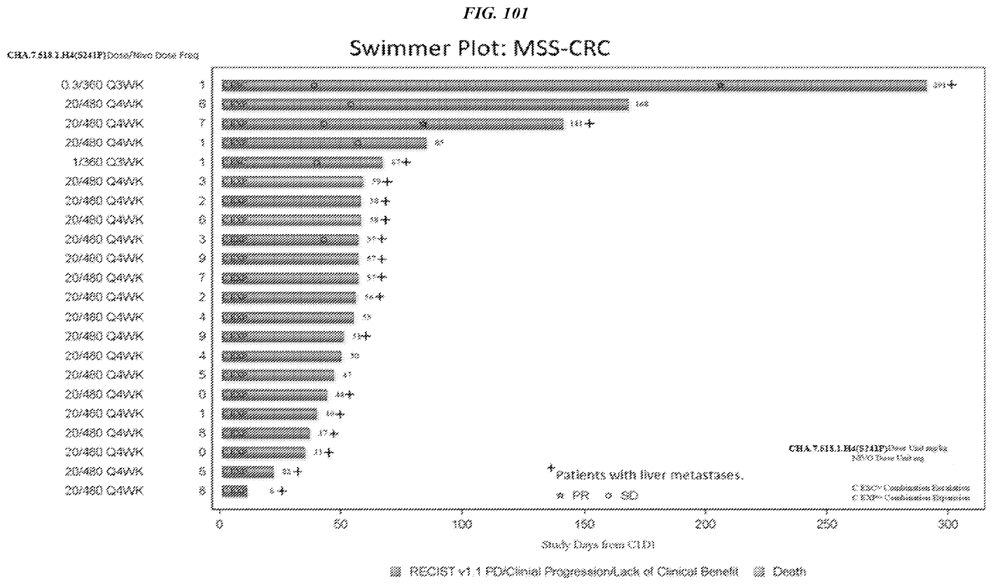

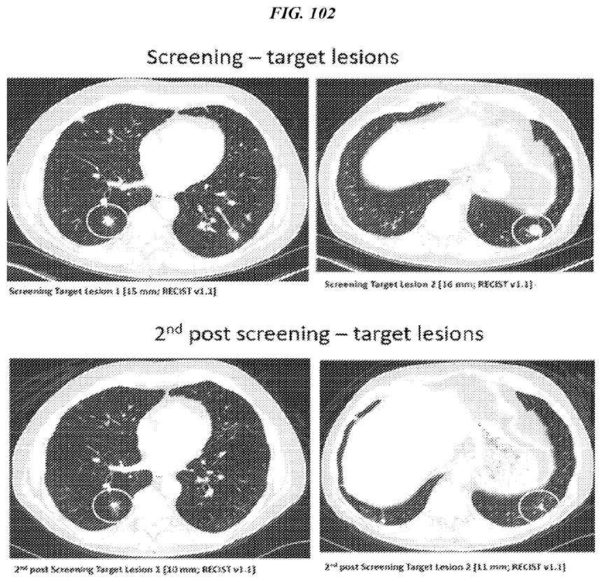

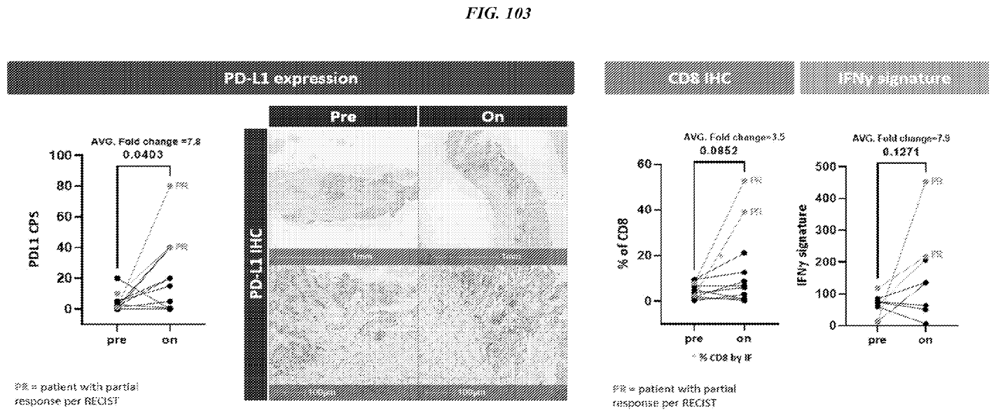

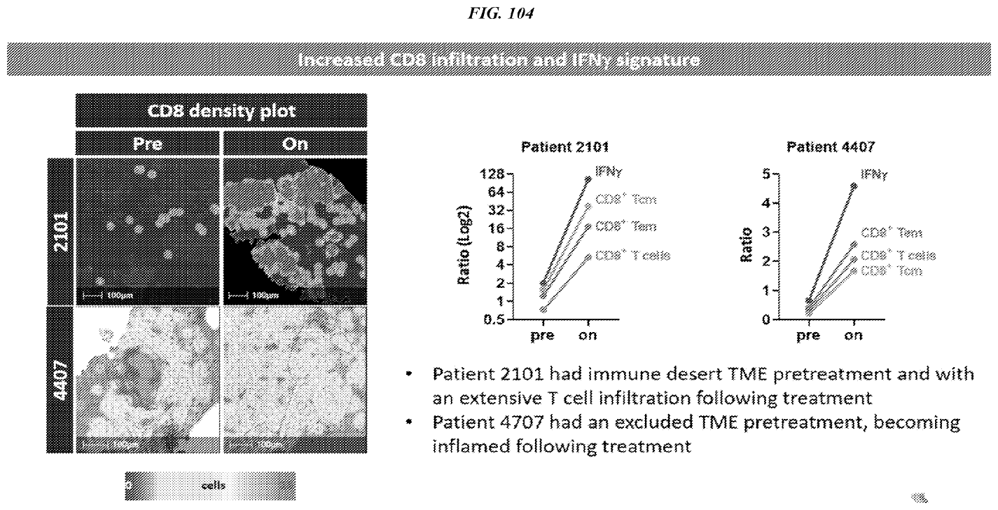

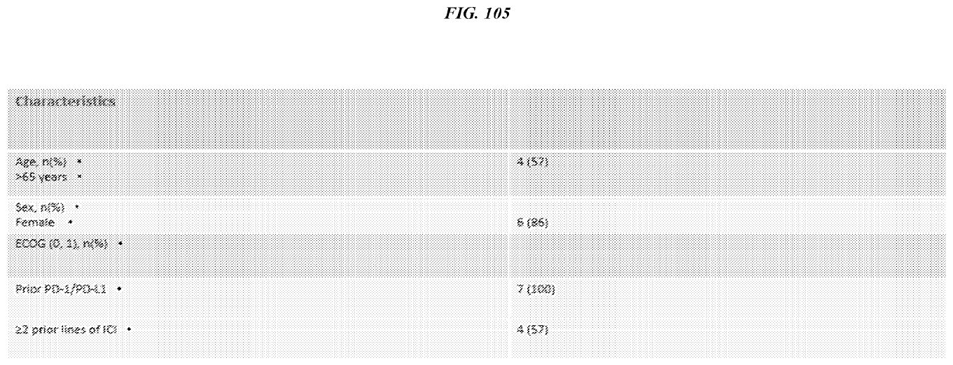

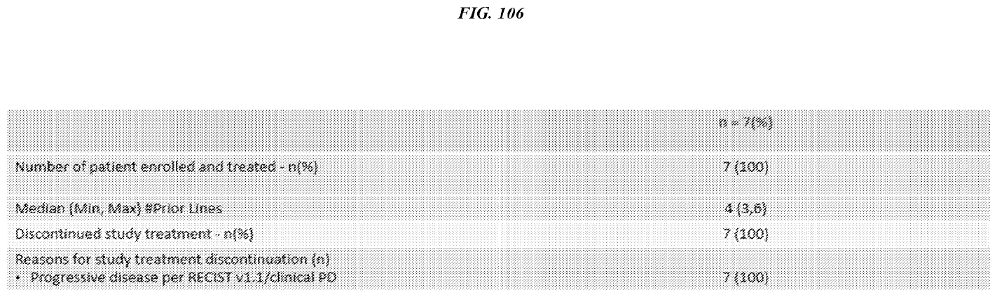

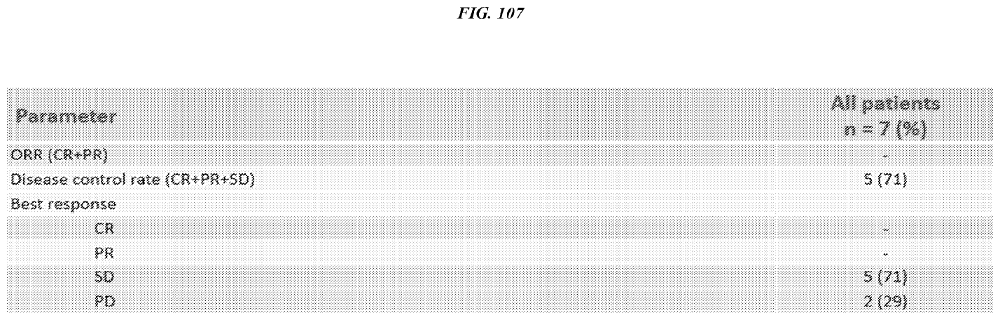

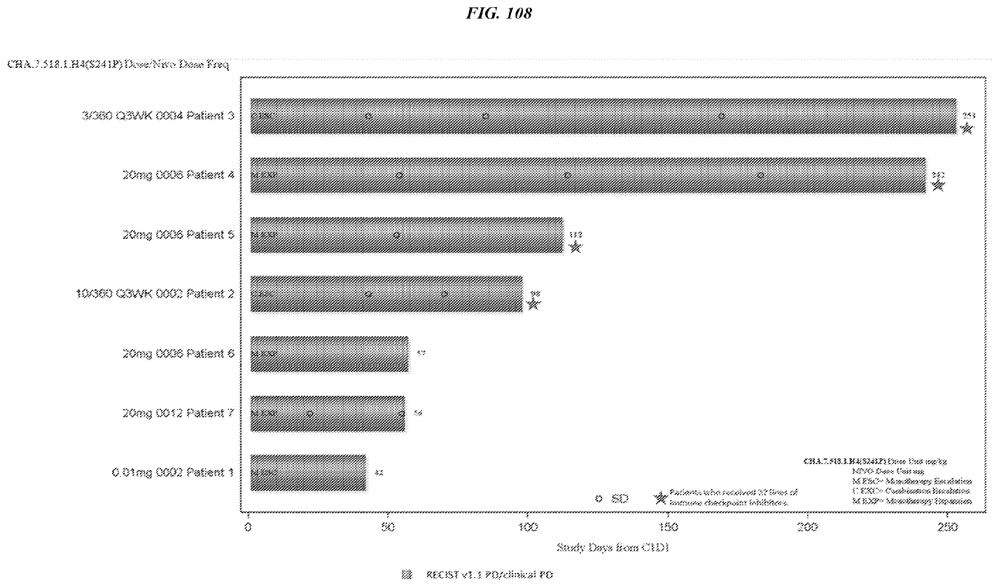

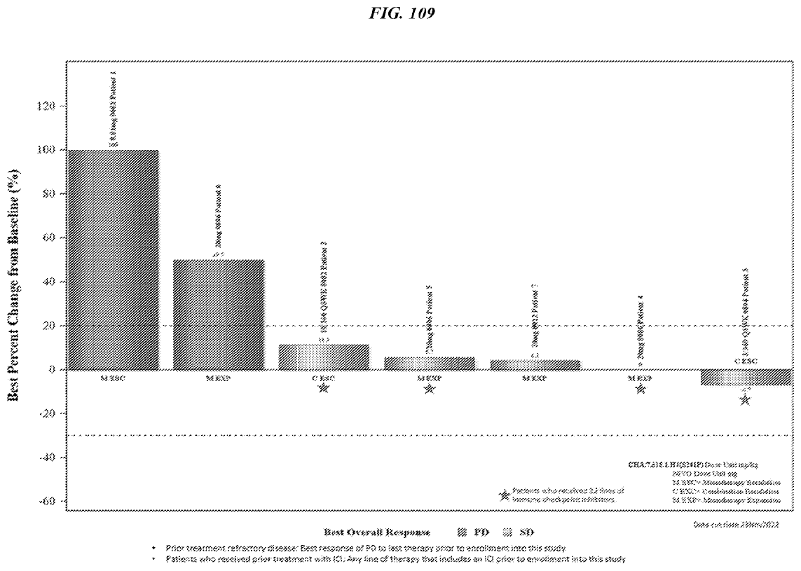

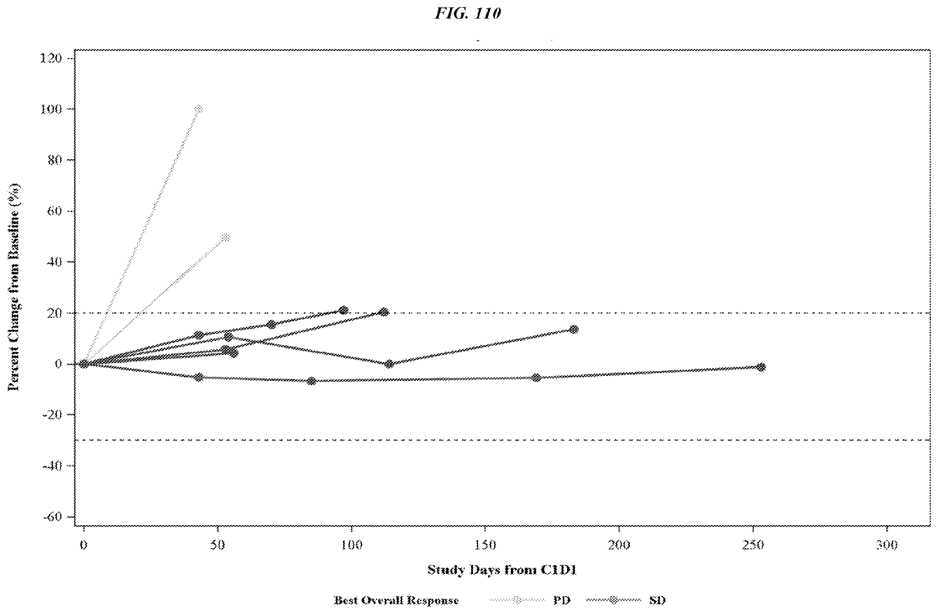

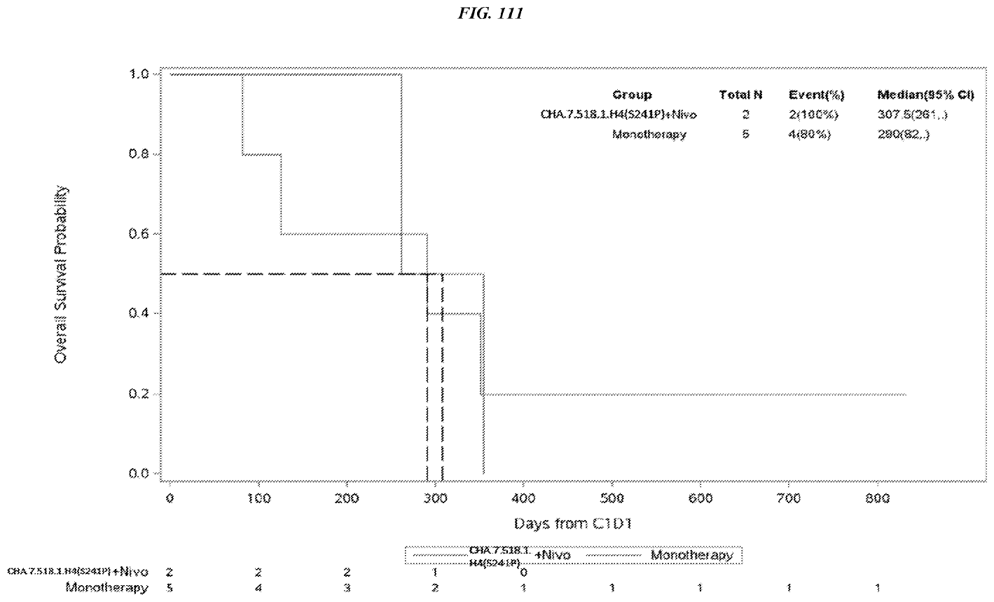

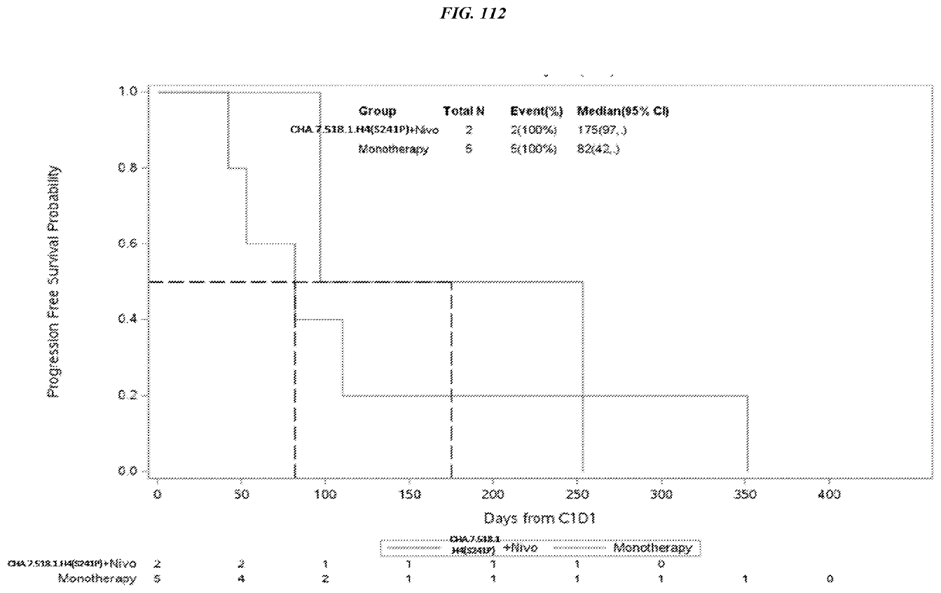

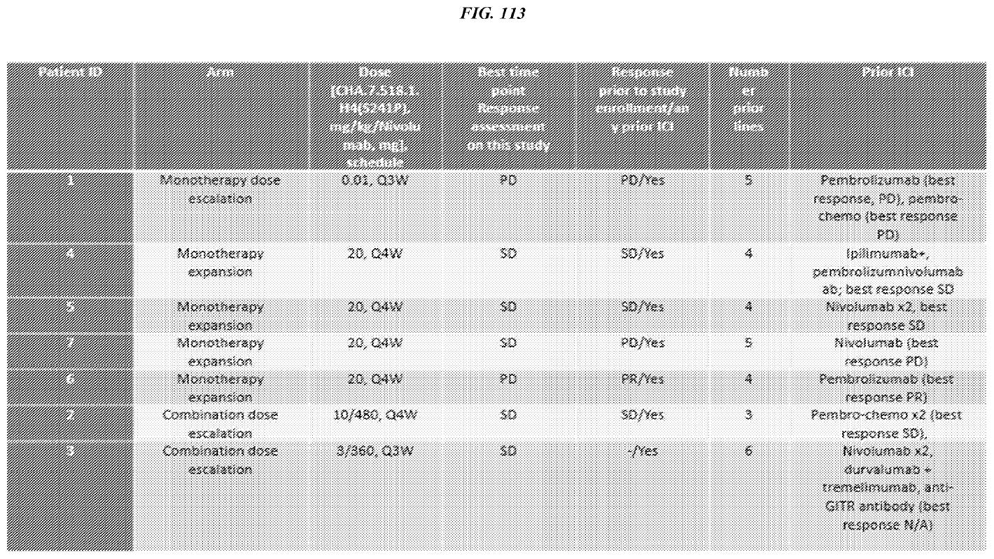

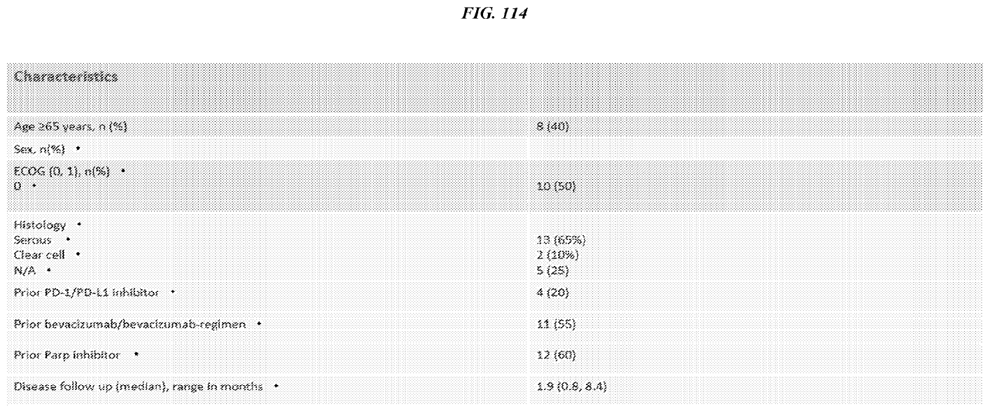

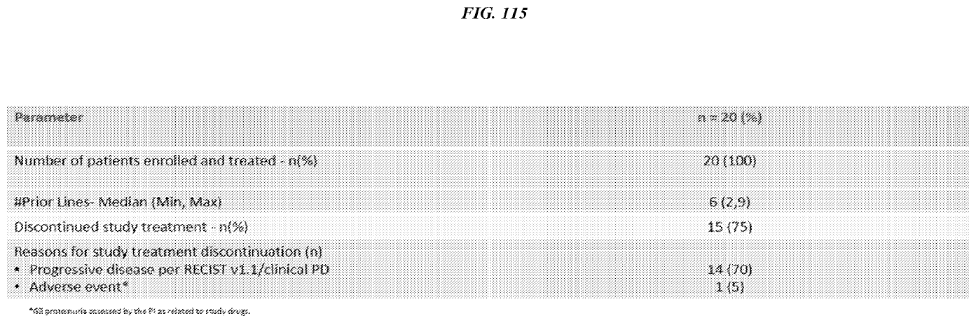

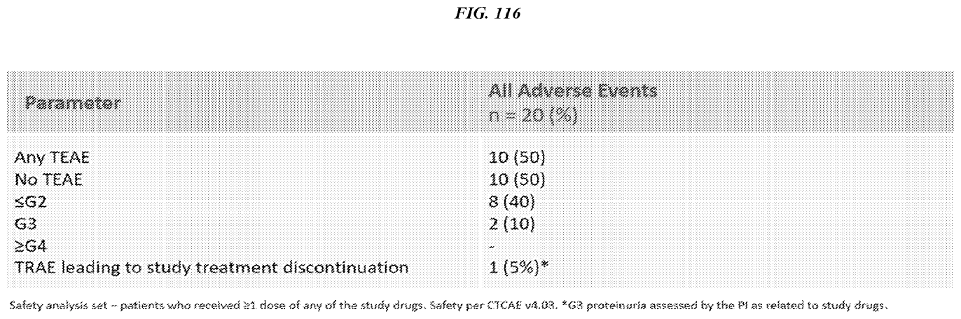

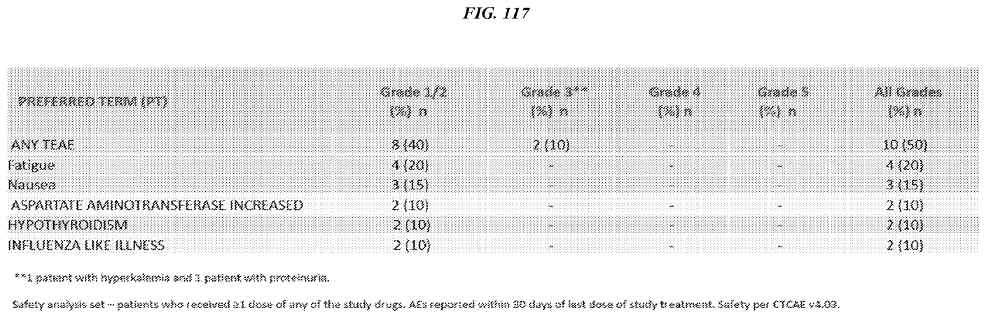

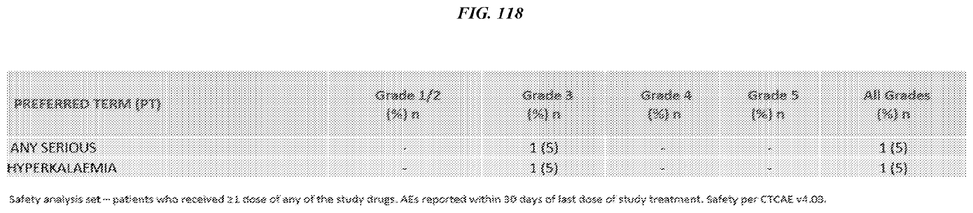

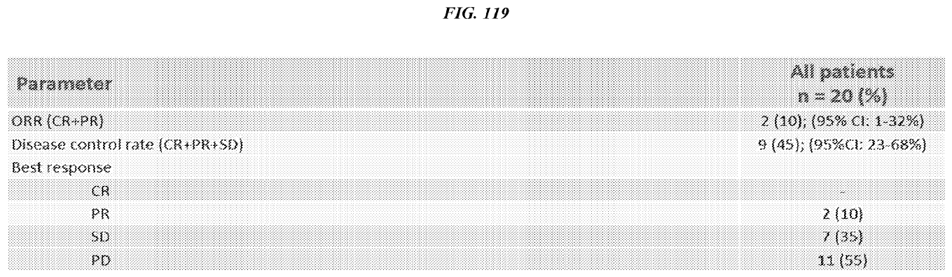

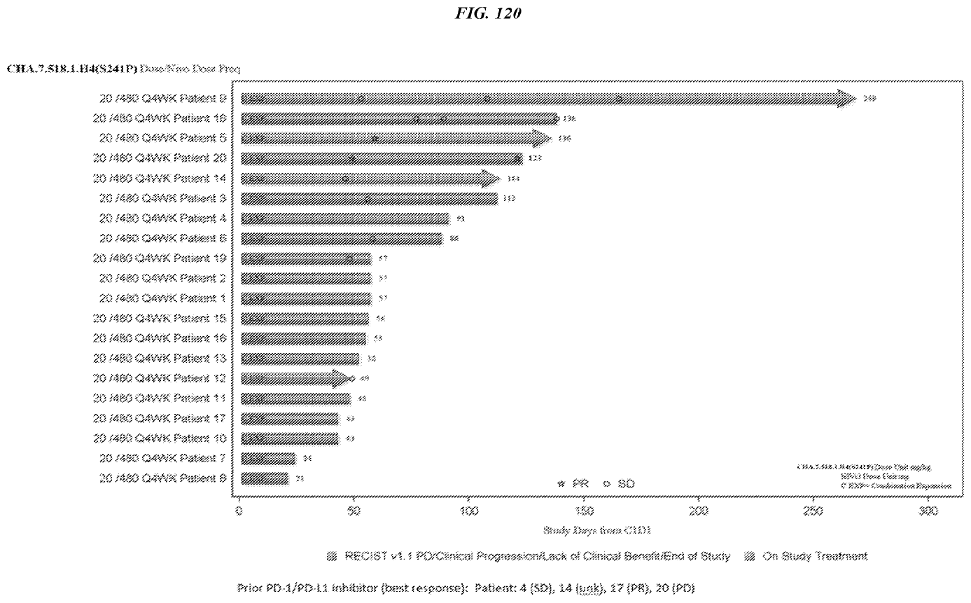

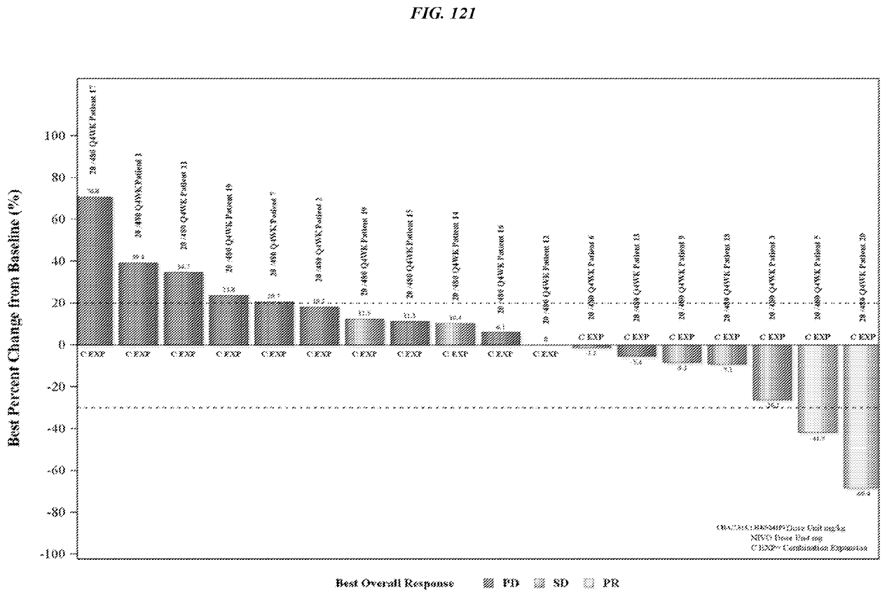

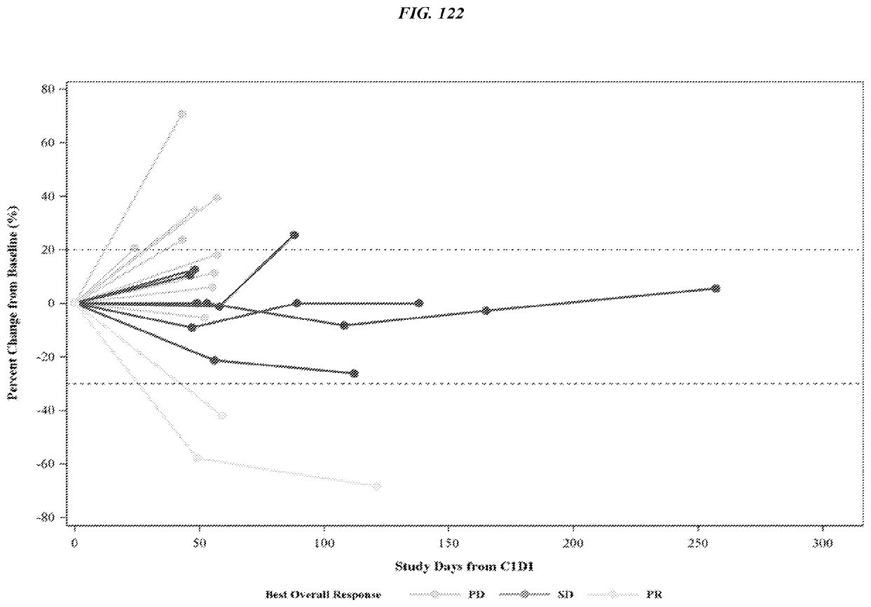

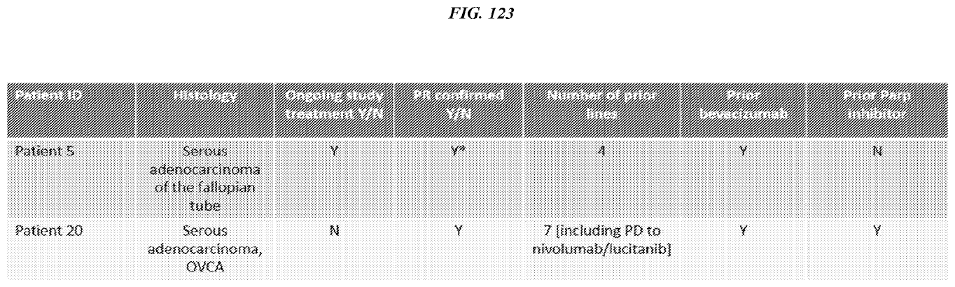

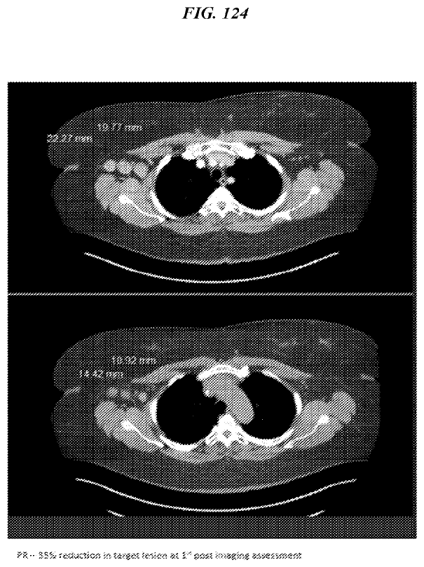

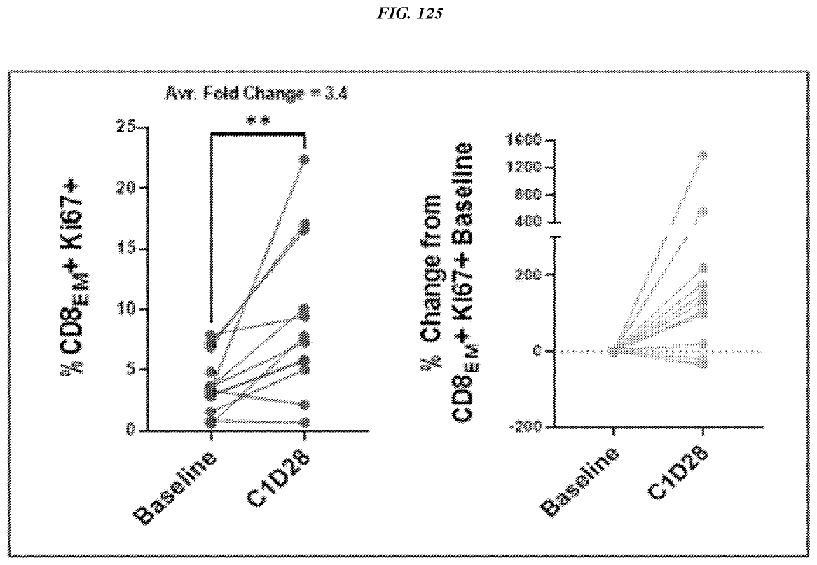

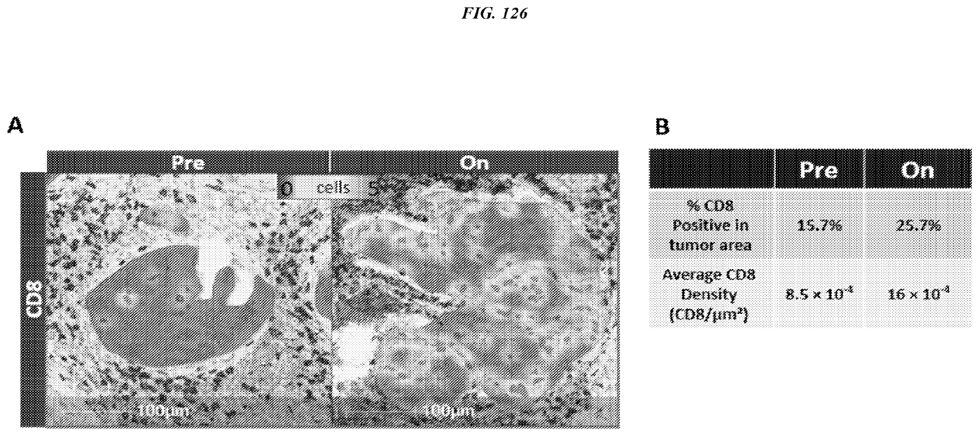

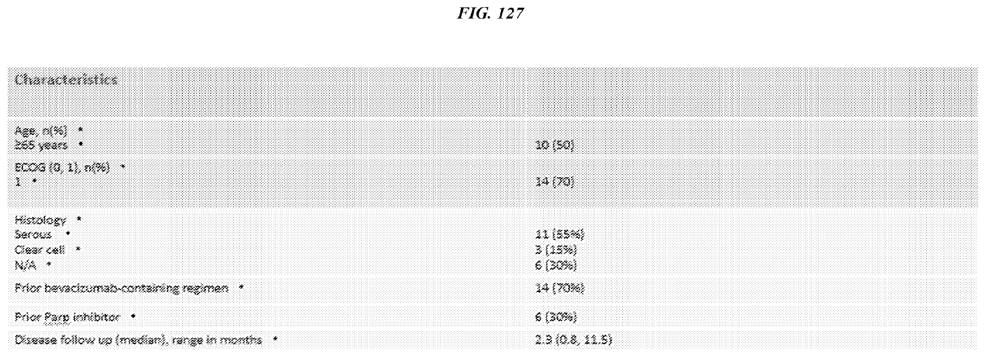

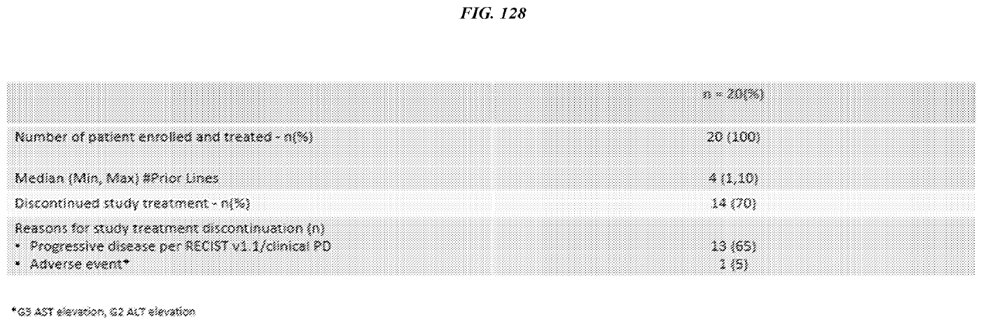

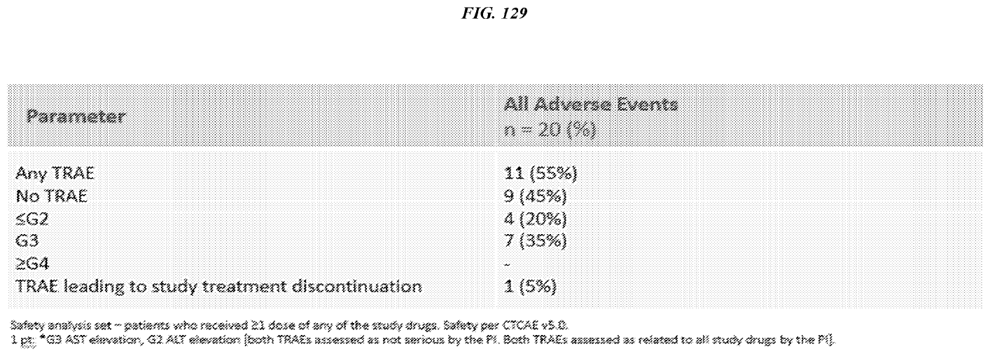

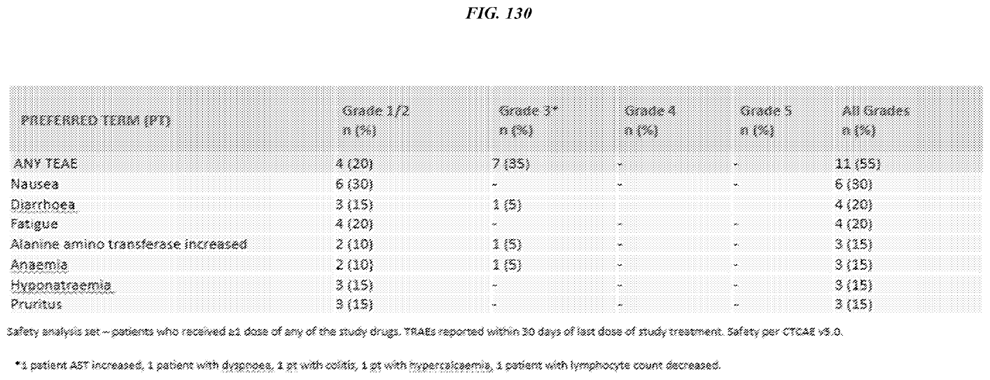

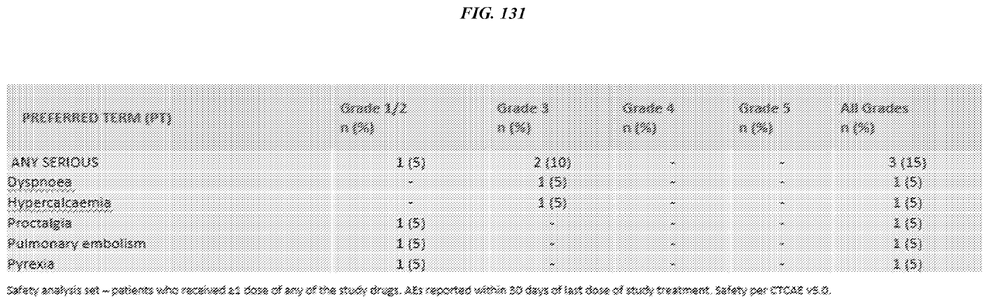

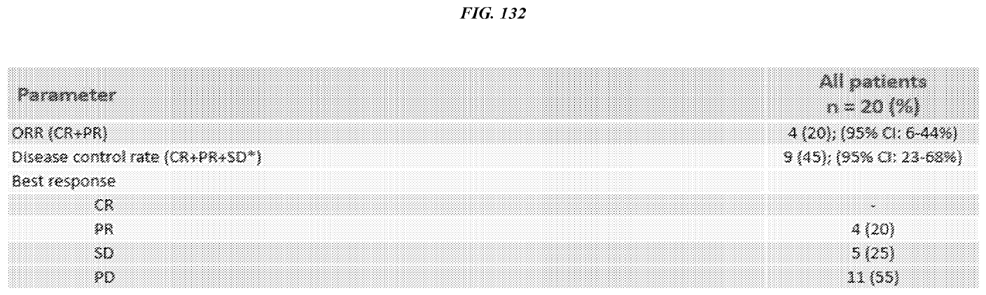

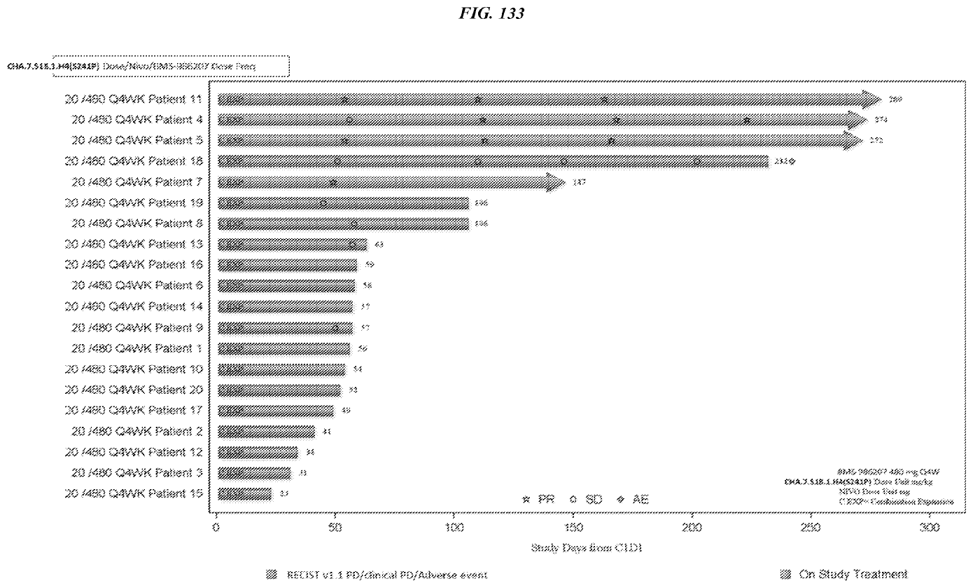

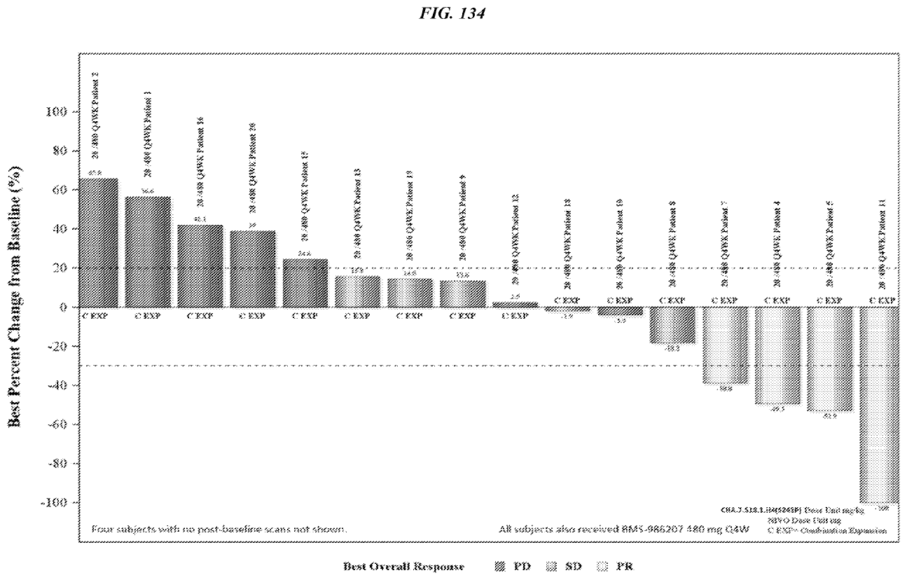

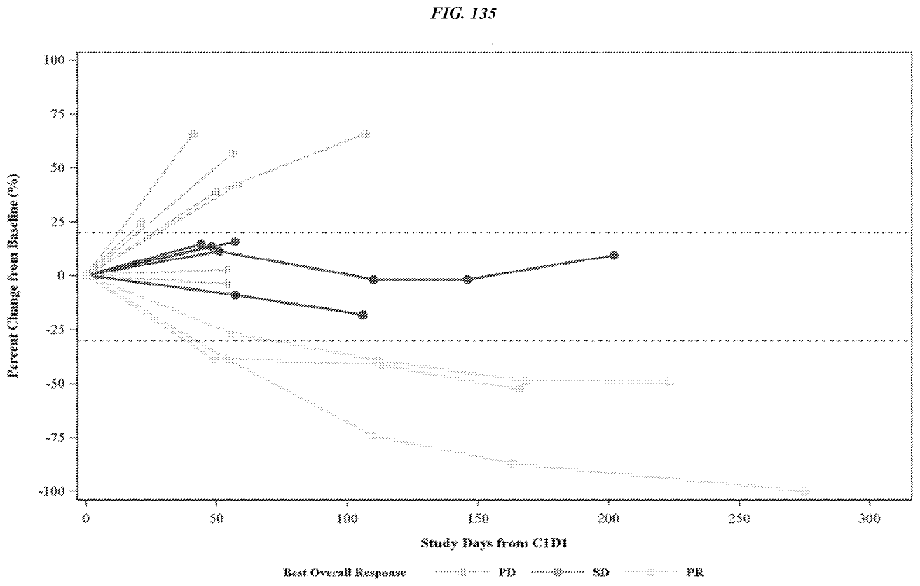

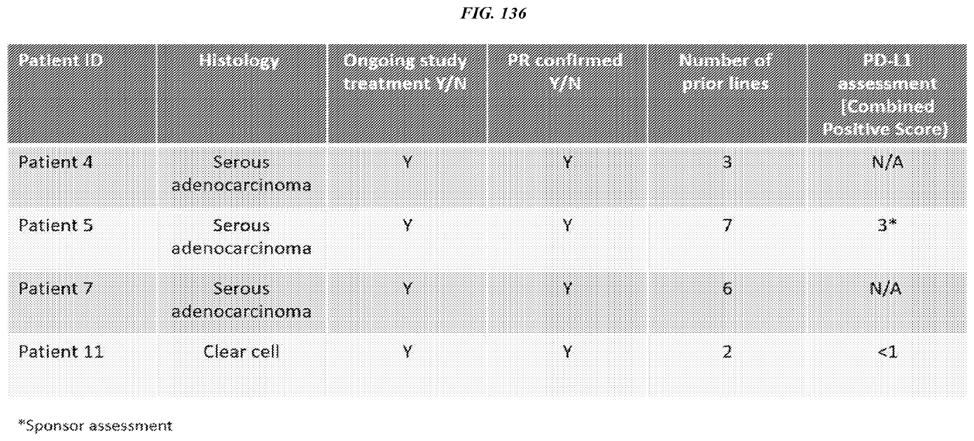

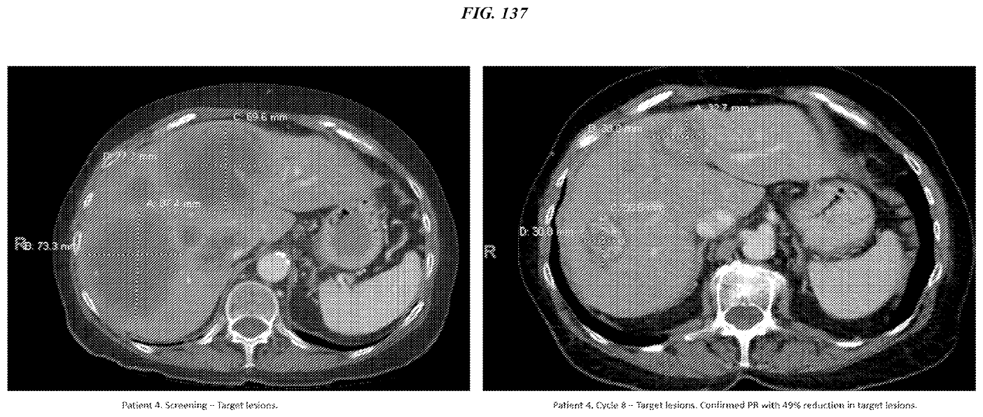

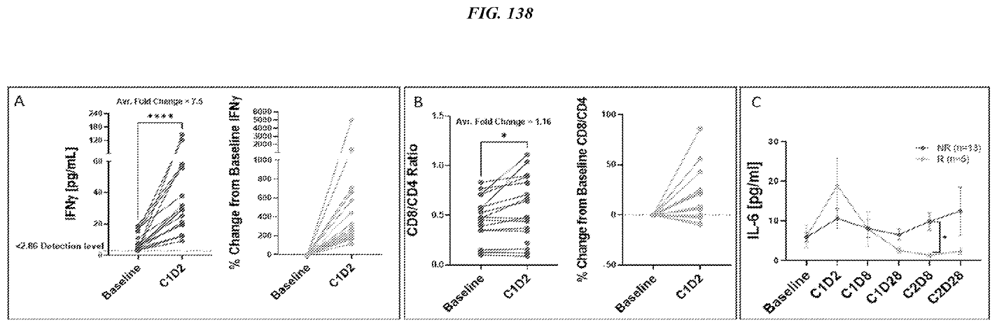

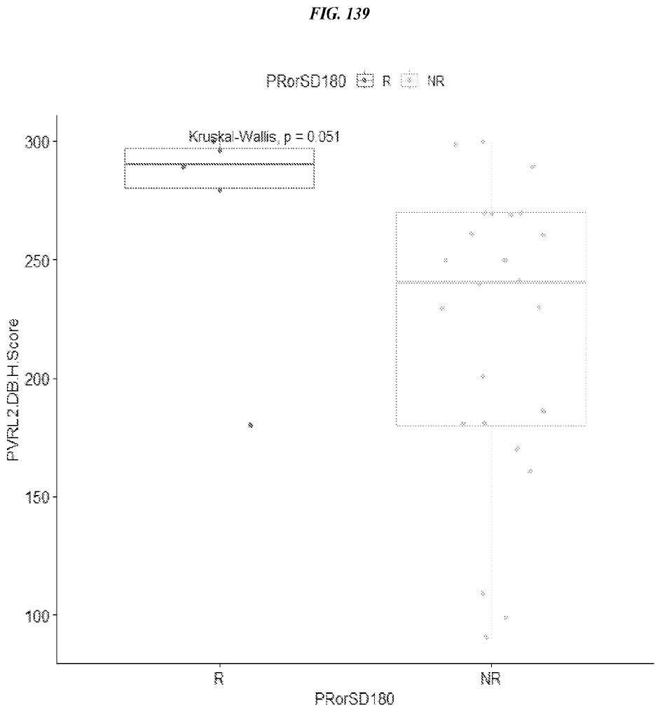

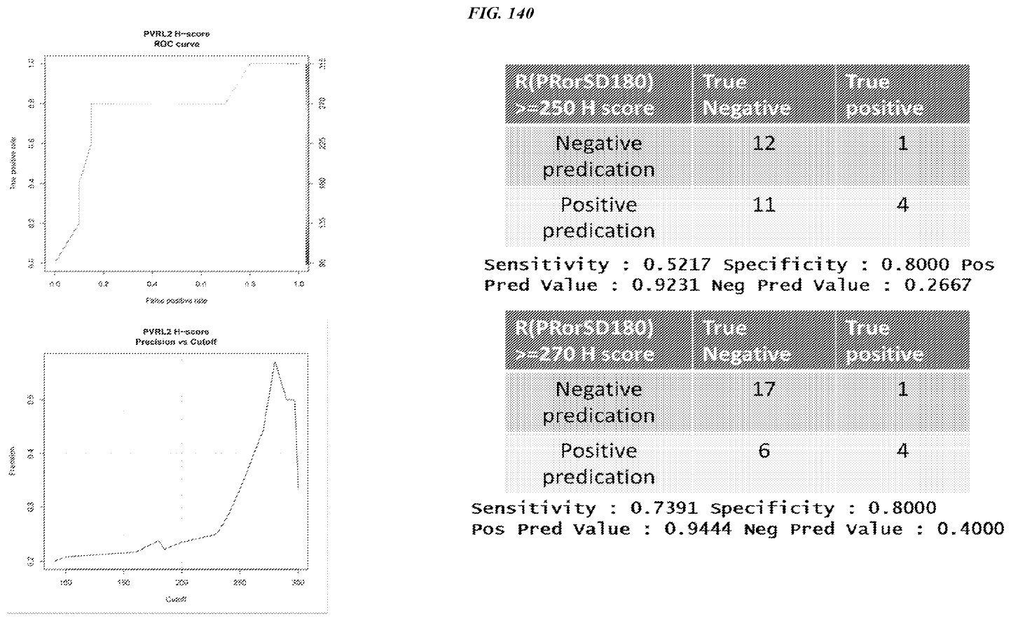

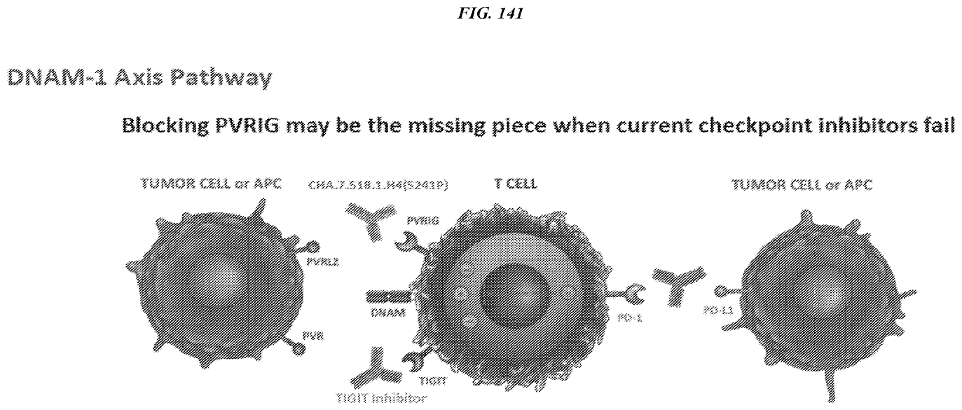

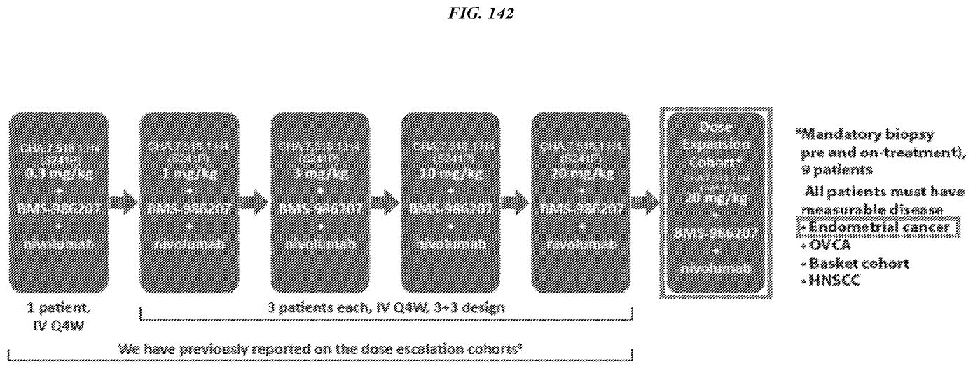

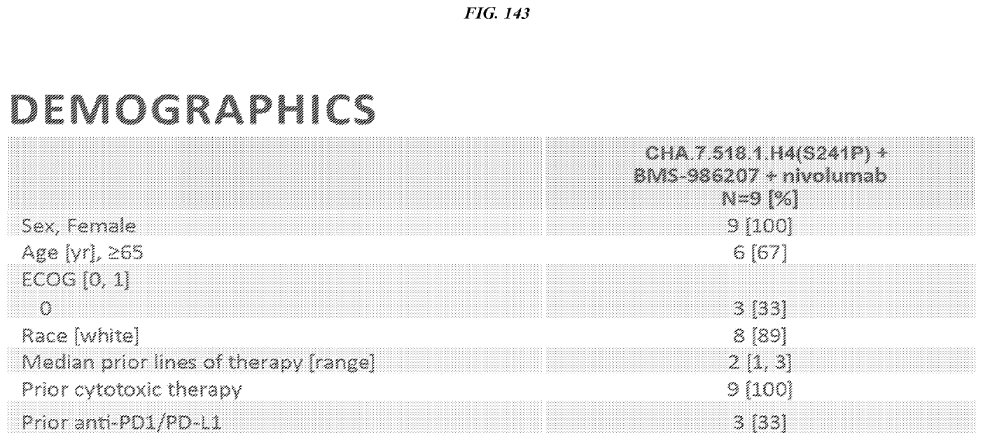

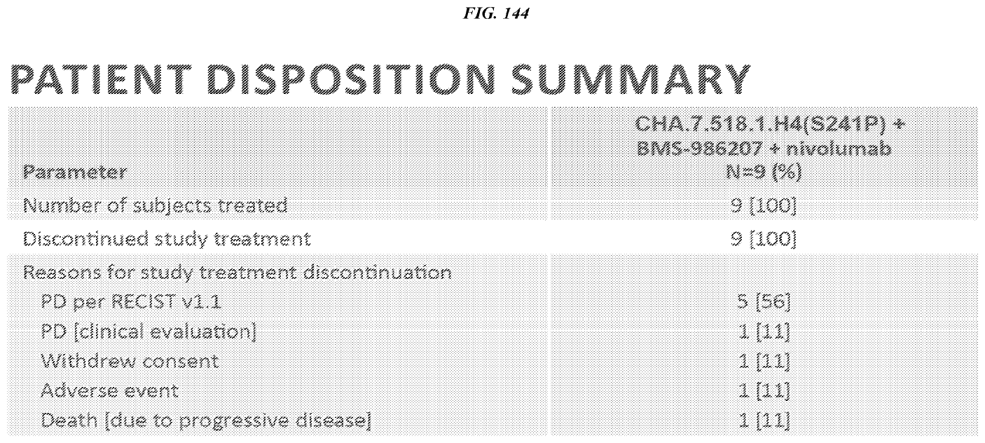

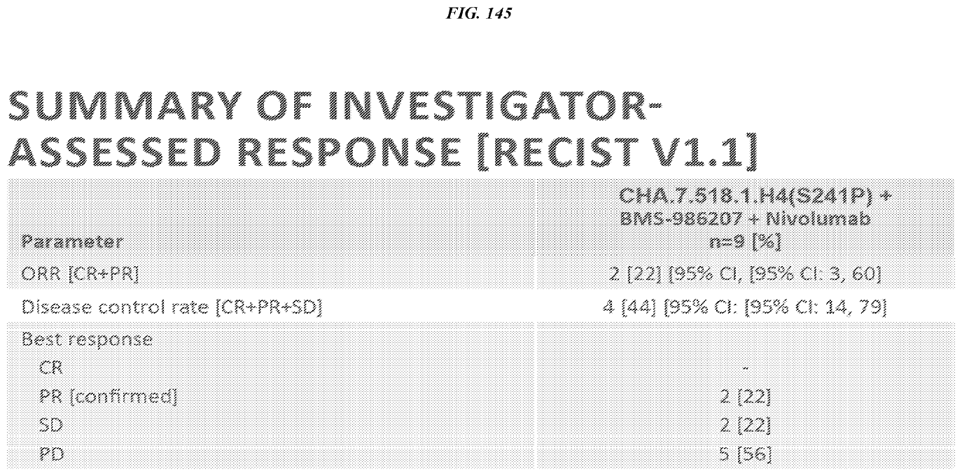

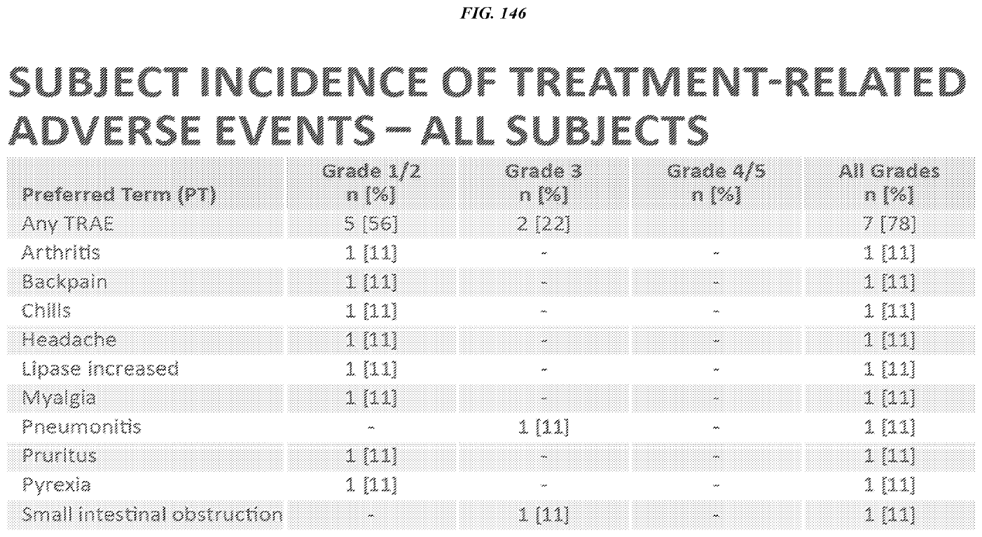

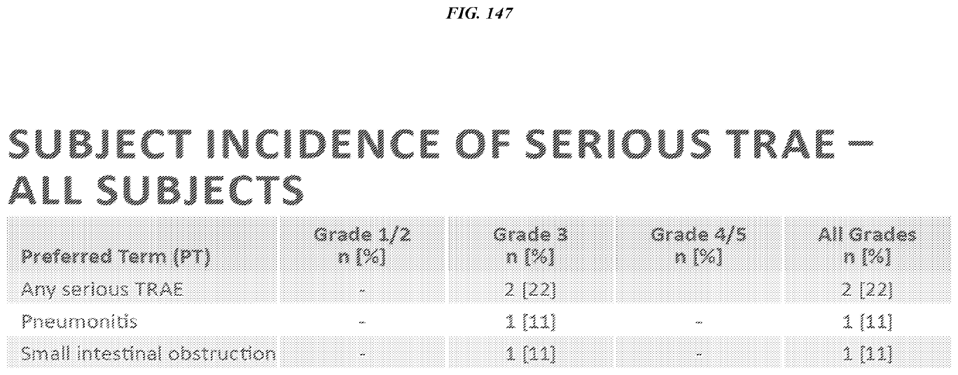

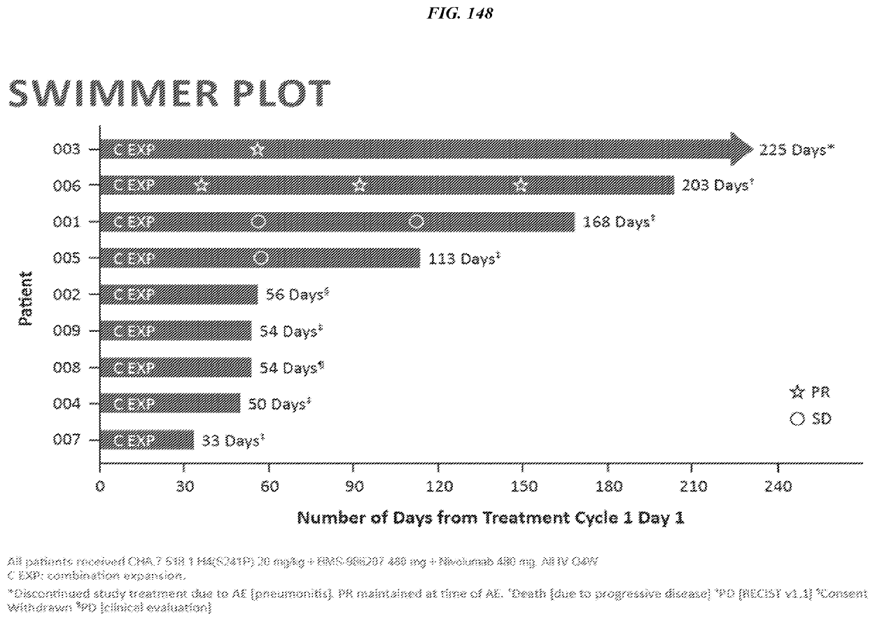

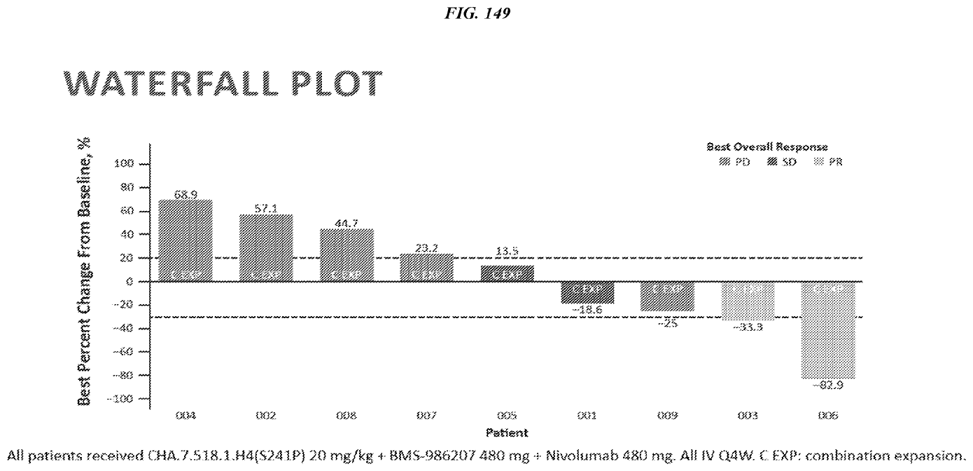

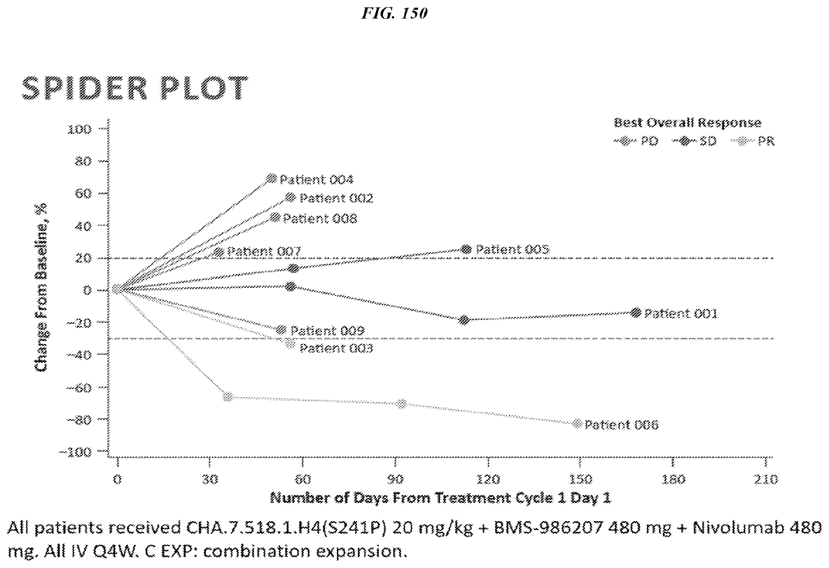

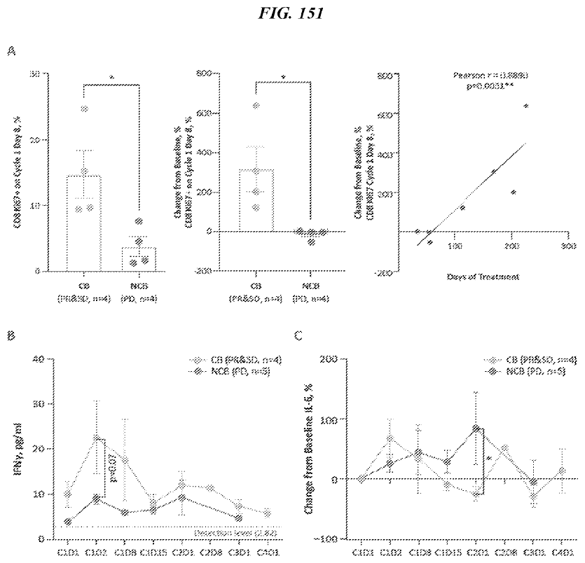

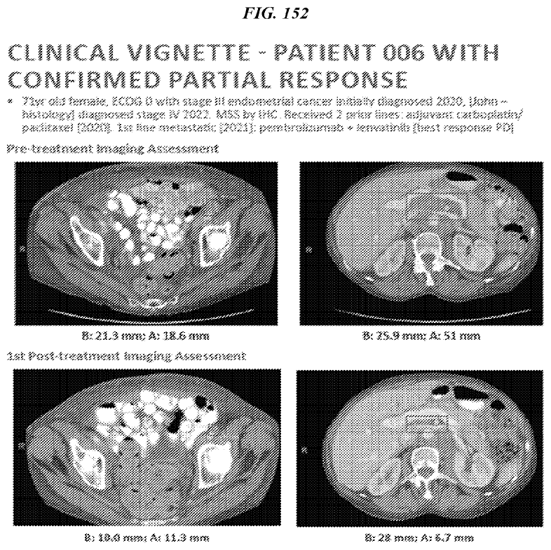

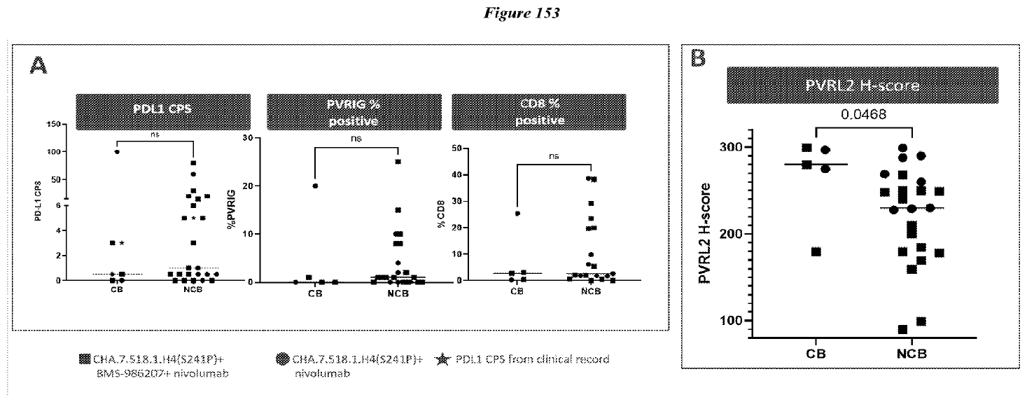

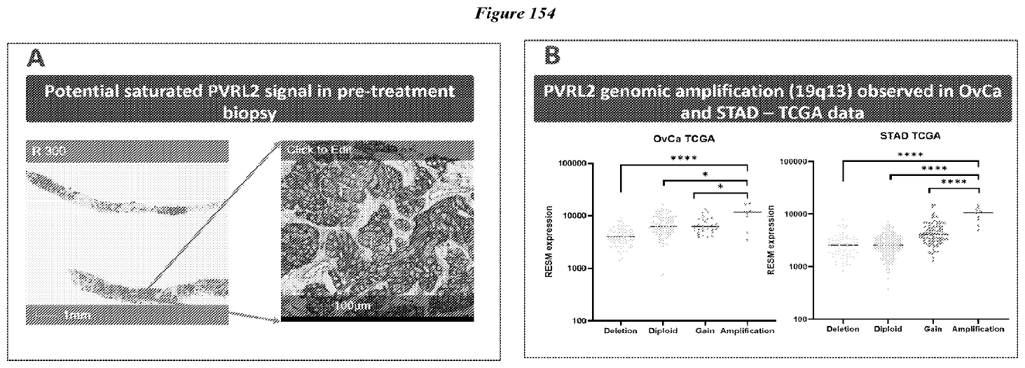

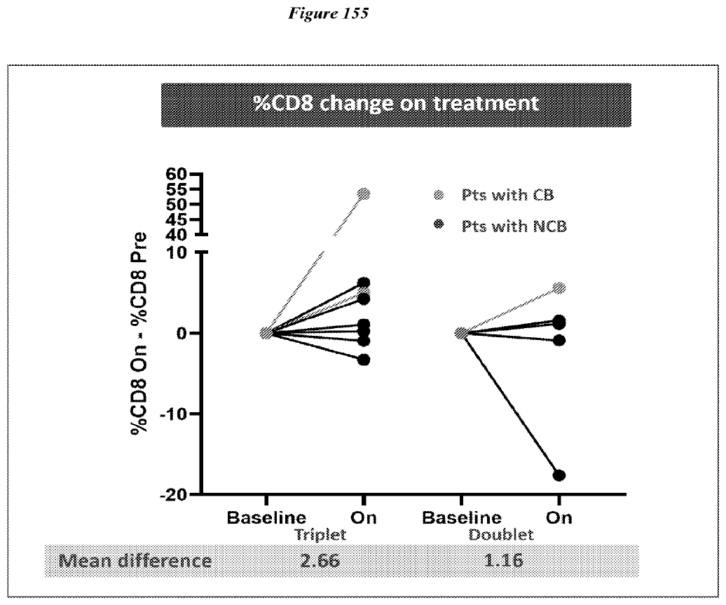

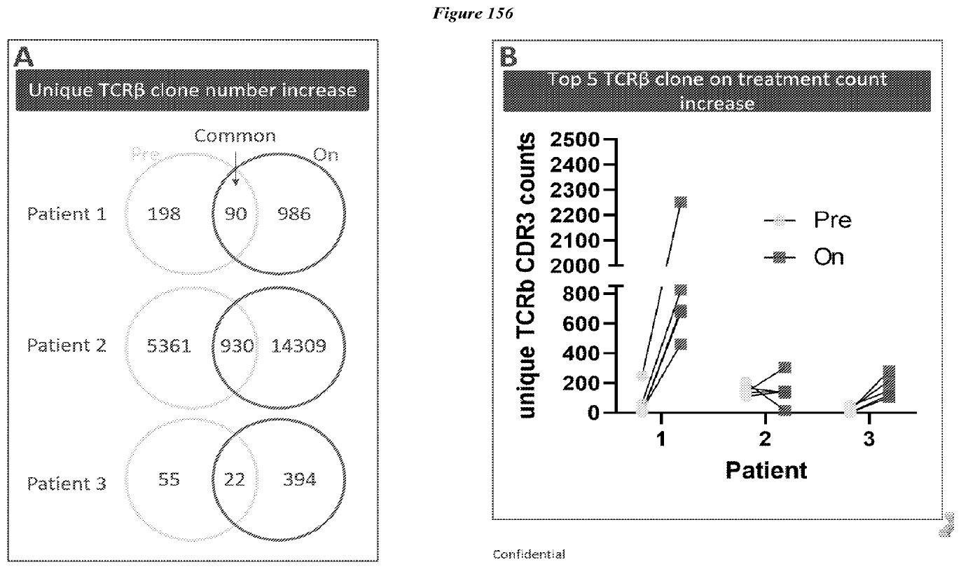

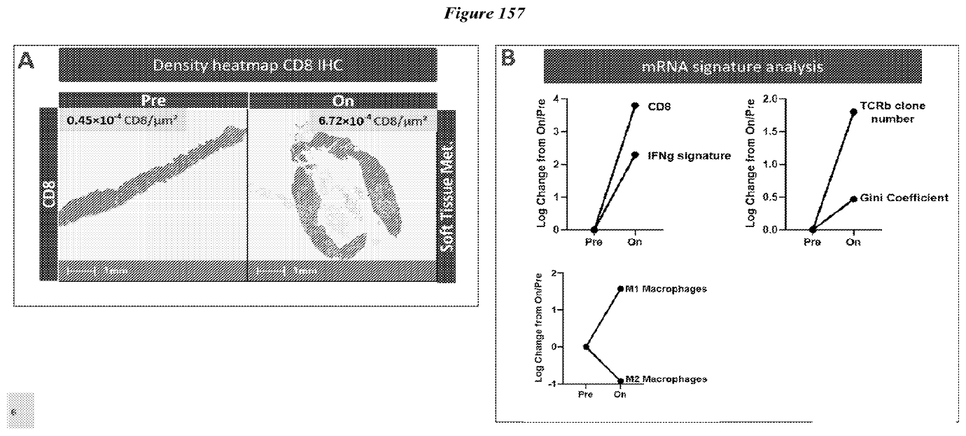

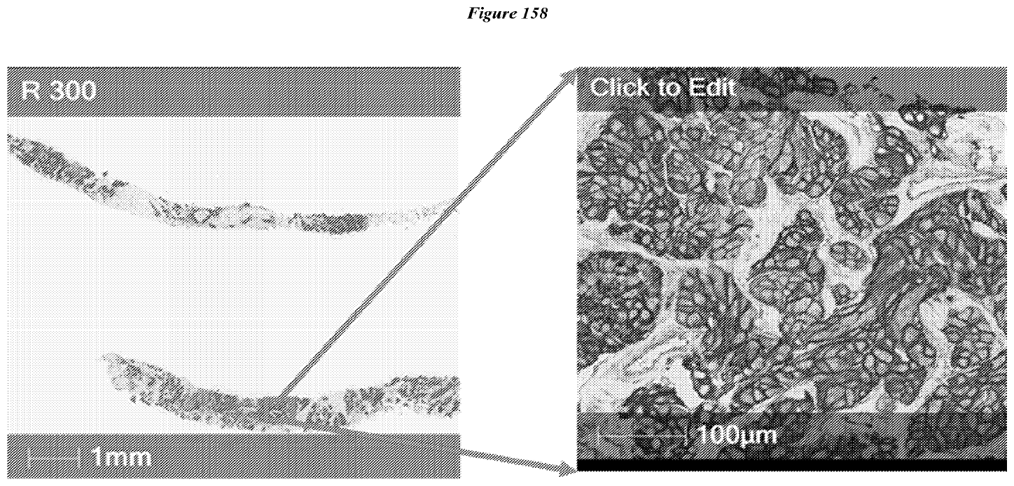

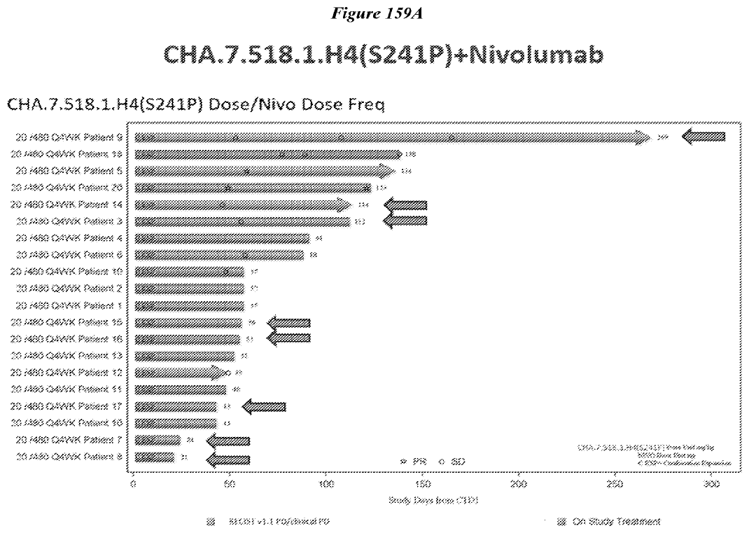

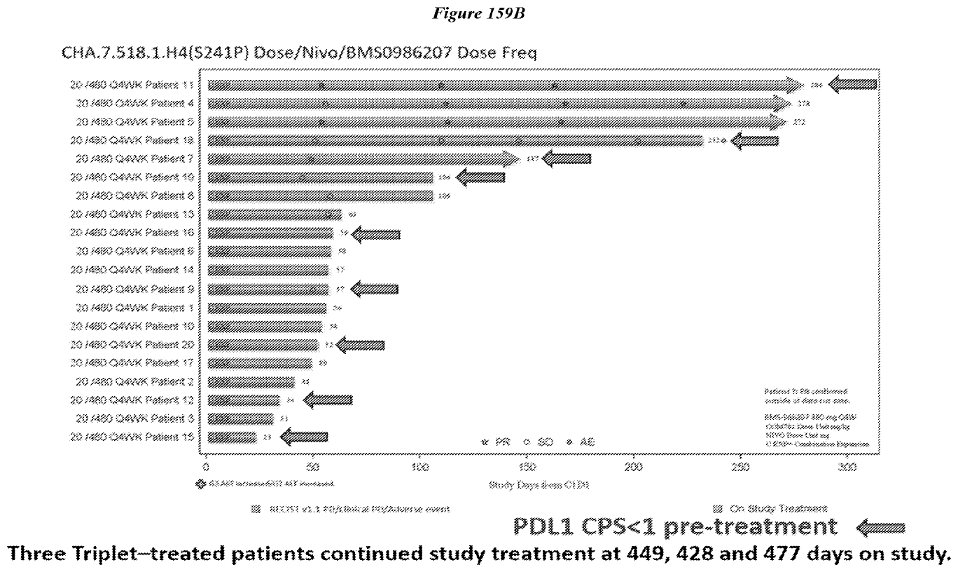

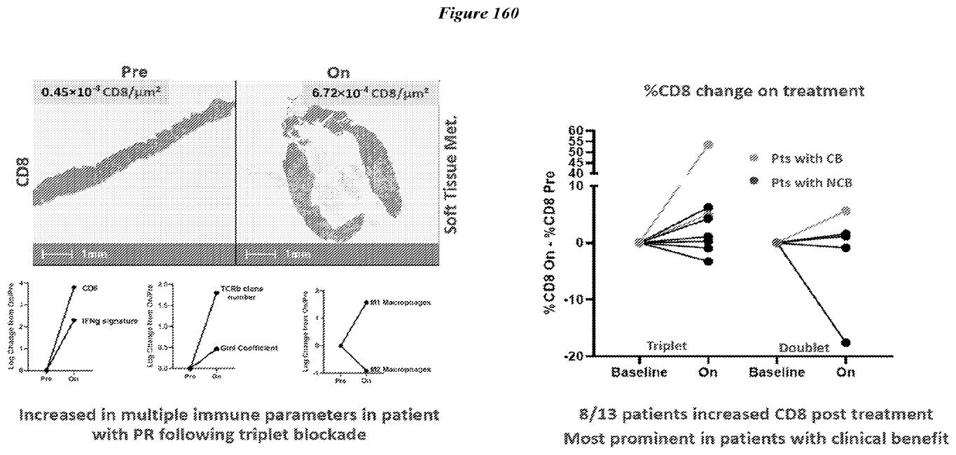

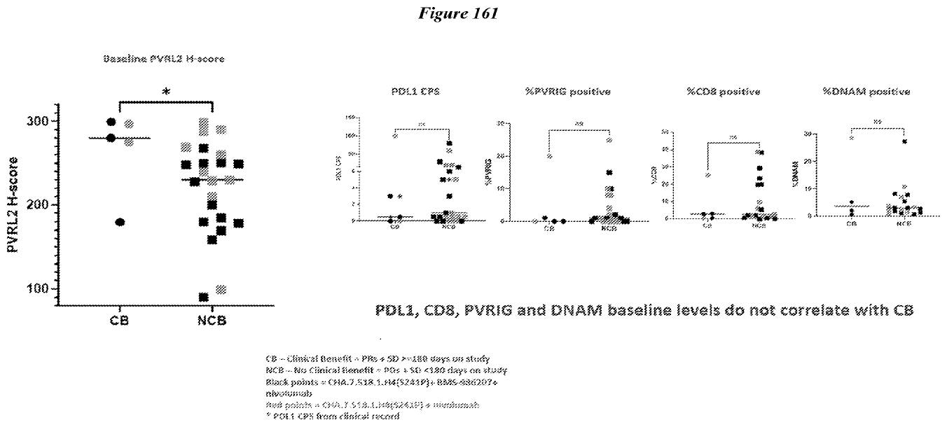

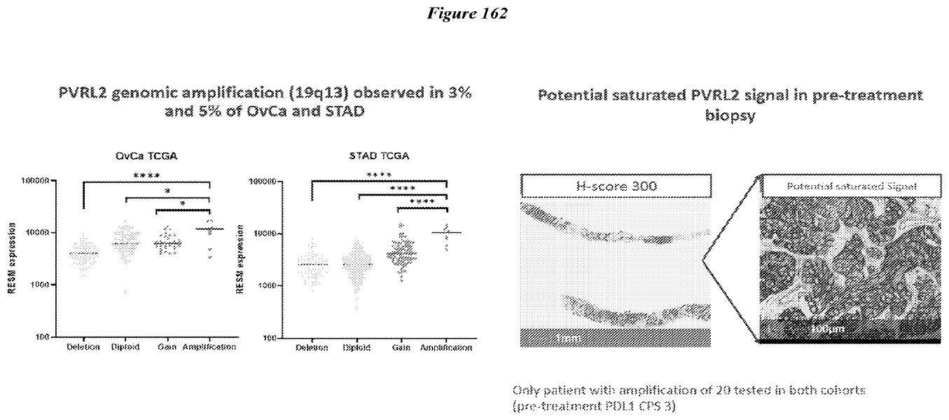

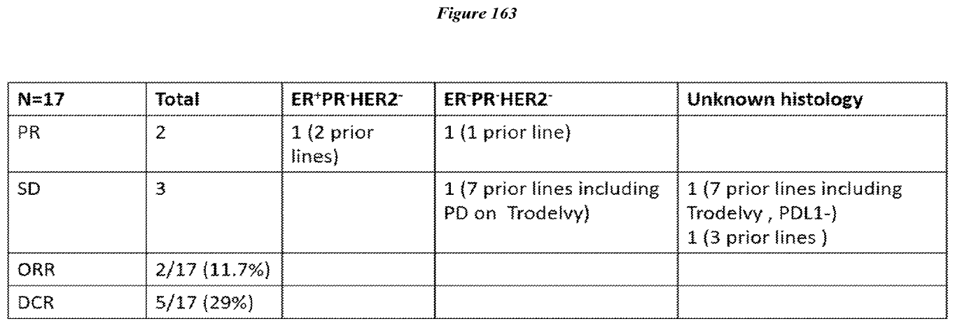

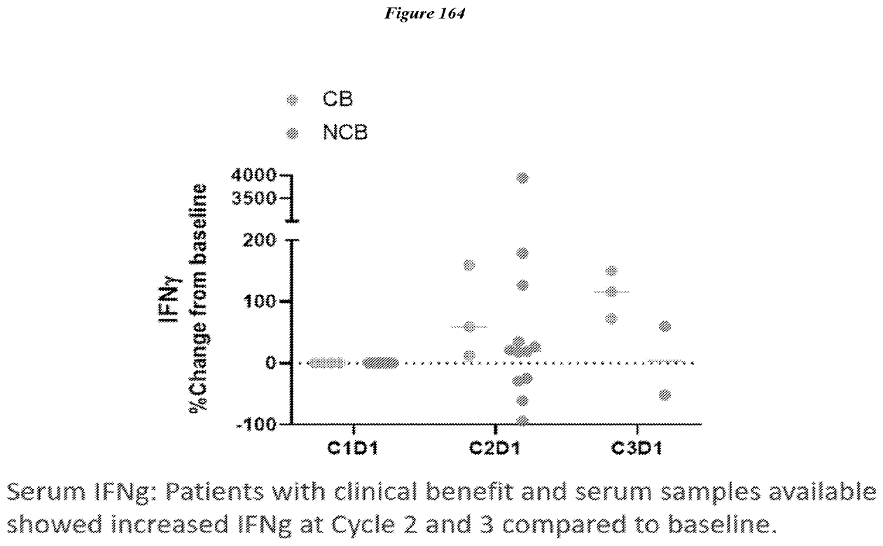

FIG. 79 illustrates increased TME immune activation and TCR clonality in patients with MSS CRC with PR to CHA. 7.518.1.H4 (S241P)±nivolumab combination therapy. Pre- and on-treatment biopsies from CHA.7.518.1.H4 (S241P)±nivolumab treated patients with MSS-CRC were subjected to Personalis®, ImmunoID NeXT analysis. Increased immune infiltration and activation in the TME post CHA.7.518.1.H4 (S241P)±nivolumab therapy was detected. Increased number of clones and increased clonal expansion as was determined by Gini coefficient in the TME post CHA.7.518.1.H4 (S241P)±nivolumab therapy. In both patients the most expanded clone was present prior to treatment initiation.

### Conclusion

By leveraging spatial and scRNA transcriptomics, PVRIG+CD28+CD8+ T cells predominantly localized within TLS interacting with PVRL2+LAMP3+DCs was identified. PVRIG blockade could therefore enhance the differentiation and expansion of stem-like CD8 T cells into effector-like cells. Accordingly, translational data shows increased T cells infiltration and immune activation in response to CHA.7.518.1.H4 (S241P) or CHA.7.518.1.H4 (S241P)±nivolumab, in patients with less-inflamed indications, normally not responsive to approved CPI treatment.

PVRIG, a novel T cell checkpoint, is preferentially expressed in TLS on stem-like memory T cells, potentially inhibiting their expansion

### Example 16: PVRIG, a Novel T Cell Checkpoint, is Preferentially Expressed in TLS on Stem-Like Memory T Cells, Potentially Inhibiting their Expansion

DNAM-1 axis plays an essential role in tumor immunology. DNAM-1 axis contains two parallel dominant complementary inhibitory pathways (PVRIG & TIGIT). TIGIT and PVRIG deliver direct inhibitory signals into T and NK cells. TIGIT/PVRIG has higher affinity to PVR/PVRL2 than DNAM-1 (decoy effect). PVRL2 and PVR expressed on PD-L1 positive and negative tumors; PVRL2 broadly expressed on Dendritic cells subtypes. A schematic diagram of the DNAM-1 axis is provided in FIG. 80.

Intersection between PVRIG/TIGIT and PD-1 pathways supports combination approach to overcome immunotherapy resistance (FIG. 81).

There is a growing body of evidence of early differentiated T stem-like memory cells' (Tscm) importance in response to checkpoint blockade (see FIG. 82). The fraction of Tscm cells is predictive of PD-1 response in melanoma. Also Anti-PD-L1 blockade expands a key population of PD-1-positive Tscm cells which also express TIGIT.

As shown by the data provided in FIG. 83, PVRIG uniquely clusters with early differentiated/Tscm genes.

Spatial transcriptomic analysis of TLS regions shows enrichment of Tscm and DCs while exhausted cells localize to the tumor, as demonstrated by the in-situ MERFISH analysis of tertiary lymphoid structures (TLS) in TME of CRC patients provided in FIG. 84. TLS region harbored a unique co-localization of B-cells, T-cell, Plasma and endothelial cells that are unique to these structures. UMAP shows the clustering of cell populations into distinct cell types based on MRFISH mRNA readout. Boxplot in FIG. 84 demonstrates the cell composition in individual TLS structures or randomly sampled tumor regions. Tscm & Naïve cells localized to TLS while exhausted cells localized to tumor region. Dotplot in FIG. 84 shows the gene expression of selected cell-state markers in a single TLS.

In-situ MERFISH analysis of tertiary lymphoid structures (TLS) in TME of CRC patients provided in FIG. 85 further demonstrates that PVRIG and other genes of the DNAM-1 axis were dominantly expressed in TLS region. Genes of the DNAM-1 axis, PVRIG, TIGIT, CD226, showed dominant expression in TLS region, whereas other immune checkpoints, such as PD-1, CTLA4, TIM3 were expressed in the tumor bed. PVRIG expressing T cells intimately interacted with LAMP3+PVRL2+DC in the TLS of CRC patient.

It is theorized that PVRIG+Tscm interacts with PVRL2+DCs. This theory supports CHA.7.518.1.H4 (S241P) activity in less inflamed PD-L1^{low} tumor types.

Immune modulation in peripheral blood of a patient with PD-L1^{neg} primary peritoneal cancer responding to CHA.7.518.1.H4 (S241P) monotherapy is demonstrated in FIG. 87.

As demonstrated by the data provided in FIGS. 88 and 89, CHA.7.518.1.H4 (S241P) monotherapy induced TME immune modulation in patients with ovarian cancer. 3 out of 4 patients showed an increase in CPS PD-L1. 2 out of 3 patients showed an increase in % CD8. PD-L1 upregulation and increased CD8 infiltration indicate on immune activation induced by CHA.7.518.1.H4 (S241P) monotherapy. Patient with ovarian cancer demonstrating shift from stromal markers towards immune activation in TME following CHA.7.518.1.H4 (S241P) monotherapy.

CHA.7.518.1.H4 (S241P)±nivolumab combination induced TME immune modulation in patients with MSS-CRC, shown by the data provided in FIG. 90. Particularly, 9 of 13 patients showed an increase in CPS PD-L1. 7 of 11 patients showed an increase in % CD8. 5 of 8 patients showed an increase in IFNγ signature.

Extensive TME modulation was observed in MSS-CRC patients partially responding to CHA.7.518.1.H4 (S241P)±nivolumab. FIG. 91 demonstrates that

CHA.7.518.1.H4 (S241P)±nivolumab induced infiltration of new clones as well as expansion of pre-existing clones in both MSS-CRC responding patients.

Combination of CHA.7.518.1.H4 (S241P)±nivolumab induced markers of activated DCs in serum of 3 responding patients. FIG. 92 shows induction of activated-DC markers in serum of 3 patients that clinically responded to CHA.7.518.1.H4 (S241P)±nivolumab compared to non-responders.

### Summary

PVRIG has a unique dominant expression on Tscm, while its ligand, PVRL2, is expressed on also dendritic cells (DCs).

Spatial transcriptomic analysis showed that Tscm and DCs preferentially localize to TLS regions while exhausted T cells localize to the tumor.

PVRIG is dominantly expressed on CD8+ T cells in TLS region.

PVRIG blockade may enhance Tscm activation by DCs in lymph-nodes and TLS, a potential mechanism which could lead to increased T cell expansion and infiltration also into less ‘inflamed’ tumors.

CHA.7.518.1.H4 (S241P)+/−nivolumab induced preliminary anti-tumor activity and TME immune-modulation in patients with MSS-CRC and ovarian carcinoma, typically not responsive to approved CPI.

### Example 17: CHA.7.518.1.H4 (S241P) Plus Nivolumab Demonstrates Preliminary Antitumor Activity and Immune Modulation of Tumor Microenvironment in Patients with Metastatic MSS-CRC and Liver Metastases

### Introduction

CHA.7.518.1.H4 (S241P) is a novel, first in-class, humanized IgG4 monoclonal antibody that binds with high affinity to PVRIG, blocking its interaction with its natural ligand PVRL2 expressed in tumor cells and antigen-presenting-cells.

It has been reported antitumor and pharmacodynamic activity of CHA.7.518.1.H4 (S241P) monotherapy and in combination in several tumor types. See, Vaena D et al., COM701±nivolumab: Results of an ongoing P1 study of safety, tolerability & preliminary antitumor activity in pts with advanced solid malig. J Clin Onco 39, 2021 (suppl 15; abst 2504), incorporated herein by reference in its entirety.

Colorectal cancer (CRC) is a serious and life-threatening cancer with 151,030 new cases in and 52,980 estimated deaths (US estimates 2022). https://cancerstatisticscenter.cancer.org/module/yg6E0ZLc

MSS-CRC patients are in urgent need of new treatment options. MSS-CRC represents 95% of metastatic CRC with limited treatment options.

SOC limited efficacy in refractory MSS-CRC^{1, 2}

| Standard of Care | ORR | mPFS | mOS |
|---|---|---|---|
| Regorafenib/TAS-102 | ~1-2% | ~2 months | ~6-7 months |

1. Van Cutsem, 2012, ASCO
2. Mayer et al., N Eng J Med. 2015; 372:1909-1919

Anti-PD-(L) 1 has shown 0% ORR (Wang et al., JAMA 2021). Approximately 70% of MSS-CRC patients have metastatic liver disease. Presence of liver mets correlates with lack of response to PD-1 combinations 0% ORR (n=54) (Wang et al., JAMA 2021). Anti-PD-1+regorafenib showed 0% ORR (n=47) (Fakih et al., 2021, ASCO). Anti-PD-1+anti-CTLA4 showed 0% ORR (n=17) (El-Khoueiry et al., 2022 ESMO GI).

The present example and accompanying FIGS. 93-104 provide clinical and translational results of the combination of CHA.7.518.1.H4 (S241P)±nivolumab combination therapy in patients with MSS-CRC, including patients with liver mets.

### Study Schema—Combination Dose Escalation and Expansion (FIG. 93)

In this example clinical data obtained from 22 patients is provided:
Combination dose escalation-2 pts [CHA.7.518.1.H4 (S241P) 0.3, 1 mg/kg+nivolumab 360 mg both IV Q3W]
Combination dose expansion-20 pts cohort [CHA.7.518.1.H4 (S241P) 20 mg/kg+nivolumab 480 mg both IV Q4W]

### Key Eligibility Criteria and Study Objectives

### Key Inclusion Criteria [MSS-CRC]:

Histologically confirmed adenocarcinoma of the colon/rectum; Stage IV disease
Prior PD-1/PD-L1 permissible
Documented MSS status by an FDA approved test e.g., genomic testing, IHC for mismatch repair proficient
Disease progression with >2 prior systemic therapies for metastatic CRC that must have included the following: fluroropyrimidines, irinotecan, and oxaliplatin.
Measurable disease [dose expansion cohort]

### Key Exclusion Criteria:

Active autoimmune disease requiring systemic treatment
History of immune-related toxicities on prior immunotherapy treatment leading to discontinuation

### Key Primary Objective:

Safety and tolerability of profile of CHA.7.518.1.H4 (S241P)±nivolumab

### Secondary Objective:

Immunogenicity of CHA.7.518.1.H4 (S241P)±nivolumab.
Antitumor activity of CHA.7.518.1.H4 (S241P)±nivolumab [combination expansion cohort]

### Exploratory Objective:

CHA.7.518.1.H4 (S241P)-mediated pharmacodynamic effect in blood [CHA.7.518.1.H4 (S241P)±nivolumab], immune-related changes [cytokines, immunophenotyping]

Demographics of the patients is provided in FIG. 94.

Patient disposition summary is provided in FIG. 95.

Treatment emergent adverse events in 4 or more patients are summarized in FIG. 96. Incidence of all serious adverse events is provided in FIG. 97.

FIG. 98 provides a summary of investigator assessed response (RECIST v1.1).

A spider plot for MSS-CRC patients following CHA.7.518.1.H4 (S241P)±nivolumab combination treatment is provided in FIG. 99.

FIG. 100 illustrates a waterfall plot for MSS-CRC patients receiving CHA.7.518.1.H4 (S241P)±nivolumab combination treatment.

FIG. 101 provides a swimmer plot for all MSS-CRC patients.

Clinical Vignette—Patient with Partial Response

A 72 yr old woman, MSS-CRC stage IV diagnosed; metastases to liver. Had 2 prior lines in the metastatic setting FOLFOX [best response CR]; FOLFIRI [best response SD]
Mutations: positive TP53 AND APC, KRAS positive
Tumor marker: CEA 36.38 U/mL [screening]; 8.16 U/mL [partial response]

At time of PD due to brain metastases-RECIST v1.1 [partial response] target lesion response maintained with 32% reduction in target lesions and non-target lesions, liver metastases were not measurable

The data of the partial response is provided in FIG. 102.

CHA.7.518.1.H4 (S241P)±Nivolumab Combination Induced TME Immune Modulation in Patients with MSS-CRC

FIG. 103 provides data regarding TME immune modulation following CHA.7.518.1.H4 (S241P)±nivolumab combination therapy for MSS-CRC. 9 of 13 patients showed an increase in CPS PD-L1. 7 of 11 patients showed an increase in % CD8. 5 of 8 patients showed an increase in IFNγ signature.

FIG. 104 provides data showing extensive TME modulation in MSS-CRC patients partially responding to CHA.7.518.1.H4 (S241P)±nivolumab.

### Conclusion

Combination of CHA.7.518.1.H4 (S241P)±nivolumab is well tolerated with a favorable safety profile.

Cumulative ORR 2/22 [9%] CHA.7.518.1.H4 (S241P)±nivolumab; in heavily pretreated subject population [median 3 prior lines].

The ORR [9%] is higher than ORR [1-2%] reported for SOC-regorafenib or TAS-102.

Exploratory analysis demonstrated encouraging preliminary antitumor activity in the patients with liver metastases-ORR 2/17 [12%].

Historically pts with liver metastases appear to be less responsive to ICI.

Translational data demonstrated potent TME immune activation, most notable in responders and consistent with CHA.7.518.1.H4 (S241P) MOA. Such modulation is not typical of CPIs treatment in “cold” indications.

Previous preclinical and clinical translational data have shown most potent immune activation with PVRIG, PD-1 and TIGIT triple blockade, suggesting that adding TIGIT blocker could increase response rate.

### Example 18: CHA.7.518.1.H4 (S241P)±Nivolumab-Preliminary Results of Antitumor Activity from a Phase 1 Study in Patients with Metastatic NSCLC Who have Received Prior PD-1/PD-L1 Inhibitor

### Background

Novel agents are urgently needed for the treatment of patients with metastatic NSCLC including post-immune checkpoint inhibitors (ICI).

CHA.7.518.1.H4 (S241P) is a novel, 1^{st}-in-class ICI that binds to PVRIG, leading to activation of T- and NK-cells.

Historical data with LungMAP, post ICI NSCLC data-1 prior line of ICI in metastatic setting: Median OS 14.5 months (80% CI: 13.9 to 16.1) for ramucirumab+pembrolizumab vs SOC 11.6 months (80% CI 9.9 to 13.0) 1.

It is hypothesized that CHA.7.518.1.H4 (S241P)±nivolumab will demonstrate antitumor activity with a favorable safety and tolerability profile in pts with heavily pretreated NSCLC

Preliminary safety and tolerability² regarding the combination has been reported previously.

The present example provides preliminary results on antitumor activity and long term follow-up.

### Methods

7 patients with NSCLC were enrolled.

5 patients received CHA.7.518.1.H4 (S241P) monotherapy as follows:
1 patient during monotherapy dose escalation was treated with CHA.7.518.1.H4 (S241P) 0.01 mg/kg IV Q3W
4 patients enrolled during monotherapy dose expansion were treated with CHA.7.518.1.H4 (S241P) 20 mg/kg IV Q4W (recommended dose for expansion)

2 patients were enrolled during combination [CHA.7.518.1.H4 (S241P)±nivolumab] dose escalation as follows:
1 patient was treated with CHA.7.518.1.H4 (S241P) 3 mg/kg+nivolumab 360 mg both study drugs IV Q3W
1 patient was treated with CHA.7.518.1.H4 (S241P) 10 mg/kg+nivolumab 480 mg both study drugs IV Q4W

Antitumor activity (per investigator) was evaluated per RECIST v1.1 with CT imaging Q 6/8W depending on schedule of study treatment or at any time point progressive disease is suspected

Study treatment for 2 years unless PD, toxicity, withdrawal of consent, PI discretion.

### Eligibility Criteria and Objectives

### Key Inclusion Criteria:

Histologically confirmed locally advanced or metastatic solid malignancy and has exhausted all available standard treatment or is not a candidate for available standard therapy
ECOG 0-1.
No limitation on the number of prior lines of therapy or prior PD-1/PD-L1 inhibitor

### Key Exclusion Criteria:

Active autoimmune disease requiring systemic treatment
Prior receipt of anti-PVRIG inhibitor
History of immune-related toxicities on prior immunotherapy treatment leading to discontinuation

### Key Primary Objectives:

Safety and tolerability of CHA.7.518.1.H4 (S241P) monotherapy and the combination
Preliminary PK profile of CHA.7.518.1.H4 (S241P)

### Key Secondary Objective:

Preliminary antitumor activity of CHA.7.518.1.H4 (S241P) monotherapy and the combination

### Key Exploratory Objectives:

Immunogenicity of CHA.7.518.1.H4 (S241P)
CHA.7.518.1.H4 (S241P)-mediated pharmacodynamic effect in blood, immune-related changes (cytokines, immunophenotyping)

### Results

Study results and summary are provided in FIGS. 105-113.

### Conclusion

CHA.7.518.1.H4 (S241P)±nivolumab demonstrates preliminary encouraging signal of antitumor activity in a heavily pretreated population of patients with NSCLC with prior ICI treatment.

Most of the patients 4/7 [57%] received≥2 prior lines of ICI.
All 4 patients with SD, with 2/4 [50%] with SD≥6 months.

Median OS (median of 4 prior lines of therapy including multiple ICI in 57% of patients):
CHA.7.518.1.H4 (S241P)±nivolumab 307 days (10 months)
CHA.7.518.1.H4 (S241P) monotherapy 290 days (9.5 months)

Historical data with LungMAP2: post ICI NSCLC data-1 prior line of ICI in metastatic setting, median OS 14.5 months (80% CI: 13.9 to 16.1) for ramucirumab+pembrolizumab vs SOC 11.6 months (80% CI 9.9 to 13.0).

The combination warrants further investigation in a similar patient population.

### REFERENCE

1. Reckamp K L, et al., Phase 2 Randomized Study of Ramucirumab and Pembrolizumab vs SOC in Advanced NSCLC Previously Treated With Immunotherapy-Lung-MAP S1800A. J Clin Oncol. 2022 Jul. 20; 40 (21): 2295-2306
2. Vaena, D A, Fleming G F et al., CHA.7.518.1.H4 (S241P) with or without nivolumab: Results of an ongoing phase 1 study of safety, tolerability and preliminary antitumor activity in patients with advanced solid malignancies (NCT03667716). J Clin Oncol 39, 2021 (suppl 15; abstr 2504).

### Example 19: CHA.7.518.1.H4 (S241P) in Combination with Nivolumab Demonstrates Preliminary Antitumor Activity in Patients with Platinum Resistant Epithelial Ovarian Cancer

### Background

CHA.7.518.1.H4 (S241P) is a novel, 1^{st}-in-class ICI-blocker that binds to PVRIG, a DNAM-1 axis member, leading to activation of T- and NK-cells.

Preliminary anti-tumor activity with CHA.7.518.1.H4 (S241P)±nivolumab has been reported including a confirmed PR with CHA.7.518.1.H4 (S241P) monotherapy in a patient with OVCA [primary peritoneal cancer] 1.

There is a high unmet medical need for the treatment of patients with platinum resistant epithelial ovarian cancer [PROC] defined as disease recurrence<6 months after completion of platinum-based therapy.

Immune checkpoint inhibitors have limited activity in patients with OVCA:
KEYNOTE-100 ORR 8.5% 2.

It was hypothesized that in patients with PROC, dual blockade of PVRIG and PD1 would demonstrate antitumor activity with a favorable safety and tolerability profile.

This example presents results on patient study of the dual combination therapy.

### Methods

20 patients were enrolled with platinum resistant epithelial OVCA, fallopian tube or primary peritoneal cancer; ECOG 0-1.

All patients received CHA.7.518.1.H4 (S241P) 20 mg/kg+nivolumab 480 mg, both IV Q4W.

Anti-tumor activity (per investigator) was evaluated per RECIST v1.1 with CT imaging every 8 weeks or at any time point progressive disease is suspected.

Study treatment for 2 yrs unless PD, toxicity, withdrawal of consent, PI discretion.

Safety assessment was evaluated per CTCAE v4.03.

### Eligibility Criteria and Objectives

### Key Inclusion Criteria:

Histologically confirmed locally advanced or metastatic solid malignancy and has exhausted all available standard treatment or is not a candidate for available standard therapy
Measurable disease
Mandatory biopsy: pre and on-treatment
No limitation on the number of prior lines of therapy or prior PD-1/PD-L1 inhibitor

### Key Exclusion Criteria:

Active autoimmune disease requiring systemic treatment
Prior receipt of anti-PVRIG inhibitor
History of immune-related toxicities on prior immunotherapy treatment leading to discontinuation

### Key Primary Objectives:

Safety and tolerability of CHA.7.518.1.H4 (S241P)±nivolumab
Preliminary PK profile of the combination

### Key Secondary Objective:

Antitumor activity of the combination

### Key Exploratory Objectives:

Immunogenicity of CHA.7.518.1.H4 (S241P)
CHA.7.518.1.H4 (S241P)-mediated pharmacodynamic effect in blood, immune-related changes (cytokines, immunophenotyping

### Results

Study results and summary are provided in FIGS. 114-126.

Clinical vignette—patient 20:

53 yr old female with OVCA, histology-high grade serous adenocarcinoma. The patient received 7 prior lines: adjuvant carboplatin/paclitaxel [2017]. 1^{st} line metastatic

carboplatin/paclitaxel/bevacizumab, bevacizumab maintenance, 2nd line: carboplatin/pegylated liposomal doxorubicin, [PLD stopped for PD] and switched to carboplatin/gemcitabine, maintenance niraparib. 3rd line: pemetrexed [best response PD]. 4th line: nivolumab/lucitanib (TKI) [best response PD]. 5th line: cyclophosphamide [best response SD]. 6th line [2022]: bevacizumab/cyclophosphamide [best response SD].

Lucitanib is an investigational angiogenesis inhibitor, which inhibits vascular endothelial growth factor receptors 1 through 3 (VEGFR 1-3), platelet-derived growth factor receptors alpha and beta (PDGFRα/β) and fibroblast growth factor receptors 1 through 3 (FGFR 1-3).

PR—35% reduction in target lesion was observed at 1^{st} post imaging assessment (FIG. 124).

Pharmacodynamic activation of the immune system with study treatment is demonstrated in FIG. 125. Translational assessment of circulating immune cells showed a positive pharmacodynamic activation of the immune system following PD1 and PVRIG blockade with Increased proliferation of CD8+CD45RA-CCR7-effector memory (EM) T cells as reflected by increase in Ki67

FIG. 126A-B provides results demonstrating TME modulation in partial responding patient [patient 20]. CD8 IHC staining of patient with HGSC treated with CHA.7.518.1.H4 (S241P) and nivolumab, who received 8 prior lines of anti-cancer therapy (including nivolumab). The biopsy taken from lymph node shows extensive CD8 staining as expected. Focusing only on tumor nests in the pre-treatment biopsy, there were very few CD8 cells infiltrating into and between the tumor cells while in the on-treatment biopsy there was a clear infiltration of CD8 into the tumor, demonstrated by both precent of positive CD8 stain in the tumor areas and CD8 density.

CD8 cell density was calculated, using HALO Density Heatmap Algorithm, for tumor region C (blue background, selected manually) biopsy site lymph node both for PRE and ON treatment

### Conclusion

The combination of CHA.7.518.1.H4 (S241P) with nivolumab is well tolerated and has a favorable safety and toxicity profile.

Encouraging preliminary antitumor activity in a heavily pretreated population of 20 patients who had exhausted all standard therapies, with a median number of 6 prior therapies, the dual combination demonstrated was observed with:
Confirmed ORR of 10%, DCR 45% in a heavily pretreated patient population (Encouraging overall response rate of 10%, with 2 partial responses and 1 ongoing at the data cut-off date, disease control rate of 45% (2 confirmed partial responses, 7 stable disease
Antitumor activity in diverse histology-serous adenocarcinoma, clear cell
A confirmed partial response in a patient previously exposed to nivolumab/lucitanib [with best response of PD to the therapy]
Translational assessment of peripheral blood, clearly showed a pharmacodynamic activation of the immune system
One patient with a partial response supported by increased infiltration of CD8 cells into the tumor microenvironment, had high grade serous adenocarcinoma, 7 prior lines of treatment including best response of progressive disease on the combination of nivolumab and lucitanib
Most frequent treatment related adverse events grade 1/2, no grade 4/5 adverse events

Increased serum interferon gamma was observed, confirming the expected immune activation induced by CHA.7.518.1.H4 (S241P) given in combination with nivolumab.

### REFERENCE

1. Vaena, DA, Fleming G F et al., CHA.7.518.1.H4 (S241P) with or without nivolumab: Results of an ongoing phase 1 study of safety, tolerability and preliminary antitumor activity in patients with advanced solid malignancies (NCT03667716). J Clin Oncol 39, 2021 (suppl 15; abstr 2504).
2. Ursula M, Shapira-Frommer R et al., Antitumor activity and safety of pembrolizumab in patients with advanced recurrent OVCA: Final results from the P2 KEYNOTE-100 study. ASCO Annual Meeting 2020.

### Example 20: Triple Blockade of the DNAM-Axis with CHA.7.518.1.H4 (S241P)+BMS-986207+Nivolumab Demonstrates Preliminary Antitumor Activity in Patients with Platinum Resistant OVCA

### Background

CHA.7.518.1.H4 (S241P) is a novel, 1^{st}-in-class ICI-blocker that binds to PVRIG, a DNAM-1 axis member, leading to activation of T- and NK-cells; BMS-986207 [anti-TIGIT inhibitor], nivolumab [PD-1 inhibitor]

Preliminary anti-tumor activity with CHA.7.518.1.H4 (S241P)±nivolumab was reported including a confirmed PR with CHA.7.518.1.H4 (S241P) monotherapy in a patient with OVCA [primary peritoneal cancer]¹.

There is a high unmet medical need for the treatment of patients with platinum resistant epithelial ovarian cancer [PROC], defined as disease recurrence<6 months after completion of platinum-based therapy.

Immune checkpoint inhibitors have limited activity in patients with platinum resistant OVCA.

Pembrolizumab monotherapy ORR 8.5%², pembrolizumab+vibostolimab [anti-TIGIT inhibitor] ORR 8%³.

It is hypothesized that in patients with PROC, triple blockade of PVRIG, TIGIT and PD-1 would demonstrate antitumor activity with a favorable safety and tolerability profile

This example presents results on patient study of the triple combination therapy.

### Methods

20 patients were enrolled with platinum resistant epithelial OVCA, fallopian tube or primary peritoneal cancer; ECOG 0-1.

All patients received CHA.7.518.1.H4 (S241P) 20 mg/kg+BMS-986207±nivolumab both 480 mg; all IV Q4W.

Anti-tumor activity (per investigator) was evaluated per RECIST v1.1 with CT imaging every 8 weeks or at any time point progressive disease is suspected

Study treatment for 2 yrs unless PD, toxicity, withdrawal of consent, PI discretion. Safety assessment was evaluated per CTCAE v5.0.

### Eligibility Criteria and Objectives

### Key Inclusion Criteria:

Histologically confirmed locally advanced or metastatic solid malignancy and has exhausted all available standard treatment or is not a candidate for available standard therapy
Measurable disease
Mandatory biopsy: pre and on-treatment.
No limitation on the number of prior lines of therapy

### Key Exclusion Criteria:

Active autoimmune disease requiring systemic treatment.
Prior receipt of anti-PVRIG, Anti-TIGIT or PD-1/PD-L1 inhibitor
History of immune-related toxicities on prior immunotherapy treatment leading to discontinuation

### Key Primary Objectives:

Safety and tolerability of the combination
Preliminary PK profile of CHA.7.518.1.H4 (S241P)

### Key Secondary Objective:

Preliminary antitumor activity of the combination

### Key Exploratory Objectives:

Immunogenicity of CHA.7.518.1.H4 (S241P)
CHA.7.518.1.H4 (S241P)-mediated pharmacodynamic effect in blood, immune-related changes (cytokines, immunophenotyping)

### Results

Study results and summary are provided in FIGS. 127-138.

Clinical vignette—patient 4:

68 yr old female with high grade serous ovarian carcinoma with a history of 3 prior lines of non-maintenance therapy: carboplatin/paclitaxel, Y90 hepatic radioembolization, carboplatin/pegylated liposomal doxorubicin/bevacizumab [best response for all: PD at 1^{st} re-staging] (FIG. 137).

On study treatment with confirmed partial response.

Pharmacodynamic activation of the immune system with study treatment is demonstrated in FIG. 138. Translational assessment of peripheral blood, including profiling of cytokines and circulating immune cells, clearly showed a positive pharmacodynamic activation of the immune system following PD1/TIGIT/PVRIG blockade. Peripheral IL-6 reduction was detected from D28 post first treatment (C1D28), among patients responding to treatment (partial response or SD>180 days per RECIST) compared to nonresponding patients. The following was observed:
(A) Increase in serum IFN in C1D2 compared to baseline
(B) Increased CD8/CD4 ratio in C1D2 compared to baseline
(C) Post treatment decrease in serum IL-6 with correlation to response

### Conclusion

The combination of CHA.7.518.1.H4 (S241P) in combination with BMS-986207 and nivolumab is well tolerated and has a favorable safety and toxicity profile.

Encouraging preliminary antitumor activity in a heavily pretreated patient population who had exhausted all standard therapies, with a median number of 4 prior therapies the triple combination demonstrated:
Confirmed ORR of 20%, DCR 45% in a heavily pretreated and heterogenous pt population; with 4 confirmed partial responses, out of which 3 are responding for at least 9 months.
Disease control rate of 45% (4 confirmed partial responses, 5 stable disease). Low PD-L1 expression in 2 of the responders [CPS<1 and 3]
Antitumor activity in diverse histology-serous, clear cell\
Translational assessment of peripheral blood, including profiling of cytokines and circulating immune cells, showed a pharmacodynamic activation of the immune system
Most frequent treatment related adverse events grade 1/2, no grade 4/5 treatment related adverse events

Pharmacodynamic changes suggest synergistic immune activation following triplet blockade, as compared to CHA.7.518.1.H4 (S241P) mono and combination results as well as published data.

55% of the patients had high-grade serous adenocarcinoma, including three of the responders

### REFERENCE

1. Vaena, DA, Fleming G F et al., CHA.7.518.1.H4 (S241P) with or without nivolumab: Results of an ongoing phase 1 study of safety, tolerability and preliminary antitumor activity in patients with advanced solid malignancies (NCT03667716). J Clin Oncol 39, 2021 (suppl 15; abstr 2504).
2. Ursula M, Shapira-Frommer R et al., Antitumor activity and safety of pembrolizumab in patients with advanced recurrent OVCA: Final results from the P2 KEYNOTE-100 study. ASCO Annual Meeting 2020.
3. Ruth, P, Gutierrez, M et al., Safety and efficacy of vibostolimab (vibo) plus pembrolizumab (pembro) and coformulation of vibo/pembro in ovarian cancer naive to PD-1/PD-L1 inhibitors. In: Proceedings of the American Association for Cancer Research Annual Meeting 2022; 2022 Apr. 8-13. Philadelphia (PA): AACR; Cancer Res 2022; 82 (12_Suppl): Abstract nr CT180.

### Example 21: CHA.7.518.1.H4 (S241P) in Dual and Triple Combination Demonstrates Preliminary Durable Anti-Tumor Activity and Immune Activation in Patients with Platinum Resistant Ovarian Cancer

CHA.7.518.1.H4 (S241P) in combination with nivolumab+BMS-986207 resulted in encouraging durable confirmed partial responses (ORR 20% triple, 10% dual and DCR 45% for both), supported by immune activation in heavily pretreated patient population and was well tolerated

Three of the patients responded to triplet therapy for at least 9 months and all triplet responders continued treatment as of the data cut-off date.

Over 80% of the patients responding across treatments had the hardest to treat high-grade serous adenocarcinoma histology.

Heavily pretreated patients was observed to have durable responses following triple combination therapy, where the disease had previously progressed on all other treatment options. It is also notable that 75% of those responding to triple therapy had a particularly hard to treat, high-grade serous adenocarcinoma histology. Also very encouraging is an improvement in quality of life as reported by patients to the investigators. With all the responders still on treatment, the median duration of response has not been reached. These encouraging data support the further clinical development of CHA.7.518.1.H4 (S241P) combinations in patients with platinum resistant ovarian cancer where there is a desperate need for new effective, durable, and safe treatment options.

Anti-tumor activity was observed in these heavily pretreated heterogeneous platinum resistant ovarian cancer patients, with a disease control rate of 45% following both triple and dual combination treatment. The combination of drugs was well tolerated with a favorable safety profile consistent with what has previously been reported for CHA.7.518.1.H4 (S241P) with nivolumab+BMS-987346.

Compelling responses were discovered in platinum resistant ovarian cancer patients, including a patient who had 7 prior lines of treatment and progressed on nivolumab but experienced a partial response after treatment with the dual combination of

CHA.7.518.1.H4 (S241P) and nivolumab. And in addition, three patients responding to triple combination therapy for at least 9 months including a patient with >90% reduction in tumor target lesions.

The data support a CHA.7.518.1.H4 (S241P) mediated mechanism of action, it demonstrates that PVRIG blockade is driving T cells into the tumor microenvironment, potentially sensitizing the tumors to PD-1 and TIGIT blockade.

### Example 22: CHA.7.518.1.H4 (S241P) in Combination with Nivolumab+/−BMS-986207 Results in Clinical Benefit which is Correlated to PVRL2 Baseline Levels and is Associated with CD8 Infiltration in Patients with Platinum Resistant Ovarian Cancer

Background: CHA. 7.518.1.H4 (S241P) is a potential 1^{st} in-class, monoclonal antibody that binds to PVRIG, a T cell checkpoint, blocking its interaction with its ligand PVRL2 expressed on tumor cells and antigen presenting cells. Initial antitumor activity of CHA.7.518.1.H4 (S241P)±nivolumab+/−BMS-986207 (anti-TIGIT) in patients (pts) with platinum resistant ovarian carcinoma (PROC) [1,2] has been reported. Checkpoint inhibitors have limited activity in PROC pts, particularly in pts with low PDL1 and reduced T cell infiltration. PVRIG has a dominant expression on stem-like memory T cells (Tscm) and thus CHA.7.518.1.H4 (S241P) could drive T cell increase and clinical response in pts with less inflamed tumors [3]. Preliminary translational assessment of pts with PROC treated with CHA.7.518.1.H4 (S241P)±nivolumab+/−BMS-986207 is presented in this example.

Methods: Pretreatment (n=28) and on treatment (n=21) biopsies (Bx) were collected from pts treated with CHA.7.518.1.H4 (S241P)±nivolumab+/−BMS-986207 Q4W (NCT03667716 and NCT04570839). Bx were subjected to IHC stain with aPDL1, aCD8, aPVRL2 and aPVRIG. Selected Bx were subjected to ImmunoID NeXT assay. Bx of pts from both studies were pooled for analysis.

Results: Pts with PR or SD>180 days (per RECIST) were defined as having clinical benefit (CB pts) vs NCB pts (PD or SD<180). Pre Bx were available for 5 CB pts and 23 NCB pts with additional 2 CB pts with PDL1 archival data.

Clinical responses were independent of PDL1, CD8 and PVRIG baseline levels: 3/7 CB pts had baseline PDL1 CPS<1, median CD8 pre levels were similar for both CB and NCB pts (2.75% and 2.01%) and similar observation was evident for PVRIG pre levels. In contrast, higher baseline PVRL2 H-score was correlated with response with median PVRL2 score of 290 in CB pts vs. 240 in NCB pts (p=0.05).

Pts with paired Bx showed a CD8 increase in 8/13 pts with most prominent increase in CB pts (avg increase of 21% vs 0.65% in NCB pts). In addition, CD8 increase was more evident in pts treated with triple blockade vs. double blockade (4.2% vs. 1.1% difference). Moreover, two CB pts had a substantial CD8 increase (5% and 53.5% difference) despite having both baseline PDL1 CPS<1 and low T cell infiltration. Paired TCR sequencing of two CB pts showed an increase of 2.4 and 5.4 in the number of TCRb clones and most dominant clones on treatment were present pretreatment and expanded in the TME following treatment.

Conclusions: These results may suggest a CHA.7.518.1.H4 (S241P)-driven effect, in line with its Tscm biology, with clinical responses and immune modulation, regardless of the tumor baseline inflammatory status. In addition, the preliminary correlation between the expression of the PVRIG ligand PVRL2 and clinical benefit may suggest the potential of PVRL2 as biomarker to enrich for responding patients.

### Example 23: The Combination of CHA.7.518.1.H4 (S241P)+BMS-986207+Nivolumab Demonstrates Preliminary Antitumor Activity in Patients with Recurrent, Metastatic MSS Endometrial Cancer. NCT04570839

Background: There is a high unmet medical need for the treatment of patients [pts] with microsatellite stable [MSS], recurrent, metastatic, endometrial cancer [EC]. Encouraging preliminary antitumor activity with the triple combination of CHA.7.518.1.H4 (S241P)+BMS-986207±nivolumab in patients with platinum resistant epithelial ovarian cancer [1]. CHA.7.518.1.H4 (S241P) is a novel, 1^{st}-in-class immune checkpoint inhibitor [ICI] that binds to PVRIG, a DNAM-1 axis member, leading to activation of T- and NK-cells; BMS-986207 is an ICI of TIGIT. Nivolumab is an ICI of PD-1 is provided in this example. While not being bound by theory, it is hypothesized that in pts with EC, the triple combination would demonstrate antitumor activity with a favorable safety and tolerability profile. This example provides preliminary results.

Methods: As part of an expansion cohort, 9 patients [pts] with EC were enrolled. All pts received CHA.7.518.1.H4 (S241P) 20 mg/kg+BMS-986207 480 mg+nivolumab 480 mg all IV Q4W. Primary objectives were safety/tolerability, with secondary objective of antitumor activity in pts with EC. Key inclusion criteria: Age≥18 yrs, measurable disease, MSS by IHC or genomic testing, ≤2 prior systemic cytotoxic therapies, prior PD-1/PD-L1 permissible. Key exclusion criteria: prior receipt of anti-PVRIG, anti-TIGIT. Investigator assessed responses were per RECIST v1.1, safety per CTCAE v5.

Results: Median age 71 yrs, median of 2 prior lines of therapy, prior PD-1/PD-L1 3/9 [33%]. All pts received prior cytotoxic therapy. Objective response rate (ORR) 2/9 [22%] pts; 2 pts with SD. Disease control rate [CR+PR+SD] 4/9 [44%]. There were 2 pts with confirmed PR; 1 of these pts was refractory to prior receipt of lenvatinib+pembrolizumab [best response assessment of progressive disease]. Treatment related AEs were reported in 6/9 [67%] the majority 4/6 [67%] were G1 [1 pt each with chills, pyrexia, back pain and pruritus, lipase increased]. No new safety signals were reported.

Conclusion: The combination of CHA.7.518.1.H4 (S241P)+BMS-986207+nivolumab demonstrated encouraging preliminary signal of antitumor activity in pts with EC including in a patient refractory to prior exposure to lenvatinib+pembrolizumab. The triplet combination had a favorable safety/tolerability profile. Additional data analyses and patient follow up are ongoing.

### REFERENCES

1. Moroney J W, Yeku O et al., Triple blockade of the DNAM-axis with CHA.7.518.1.H4 (S241P)+BMS-986207±nivolumab demonstrates preliminary antitumor activity in patients with platinum resistant OVCA. Annals of Oncology (2022) 16 (suppl1): 100104-100104. 10.1016/iotech/iotech100104.

### Example 24: The Combination of CHA.7.518.1.H4 (S241P)+BMS-986207+Nivolumab Demonstrates Preliminary Antitumor Activity in Patients with Recurrent, Metastatic MSS Endometrial Cancer. NCT04570839

Background: CHA.7.518.1.H4 (S241P) is a novel 1^{st} in-class immune checkpoint inhibitor [ICI] that binds to poliovirus receptor related immunoglobulin domain containing [PVRIG] leading to enhanced activation of T and NK-cells.

BMS-986207 is an ICI and anti-TIGIT antibody.

Nivolumab is an ICI that has been well described.

It has been previously reported that the triplet [CHA.7.518.1.H4 (S241P)+BMS-986207±nivolumab] is very well tolerated, has a favorable safety profile and demonstrates preliminary antitumor activity (Dumbrava E E, et al., SITC 2021; Moroney J, et al., ESMO-IO 2022).

There is a high unmet medical need for the treatment of patients with microsatellite stable [MSS], recurrent, metastatic, endometrial cancer.

It was hypothesized that in patients with MSS-endometrial cancer blocking the DNAM-1 axis with a triplet combination consisting of CHA.7.518.1.H4 (S241P), nivolumab and BMS-986207 [an inhibitor of TIGIT] will have an acceptable safety/tolerability profile and also demonstrate antitumor activity.

This example provides preliminary results on safety, tolerability, translational pharmacodynamic readouts and antitumor activity.

### DNAM-1 Axis Pathway

Blocking PVRIG may be the missing piece when current checkpoint inhibitors fail.

Two parallel and complementary inhibitory pathways (PVRIG & TIGIT)-discovered using a discovery platform.

Potential intersection between PVRIG/TIGIT and PD-1 pathway.

CT imaging Q2 cycles-All pts.

Study treatment for 2 yrs unless PD, toxicity, withdrawal of consent, PI discretion-all pts. This example reports on 9 patients [red boxes; right hand panel of FIG. 142]: Combination dose expansion—in addition to CHA.7.518.1.H4 (S241P); all patients received BMS-986207±nivolumab both at 480 mg all study treatment IV Q4W.

Methods: As part of an expansion cohort, 9 patients were enrolled with MSS, recurrent, metastatic endometrial cancer.

All pts received CHA.7.518.1.H4 (S241P) 20 mg/kg+BMS-986207 480 mg+nivolumab 480 mg. All IV Q4W.

Antitumor activity (per investigator) was evaluated per RECIST v1.1 with CT imaging every 8 weeks starting from the first dose during the first 6 cycles of the study and every 16 weeks thereafter (or at any time point progressive disease is suspected).

Safety per CTCAE v5.0

Study treatment for 2 yrs unless PD, toxicity, withdrawal of consent, PI discretion

### Key Eligibility Criteria and Study Objectives [MSS-EC Cohort]

### Key Inclusion Criteria:

Age≥18 yrs
Histologically confirmed endometrial cancer
Documented MSS status by an FDA approved test e.g., genomic testing or IHC
Disease progression with≤2 prior systemic cytotoxic therapies
Prior PD-1/PD-L1 permissible
Measurable disease

### Key Exclusion Criteria:

Active autoimmune disease requiring systemic treatment
History of immune-related toxicities on prior immunotherapy treatment leading to discontinuation
Prior receipt of anti-PVRIG antibody
Prior receipt of Anti-TIGIT antibody

### Key Primary Objective:

Safety and tolerability of profile of the triplet combination

### Secondary Objectives:

Immunogenicity of the triplet
Antitumor activity of the triplet

### Exploratory Objective:

### Pharmacodynamic Activity of the Triplet

### Translational Pharmacodynamic Readouts

Translational assessment of peripheral blood, including profiling of cytokines and circulating immune cells, clearly showed a positive pharmacodynamic activation of the immune system following PD1/TIGIT/PVRIG blockade, which correlates with days of treatment and clinical benefit (CB), i.e., PR and SD>100 days.
(A) Increased proliferation of CD8 T cells, as reflected by increase in Ki67, 8 days post first treatment (C1D8), both by raw values and percent of change compared to baseline, among patients with clinical benefit compared to patients with progressive disease as best response, which also positively correlates to days of treatment.
(B) Overall increase in serum IFNγ (C1D2, C1D8) compared to baseline, which trends to be higher among patients with clinical benefit.
(C) Serum IL-6 reduction at D28 post first treatment (C2D1), among patients with clinical benefit compared to patients with progressive disease.

### Conclusions

The triplet combination of CHA. 7.518.1.H4 (S241P) with nivolumab and BMS-986207 is well tolerated and has a favorable safety and toxicity profile with no new safety signal

The triplet combination demonstrates encouraging preliminary signal of antitumor activity in pts with microsatellite stable recurrent, metastatic endometrial cancer.
Confirmed partial responses in 2/9 [ORR 22%] patients.
Disease control rate 4/9 [44%] patients.
Patients with clinical benefit (PR+SD>100 days) had early pharmacodynamic activation as shown by peripheral IFNγ and CD8.

T cell proliferation increase at first cycle and IL6 decrease at cycle 2 compared to patients with progressive disease.

Confirmed partial response reported with the triplet combination in a patient with prior treatment refractory disease to standard of care treatment with lenvatinib+pembrolizumab.
On study treatment with the triplet combination for 203 days

The data adds to prior disclosures on antitumor activity in multiple tumor types [anal SCC, NSCLC, MSS-CRC with liver metastases, OVCA] of CHA.7.518.1.H4 (S241P) in combination with nivolumab+/−BMS-986207 in patients with prior treatment refractory disease or PD after prior exposure to ICI (Moroney J, et al., ESMO-IO 2022; Yeku O, et al., ESMO-IO 2022; Sullivan R, et al., ESMO-IO, 2022; Overman M J, et al., SITC 2022; Vaena D A, et al., ASCO 2021; and Sullivan R, et al., AACR, 2020)

This data supports the DNAM-1 axis hypothesis and strengthens the need for further development as cancer immunotherapy

### Example 25: Immune Modulation and Baseline Biomarker Correlation with Clinical Benefit Following Treatment with CHA.7.518.1.H4 (S241P)+Nivolumab+/−BMS-986207 in Patients with Platinum Resistant Ovarian Carcinoma

### Background

CHA.7.518.1.H4 (S241P) is a 1^{st} in-class, T-cell checkpoint-inhibitor that binds to PVRIG, blocking its interaction with PVRL2 expressed on tumor and antigen-presenting cells. Initial anti-tumor activity of CHA.7.518.1.H4 (S241P)±nivolumab+/−BMS-986207 (anti-TIGIT) in patients with platinum-resistant ovarian carcinoma (PROC) has been reported [1,2]. Checkpoint inhibitors have limited activity in PROC patients, particularly in patients with reduced PD-L1 and T cell infiltration [3]. This example provides preliminary translational assessment of PROC patients treated with CHA.7.518.1.H4 (S241P)±nivolumab+/−BMS-986207.

### Methods

Pretreatment (n=28) and on-treatment (n=21) biopsies were collected from patients treated with CHA.7.518.1.H4 (S241P)±nivolumab+/−BMS-986207 Q4W (NCT03667716 and NCT04570839) and subjected to IHC stain with anti-PD-L1, anti-CD8, anti-PVRL2 and anti-PVRIG. Selected biopsies were subjected to ImmunoID NeXT assay. Patient IHC data from both studies were pooled for analysis.

Genomic amplification was evaluated by Personalis ImmunoID NeXT assay and algorithms (see the World Wide Web at eacr.org/user_uploads/files/2020-Event-Websites/vLibi20/1_ImmunoID_NeXT_101_535_B.pdf). PVRL2 IHC staining was done using clone 181H3L2 at 0.3 ug/mL, on a Ventana platform and Opti View DAB IHC Detection Kit. Stained images were evaluated by a certified pathologist for tumor H-score and % immune positivity. H-score was calculated by adding the precent of positive cells multiplied for each staining intensity.

### Results

Patients with PR or SD>180 days (per RECIST) were defined as having clinical benefit (CB) versus NCB patients (PD or SD<180).

Clinical responses were independent of PD-L1, CD8 and PVRIG baseline expression: 3/7 CB patients had baseline PD-L1 CPS<1; median CD8 and PVRIG pre-levels were similar for both CB and NCB patients (FIG. 453A). In contrast, higher baseline PVRL2 H-score was correlated with response with median PVRL2 score of 290 in CB versus 240 NCB patients (p=0.05, FIG. 153B). Examining tumor structural genomic-variants (by exome-DNAseq) revealed one responding patient (PR) with a genomic PVRL2-amplification and baseline PVRL2 H-score of 300 (FIG. 156A). TCGA analysis revealed that ovarian and gastric-tumors have an amplification of PVRL2 rate of ˜3-5% which is correlated with higher mRNA expression (FIG. 154B).

Investigating immune modulation, in 8/13 patients with paired biopsies, CD8 increase was shown, with prominent increase in CB patients and trend for stronger CD8 increase in patients treated with triple versus double blockade (FIG. 155). Paired TCR sequencing of three CB patients demonstrated an increase in the number of TCRb clones and most dominant clones on treatment were present pretreatment and expanded in the TME following treatment (FIG. 156). In one patient with PR, CD8 increase demonstrated by IHC and mRNA (deconvolution-score) was accompanied by increase in T-cell clone numbers and clonality and increase in M1 macrophages, while M2 macrophages mRNA-signature decreased (FIG. 157).

Exome DNAseq was performed on 20 ovarian biopsies of patients treated either with CHA.7.518.1.H4 (S241P)+Nivolumab or CHA.7.518.1.H4 (S241P)+BMS-986207±nivolumab (NCT03667716 and NCT04570839). Out of the 20 sample, one responding patient (partial response by RECIST1.1, treated with CHA.7.518.1.H4 (S241P)+BMS-986207±nivolumab) had genomic amplification of the PVRL2 locus (19q13.31/2), with 10-12 predicted copies. Pathological tumor H-score of PVRL2 IHC of the patient is 300, which is the maximal score by this evaluation (FIG. 158).

This patient examples supports the observation that PVRL2 genomic amplification is correlated with high expression of PVRL2, which is further correlated with response to treatment with the triple combination of aPVRIG+aTIGIT+aPD1. Therefore genomic evaluation of PVRL2 gain or amplification can serve as a selection criteria for patients eligible for treatment with aPVRIG+aTIGIT+aPD1. See, FIG. 158.

### Conclusions

These results demonstrate the efficacy of CHA.7.518.1.H4 (S241P) combinations treatment in terms of clinical responses and immune modulation, regardless of the tumor baseline inflammatory status. In addition, the preliminary correlation between the expression of the PVRIG ligand, PVRL2, and clinical benefit may suggest the potential of baseline PVRL2 as biomarker to enrich for responding patients.

### REFERENCES

1. Abstract #159P; ESMO-IO 2022
2. Abstract #158P; ESMO-IO; 2022
3. J Clin Oncol. 2021 Nov. 20; 39 (33): 3671-3681

### Example 26: The Combination of CHA.7.518.1.H4 (S241P)+Nivolumab Demonstrates Preliminary Antitumor Activity in Patients with Metastatic Breast Cancer

Background: CHA.7.518.1.H4 (S241P), a novel, first in-class immune checkpoint inhibitor (ICI) anti-poliovirus receptor related immunoglobulin (PVRIG), that leads to activation of T-cells. PVRL2, the ligand of PVRIG, is highly expressed in breast cancer. Preclinical results showed that the combination of CHA.7.518.1.H4 (S241P) with anti-PD-1 inhibitors synergistically increases T-cell function. Preliminary antitumor activity with objective responses in patients with solid tumors [MSS-CRC, OVCA, anal squamous CA, endometrial cancer] who received CHA.7.518.1.H4 (S241P)+/−BMS986207 (anti-TIGIT antibody)±nivolumab [1,2] was previously reported. While not being bound by theory, it is hypothesized that CHA.7.518.1.H4 (S241P)±nivolumab will be well tolerated and will demonstrate antitumor efficacy. Results from the ongoing dose expansion cohort with CHA.7.518.1.H4 (S241P)±nivolumab in patients with metastatic breast cancer (NCT03667716) are provided in this example.

Methods: A total of 17 patients with metastatic breast cancer received CHA.7.518.1.H4 (S241P) 20 mg/kg+nivolumab 480 mg, both IV Q4 weeks. Primary objectives were to determine safety and tolerability and secondary objective was to evaluate preliminary antitumor activity. Key inclusion criteria: Age≥18 years, histologically confirmed locally advanced or metastatic breast cancer (regardless of ER/PR and HER2 status) with measurable disease, who exhausted all available standard treatments. Prior treatment with anti-PD (L)-1, anti-CTLA-4 ICI was permissible. Key exclusion criteria: history of immune-related events that led to immunotherapy treatment discontinuation, history of pneumonitis. Safety was evaluated per CTCAE v4.03 and investigator responses per RECIST v1.1.

Results: No new safety signal was reported, and no dose-limiting toxicities were observed. Objective response rate 2/17 patients [12%]—a complete response was achieved in a patient with metastatic ER+/PR−, HER2-invasive ductal carcinoma with pretreatment biopsy with PDL1 CPS of 3 and PVRL2 tumor H-score of 300. The patient was also TMB-low (5 Mut/Mb) and received 3 prior lines of therapy and continues the study treatment (567 days). Another patient with metastatic triple-negative breast cancer, PD-L1 negative, TMB-low (1 Mut/Mb), with 4 prior lines of therapy with confirmed partial response (PR) remained on study treatment (296 days), 3 pts with stable disease (SD); Disease control rate [CR+PR+SD] 5/17 [29%].

Conclusion: Encouraging preliminary antitumor activity with PR and CR are reported in heavily pretreated patients with TMB-low metastatic breast cancer with the combination of CHA.7.518.1.H4 (S241P)±nivolumab. The combination is well tolerated with no new safety signal.

### REFERENCES

1. Moroney J A, Yeku O et al: Triple blockade of the DNAM-axis with COM701+BMS-986207±nivolumab demonstrates preliminary antitumor activity in patients with platinum resistant OVCA. Annals of Oncology (2022) 16 (suppl_1): 100104-100104. 10.1016/iotech/iotech 100104
2. Drew Rasco, Ecaterina Dumbrava et al. COM701 plus nivolumab demonstrates preliminary antitumor activity and immune modulation of tumor microenvironment in patients with metastatic MSS-CRC and liver metastases. Journal for Immunotherapy of Cancer November 2022, 10 (Suppl 2) A690; DOI: 10.1136/jitc-2022-SITC2022.0659

### Example 27: Durable Responses with Triple Blockade of the DNAM-1 Axis with CHA.7.518.1.H4 (S241P)+BMS-986207+Nivolumab in Patients with Platinum Resistant Ovarian Cancer (OVCA)

Background: Treatment options for patients with platinum resistant ovarian cancer [PROC] are limited. Immune checkpoint inhibitors (ICI) have limited activity in PROC, therefore clinical studies evaluating novel therapies are urgently needed. Durable partial responses and a complete response have been previously reported with

CHA.7.518.1.H4 (S241P)+/−BMS986207±nivolumab1. CHA.7.518.1.H4 (S241P) is a novel, 1^{st} in-class ICI binding to PVRIG (anti-PVRIG), that leads to activation of T-cells. BMS-986207 is an ICI blocker of TIGIT (anti-TIGIT). Longer term follow-up showing continued durable responses in pts with PROC treated with a triple immunotherapy combination blocking the DNAM-1 axis with CHA.7.518.1.H4 (S241P)+BMS-986207±nivolumab is reported in this example.

Methods: 20 patients with PROC were enrolled and treated with CHA.7.518.1.H4 (S241P) 20 mg/kg+BMS-986207 480 mg+nivolumab 480 mg IV Q4W. Primary objectives [obj] were safety/tolerability; secondary obj was antitumor activity. Key inclusion criteria: Age≥18 yrs, histologically confirmed advanced malignancies and exhausted all available standard tx. Key exclusion criteria: prior receipt of any inhibitor of PVRIG, TIGIT, or PD-(L)-1. Investigator assessed responses per RECIST v1.1, safety per CTCAE v5.0.

Results: No new safety signals are reported. The combination was well tolerated. There were 4/20 [20%] patients with confirmed PR and 5 pts with SD with a DCR [CR+PR+SD] 9/20 [45%], no CRs. Median [med] age 61 yr, med number of prior lines of therapy-4 [range 1-10]. Histology of patients with PR-high grade serous adenoCA [3 pts], clear cell histology [1 pt]. Three pts continued study treatment at 449, 428 and 477 days, 1 pt with high grade serous adenoCA was on study treatment for 222 days.

Conclusion: Continued durable confirmed partial responses by blocking the DNAM-1 axis with the combination of CHA.7.518.1.H4 (S241P)+BMS-986207±nivolumab in pts with heavily pre-treated PROC.

### REFERENCES

1. Moroney J A, Yeku O et al: Triple blockade of the DNAM-axis with COM701+BMS-986207±nivolumab demonstrates preliminary antitumor activity in patients with platinum resistant OVCA. Annals of Oncology (2022) 16 (suppl_1): 100104-100104. 10.1016/iotech/iotech100104

### Example 28: Clinical Study of CHA.7.518.1.H4 (S241P) and Combination Therapies

### Summary

PVRIG is a T and NK checkpoint inhibitor, part of the DNAM-1 axis. PVRIG binds to PVRL2, that is expressed on cancer and myeloid cells. PVRIG has a unique dominant expression on early differentiated stem-cell like memory T cells (Tscm) in TLS. While not being limited by theory, PVRIG blockade may enhance Tscm activation by DCs, a potential mechanism which could lead to increased T cell expansion and infiltration into less “inflamed” tumors

Translational data demonstrates that CHA.7.518.1.H4 (S241P) increases infiltration and activation of T cells in TME in patients with PVRL2+PD-L1^{low} tumors including MSS CRC and OVCA patients.

CHA.7.518.1.H4 (S241P) displays favorable safety and tolerability profile with nivolumab and BMS-986207.

Promising antitumor activity of CHA.7.518.1.H4 (S241P)+/−BMS-986207+nivolumab was observed with the following: tumor types with low PD-L1 expression (“cold tumors”); subjects with prior treatment refractory disease; subjects with prior exposure to ICI.

This translational data supports mechanism of action (MOA).
Breast cancer data-CHA.7.518.1.H4 (S241P)±nivolumab

A total of 17 subjects were enrolled, with 2 responders and 3 best response of SD to the treatment with CHA.7.518.1.H4 (S241P) and nivolumab.

Confirmed CR (confirmed PR to CR):
ER+/PR−, HER2-invasive ductal carcinoma, had 3 prior lines and refractory to last prior therapy [cape+irinotecan+abemaciclib+fulvestrant]
PVRL2 H-score 300, PDL1 CPS 3, TMB low

Confirmed PR (TNBC, 4 prior lines, PD-L1 negative, TMB-low).

A disease control rate (DCR) of 5/17 (29%) was observed.

3 cases of SD were observed: all of which are PDL1 CPS≤1, TMB-low. One measured for baseline PVR2: H-score 297.

One patient was observed with G3 TRAE of pneumonitis (recovered). No greater than G4 TRAEs were observed.

A total of 17 subjects enrolled with 2 responders and 3 best response of SD

Confirmed PR was observed.

Confirmed CR (confirmed PR to CR) was observed in a 46 year old white female diagnosed with adenocarcinoma.
Clinical vignette [P1b expansion breast cohort]—subject with CR in response to treatment

46 year old white female diagnosed with adenocarcinoma of the breast (grade unknown) with s/p bilateral mastectomies; TAH+BSO; and ECOG 1.

Prior treatment includes Adriamycin (doxorubicin), cyclophosphamide, paclitaxel (taxol) (AC-T) as adjuvant therapy with tamoxifen. The patent was refractory to last prior therapy with capecitabine+irinotecan+abemaciclib+fulvestrant.

Metastatic setting: TAC (Taxotere, adriamycin, cyclophosphamide) (best response*).

Metastatic setting: cape+irinotecan+abemaciclib+fulvestrant (best response PD*).

At screening: abdominal metastases [colon, peritoneum]; Mutation status: BRCA negative; TMB 5; prior PD-L1+ (50%), TP53+, ER+, PR−, HER2−

Patient status: C1D1 (1^{st} cycle, 1^{st} day of study treatment with CHA.7.518.1.H4 (S241P)±nivolumab).

Sponsor Baseline Biopsy: PDL1 CPS=3, PVRL2 H-score=300 Confirmed PR now with CR was observed.

Clinical vignette [P1b expansion breast cohort]-subject with PR in response to treatment

65 year old female diagnosed with ductal carcinoma of breast that has progressed from stage IIIb to stage IV. The patient is refractory to the last prior therapy with carboplatin/gemcitabine.

Adjuvant: AC-T [best response unknown]; capecitabine [best response unknown]

Metastatic: capecitabine [best response PD]; carboplatin/gemcitabine [best response PD]

At screening: ECOG 1; metastasis observed in lung, nodes, and pleural effusion. PD-L1 negative. The patient has a TMB of 1 and triple negative breast cancer (TNBC).

Patient status: C1D1 (1^{st} cycle, 1^{st} day of study treatment with CHA.7.518.1.H4 (S241P)±nivolumab).

PR was observed after the treatment of the patient.

CHA.7.518.1.H4 (S241P)+nivo-subjects with SD in response to treatment [clinical vignette]

4-4116 [PD-L1 negative; TNBC, 7 prior lines in met setting. Confirmed SD on study [on study treatment for 7 cycles then PD]. Best response of SD to prior ICI [atezolizumab]. Refractory to last prior therapy [trodelvy]
AC-T [Adj]. Metastatic setting: gemcitabine/carboplatin [PD], etoposide/cisplatin [PR], abraxane/atezolizumab [SD], atezolizumab [SD]; capecitabine [pd]; doxil/bevacizumab/everolimus [PR]; trodelvy [PD]

7-4110 [1^{st} imaging SD [then withdrew consent]]; PD-L1 negative. 7 prior lines.
AC-T [unknown], abraxane [unknown]; AC [CR]; anastrozole [unknown]; xeloda [unknown]; trodelvy; paraplatin

2-4109 [SD; on study treatment for 5 cycles before PD]. 3 prior lines metastatic setting. Refractory to last prior therapy [cytoxan/docetaxel]
Adjuvant therapy with Taxotere/Cytoxan/avastin/aredia [PD]. Metastatic: 5FU [SD]; faslodex [SD]; Cytoxan/docetaxel [PD]

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

What is claimed:

1. A method of treatment for cancer comprising administering nivolumab and an anti-PVRIG antibody, wherein said anti-PVRIG antibody is administered as a stable liquid pharmaceutical formulation and, wherein the stable liquid pharmaceutical formulation of the anti-PVRIG antibody comprises:
(a) an anti-PVRIG antibody, wherein said anti-PVRIG antibody comprises:
i) a heavy chain variable domain comprising the vhCDR1, vhCDR2, and vhCDR3 from the heavy chain of CHA. 7.518.1.H4 (S241P) (SEQ ID NO:4), and
ii) a light chain variable domain comprising the vlCDR1, vlCDR2, and vlCDR3 from the light chain of CHA.7.518.1.H4 (S241P) (SEQ ID NO:9);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the formulation has a pH from 5.5 to 7.0.

2. The method of treatment according to claim 1, wherein said anti-PVRIG antibody comprises a CH1-hinge-CH2-CH3 sequence of IgG4 (SEQ ID NO:17 or SEQ ID NO:50), wherein said hinge region optionally comprises mutations.

3. The method of treatment according to claim 1 or 2, wherein said anti-PVRIG antibody comprises the CH1-hinge-CH2-CH3 region from IgG1, IgG2, IgG3, or IgG4, wherein said hinge region optionally comprises mutations.

4. The method of treatment according to any one of claims 1-3, wherein said heavy chain variable domain is from the heavy chain of CHA.7.518.1.H4 (S241P) (SEQ ID NO:4) and said light chain variable domain is from the light chain of CHA.7.518.1.H4 (S241P) (SEQ ID NO: 9).

5. The method of treatment according to any one of claims 1-4, wherein said anti-PVRIG antibody comprises a CL region of human kappa 2 light chain.

6. The method of treatment according to any one of claims 1-5, wherein said pharmaceutical formulation comprises from 10 mM to 80 mM histidine, from 15 mM to 70 mM histidine, from 20 mM to 60 mM histidine, from 20 mM to 50 mM histidine, or from 20 mM to 30 mM histidine.

7. The method of treatment according to any one of claims 1-6, wherein said pharmaceutical formulation comprises about 25 mM histidine.

8. The method of treatment according to any one of claims 1-7, wherein said pharmaceutical formulation comprises from 30 mM to 100 mM NaCl, from 30 mM to 90 mM NaCl, from 40 mM to 80 mM NaCl, from 30 mM to 70 mM NaCl, or from 45 mM to 70 mM NaCl.

9. The method of treatment according to any one of claims 1-8, wherein said pharmaceutical formulation comprises about 60 mM NaCl.

10. The method of treatment according to any one of claims 1-9, wherein said pharmaceutical formulation comprises from 20 mM to 140 mM L-arginine, from 30 mM to 140 mM L-arginine, from 40 mM to 130 mM L-arginine, from 50 mM to 120 mM L-arginine, from 60 mM to 110 mM L-arginine, from 70 mM to 110 mM L-arginine, from 80 mM to 110 mM L-arginine, or from 90 mM to 110 mM L-arginine.

11. The method of treatment according to any one of claims 1-10, wherein said pharmaceutical formulation comprises about 100 mM L-arginine.

12. The method of treatment according to any one of claims 1-11, wherein said pharmaceutical formulation comprises from 0.006% to 0.1% w/v polysorbate 80, from 0.007% to 0.09% w/v polysorbate 80, from 0.008% to 0.08% w/v polysorbate 80, from 0.009% to 0.09% w/v polysorbate 80, from 0.01% to 0.08% w/v polysorbate 80, from 0.01% to 0.07% w/v polysorbate 80, from 0.01% to 0.07% w/v polysorbate 80, or from 0.01% to 0.06% w/v polysorbate 80, or from 0.009% to 0.05% w/v polysorbate 80.

13. The method of treatment according to any one of claims 1-12, wherein said pharmaceutical formulation comprises about 0.01% polysorbate 80.

14. The method of treatment according to any one of claims 1-13, wherein said pH is from 6 to 7.0.

15. The method of treatment according to any one of claims 1-14, wherein said pH is from 6.3 to 6.8.

16. The method of treatment according to any one of claims 1-15, wherein said pH is 6.5+/−0.2.

17. The method of treatment according to any one of claims 1-16, wherein said anti-PVRIG antibody is at a concentration of from 10 mg/mL to 40 mg/mL, 15 mg/mL to 40 mg/mL, 15 mg/mL to 30 mg/mL, 10 mg/mL to 25 mg/mL, or 15 mg/mL to 25 mg/mL.

18. The method of treatment according to any one of claims 1-17, wherein said formulation is stable at 2° C. to 8° C. for at least 1 week, 2 weeks, 3 weeks, 4 weeks, 5 weeks, 6 weeks, 7 weeks, 8 weeks, 9 weeks, or 10 weeks.

19. The method of treatment according to any one of claims 1-18, wherein said formulation is stable at about 20° C. to 25° C. for at least 1 week, 2 weeks, 3 weeks, 4 weeks, 5 weeks, or 6 weeks.

20. The method of treatment according to any one of claims 1-19, wherein said formulation is stable at 35° C. to 40° C. for at least 1 week, 2 weeks, 3 weeks, 4 weeks, or 5 weeks.

21. The method of treatment according to any one of claims 1-20, wherein said anti-PVRIG antibody is at a concentration of about 20 mg/mL.

22. The method of treatment according to any one of claims 1-21, wherein said anti-PVRIG antibody formulation comprises:
a) a heavy chain comprising:
i) a VH-CH1-hinge-CH2-CH3, wherein the VH is from CHA.7.518.1.H4 (S241P) (SEQ ID NO:4) and wherein the CH1-hinge-CH2-CH3 region is from IgG4; and
b) a light chain comprising:
i) a VL-CL, wherein the VL from CHA.7.518.1.H4 (S241P) (SEQ ID NO: 9) and wherein the CL region is from human kappa 2 light chain.

23. The method of treatment according to claim 24, wherein said hinge region optionally comprises mutations.

24. The method of treatment according to claim 23, wherein said hinge region optionally comprises substitutions.

25. The method of treatment according to any one of claims 1-24, wherein said anti-PVRIG antibody formulation comprises:
i) a heavy chain comprising the heavy chain from CHA.7.518.1.H4 (S241P) (SEQ ID NO:8); and
ii) a light chain comprising the light chain from CHA.7.518.1.H4 (S241P) (SEQ ID NO:13).

26. The method of treatment according to any one of claims 1-21, said anti-PVRIG antibody formulation comprising:
(a) an anti-PVRIG antibody, wherein said anti-PVRIG antibody comprises:
i) a heavy chain variable domain comprising the vhCDR1, vhCDR2, and vhCDR3 from the heavy chain of CHA.7.518.1.H4 (S241P) (SEQ ID NO:4), and
ii) a light chain variable domain comprising the vlCDR1, vlCDR2, and vlCDR3 from the light chain of CHA.7.518.1.H4 (S241P) (SEQ ID NO:9);
(b) about 25 mM histidine;
(c) about 60 mM NaCl;
(d) about 100 mM L-Arginine; and
(e) about 0.01% % w/v polysorbate 80,
wherein the formulation has a pH from 6.5+/−0.2.

27. The method of treatment according to any one of claims 1-26, said anti-PVRIG antibody formulation comprising:
(a) an anti-PVRIG antibody, wherein said anti-PVRIG antibody comprises:
i) a heavy chain comprising the heavy chain from CHA.7.518.1.H4 (S241P) (SEQ ID NO:8); and
ii) a light chain comprising the light chain from CHA.7.518.1.H4 (S241P) (SEQ ID NO: 13);
(b) about 25 mM histidine;
(c) about 60 mM NaCl;
(d) about 100 mM L-Arginine; and
(e) about 0.01% % w/v polysorbate 80,
wherein the formulation has a pH from 6.5+/−0.2.

28. The method of treatment according to any one of claims 1-27, wherein said anti-PVRIG antibody is administered at a dosage of about 0.01 mg/kg to about 20 mg/kg of the anti-PVRIG antibody or about 0.01 mg/kg to about 10 mg/kg of the anti-PVRIG antibody.

29. The method of treatment according to any one of claims 1-27, wherein said anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, or 20 mg/kg of the anti-PVRIG antibody.

30. The method of treatment according to any one of claims 1-29, wherein said nivolumab is administered at a dosage of about 360 mg of nivolumab or 480 mg of nivolumab.

31. The method of treatment according to any one of claims 1-30, wherein said anti-PVRIG antibody is administered 20 mg/kg every 4 weeks.

32. The method of treatment according to any one of claims 1-31, wherein said cancer is selected from the group consisting of prostate cancer, liver cancer (HCC), rectal cancer, colorectal cancer (CRC), colorectal cancer MSS (MSS-CRC; microsatellite stable colorectal carcinoma/carcinoma), metastatic MSS-CRC, refractory metastatic MSS-CRC, refractory MSS colorectal, CRC (MSS unknown), ovarian cancer (including ovarian carcinoma), platinum resistant ovarian cancer, high grade serous adenocarcinoma, endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), uveal melanoma, glioma, renal cell cancer (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cell cancer, MSI-high cancer, KRAS mutant tumors, adult T-cell leukemia/lymphoma, pleural mesothelioma, anal SCC, neuroendocrine lung cancer (including neuroendocrine lung carcinoma), small cell lung cancer, NSCLC, NSCLC large cell, NSCLC squamous cell, NSCLC adenocarcinoma, atypical carcinoid lung cancer, NSCLC with PDL1>=50% TPS, cervical SCC, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, Myelodysplastic syndromes (MDS), HNSCC, PD1 refractory or relapsing cancer, gastroesophageal junction cancer, gastric cancer, chordoma, sarcoma, endometrial sarcoma, chondrosarcoma, uterine sarcoma, plasma cell disorders, multiple myeloma, amyloidosis, AL-amyloidosis, glioblastoma, astrocytoma and fallopian tube cancer.

33. The nivolumab and an anti-PVRIG antibody combination treatment according to any one of the method of treatment claims 1-31, for use in a method of treating cancer.

34. Use of nivolumab and an anti-PVRIG antibody in the manufacture of a medicament for the treatment of cancer, wherein the anti-PVRIG antibody is formulated as the stable liquid pharmaceutical formulation according to any of the claims 1-31.

35. The use according to claim 33 or 34, wherein said cancer is selected from the group consisting of prostate cancer, liver cancer (HCC), rectal cancer, colorectal cancer (CRC), colorectal cancer MSS (MSS-CRC; microsatellite stable colorectal carcinoma/carcinoma), metastatic MSS-CRC, refractory metastatic MSS-CRC, refractory MSS colorectal, CRC (MSS unknown), ovarian cancer (including ovarian carcinoma), platinum resistant ovarian cancer, high grade serous adenocarcinoma, endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), uveal melanoma, glioma, renal cell cancer (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cell cancer, MSI-high cancer, KRAS mutant tumors, adult T-cell leukemia/lymphoma, pleural mesothelioma, anal SCC, neuroendocrine lung cancer (including neuroendocrine lung carcinoma), small cell lung cancer, NSCLC, NSCLC large cell, NSCLC squamous cell, NSCLC adenocarcinoma, atypical carcinoid lung cancer, NSCLC with PDL1>=50% TPS, cervical SCC, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, Myelodysplastic syndromes (MDS), HNSCC, PD1 refractory or relapsing cancer, gastroesophageal junction cancer, gastric cancer, chordoma, sarcoma, endometrial sarcoma, chondrosarcoma, uterine sarcoma, plasma cell disorders, multiple myeloma, amyloidosis, AL-amyloidosis, glioblastoma, astrocytoma and fallopian tube cancer.

36. A method for determining or predicting the efficacy of treatment with nivolumab and an anti-PVRIG antibody in a cancer patient, the method comprising:
(a) measuring the level of one or more cellular components comprising activated DC cells, effector memory CD8 positive T cells, CD8 positive T cells, and NK-T cells, in a biological sample from of a cancer patient;
(b) quantitating the measurement of the level of the one or more cellular components; and
(c) correlating the level of the one or more cellular components with the efficacy of treatment.

37. A method for determining a cancer patient population for treatment with nivolumab and an anti-PVRIG antibody, the method comprising:
(a) detecting the presence in a biological sample from the cancer patient one or more cellular components comprising activated DC cells, effector memory CD8 positive T cells, CD8 positive T cells, and NK-T cells;
(b) quantitating the measurement of the level of the one or more cellular components;
(c) treating the cancer patient with nivolumab and an anti-PVRIG antibody when the level of the one or more cellular components are present at an increased level as compared to a control or a patient that does not have detectable levels of the cells.

38. A method for determining or predicting the efficacy of treatment with nivolumab and an anti-PVRIG antibody in a cancer patient, the method comprising:
(a) measuring the level of one or more biomarkers in a biological sample from the cancer patient;
(b) quantitating the measurement of the level of the one or more biomarkers; and
(c) correlating the level of the one or more biomarkers with the efficacy of treatment.

39. A method for determining a cancer patient population for treatment with nivolumab and an anti-PVRIG antibody, the method comprising:
(a) detecting the presence in a biological sample from the cancer patient one or more biomarkers;
(b) quantitating the measurement of the level of the one or more biomarkers;
(c) treating the cancer patient with nivolumab and an anti-PVRIG antibody when the level of the one or more biomarkers is present at an increased or decreased level as compared to a control, pre-treatment sample, or a patient that does not have detectable levels of the one or more biomarkers.

40. The method according to claim 38 or 39, wherein the biomarkers are proteins or protein levels and/or mRNAs or mRNA levels.

41. The method according to any one of claims 36-40, wherein the biological sample is obtained from a tumor, tumor microenvironment, and/or peripheral blood from the cancer patient.

42. The method according to any one of claims 36-37, 41, wherein the activated DC cells being present and/or being present at an increased level as compared to a control, pre-treatment sample, or a patient that does not have detectable levels of the activated DC cells, is indicative of treatment efficacy for treatment with an anti-PVRIG antibody in combination with nivolumab.

43. The method according to any one of claims 36-37, 41-42, wherein the activated DC cells express one or more biomarkers selected from the group consisting of LAMP3, HLA-DR and CD83.

44. The method according to any one of claims 36-37, 41-43, wherein measuring the level of the activated DC cells comprises measuring expression level of one or more biomarkers selected from the group consisting of LAMP3, HLA-DR and CD83.

45. The method according to claim any one of claims 36-37, 41-44, wherein the level of the activated DC cells is indicative of treatment efficacy for treatment with an anti-PVRIG antibody in combination with nivolumab when the level of the activated DC cells is increased by at least 1-fold, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, or 30-fold as compared to a control, pre-treatment sample, or a patient that does not respond to treatment with an anti-PVRIG antibody in combination with nivolumab.

46. The method according to any one of claims 36-37, 41, wherein the effector memory CD8 positive T cells, or CD8 positive T cells, being present and/or being present at an increased level in the biological sample of the patient as compared to a control, pre-treatment sample, or a patient that does not have detectable levels of the effector memory CD8 positive T cells, or CD8 positive T cells, is indicative of treatment efficacy for treatment with an anti-PVRIG antibody in combination with nivolumab.

47. The method according to any one of claims 36-37, 41, wherein the effector memory CD8 positive T cells are CD8+CD45RA-CCR7−.

48. The method according to any one of claims 36-37, 41, 47, wherein measuring the level of the effector memory CD8 positive T cells comprises measuring the expression level of one or more of CD8, CD45RA and CCR7.

49. The method according to claim 48, wherein the expression level of CD8 is indicative of treatment efficacy for treatment with an anti-PVRIG antibody in combination with nivolumab, when the expression level of CD8 is increased by at least 1-fold, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, or 30-fold as compared to a control, pre-treatment sample, or a patient that does not respond to treatment with an anti-PVRIG antibody in combination with nivolumab.

50. The method according to claim 48, wherein the expression level of CD45RA is indicative of treatment efficacy for treatment with an anti-PVRIG antibody in combination with nivolumab, when the expression level of CD45RA is decreased by at least 5%, 10%, 15%, 20%, 25%, 30%, 35%, 40%, 45%, 50%, 55%, 60%, 65%, 70%, 75%, 80%, 85%, 90%, 95%, or more as compared to a control, pre-treatment sample, or a patient that does not respond to treatment with an anti-PVRIG antibody in combination with nivolumab.

51. The method according to 48, wherein the expression level of CCR7 is indicative of treatment efficacy for treatment with an anti-PVRIG antibody in combination with nivolumab, when the expression level of CCR7 is decreased by at least 5%, 10%, 15%, 20%, 25%, 30%, 35%, 40%, 45%, 50%, 55%, 60%, 65%, 70%, 75%, 80%, 85%, 90%, 95%, or more as compared to a control, pre-treatment sample, or a patient that does not respond to treatment with an anti-PVRIG antibody in combination with nivolumab.

52. The method according to any one of claims 36-37, 41, 46-51, wherein the level of the effector memory CD8 positive T cells, or CD8 positive T cells is indicative of treatment efficacy for treatment with an anti-PVRIG antibody in combination with nivolumab when the level of effector memory CD8 positive T cells, or CD8 positive T cells is increased by at least 1-fold, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, or 50-fold, as compared to a control, pre-treatment sample, or a patient that does not respond to treatment with an anti-PVRIG antibody in combination with nivolumab.

53. The method according to any one of claims 38-41, wherein the one or more biomarkers are selected from the group consisting of LAMP3, HLA-DR and CD83.

54. The method according to any one of claims 38-41, 53, comprising measuring the expression level of one or more of LAMP3, HLA-DR and CD83.

55. The method according to any one of claims 38-41, 53-54, wherein the level of the one or more biomarkers selected from the group consisting of LAMP3, HLA-DR and CD83 is indicative of treatment efficacy for treatment with an anti-PVRIG antibody in combination with nivolumab, when the expression level of the one or more of these biomarkers is increased by at least 1-fold, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, or 30-fold as compared to a control, pre-treatment sample, or a patient that does not respond to treatment with an anti-PVRIG antibody in combination with nivolumab.

56. The method according to any one of claims 38-41, wherein the biomarker is KI67.

57. The method according to any one of claims 38-41, 56, comprising measuring the expression level of KI67.

58. The method according to any one of claims 38-41, 56-57, wherein the level of the biomarker, is indicative of treatment efficacy for treatment with an anti-PVRIG antibody in combination with nivolumab when the expression level of KI67 is increased by at least 1-fold, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, or 50-fold, as compared to a control, pre-treatment sample, or a patient that does not respond to treatment with an anti-PVRIG antibody in combination with nivolumab.

59. The method according to any one of claims 38-41, wherein the biomarker is CD8/CD4 ratio.

60. The method according to any one of claims 38-41, 59, comprising measuring the CD8/CD4 ratio.

61. The method of treatment according to any one of claims 38-41, 59-60, wherein the level of the CD8/CD4 ratio is indicative of treatment efficacy for treatment with an anti-PVRIG antibody in combination with nivolumab when the CD8/CD4 ratio is increased by at least about 10%, 20%, 30%, 40%, 50%, 60%, 70%, 80%, 90%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, or 1000%, as compared to a control, pre-treatment sample, or a patient that does not respond to treatment with an anti-PVRIG antibody in combination with nivolumab.

62. The method of treatment according to any one of claims 38-41, 59-61, wherein the level of the CD8/CD4 ratio is indicative of treatment efficacy for treatment with an anti-PVRIG antibody in combination with nivolumab when the CD8/CD4 ratio is increased by about 1.1-fold, 1.2-fold, 1.3-fold, 1.4-fold, 1.5-fold, 1.6-fold, 1.7-fold, 1.8-fold, 1.9-fold, 2-fold, 2.25-fold, 2.5-fold, 2.75-fold, 3-fold, 3.25-fold, 3.5-fold, 3.75-fold, 4-fold, 4.25-fold, 4.5-fold, 4.75-fold, 5-fold, 5.25-fold, 5.5-fold, 5.75-fold, 6-fold, 6.25-fold, 6.5-fold, 6.75-fold, 7-fold, 7.25-fold, 7.5-fold, 7.75-fold, 8-fold, 8.25-fold, 8.5-fold, 8.75-fold, 9-fold, 9.25-fold, 9.5-fold, 9.75-fold, 10-fold, 10.25-fold, 10.5-fold, 10.75-fold or 11-fold, as compared to a control, pre-treatment sample, or a patient that does not respond to treatment with an anti-PVRIG antibody in combination with nivolumab.

63. The method according to any one of claims 36-37, 41, wherein the NK-T cells being present and/or being present at an increased level in the biological sample of the patient as compared to a control, pre-treatment sample, or a patient that does not have detectable levels of the NK-T cells, is indicative of treatment efficacy for treatment with an anti-PVRIG antibody in combination with nivolumab.

64. The method according to any one of claims 36-37, 41, 63, wherein the level of the NK-T cells is indicative of treatment efficacy for treatment with an anti-PVRIG antibody in combination with nivolumab when the level of the NK-T cells is increased by at least 1-fold, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, or 50-fold, as compared to a control, pre-treatment sample, or a patient that does not respond to treatment with an anti-PVRIG antibody in combination with nivolumab.

65. The method according to any one of claims 36-39, wherein the biological sample is from and/or includes the tumor microenvironment (TME).

66. The method according to claim 65, wherein the biological sample comprises a Tertiary Lymphoid Structure (TLS) within the tumor microenvironment.

67. The method according to claim 65 or 66, wherein the activated DC cells express LAMP3 and/or PVRL2.

68. The method according to claim 65 or 66, wherein measuring the level of the activated DC cells comprises measuring the level of LAMP3 and/or PVRL2.

69. The method according to claim 65 or 66, wherein the CD8 positive T cells express one or more of PVRIG, TIGIT, CD28 and CD226.

70. The method according to claim 65 or 66, wherein measuring the level of the CD8 positive T cells comprises measuring the level of one or more of PVRIG, TIGIT, CD28 and CD226.

71. The method according to claim 65 or 66, wherein the cellular components further comprise B cells, early differentiated T stem cell-like memory cells (Tscm) and Naïve T cells.

72. The method according to claim 65 or 66, wherein the one or more biomarkers comprise PVRIG, TIGIT, CD28, CD226, DNAM-1 axis, FOXP3, CD4, LAMP3, or PVRL2.

73. A method for determining or predicting the efficacy of treatment with nivolumab and an anti-PVRIG antibody, the method comprising:
(a) measuring the level of TCR diversity and repertoire value in a biological sample of a cancer patient;
(b) quantitating the measurement of the level of TCR diversity and repertoire value; and
(c) correlating the level of the TCR diversity and repertoire value with the efficacy of treatment.

74. A method for determining a cancer patient population for treatment with nivolumab and an anti-PVRIG antibody, the method comprising:
(a) measuring the level of TCR diversity and repertoire value in a biological sample from the cancer patient;
(b) quantitating the measurement of the level of TCR diversity and repertoire value;
(c) treating the cancer patient with an anti-PVRIG antibody in combination with nivolumab when there is a decrease in the TCR diversity index and increase in the number of expanded TCR repertoire level as compared to a control, pre-treatment sample, or a patient that does not respond to treatment with an anti-PVRIG antibody in combination with nivolumab.

75. The method according to claim 73 or 74, wherein the biological sample is obtained from a tumor, tumor microenvironment, and/or peripheral blood from the cancer patient.

76. The method according to any one of claims 73-75, wherein the TCR diversity and repertoire value is measured before and/or during the treatment.

77. The method according to any one of claims 76-76, wherein the TCR diversity and repertoire value is indicated by number of unique clones of TCRα and/or TCRβ.

78. The method according to any one of claims 76-77, wherein measuring the TCR diversity and repertoire value comprises calculating a TCR diversity index from TCR repertoire analysis of T cells in the biological sample.

79. the method according to claim 78, comprising calculating the TCR diversity index from TCR repertoire analysis of the T cells in a biopsy taken between cycle 2 and cycle 3 of the treatment.

80. The method according to claim 78 or 79, wherein the TCR diversity index and/or repertoire is selected from the group consisting of a Shannon index, a Simpson index, an inverse Simpson index, a normalized Shannon index, a Unique50 index, a DE30 index, a DE80 index, Gini Coefficient, proportion of TCR clone reads out of total, and a DE50 index.

81. The method according to claim 78 or 79, wherein the TCR diversity index is a Gini Coefficient.

82. The method according to claim 81, wherein a Gini Coefficient of about 0.6 or more is indicative of treatment efficacy for treatment with the an anti-PVRIG antibody in combination with nivolumab.

83. The method according to claim 81 or 82, wherein an increase of a Gini Coefficient of about 0.3 or more during the treatment, is indicative of treatment efficacy for treatment with an anti-PVRIG antibody in combination with nivolumab.

84. The method according to claim 81, wherein an increase of top unique TCR clones (by ranking of TCR reads) the top deciles comprise about 50% or more of total TCR reads during the treatment is indicative of treatment efficacy for treatment with an anti-PVRIG antibody in combination with nivolumab.

85. The method according to any one of claims 73-84, wherein the TCR is TCRα and/or TCRβ.

86. A method for determining or predicting the efficacy of treatment with nivolumab and an anti-PVRIG antibody, the method comprising:
(a) measuring the expression level of IFNγ in a biological sample of the cancer patient;
(b) quantitating the measurement of the expression level of IFNγ; and
(c) correlating the expression level of IFNγ with the efficacy of treatment.

87. A method for determining a cancer patient population for treatment with nivolumab and an anti-PVRIG antibody, the method comprising:
(a) measuring the expression level of IFNγ in a biological sample from the cancer patient;
(b) quantitating the measurement of the expression level of IFNγ;
(c) treating the cancer patient with nivolumab and an anti-PVRIG antibody in combination with nivolumab when there is an increase the expression level of IFNγ as compared to a control, pre-treatment sample, or a patient that does not respond to treatment with an anti-PVRIG antibody in combination with nivolumab.

88. The method according to claim 86 or 87, wherein measuring the expression level of IFNγ comprises measuring the expression level of IFNγ signature and/or Lymphoid signature.

89. The method according to claim 88, wherein the IFNγ signature comprises one or more of CXCL10, CXCL9, IDO1, STAT1, HLA_DRA, and IFNG.

90. The method according to claim 88, wherein the Lymphoid signature comprises one of more of PRF1, GZMB, CD8A, CD8B, CD3G, CD4, CD3D, and CD3E.

91. The method according to any one of claims 86-90, wherein measuring the expression level of IFNγ comprises measuring the expression level of one or more of the CXCL10, CXCL9, CD137, HLA_DRA, IFNG, PRF1, GZMB, GZMA, GZMH proteins or protein levels and/or mRNAs or mRNA levels.

92. The method according to any one of claims 88-91, wherein the expression level of the IFNγ, IFNγ signature and/or Lymphoid signature, is indicative of treatment efficacy for treatment with an anti-PVRIG antibody in combination with nivolumab when the expression level of any of the IFNγ, IFNγ signature and/or Lymphoid signature is increased by at least 1-fold, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, or 10-fold, as compared to a control, pre-treatment sample, or a patient that does not respond to treatment with an anti-PVRIG antibody in combination with nivolumab.

93. The method according to claim 91, wherein the expression level of any one of the CXCL10, CXCL9, CD137, HLA_DRA, IFNG, PRF1, GZMB, GZMA, GZMH proteins or protein levels and/or mRNAs or mRNA levels, is indicative of treatment efficacy for treatment with an anti-PVRIG antibody in combination with nivolumab when the expression level of any of the of any one of the CXCL10, CXCL9, CD137, HLA_DRA, IFNG, PRF1, GZMB, GZMA, GZMH proteins or protein levels and/or mRNAs or mRNA levels is increased by at least 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, or 10-fold, as compared to a control, pre-treatment sample, or a patient that does not respond to treatment with an anti-PVRIG antibody in combination with nivolumab.

94. The method according to any one of claims 36-93, wherein the anti-PVRIG antibody comprises:
i) a heavy chain variable domain comprising the vhCDR1, vhCDR2, and vhCDR3 from the heavy chain of CHA.7.518.1.H4 (S241P) (SEQ ID NO:4), and
ii) a light chain variable domain comprising the vlCDR1, vlCDR2, and vlCDR3 from the light chain of CHA.7.518.1.H4 (S241P) (SEQ ID NO:9).

95. The method according to claim 94, wherein said anti-PVRIG antibody comprises a CH1-hinge-CH2-CH3 sequence of IgG4 (SEQ ID NO:17 or SEQ ID NO:50), wherein said hinge region optionally comprises mutations.

96. The method according to claim 94, wherein said anti-PVRIG antibody comprises the CH1-hinge-CH2-CH3 region from IgG1, IgG2, IgG3, or IgG4, wherein said hinge region optionally comprises mutations.

97. The method according to any one of claims 94-96, wherein said heavy chain variable domain is from the heavy chain of CHA.7.518.1.H4 (S241P) (SEQ ID NO:4) and said light chain variable domain is from the light chain of CHA.7.518.1.H4 (S241P) (SEQ ID NO:9).

98. The method according to any one of claims 94-97, wherein said anti-PVRIG antibody comprises a CL region of human kappa 2 light chain.

99. The method according to any one of claims 36-98, wherein the anti-PVRIG antibody is administered as a stable liquid pharmaceutical formulation.

100. The method according to claim 99, wherein the anti-PVRIG antibody is administered as a stable liquid pharmaceutical formulation of any one of claims 1-33.

101. The stable liquid pharmaceutical formulation according to any one of claims 1-33, wherein upon administration to a cancer patient, an anti-PVRIG antibody in combination with nivolumab induce proliferation of one or more types of cells selected from the group consisting of activated DC cells, effector memory CD8 positive T cells, CD8 positive T cells, and NK-T cells.

102. The stable liquid pharmaceutical formulation according to any one of claims 1-33, wherein upon administration to a cancer patient, an anti-PVRIG antibody in combination with nivolumab there is a decrease in the TCR diversity index and increase in the number of expanded TCR repertoire value, and/or an increase in the expression level of IFNγ.

103. A method for increasing infiltration of T cells in a tumor in a patient with PD-L1^{low} cancer, the method comprising administering to the patient an anti-PVRIG antibody in combination with nivolumab.

104. The method according to claim 103, wherein the tumor has a PD-L1 CPS of less than 1, 2, or 3.

105. The method according to claim 103 or 104, wherein the tumor is PVRL2+.

106. The method according to claim 105, wherein the tumor has a PVRL2 H-score of at least 250, 255, 260, 265, 270, 275, 280, 285, 290, 295, 300, or more.

107. The method according to any one of claims 103-106, wherein the T cells comprise effector memory CD8 positive T cells and/or CD8 positive T cells.

108. The method according to any one of claims 103-107, wherein the increased infiltration of T cells in a tumor is characterized by one or more selected from the group consisting of
(a) presence of and/or presence of an increased level of effector memory CD8 positive T cells, or CD8 positive T cells in the tumor;
(b) an increase in TCR diversity index in the tumor; and
(c) an increase in the expression level of IFNγ signature in the tumor; as compared to a control, pre-treatment sample, or a patient that does not respond to treatment with an anti-PVRIG antibody in combination with nivolumab.

109. The method according to any one of claims 103-108, wherein the anti-PVRIG antibody comprises:
i) a heavy chain variable domain comprising the vhCDR1, vhCDR2, and vhCDR3 from the heavy chain of CHA.7.518.1.H4 (S241P) (SEQ ID NO:4), and
ii) a light chain variable domain comprising the vlCDR1, vlCDR2, and vlCDR3 from the light chain of CHA. 7.518.1.H4 (S241P) (SEQ ID NO:9).

110. The method according to any one of claims 103-109, wherein said anti-PVRIG antibody comprises a CH1-hinge-CH2-CH3 sequence of IgG4 (SEQ ID NO:17 or SEQ ID NO:50), wherein said hinge region optionally comprises mutations.

111. The method according to any one of claims 103-110, wherein said anti-PVRIG antibody comprises the CH1-hinge-CH2-CH3 region from IgG1, IgG2, IgG3, or IgG4, wherein said hinge region optionally comprises mutations.

112. The method according to 109-111, wherein said heavy chain variable domain is from the heavy chain of CHA. 7.518.1.H4 (S241P) (SEQ ID NO:4) and said light chain variable domain is from the light chain of CHA.7.518.1.H4 (S241P) (SEQ ID NO:9).

113. The method according to any one of claims 103-112, wherein said anti-PVRIG antibody comprises a CL region of human kappa 2 light chain.

114. The method according to any one of claims 103-113, wherein the anti-PVRIG antibody is administered as a stable liquid pharmaceutical formulation.

115. The method according to any one of claims 103-114, wherein the anti-PVRIG antibody is administered as a stable liquid pharmaceutical formulation of any one of claims 1-33.

116. The method according to any one of claims 103-115, wherein the cancer is selected from the group consisting of prostate cancer, liver cancer (HCC), rectal cancer, colorectal cancer (CRC), colorectal cancer MSS (MSS-CRC; microsatellite stable colorectal carcinoma/carcinoma), metastatic MSS-CRC, refractory metastatic MSS-CRC, refractory MSS colorectal, CRC (MSS unknown), ovarian cancer (including ovarian carcinoma), platinum resistant ovarian cancer, high grade serous adenocarcinoma, endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), uveal melanoma, glioma, renal cell cancer (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cell cancer, MSI-high cancer, KRAS mutant tumors, adult T-cell leukemia/lymphoma, pleural mesothelioma, anal SCC, neuroendocrine lung cancer (including neuroendocrine lung carcinoma), small cell lung cancer, NSCLC, NSCLC large cell, NSCLC squamous cell, NSCLC adenocarcinoma, atypical carcinoid lung cancer, NSCLC with PDL1>=50% TPS, cervical SCC, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, Myelodysplastic syndromes (MDS), HNSCC, PD1 refractory or relapsing cancer, gastroesophageal junction cancer, gastric cancer, chordoma, sarcoma, endometrial sarcoma, chondrosarcoma, uterine sarcoma, plasma cell disorders, multiple myeloma, amyloidosis, AL-amyloidosis, glioblastoma, astrocytoma and fallopian tube cancer.

117. The method or use according to any one of the preceding claims, further comprising administering an anti-TIGIT antibody to the patient, wherein the anti-TIGIT antibody comprises:
i) a heavy chain variable domain comprising the vhCDR1, vhCDR2, and vhCDR3 from the heavy chain of BMS-986207, and
ii) a light chain variable domain comprising the vlCDR1, vlCDR2, and vlCDR3 from the light chain of BMS-986207.

118. The method according to claim 117, wherein the anti-TIGIT antibody comprises:
i) a heavy chain variable domain comprising a vhCDR1 comprising the sequence of SEQ ID NO:2000, a vhCDR2 comprising the sequence of SEQ ID NO:2001, and a vhCDR3 comprising the sequence of SEQ ID NO:2002, and
ii) a light chain variable domain comprising a vlCDR1 comprising the sequence of SEQ ID NO:2003, a vlCDR2 comprising the sequence of SEQ ID NO:2004, and a vlCDR3 comprising the sequence of SEQ ID NO:2005.

119. The formulation, method, or use of any one of claims 1-118, wherein the patient has received prior treatment with lenvatinib, pembrolizumab, or a combination treatment comprising lenvatinib and pembrolizumab.

120. The formulation, method, or use of any one of claims 1-119, wherein the patient has received prior treatment with one or more of capecitabine, irinotecan, abemaciclib, fulvestrant, tamoxifen, taxotere, adriamycin, cyclophosphamide, carboplatin, gemcitabine, etoposide, cisplatin, atezolizumab, doxil, bevacizumab, everolimus, abraxane, anastrozole, trodelvy, 5FU (fluorouracil), and any combination thereof.
